SEQUENCE LISTING (134 sequences)

SEQ ID NO: 1 - DNA, 467 bp, synthetic construct
  misc_feature 1..467: note = Description of Artificial Sequence: Synthetic polynucleotide
  source 1..467: mol_type = other DNA
atagtgagtcgtattaacgtaccaacaacaaggaagtgctaaagaaacttgttctttagcacttccttgtttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 2 - DNA, 615 bp, synthetic construct
  misc_feature 1..615: note = Description of Artificial Sequence: Synthetic polynucleotide
  source 1..615: mol_type = other DNA
atagtgagtcgtattaacgtaccaacaacaaggagtgctaaagaaacttgttctttagcactccttgtttatcttagaggcatatccctacgtaccaacaagagagtgattgagagtggacttgccactctcaatcactctctttatcttagaggcatatccctacgtaccaacaagagagctctgtctggaccacttgggtccagacagagctctctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 3 - DNA, 629 bp, synthetic construct
  misc_feature 1..629: note = Description of Artificial Sequence: Synthetic polynucleotide
  source 1..629: mol_type = other DNA
atagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 4 - DNA, 978 bp, synthetic construct
  misc_feature 1..978: note = Description of Artificial Sequence: Synthetic polynucleotide
  source 1..978: mol_type = other DNA
atagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctacgtaccaacaagcaatgaggaccctgagagatacttgatctctcagggtcctcattgctttatcttagaggcatatccctacgtaccaacaagctgatgggaacgtggactaacttgtagtccacgttcccatcagctttatcttagaggcatatccctacgtaccaacaaggtcctcagattactacaaacttgttgtagtaatctgaggacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccct

SEQ ID NO: 5 - DNA, 741 bp, synthetic construct
  misc_feature 1..741: note = Description of Artificial Sequence: Synthetic polynucleotide
  source 1..741: mol_type = other DNA
atagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccct

SEQ ID NO: 6 - DNA, 741 bp, synthetic construct
  misc_feature 1..741: note = Description of Artificial Sequence: Synthetic polynucleotide
  source 1..741: mol_type = other DNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccct

SEQ ID NO: 7 - DNA, 585 bp, synthetic construct
  misc_feature 1..585: note = Description of Artificial Sequence: Synthetic polynucleotide
  source 1..585: mol_type = other DNA
atagtgagtcgtattaacgtaccaacaaggcgtggagctgagagataaacttgttatctctcagctccacgcctttatcttagaggcatatccctgccaccatgggcctgacatctcagttgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctagtttatcttagaggcatatccct

SEQ ID NO: 8 - DNA, 585 bp, synthetic construct
  misc_feature 1..585: note = Description of Artificial Sequence: Synthetic polynucleotide
  source 1..585: mol_type = other DNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatcccttttatcttagaggcatatccct

SEQ ID NO: 9 - DNA, 651 bp, synthetic construct
  misc_feature 1..651: note = Description of Artificial Sequence: Synthetic polynucleotide
  source 1..651: mol_type = other DNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaacaacaaggcgtggagctgagagataaacttgttatctctcagctccacgccacaacaagggcctgtacctcatctactacttgagtagatgaggtacaggcccacaacaaggtatgagcccatctatctacttgagatagatgggctcataccacaacaatttatcttagaggcatatccct

SEQ ID NO: 10 - DNA, 482 bp, synthetic construct
  misc_feature 1..482: note = Description of Artificial Sequence: Synthetic polynucleotide
  source 1..482: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatcccttttatcttagaggcatatccct

SEQ ID NO: 11 - DNA, 563 bp, synthetic construct
  misc_feature 1..563: note = Description of Artificial Sequence: Synthetic polynucleotide
  source 1..563: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccct

SEQ ID NO: 12 - DNA, 644 bp, synthetic construct
  misc_feature 1..644: note = Description of Artificial Sequence: Synthetic polynucleotide
  source 1..644: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccct

SEQ ID NO: 13 - DNA, 2808 bp, synthetic construct
  misc_feature 1..2808: note = Description of Artificial Sequence: Synthetic polynucleotide
  source 1..2808: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaacaaggaagtgctaaagaaacttgttctttagcacttccttgtttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 14 - DNA, 2956 bp, synthetic construct
  misc_feature 1..2956: note = Description of Artificial Sequence: Synthetic polynucleotide
  source 1..2956: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaacaaggagtgctaaagaaacttgttctttagcactccttgtttatcttagaggcatatccctacgtaccaacaagagagtgattgagagtggacttgccactctcaatcactctctttatcttagaggcatatccctacgtaccaacaagagagctctgtctggaccacttgggtccagacagagctctctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 15 - DNA, 2970 bp, synthetic construct
  misc_feature 1..2970: note = Description of Artificial Sequence: Synthetic polynucleotide
  source 1..2970: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctagtttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 16 - DNA, 3319 bp, synthetic construct
  misc_feature 1..3319: note = Description of Artificial Sequence: Synthetic polynucleotide
  source 1..3319: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctacgtaccaacaagcaatgaggaccctgagagatacttgatctctcagggtcctcattgctttatcttagaggcatatccctacgtaccaacaagctgatgggaacgtggactaacttgtagtccacgttcccatcagctttatcttagaggcatatccctacgtaccaacaaggtcctcagattactacaaacttgttgtagtaatctgaggacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 17 - DNA, 3082 bp, synthetic construct
  misc_feature 1..3082: note = Description of Artificial Sequence: Synthetic polynucleotide
  source 1..3082: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 18 - DNA, 3082 bp, synthetic construct
  misc_feature 1..3082: note = Description of Artificial Sequence: Synthetic polynucleotide
  source 1..3082: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 19 - DNA, 2926 bp, synthetic construct
  misc_feature 1..2926: note = Description of Artificial Sequence: Synthetic polynucleotide
  source 1..2926: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaaggcgtggagctgagagataaacttgttatctctcagctccacgcctttatcttagaggcatatccctgccaccatgggcctgacatctcagttgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctagtttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 20 - DNA, 2926 bp, synthetic construct
  misc_feature 1..2926: note = Description of Artificial Sequence: Synthetic polynucleotide
  source 1..2926: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 21 - DNA, 2992 bp, synthetic construct
  misc_feature 1..2992: note = Description of Artificial Sequence: Synthetic polynucleotide
  source 1..2992: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaacaacaaggcgtggagctgagagataaacttgttatctctcagctccacgccacaacaagggcctgtacctcatctactacttgagtagatgaggtacaggcccacaacaaggtatgagcccatctatctacttgagatagatgggctcataccacaacaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 22 - DNA, 2618 bp, synthetic construct
  misc_feature 1..2618: note = Description of Artificial Sequence: Synthetic polynucleotide
  source 1..2618: mol_type = other DNA
cttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 23 - DNA, 2904 bp, synthetic construct
  misc_feature 1..2904: note = Description of Artificial Sequence: Synthetic polynucleotide
  source 1..2904: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 24 - DNA, 2985 bp, synthetic construct
  misc_feature 1..2985: note = Description of Artificial Sequence: Synthetic polynucleotide
  source 1..2985: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 25 - skipped

SEQ ID NO: 26 - DNA, 17 bp, synthetic construct
  misc_feature 1..17: note = Description of Artificial Sequence: Synthetic oligonucleotide
  source 1..17: mol_type = other DNA
taatacgactcactata

SEQ ID NO: 27 - DNA, 28 bp, synthetic construct
  misc_feature 1..28: note = Description of Artificial Sequence: Synthetic oligonucleotide
  source 1..28: mol_type = other DNA
atagtgagtcgtattaacgtaccaacaa

SEQ ID NO: 28 - DNA, 34 bp, synthetic construct
  misc_feature 1..34: note = Description of Artificial Sequence: Synthetic oligonucleotide
  source 1..34: mol_type = other DNA
tttatcttagaggcatatccctacgtaccaacaa

SEQ ID NO: 29 - DNA, 20 bp, synthetic construct
  misc_feature 1..20: note = Description of Artificial Sequence: Synthetic primer
  source 1..20: mol_type = other DNA
gctgcaaggcgattaagttg

SEQ ID NO: 30 - DNA, 143 bp, synthetic construct
  misc_feature 1..143: note = Description of Artificial Sequence: Synthetic primer
  misc_feature 1..143: note = Description of Combined DNA/RNA Molecule: Synthetic primer
  source 1..143: mol_type = other DNA
  misc_feature 1..3: note = RNA fragment
  misc_feature 4..143: note = DNA fragment
  modified_base 1..3: mod_base = um
ttttttttttttttttttttttttttttttttttttttttttttttttttttttttttttttttttttttttttttttttttttttttttttttttttttttttttttttttttttttttcagctatgaccatgttaatgcag

SEQ ID NO: 31 - DNA, 615 bp, Homo sapiens
  source 1..615: mol_type = genomic DNA
atcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaa

SEQ ID NO: 32 - protein, 153 aa, Homo sapiens [sequence omitted]

SEQ ID NO: 33 - DNA, 462 bp, Homo sapiens
  source 1..462: mol_type = genomic DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 34 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 35 - DNA, 348 bp, synthetic construct
  misc_feature 1..348: note = Description of Artificial Sequence: Synthetic polynucleotide
  source 1..348: mol_type = other DNA
atgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaa

SEQ ID NO: 36 - protein, 115 aa, synthetic construct
  REGION 1..115: note = Description of Artificial Sequence: Synthetic polypeptide
MTILFLTMVISYFGCMKAVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSA

SEQ ID NO: 37 - DNA, 300 bp, Homo sapiens
  source 1..300: mol_type = genomic DNA
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataa

SEQ ID NO: 38 - DNA, 810 bp, Homo sapiens
  source 1..810: mol_type = genomic DNA
atggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 39 - DNA, 468 bp, Homo sapiens
  source 1..468: mol_type = genomic DNA
atgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaa

SEQ ID NO: 40 - DNA, 702 bp, Homo sapiens
  source 1..702: mol_type = genomic DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 41 - DNA, 699 bp, Pontellina plumata
  source 1..699: mol_type = other DNA
atggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataa

SEQ ID NO: 42 - DNA, 453 bp, synthetic construct
  misc_feature 1..453: note = Description of Artificial Sequence: Synthetic polynucleotide
  source 1..453: mol_type = other DNA
gccaccatgttgctgctgcctctgttcttcctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctga

SEQ ID NO: 43 - protein, 148 aa, synthetic construct
  REGION 1..148: note = Description of Artificial Sequence: Synthetic polypeptide
MLLLPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 44 - protein, 19 aa, synthetic construct
  REGION 1..19: note = Description of Artificial Sequence: Synthetic peptide
MLILLLPLLLFKCFCDFLK

SEQ ID NO: 45 - DNA, 57 bp, synthetic construct
  misc_feature 1..57: note = Description of Artificial Sequence: Synthetic oligonucleotide
  source 1..57: mol_type = other DNA
atgctgattctgctgctgcccctgctgctgttcaagtgcttctgcgacttcctgaaa

SEQ ID NO: 46 - protein, 18 aa, synthetic construct
  REGION 1..18: note = Description of Artificial Sequence: Synthetic peptide
MLFYLALCLLTFTSSATA

SEQ ID NO: 47 - DNA, 54 bp, synthetic construct
  misc_feature 1..54: note = Description of Artificial Sequence: Synthetic oligonucleotide
  source 1..54: mol_type = other DNA
atgctgttctatctggccctgtgcctgctgacctttaccagctctgctaccgcc

SEQ ID NO: 48 - protein, 10 aa, synthetic construct
  REGION 1..10: note = Description of Artificial Sequence: Synthetic peptide
VKMHTMSSSH

SEQ ID NO: 49 - protein, 21 aa, Homo sapiens
MGKISSLPTQLFKCCFCDFLK

SEQ ID NO: 50 - DNA, 63 bp, Homo sapiens
  source 1..63: mol_type = other DNA
atgggaaaaatcagcagtcttccaacccaattatttaagtgctgcttttgtgatttcttgaag

SEQ ID NO: 51 - protein, 18 aa, synthetic construct
  REGION 1..18: note = Description of Artificial Sequence: Synthetic peptide
MTILFLTMVISYFGCMKA

SEQ ID NO: 52 - DNA, 54 bp, synthetic construct
  misc_feature 1..54: note = Description of Artificial Sequence: Synthetic oligonucleotide
  source 1..54: mol_type = other DNA
atgaccatcctgtttctgacaatggtcatcagctacttcggctgcatgaaggcc

SEQ ID NO: 53 - protein, 24 aa, Homo sapiens
MGLTSQLLPPLFFLLACAGNFVHG

SEQ ID NO: 54 - DNA, 72 bp, Homo sapiens
  source 1..72: mol_type = other DNA
atgggtctcacctcccaactgcttccccctctgttcttcctgctagcatgtgccggcaactttgtccacgga

SEQ ID NO: 55 - protein, 19 aa, synthetic construct
  REGION 1..19: note = Description of Artificial Sequence: Synthetic peptide
MLLLPLFFLLACAGNFVHG

SEQ ID NO: 56 - DNA, 57 bp, synthetic construct
  misc_feature 1..57: note = Description of Artificial Sequence: Synthetic oligonucleotide
  source 1..57: mol_type = other DNA
atgttgctgctgcctctgttcttcctgctggcctgcgccggcaattttgtgcacggc

SEQ ID NO: 57 - DNA, 18 bp, synthetic construct
  misc_feature 1..18: note = Description of Artificial Sequence: Synthetic oligonucleotide
  source 1..18: mol_type = other DNA
caaggaagtgctaaagaa

SEQ ID NO: 58 - DNA, 17 bp, synthetic construct
  misc_feature 1..17: note = Description of Artificial Sequence: Synthetic oligonucleotide
  source 1..17: mol_type = other DNA
caaggagtgctaaagaa

SEQ ID NO: 59 - DNA, 18 bp, synthetic construct
  misc_feature 1..18: note = Description of Artificial Sequence: Synthetic oligonucleotide
  source 1..18: mol_type = other DNA
gagagtgattgagagtgg

SEQ ID NO: 60 - DNA, 18 bp, synthetic construct
  misc_feature 1..18: note = Description of Artificial Sequence: Synthetic oligonucleotide
  source 1..18: mol_type = other DNA
gagagctctgtctggacc

SEQ ID NO: 61 - DNA, 21 bp, synthetic construct
  misc_feature 1..21: note = Description of Artificial Sequence: Synthetic oligonucleotide
  source 1..21: mol_type = other DNA
gaaagatgataagcccactct

SEQ ID NO: 62 - DNA, 21 bp, synthetic construct
  misc_feature 1..21: note = Description of Artificial Sequence: Synthetic oligonucleotide
  source 1..21: mol_type = other DNA
ggtgatgtctggtccatatga

SEQ ID NO: 63 - DNA, 18 bp, synthetic construct
  misc_feature 1..18: note = Description of Artificial Sequence: Synthetic oligonucleotide
  source 1..18: mol_type = other DNA
gatgataagcccactcta

SEQ ID NO: 64 - DNA, 20 bp, synthetic construct
  misc_feature 1..20: note = Description of Artificial Sequence: Synthetic oligonucleotide
  source 1..20: mol_type = other DNA
ggcgtggagctgagagataa

SEQ ID NO: 65 - DNA, 20 bp, synthetic construct
  misc_feature 1..20: note = Description of Artificial Sequence: Synthetic oligonucleotide
  source 1..20: mol_type = other DNA
gggcctgtacctcatctact

SEQ ID NO: 66 - DNA, 19 bp, synthetic construct
  misc_feature 1..19: note = Description of Artificial Sequence: Synthetic oligonucleotide
  source 1..19: mol_type = other DNA
ggtatgagcccatctatct

SEQ ID NO: 67 - DNA, 21 bp, synthetic construct
  misc_feature 1..21: note = Description of Artificial Sequence: Synthetic oligonucleotide
  source 1..21: mol_type = other DNA
gcaatgaggaccctgagagat

SEQ ID NO: 68 - DNA, 20 bp, synthetic construct
  misc_feature 1..20: note = Description of Artificial Sequence: Synthetic oligonucleotide
  source 1..20: mol_type = other DNA
gctgatgggaacgtggacta

SEQ ID NO: 69 - DNA, 19 bp, synthetic construct
  misc_feature 1..19: note = Description of Artificial Sequence: Synthetic oligonucleotide
  source 1..19: mol_type = other DNA
ggtcctcagattactacaa

SEQ ID NO: 70 - DNA, 20 bp, synthetic construct
  misc_feature 1..20: note = Description of Artificial Sequence: Synthetic oligonucleotide
  source 1..20: mol_type = other DNA
aacaagatgaagagcaccaa

SEQ ID NO: 71 - DNA, 18 bp, synthetic construct
  misc_feature 1..18: note = Description of Artificial Sequence: Synthetic oligonucleotide
  source 1..18: mol_type = other DNA
ttctttagcacttccttg

SEQ ID NO: 72 - DNA, 17 bp, synthetic construct
  misc_feature 1..17: note = Description of Artificial Sequence: Synthetic oligonucleotide
  source 1..17: mol_type = other DNA
ttctttagcactccttg

SEQ ID NO: 73 - DNA, 18 bp, synthetic construct
  misc_feature 1..18: note = Description of Artificial Sequence: Synthetic oligonucleotide
  source 1..18: mol_type = other DNA
ccactctcaatcactctc

SEQ ID NO: 74 - DNA, 18 bp, synthetic construct
  misc_feature 1..18: note = Description of Artificial Sequence: Synthetic oligonucleotide
  source 1..18: mol_type = other DNA
ggtccagacagagctctc

SEQ ID NO: 75 - DNA, 21 bp, synthetic construct
  misc_feature 1..21: note = Description of Artificial Sequence: Synthetic oligonucleotide
  source 1..21: mol_type = other DNA
agagtgggcttatcatctttc

SEQ ID NO: 76 - DNA, 21 bp, synthetic construct
  misc_feature 1..21: note = Description of Artificial Sequence: Synthetic oligonucleotide
  source 1..21: mol_type = other DNA
tcatatggaccagacatcacc

SEQ ID NO: 77 - DNA, 18 bp, synthetic construct
  misc_feature 1..18: note = Description of Artificial Sequence: Synthetic oligonucleotide
  source 1..18: mol_type = other DNA
tagagtgggcttatcatc

SEQ ID NO: 78 - DNA, 20 bp, synthetic construct
  misc_feature 1..20: note = Description of Artificial Sequence: Synthetic oligonucleotide
  source 1..20: mol_type = other DNA
ttatctctcagctccacgcc

SEQ ID NO: 79 - DNA, 20 bp, synthetic construct
  misc_feature 1..20: note = Description of Artificial Sequence: Synthetic oligonucleotide
  source 1..20: mol_type = other DNA
agtagatgaggtacaggccc

SEQ ID NO: 80 - DNA, 19 bp, synthetic construct
  misc_feature 1..19: note = Description of Artificial Sequence: Synthetic oligonucleotide
  source 1..19: mol_type = other DNA
agatagatgggctcatacc

SEQ ID NO: 81 - DNA, 21 bp, synthetic construct
  misc_feature 1..21: note = Description of Artificial Sequence: Synthetic oligonucleotide
  source 1..21: mol_type = other DNA
atctctcagggtcctcattgc

SEQ ID NO: 82 - DNA, 20 bp, synthetic construct
  misc_feature 1..20: note = Description of Artificial Sequence: Synthetic oligonucleotide
  source 1..20: mol_type = other DNA
tagtccacgttcccatcagc

SEQ ID NO: 83 - DNA, 19 bp, synthetic construct
  misc_feature 1..19: note = Description of Artificial Sequence: Synthetic oligonucleotide
  source 1..19: mol_type = other DNA
ttgtagtaatctgaggacc

SEQ ID NO: 84 - DNA, 21 bp, synthetic construct
  misc_feature 1..21: note = Description of Artificial Sequence: Synthetic oligonucleotide
  source 1..21: mol_type = other DNA
ttggtgctcttcatcttgttg

SEQ ID NO: 85 - skipped

SEQ ID NO: 86 - DNA, 16 bp, synthetic construct
  misc_feature 1..16: note = Description of Artificial Sequence: Synthetic oligonucleotide
  source 1..16: mol_type = other DNA
atagtgagtcgtatta

SEQ ID NO: 87 - DNA, 18 bp, synthetic construct
  misc_feature 1..18: note = Description of Artificial Sequence: Synthetic oligonucleotide
  source 1..18: mol_type = other DNA
atccctacgtaccaacaa

SEQ ID NO: 88 - DNA, 12 bp, synthetic construct
  misc_feature 1..12: note = Description of Artificial Sequence: Synthetic oligonucleotide
  source 1..12: mol_type = other DNA
acgtaccaacaa

SEQ ID NO: 89 - skipped

SEQ ID NO: 90 - DNA, 11 bp, synthetic construct
  misc_feature 1..11: note = Description of Artificial Sequence: Synthetic oligonucleotide
  source 1..11: mol_type = other DNA
acaacaatccc

SEQ ID NO: 91 - skipped

SEQ ID NO: 92 - DNA, 20 bp, synthetic construct
  misc_feature 1..20: note = Description of Artificial Sequence: Synthetic oligonucleotide
  source 1..20: mol_type = other DNA
atagtgagtcgtattatccc

SEQ ID NO: 93 - DNA, 27 bp, synthetic construct
  misc_feature 1..27: note = Description of Artificial Sequence: Synthetic oligonucleotide
  source 1..27: mol_type = other DNA
atagtgagtcgtattaacaacaatccc

SEQ ID NO: 94 - DNA, 23 bp, synthetic construct
  misc_feature 1..23: note = Description of Artificial Sequence: Synthetic oligonucleotide
  source 1..23: mol_type = other DNA
atagtgagtcgtattaacaacaa

SEQ ID NO: 95 - DNA, 34 bp, synthetic construct
  misc_feature 1..34: note = Description of Artificial Sequence: Synthetic oligonucleotide
  source 1..34: mol_type = other DNA
atagtgagtcgtattaatccctacgtaccaacaa

SEQ ID NO: 96 - DNA, 77 bp, synthetic construct
  misc_feature 1..77: note = Description of Artificial Sequence: Synthetic oligonucleotide
  source 1..77: mol_type = other DNA
aacaaagcaccagtggtctagtggtagaatagtaccctgccacggtacagacccgggttcgattcccggctggtgca

SEQ ID NO: 97 - RNA, 467 nt, synthetic construct
  misc_feature 1..467: note = Description of Artificial Sequence: Synthetic polynucleotide
  source 1..467: mol_type = other RNA
atagtgagtcgtattaacgtaccaacaacaaggaagtgctaaagaaacttgttctttagcacttccttgtttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 98 - RNA, 615 nt, synthetic construct
  misc_feature 1..615: note = Description of Artificial Sequence: Synthetic polynucleotide
  source 1..615: mol_type = other RNA
atagtgagtcgtattaacgtaccaacaacaaggagtgctaaagaaacttgttctttagcactccttgtttatcttagaggcatatccctacgtaccaacaagagagtgattgagagtggacttgccactctcaatcactctctttatcttagaggcatatccctacgtaccaacaagagagctctgtctggaccacttgggtccagacagagctctctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 99 - RNA, 629 nt, synthetic construct
  misc_feature 1..629: note = Description of Artificial Sequence: Synthetic polynucleotide
  source 1..629: mol_type = other RNA
atagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 100 - RNA, 978 nt, synthetic construct
  misc_feature 1..978: note = Description of Artificial Sequence: Synthetic polynucleotide
  source 1..978: mol_type = other RNA
atagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctacgtaccaacaagcaatgaggaccctgagagatacttgatctctcagggtcctcattgctttatcttagaggcatatccctacgtaccaacaagctgatgggaacgtggactaacttgtagtccacgttcccatcagctttatcttagaggcatatccctacgtaccaacaaggtcctcagattactacaaacttgttgtagtaatctgaggacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccct

SEQ ID NO: 101 - RNA, 741 nt, synthetic construct
  misc_feature 1..741: note = Description of Artificial Sequence: Synthetic polynucleotide
  source 1..741: mol_type = other RNA
atagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccct

SEQ ID NO: 102 - RNA, 741 nt, synthetic construct
  misc_feature 1..741: note = Description of Artificial Sequence: Synthetic polynucleotide
  source 1..741: mol_type = other RNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccct

SEQ ID NO: 103 - RNA, 585 nt, synthetic construct
  misc_feature 1..585: note = Description of Artificial Sequence: Synthetic polynucleotide
  source 1..585: mol_type = other RNA
atagtgagtcgtattaacgtaccaacaaggcgtggagctgagagataaacttgttatctctcagctccacgcctttatcttagaggcatatccctgccaccatgggcctgacatctcagttgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctagtttatcttagaggcatatccct

SEQ ID NO: 104 - RNA, 585 nt, synthetic construct
  misc_feature 1..585: note = Description of Artificial Sequence: Synthetic polynucleotide
  source 1..585: mol_type = other RNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatcccttttatcttagaggcatatccct

SEQ ID NO: 105 - RNA, 651 nt, synthetic construct
  misc_feature 1..651: note = Description of Artificial Sequence: Synthetic polynucleotide
  source 1..651: mol_type = other RNA
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaacaacaaggcgtggagctgagagataaacttgttatctctcagctccacgccacaacaagggcctgtacctcatctactacttgagtagatgaggtacaggcccacaacaaggtatgagcccatctatctacttgagatagatgggctcataccacaacaatttatcttagaggcatatccct

SEQ ID NO: 106 - RNA, 482 nt, synthetic construct
  misc_feature 1..482: note = Description of Artificial Sequence: Synthetic polynucleotide
  source 1..482: mol_type = other RNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatcccttttatcttagaggcatatccct

SEQ ID NO: 107 - RNA, 563 nt, synthetic construct
  misc_feature 1..563: note = Description of Artificial Sequence: Synthetic polynucleotide
  source 1..563: mol_type = other RNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccct

SEQ ID NO: 108 - RNA, 644 nt, synthetic construct
  misc_feature 1..644: note = Description of Artificial Sequence: Synthetic polynucleotide
  source 1..644: mol_type = other RNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatccctacgtaccaacaacaacaagatgaagagcaccaaacttgttggtgctcttcatcttgttgtttatcttagaggcatatcccttttatcttagaggcatatccct

SEQ ID NO: 109 - RNA, 467 nt, synthetic construct; identical to SEQ ID NO: 97
  misc_feature 1..467: note = Description of Artificial Sequence: Synthetic polynucleotide
  source 1..467: mol_type = other RNA
  misc_difference 1..467: note = T in any of positions 1-467 are replaced with 1-methylpseudouridine.
  modified_base 1..467: mod_base = m1f

SEQ ID NO: 110 - RNA, 615 nt, synthetic construct; identical to SEQ ID NO: 98
  misc_feature 1..615: note = Description of Artificial Sequence: Synthetic polynucleotide
  source 1..615: mol_type = other RNA
  misc_difference 1..615: note = T in any of positions 1-615 are replaced with 1-methylpseudouridine.
  modified_base 1..615: mod_base = m1f

SEQ ID NO: 111 - RNA, 629 nt, synthetic construct; identical to SEQ ID NO: 99
  misc_feature 1..629: note = Description of Artificial Sequence: Synthetic polynucleotide
  source 1..629: mol_type = other RNA
  misc_difference 1..629: note = T in any of positions 1-629 are replaced with 1-methylpseudouridine.
  modified_base 1..629: mod_base = m1f

SEQ ID NO: 112 - RNA, 978 nt, synthetic construct; identical to SEQ ID NO: 100
  misc_feature 1..978: note = Description of Artificial Sequence: Synthetic polynucleotide
  source 1..978: mol_type = other RNA
  misc_difference 1..978: note = T in any of positions 1-978 are replaced with 1-methylpseudouridine.
  modified_base 1..978: mod_base = m1f

SEQ ID NO: 113 - RNA, 741 nt, synthetic construct; identical to SEQ ID NO: 101
  misc_feature 1..741: note = Description of Artificial Sequence: Synthetic polynucleotide
  source 1..741: mol_type = other RNA
  misc_difference 1..741: note = T in any of positions 1-741 are replaced with 1-methylpseudouridine.
  modified_base 1..741: mod_base = m1f

SEQ ID NO: 114 - RNA, 741 nt, synthetic construct; identical to SEQ ID NO: 102
  misc_feature 1..741: note = Description of Artificial Sequence: Synthetic polynucleotide
  source 1..741: mol_type = other RNA
  misc_difference 1..741: note = T in any of positions 1-741 are replaced with 1-methylpseudouridine.
  modified_base 1..741: mod_base = m1f

SEQ ID NO: 115 - RNA, 585 nt, synthetic construct; identical to SEQ ID NO: 103
  misc_feature 1..585: note = Description of Artificial Sequence: Synthetic polynucleotide
  source 1..585: mol_type = other RNA
  misc_difference 1..585: note = T in any of positions 1-585 are replaced with 1-methylpseudouridine.
  modified_base 1..585: mod_base = m1f

SEQ ID NO: 116 - RNA, 585 nt, synthetic construct; identical to SEQ ID NO: 104
  misc_feature 1..585: note = Description of Artificial Sequence: Synthetic polynucleotide
  source 1..585: mol_type = other RNA
  misc_difference 1..585: note = T in any of positions 1-585 are replaced with 1-methylpseudouridine.
  modified_base 1..585: mod_base = m1f

SEQ ID NO: 117 - RNA, 651 nt, synthetic construct; identical to SEQ ID NO: 105
  misc_feature 1..651: note = Description of Artificial Sequence: Synthetic polynucleotide
  source 1..651: mol_type = other RNA
  misc_difference 1..651: note = T in any of positions 1-651 are replaced with 1-methylpseudouridine.
  modified_base 1..651: mod_base = m1f

SEQ ID NO: 118 - RNA, 482 nt, synthetic construct; identical to SEQ ID NO: 106
  misc_feature 1..482: note = Description of Artificial Sequence: Synthetic polynucleotide
  source 1..482: mol_type = other RNA
  misc_difference 1..482: note = T in any of positions 1-482 are replaced with 1-methylpseudouridine.
  modified_base 1..482: mod_base = m1f

SEQ ID NO: 119 - RNA, 563 nt, synthetic construct; identical to SEQ ID NO: 107
  misc_feature 1..563: note = Description of Artificial Sequence: Synthetic polynucleotide
  source 1..563: mol_type = other RNA
  misc_difference 1..563: note = T in any of positions 1-563 are replaced with 1-methylpseudouridine.
  modified_base 1..563: mod_base = m1f

SEQ ID NO: 120 - RNA, 644 nt, synthetic construct; identical to SEQ ID NO: 108
  misc_feature 1..644: note = Description of Artificial Sequence: Synthetic polynucleotide
  source 1..644: mol_type = other RNA
  misc_difference 1..644: note = T in any of positions 1-644 are replaced with 1-methylpseudouridine.
  modified_base 1..644: mod_base = m1f

SEQ ID NO: 121 - RNA, 363 nt, synthetic construct
  misc_feature 1..363: note = Description of Artificial Sequence: Synthetic polynucleotide
  source 1..363: mol_type = other RNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaa

SEQ ID NO: 122 - RNA, 453 nt, synthetic construct
  misc_feature 1..453: note = Description of Artificial Sequence: Synthetic polynucleotide
  source 1..453: mol_type = other RNA
gccaccatgttgctgctgcctctgttcttcctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctga

SEQ ID NO: 123 - RNA, 363 nt, synthetic construct; identical to SEQ ID NO: 121
  misc_feature 1..363: note = Description of Artificial Sequence: Synthetic polynucleotide
  source 1..363: mol_type = other RNA
  misc_difference 1..363: note = T in any of positions 1-363 are replaced with 1-methylpseudouridine.
  modified_base 1..363: mod_base = m1f

SEQ ID NO: 124 - RNA, 453 nt, synthetic construct; identical to SEQ ID NO: 122
  misc_feature 1..453: note = Description of Artificial Sequence: Synthetic polynucleotide
  source 1..453: mol_type = other RNA
  misc_difference 1..453: note = T in any of positions 1-453 are replaced with 1-methylpseudouridine.
  modified_base 1..453: mod_base = m1f

SEQ ID NO: 125 - DNA, 363 bp, synthetic construct
  misc_feature 1..363: note = Description of Artificial Sequence: Synthetic polynucleotide
  source 1..363: mol_type = other DNA
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaa

SEQ ID NO: 126 - protein, 118 aa, synthetic construct
  REGION 1..118: note = Description of Artificial Sequence: Synthetic polypeptide
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSA

SEQ ID NO: 127 - DNA, 2749 bp, synthetic construct
  misc_feature 1..2749: note = Description of Artificial Sequence: Synthetic polynucleotide
  source 1..2749: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcggatccgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaagaattcggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 128 - DNA, 2827 bp, synthetic construct
  misc_feature 1..2827: note = Description of Artificial Sequence: Synthetic polynucleotide
  source 1..2827: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgttgctgctgcctctgttcttcctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 129-130 - skipped (2 entries)

SEQ ID NO: 131 - RNA, 220 nt, synthetic construct
  misc_feature 1..220: note = Description of Artificial Sequence: Synthetic polynucleotide
  misc_feature 1..220: note = This sequence may encompass 1-220 nucleotides
  misc_feature 1..220: note = See specification as filed for detailed description of substitutions and preferred embodiments
  source 1..220: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 132 - RNA, 200 nt, synthetic construct
  misc_feature 1..200: note = Description of Artificial Sequence: Synthetic polynucleotide
  misc_feature 1..200: note = This sequence may encompass 1-200 nucleotides
  misc_feature 1..200: note = See specification as filed for detailed description of substitutions and preferred embodiments
  source 1..200: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 133 - RNA, 120 nt, synthetic construct
  misc_feature 1..120: note = Description of Artificial Sequence: Synthetic polynucleotide
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 134 - DNA, 120 bp, synthetic construct
  misc_feature 1..120: note = Description of Artificial Sequence: Synthetic polynucleotide
  source 1..120: mol_type = other DNA
tttttttttttttttttttttttttttttttttttttttttttttttttttttttttttttttttttttttttttttttttttttttttttttttttttttttttttttttttttttttt

(19) United States
(12) Patent Application Publication
(10) Pub. No.: US 2024/0117361 A1
(43) Pub. Date: Apr. 11, 2024

(54) COMPOSITIONS AND METHODS FOR MODULATING EXPRESSION OF GENES

(71) Applicant: Versameb AG, Basel (CH)

(72) Inventors: Justin ANTONY SELVARAJ, Weil am Rhein (DE); Klaas Pieter ZUIDEVELD, Riehen (CH); Petra HILLMANN-WÜLLNER, Zurich (CH); Friedrich METZGER, Freiburg (DE)

(21) Appl. No.: 18/542,975

(22) Filed: Dec. 18, 2023

Related U.S. Application Data
(63) Continuation of application No. PCT/IB2022/000358, filed on Jun. 22, 2022.
(60) Provisional application No. 63/213,829, filed on Jun. 23, 2021.

(51) Int. Cl. C12N 15/113; A61P 17/02; A61P 37/06; C07K 14/54; C07K 14/65; C12N 9/16

(52) U.S. Cl. CPC C12N 15/1138; A61P 17/02; A61P 37/06; C07K 14/5406; C07K 14/65; C12N 9/16; C12N 2310/14; C12Y 301/03001

(57) ABSTRACT

The present invention relates to compositions and methods for modulating expression of genes, comprising recombinant polynucleic acid or RNA constructs comprising a first RNA sequence encoding a gene of interest, and a second RNA sequence comprising at least two genetic elements that modulate expression of one or more target RNAs. The recombinant RNA constructs described herein induce an immune response in a human cell that is lower than the immune response induced by a corresponding recombinant RNA construct comprising the first RNA sequence encoding a gene of interest and a corresponding second RNA sequence comprising at most one of the at least two genetic elements. Also disclosed herein is the use of the compositions in treating diseases and in modulating expression of two or more genes.

### CROSS-REFERENCE TO RELATED APPLICATIONS

This application is a continuation of International Application No. PCT/IB2022/000358, filed Jun. 22, 2022, which claims the benefit of U.S. Provisional Application No. 63/213,829, filed Jun. 23, 2021, each of which is incorporated by reference herein in its entirety.

### SEQUENCE LISTING

The instant application contains a Sequence Listing which has been submitted electronically in XML format and is hereby incorporated by reference in its entirety. Said XML copy, created on Dec. 5, 2023, is named 57623-709_301_SL.xml and is 249,712 bytes in size.

### BACKGROUND

Numerous human diseases and disorders are caused by combinations of higher and/or lower expression levels of certain proteins compared to the expression levels of these proteins in humans without the disease or disorder. RNA therapeutics that can selectively increase the expression of a target protein and decrease the expression of another, different target protein, with a single construct, may have a therapeutic effect. However, exogenous RNAs can trigger undesirable innate immune response. Accordingly, there is a need to develop RNA therapeutics with reduced immunogenicity to avoid unwanted innate immune activation.

### BRIEF SUMMARY

Provided herein are compositions and methods for simultaneously modulating expression of two or three or more proteins using one recombinant polynucleic acid or RNA construct. For example, the compositions and methods provided herein can be used to increase expression of a first protein and decrease expression of a second protein. For example, the compositions and methods provided herein can be used to increase expression of a first protein, decrease expression of a second protein and decrease expression of a third protein.

In some aspects, provided herein, is a composition comprising a recombinant RNA construct comprising: (i) a first RNA sequence encoding a gene of interest, and (ii) a second RNA sequence comprising at least two genetic elements that modulate expression of one or more target RNAs, wherein contacting a human cell with the recombinant RNA construct results in an immune response that is lower than the immune response of the human cell contacted with a corresponding recombinant RNA construct comprising the first RNA sequence of (i) and a corresponding second RNA sequence of (ii) with at most one of the at least two genetic elements. In some embodiments, the recombinant RNA construct comprises one or more uridines. In some embodiments, the recombinant RNA construct does not comprise a modified uridine. In some embodiments, the nucleotide variant comprises a modified uridine. In some embodiments, the modified uridine comprises a N1-methylpseudouridine.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct does not comprise a nucleotide variant.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct does not comprise a modified uridine.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct does not comprise a N1-methylpseudouridine.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct comprises solely unmodified nucleotides or natural nucleotides.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct comprises uridines, wherein (a) all uridines comprised by the recombinant RNA constructs are unmodified or natural nucleotide(s); or (b) at least one of the uridines comprised by the recombinant RNA constructs is an unmodified uridine.

In some aspects, provided herein, is a composition for use in modulating the expression of two or more genes in a cell. In some aspects, provided herein, is a pharmaceutical composition comprising a therapeutically effective amount of any one of the compositions described herein and a pharmaceutically acceptable excipient. In some aspects, provided herein, is a vector comprising a recombinant polynucleic acid construct encoding any one of the compositions described herein. In some aspects, provided herein, is a cell comprising any one of the compositions described herein or any one of the vectors described herein.

In some aspects, provided herein, is a method of simultaneously expressing an siRNA and an mRNA from a single RNA transcript in a cell, comprising introducing into the cell any one of the compositions described herein, or any one of the vectors described herein.

In some aspects, provided herein, is a method of treating a disease or condition comprising administering to a subject in need thereof any one of the compositions described herein or any one of the pharmaceutical compositions described herein.

In some aspects, provided herein, is a composition comprising a recombinant RNA construct comprising: (i) a messenger RNA (mRNA) encoding Insulin-like Growth Factor 1 (IGF-1), and (ii) at least two small interfering RNAs (siRNAs) capable of binding to an Interleukin-8 (IL-8) mRNA, wherein contacting a human cell with the recombinant RNA construct results in an immune response that is lower than the immune response of the human cell contacted with a corresponding recombinant RNA construct comprising the mRNA encoding IGF-1 of (i) and at most one of the at least two siRNAs capable of binding to the IL-8 mRNA of (ii).

In some aspects, provided herein, is a composition comprising recombinant RNA construct comprising: (i) a messenger RNA (mRNA) encoding Insulin-like Growth Factor 1 (IGF-1), and (ii) at least two small interfering RNAs (siRNAs) capable of binding to a Interleukin-1 beta (IL-1 beta) mRNA, wherein contacting a human cell with the recombinant RNA construct results in an immune response that is lower than the immune response of the human cell contacted with a corresponding recombinant RNA construct comprising the mRNA encoding IGF-1 of (i) and at most one of the at least two siRNAs capable of binding to the IL-1beta mRNA of (ii).

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA, wherein contacting a human cell with the recombinant RNA construct results in an immune response that is lower than the immune response of the human cell contacted with a corresponding recombinant RNA construct comprising the mRNA encoding IL-4 of (i) and at most one of the at least two siRNAs capable of binding to the TNF-alpha mRNA of (ii).

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA and Interleukin 17 (IL-17), wherein contacting a human cell with the recombinant RNA construct results in an immune response that is lower than the immune response of the human cell contacted with a corresponding recombinant RNA construct comprising the mRNA encoding IL-4 of (i) and at most one of the at least two siRNAs capable of binding to the TNF-alpha mRNA and IL-17 mRNA of (ii).

In some aspects, provided herein, is a composition comprising a recombinant polynucleic acid construct comprising a nucleic acid sequence selected from the group consisting of SEQ ID NOs: 1-24, 42, 125, 97-108, 121-122, and 127-128.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic representation of construct design. A polynucleic acid (e.g., DNA) construct may comprise a T7 promoter sequence upstream of the gene of interest sequence for T7 RNA polymerase binding and successful in vitro transcription of both the gene of interest (e.g., IGF-1 or IL-4) and siRNA in a single RNA transcript. Signal peptide of the gene of interest is highlighted in a grey box. Linkers to connect mRNA to siRNA or siRNA to siRNA are indicated with boxes with horizontal stripes or boxes with checkered stripes, respectively. T7: T7 promoter, siRNA: small interfering RNA.

FIG. 2A is a plot for activation of the NF-κB pathway in HEK-Blue™ hTLR7 cells by transfection with modified or unmodified compounds (Cpd.1 to Cpd.6; 0.3 μg/well). The X-axis indicates different construct samples used to stimulate HEK-Blue™ hTLR7 cells. R848 was used as a positive control. The IL-4 mRNA without siRNA structure was used for comparison (modified and unmodified). Untransfected samples were used as background (BG) controls. The Y-axis indicates OD₆₂₀ BG corrected to show activation level of NF-κB pathway in HEK-Blue™ hTLR7 cells. Data represent means±standard error of the mean of 4 replicates per Cpd. Cpd. with modification are presented as black bar and unmodified Cpd. are presented as dotted bar.

FIG. 2B is a plot for activation of the JAK-STAT and ISG3 pathway in HEK-Blue™ IFN-α/β cells by supernatant of human embryonic kidney (HEK293) cells that had been transfected with modified or unmodified compounds (Cpd.1 to Cpd.6; 0.6 μg/well). The X-axis indicates different samples used to stimulate HEK-Blue™ IFN-α/β cells. IFN-alpha was used as a positive control. Supernatant of untransfected HEK293 cells was used as background control. The Y-axis indicates OD₆₂₀ BG corrected to show activation level of the JAK-STAT and ISG3 pathway in HEK-Blue™ IFN-α/β cells. Data represent means±standard error of the mean of 4 replicates per Cpd. Cpd. with modification are presented as black bar and unmodified Cpd. are presented as dotted bar.

FIG. 3A is a plot for activation of the NF-κB pathway in HEK-Blue™ hTLR7 cells by transfection with modified or unmodified compounds (Cpd.4, Cpd.6 to Cpd.9; 0.3 μg/well). Untransfected samples were used as background (BG) controls. R848 was used as a positive control. The X-axis indicates different construct samples used to stimulate HEK-Blue™ hTLR7 cells and the Y-axis indicates OD₆₂₀ (BG corrected) to show activation level of NF-κB pathway. Data represent means±standard error of the mean of 4 replicates per Cpd. Cpd. with modification are presented as black bar and unmodified Cpd. are presented as dotted bar. Significance (***, <0.001) was assessed by Student's t-test of Cpd.7 unmod. (1×siRNA at 5′ position) and Cpd. 8 unmod. (1×siRNA at 3′ position) for NF-κB pathway activation in relation to siRNA position.

FIG. 3B is a plot for activation of the JAK-STAT and ISG3 pathway in HEK-Blue™ IFN-α/β cells by supernatant of human embryonic kidney (HEK293) cells that had been transfected with modified or unmodified compounds (Cpd.4, Cpd.6 to Cpd.9; 0.3 μg/well). Supernatant of untransfected HEK293 cells was used as background (BG) control. IFN-alpha was used as a positive control. The X-axis indicates different samples used to stimulate HEK-Blue™ IFN-α/β cells and the Y-axis indicates OD₆₂₀ (BG corrected) to show activation level of the JAK-STAT and ISG3 pathway. Data represent means±standard error of the mean of 4 replicates per Cpd. Cpd. with modification are presented as black bar and unmodified Cpd. are presented as dotted bar. Significance (***, <0.001) was assessed by Student's t-test of Cpd.7 unmod. and Cpd.8 unmod. for JAK-STAT and ISG3 pathway activation in relation to siRNA position.

FIG. 4A is a plot for activation of the NF-κB pathway in HEK-Blue™ hTLR7 cells by transfection with modified or unmodified compounds (Cpd.10 to Cpd.12; 0.45 μg/well). Untransfected samples were used as background controls. R848 was used as a positive control. The X-axis indicates different construct samples used to stimulate HEK-Blue™ hTLR7 cells and the Y-axis indicates OD₆₂₀ (BG corrected) to show activation level of NF-κB pathway. Data represent means±standard error of the mean of 4 replicates per Cpd. Cpd. with modification are presented as black bar and unmodified Cpd. are presented as dotted bar.

FIG. 4B is a plot for activation of the JAK-STAT and ISG3 pathway in HEK-Blue™ IFN-α/β cells by supernatant of human embryonic kidney (HEK293) cells that had been transfected with modified or unmodified compounds (Cpd.10 to Cpd.12; 0.6 μg/well). Supernatant of untransfected HEK293 cells was used as background control. IFN-alpha was used as a positive control. The X-axis indicates different samples used to stimulate HEK-Blue™ IFN-α/β cells and the Y-axis indicates OD₆₂₀ (background corrected) to show activation level of the JAK-STAT and ISG3 pathway. Data represent means±standard error of the mean of 4 replicates per Cpd. Cpd. with modification are presented as black bar and unmodified Cpd. are presented as dotted bar.

FIG. 5A is a plot for activation of the NF-κB and AP-1 signaling pathway in HEK-Blue™ hTLR3 cells by transfection with modified or unmodified compounds (Cpd.3, Cpd.13 and Cpd.14; 0.6 μg/well). Untransfected samples were used as background (BG) controls. Poly(I.C) HMW was used as a positive control. The X-axis indicates different construct samples used to stimulate HEK-Blue™ hTLR3 cells and the Y-axis indicates OD₆₂₀ (BG corrected) to show activation level of NF-κB/AP1 pathway. Data represent means±standard error of the mean of 8 replicates per Cpd. Cpd. with modification are presented as black bar and unmodified Cpd. are presented as dotted bar. Significance (***, <0.001) was assessed by Student's t-test of Cpd.3 unmod vs. Cpd.13 unmod. for TLR3 mediated NF-κB/AP1 activation in relation to presence or absence of siRNA. Significance (*, <0.05) was assessed by Student's t-test of Cpd.3 unmod vs. Cpd.14 unmod. for TLR3 mediated NF-κB/AP1 activation in relation to presence or absence of siRNA.

FIG. 5B is a plot for activation of the NF-κB/AP-1 and IRF mediated-signaling pathway in HEK-Blue™ hTLR8 cells by transfection with modified or unmodified compounds (Cpd.3, Cpd.13 and Cpd.14; 0.6 μg/well). Untransfected samples were used as background (BG) controls. R848 was used as a positive control. The X-axis indicates different construct samples used to stimulate HEK-Blue™ hTLR8 cells and the Y-axis indicates OD₆₂₀ (BG corrected) to show activation level of NF-κB/AP1 and IRF pathway. Data represent means±standard error of the mean of 8 replicates per Cpd. Cpd. with modification are presented as black bar and unmodified Cpd. are presented as dotted bar. Significance (***, <0.001) was assessed by Student's t-test of Cpd.3 unmod vs. Cpd.13 unmod. for TLR8 mediated NF-κB/AP1/IRF activation in relation to presence or absence of siRNA. Significance (*, <0.05) was assessed by Student's t-test of Cpd.3 unmod vs. Cpd.14 unmod. for TLR3 mediated NF-κB/AP1/IRF activation in relation to presence or absence of siRNA.

FIG. 6A is a plot for IL-6 expression in Human Caucasian lung carcinoma (A549) cells as an immune response to the transfection of modified or unmodified compounds post 24 hours (Cpd.3, Cpd.4, Cpd.13 and Cpd.14; 0.3 μg/well). The X-axis indicates different construct samples used to stimulate IL-6 expression in A549 cells and the Y-axis indicates human IL-6 levels (μg/mL). Data represent means±standard error of the mean of 8 replicates per Cpd. Cpd. with modification are presented as black bar and unmodified Cpd. are presented as dotted bar. Significance (***, <0.001) was assessed by Student's t-test of Cpd.3 unmod. and Cpd. 13 unmod. for IL-6 expression as an immune response in relation to presence or absence of siRNA. Significance (***, <0.001) was assessed by Student's t-test of Cpd.4 unmod. and Cpd. 14 unmod for IL-6 expression as an immune response in relation to presence or absence of siRNA.

FIG. 6B is a plot for activation of the STAT-3 pathway in HEK-Blue™ Human IL-6 Reporter cells by equivalent volume (20 μl) of supernatant derived from Human Caucasian lung carcinoma (A549) cells that had been transfected with modified or unmodified compounds (Cpd.3, Cpd.4, Cpd.13 and Cpd.14; 0.3 μg/well). Supernatant (20 μl) of untransfected A549 cells was used as background (BG) control. Recombinant IL-6 (100 ng/mL) was used as a positive control. The X-axis indicates different samples used to stimulate HEK-Blue™ Human IL-6 Reporter cells and the Y-axis indicates OD₆₂₀ (BG corrected) to show activation level of STAT-3 pathway. Data represent means±standard error of the mean of 8 replicates per Cpd. Cpd. with modification are presented as black bar and unmodified Cpd. are presented as dotted bar. Significance (**, <0.01) was assessed by Student's t-test of Cpd.3 unmod. and Cpd. 13 unmod. for STAT3 pathway activation in relation to presence or absence of siRNA. Significance (***, <0.001) was assessed by Student's t-test of Cpd.4 unmod. and Cpd. 14 unmod. for STAT3 pathway activation in relation to presence or absence of siRNA.

FIG. 7A is a plot for IL-6 expression in Human Blood Monocytes (THP-1) cells as an immune response to the transfection of modified or unmodified compounds post 24 hours (Cpd.3, Cpd.4, Cpd.13 and Cpd.14; 0.3 μg/well). The X-axis indicates different construct samples used to stimulate IL-6 expression in THP-1 cells and the Y-axis indicates human IL-6 levels (μg/mL). Data represent means±standard error of the mean of 8 replicates per Cpd. Cpd. with modification are presented as black bar and unmodified Cpd. are presented as dotted bar. Significance (***, <0.001) was assessed by Student's t-test of Cpd.3 unmod. and Cpd. 13 unmod. for IL-6 expression as an immune response in relation to presence or absence of siRNA. Significance (***, <0.001) was assessed by Student's t-test of Cpd.4 unmod. and Cpd. 14 unmod for IL-6 expression as an immune response in relation to presence or absence of siRNA.

FIG. 7B is a plot for activation of the STAT-3 pathway in HEK-Blue™ Human IL-6 Reporter cells by equivalent volume (20 μl) of supernatant derived from Human Blood Monocytes (THP-1) cells that had been reverse transfected with modified or unmodified compounds (Cpd.3, Cpd.4, Cpd.13 and Cpd.14; 0.3 μg/well). Supernatant of untransfected THP1 cells was used as background (BG) control. Recombinant IL-6 (100 ng/mL) was used as a positive control. The X-axis indicates different samples used to stimulate HEK-Blue™ Human IL-6 Reporter cells and the Y-axis indicates OD₆₂₀ (BG corrected) to show activation level of STAT-3 pathway. Data represent means±standard error of the mean of 8 replicates per Cpd. Cpd. with modification are presented as black bar and unmodified Cpd. are presented as dotted bar. Significance (***, <0.001) was assessed by Student's t-test of Cpd.3 unmod. and Cpd. 13 unmod. for STAT3 pathway activation in relation to presence or absence of siRNA. Significance (***, <0.001) was assessed by Student's t-test of Cpd.4 unmod. and Cpd. 14 unmod. for STAT3 pathway activation in relation to presence or absence of siRNA.

### DETAILED DESCRIPTION

Provided herein are compositions and methods for modulating expression of two or more genes, comprising recombinant polynucleic acid or RNA constructs comprising at least one nucleic acid sequence encoding a gene of interest and at least two nucleic acid sequences each encoding or comprising a genetic element that modulates expression of a target RNA. A cell or cells contacted with compositions comprising recombinant polynucleic acid or RNA constructs described herein can have reduced level of innate immune activity and lower immune response and can increase the efficiency of modulating expression of two or more genes in the cell or cells.

Recombinant polynucleic acid or RNA construct compositions provided herein may further comprise one or more linkers. In one instance, recombinant polynucleic acid or RNA constructs may comprise nucleic acid sequences encoding or comprising at least two genetic elements that modulate expression of one or more target RNAs and one or more linkers, wherein a linker may be present between each of at least two genetic elements that modulate expression of one or more target RNAs. In another instance, recombinant polynucleic acid or RNA constructs may comprise nucleic acid sequences encoding one or more genes of interest and one or more linkers, wherein a linker may be present between each of one or more genes of interest. In some instances, recombinant polynucleic acid or RNA constructs may comprise nucleic acid sequences encoding one or more genes of interest, nucleic acid sequences encoding or comprising at least two genetic elements that modulate expression of one or more target RNAs, and one or more linkers, wherein a linker may be present between nucleic acid sequences encoding one or more genes of interest and nucleic acid sequences encoding or comprising at least two genetic elements that modulate expression of one or more target RNAs, between each of at least two genetic elements that modulate expression of one or more target RNAs, and/or between each of one or more genes of interest. In some embodiments, recombinant polynucleic acid or RNA constructs described herein may comprise at least three genetic elements that modulate expression of one or more target RNAs. In some embodiments, recombinant polynucleic acid or RNA constructs described herein may comprise at least six genetic elements that modulate expression of one or more target RNAs.

Also provided herein are vectors comprising recombinant polynucleic acid constructs described herein or encoding recombinant RNA constructs described herein. Provided herein are cells comprising recombinant polynucleic acid or RNA construct composition or vectors described herein. Recombinant polynucleic acid or RNA construct compositions described herein can be formulated into pharmaceutical compositions. Further provided herein are compositions and methods to modulate expression of two or more genes in parallel.

Provided herein are compositions and methods for treating a disease or a condition comprising administering to a subject in need thereof compositions or pharmaceutical compositions described herein. Recombinant polynucleic acid or RNA construct compositions provided herein may comprise a first RNA sequence and a second RNA sequence. In one example, the first RNA sequence or the second RNA sequence may comprise one or more messenger RNAs (mRNAs) and can increase the level of proteins encoded by mRNAs. In another example, the first RNA sequence or the second RNA sequence may be a genetic element that modulates expression of a target RNA. For example, the first RNA sequence or the second RNA sequence may comprise small interfering RNAs (siRNAs) capable of binding to one or more target RNAs and can downregulate the levels of protein encoded by target RNAs. For example, mRNAs and target RNAs may be of genes associated diseases and conditions described herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Definitions

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “RNA” as used herein includes RNA which encodes an amino acid sequence (e.g., mRNA, etc.) as well as RNA which does not encode an amino acid sequence (e.g., siRNA, shRNA, miRNA etc.). The RNA as used herein may be a coding RNA, i.e., an RNA which encodes an amino acid sequence. Such RNA molecules are also referred to as mRNA (messenger RNA) and are single-stranded RNA molecules. The RNA as used herein may be a non-coding RNA, i.e., an RNA which does not encode an amino acid sequence or is not translated into a protein. A non-coding RNA can include, but is not limited to, a small interfering RNA (siRNA), a short or small harpin RNA (shRNA), a microRNA (miRNA), a piwi-interacting RNA (piRNA), and a long non-coding RNA (IncRNA). siRNAs as used herein may comprise a double-stranded RNA (dsRNA) region, a hairpin structure, a loop structure, or any combinations thereof. In some embodiments, siRNAs may comprise at least one shRNA, at least one dsRNA region, or at least one loop structure. In some embodiments, siRNAs may be processed from a dsRNA or an shRNA. In some embodiments, siRNAs may be processed or cleaved by an endogenous protein, such as DICER, from an shRNA. In some embodiments, a hairpin structure or a loop structure may be cleaved or removed from an siRNA. For example, a hairpin structure or a loop structure of an shRNA may be cleaved or removed. In some embodiments, RNAs described herein may be made by synthetic, chemical, or enzymatic methodology known to one of ordinary skill in the art, made by recombinant technology known to one of ordinary skill in the art, or isolated from natural sources, or made by any combinations thereof. The RNA may comprise modified or unmodified nucleotides or mixtures thereof, e.g., the RNA may optionally comprise chemical and naturally occurring nucleoside modifications known in the art (e.g., N¹-Methylpseudouridine also referred herein as methylpseudouridine).

The terms “nucleic acid sequence,” “polynucleic acid sequence,” and “nucleotide sequence” are used herein interchangeably and have the identical meaning herein and refer to DNA or RNA. In some embodiments, a nucleic acid sequence is a polymer comprising or consisting of nucleotide monomers, which are covalently linked to each other by phosphodiester-bonds of a sugar/phosphate-backbone. The terms “nucleic acid sequence,” “polynucleic acid sequence,” and “nucleotide sequence” may encompass unmodified nucleic acid sequences, i.e., comprise unmodified nucleotides or natural nucleotides The terms “nucleic acid sequence,” “polynucleic acid sequence,” and “nucleotide sequence” may also encompass modified nucleic acid sequences, such as base-modified, sugar-modified or backbone-modified etc., DNA or RNA.

The terms “natural nucleotide” and “canonical nucleotide” are used herein interchangeably and have the identical meaning herein and refer to the naturally occurring nucleotide bases adenine (A), guanine (G), cytosine (C), uracil (U), thymine (T).

The term “unmodified nucleotide” is used herein to refer to natural nucleotides which are not naturally modified e.g., which are not epigenetically or post-transcriptionally modified in vivo. Preferably the term “unmodified nucleotides” is used herein to refer to natural nucleotides which are not naturally modified e.g., which are not epigenetically or post-transcriptionally modified in vivo and which are not chemically modified e.g., which are not chemically modified in vitro.

The term “modified nucleotide” is used herein to refer to naturally modified nucleotides such as epigenetically or post-transcriptionally modified nucleotides and to chemically modified nucleotides e.g., nucleotides which are chemically modified in vitro.

### Recombinant RNA Constructs

Provided herein are compositions comprising recombinant RNA constructs comprising at least one nucleic acid sequence encoding a gene of interest and/or at least two nucleic acid sequences each comprising a genetic element that modulate expression of a target RNA. In some instances, a genetic element that modulates the expression of one or more target RNAs, e.g., a siRNA, may comprise a nucleic acid sequence comprising a sense siRNA strand and an anti-sense siRNA strand. For example, in some instances, recombinant RNA constructs may comprise at least 1 species of a genetic element that modulates the expression of one or more target RNAs, e.g., at least 1 species of siRNA, such as a nucleic acid sequence comprising a sense strand of siRNA and a nucleic acid sequence comprising an anti-sense strand of siRNA. 1 species of a genetic element that modulates the expression of one or more target RNAs, e.g., 1 species of siRNA, as described herein, can refer to 1 species of sense strand siRNA and 1 species of anti-sense strand siRNA. In some instances, a recombinant RNA construct may comprise at least two species of genetic elements that modulate the expression of one or more target RNAs, e.g., at least two species of siRNA. For example, a recombinant RNA construct may comprise 2, 3, 4, 5, 6, 7, 8, 9, 10, or more species of genetic elements that modulate the expression of one or more target RNAs, e.g., 2, 3, 4, 5, 6, 7, 8, 9, 10, or more species of siRNA comprising a sense strand of siRNA and an anti-sense strand of siRNA. In some embodiments, recombinant RNA constructs may comprise 1 to 20 species of genetic elements that modulate the expression of one or more target RNAs, e.g., 1 to 20 species of siRNA. In some embodiments, recombinant RNA constructs may comprise at least 1, 2, 3, 4, 5, 6, 7, 8, 9, or at least 10 species of genetic elements that modulate the expression of one or more target RNAs, e.g., at least 1, 2, 3, 4, 5, 6, 7, 8, 9, or at least 10 species of siRNA. In some embodiments, recombinant RNA constructs may comprise at most 2, 3, 4, 5, 6, 7, 8, 9, 10, 11, 12, 13, 14, 15, 16, 17, 18, 19, or at most 20 species of genetic elements that modulate the expression of one or more target RNAs, e.g., at most 2, 3, 4, 5, 6, 7, 8, 9, 10, 11, 12, 13, 14, 15, 16, 17, 18, 19, or at most 20 species of siRNA. In some embodiments, recombinant RNA constructs may comprise at most 3, 4, 5, 6, 7, 8, 9, 10, 11, 12, 13, 14, 15, 16, 17, 18, 19, or at most 20 species of genetic elements that modulate the expression of one or more target RNAs, e.g., at most 3, 4, 5, 6, 7, 8, 9, 10, 11, 12, 13, 14, 15, 16, 17, 18, 19, or at most 20 species of siRNA.

In some embodiments, recombinant RNA constructs may comprise between 2 genetic elements that modulate the expression of one or more target RNAs and 10 genetic elements that modulate the expression of one or more target RNAs, between 3 genetic elements that modulate the expression of one or more target RNAs and 10 genetic elements that modulate the expression of one or more target RNAs, between 4 genetic elements that modulate the expression of one or more target RNAs and 10 genetic elements that modulate the expression of one or more target RNAs, between 5 genetic elements that modulate the expression of one or more target RNAs and 10 genetic elements that modulate the expression of one or more target RNAs, between 6 genetic elements that modulate the expression of one or more target RNAs and 10 genetic elements that modulate the expression of one or more target RNAs, between 7 genetic elements that modulate the expression of one or more target RNAs and 10 genetic elements that modulate the expression of one or more target RNAs, between 9 genetic elements that modulate the expression of one or more target RNAs and 10 genetic elements that modulate the expression of one or more target RNAs, preferably between 2 genetic elements that modulate the expression of one or more target RNAs and 6 genetic elements that modulate the expression of one or more target RNAs, between 3 genetic elements that modulate the expression of one or more target RNAs and 6 genetic elements that modulate the expression of one or more target RNAs, or between 4 genetic elements that modulate the expression of one or more target RNAs and 6 genetic elements that modulate the expression of one or more target RNAs. In a preferred embodiment, recombinant RNA constructs described herein comprise at least 2 species of genetic elements that modulate the expression of one or more target RNAs. In another preferred embodiment, recombinant RNA constructs described herein comprise at least 3 species of genetic elements that modulate the expression of one or more target RNAs. In yet another preferred embodiment, recombinant RNA constructs described herein comprise at least 6 species of genetic elements that modulate the expression of one or more target RNAs. For example, recombinant RNA constructs may comprise between 2 siRNAs and 10 siRNAs, between 3 siRNAs and 10 siRNAs, between 4 siRNAs and 10 siRNAs, between 5 siRNAs and 10 siRNAs, between 6 siRNAs and 10 siRNAs, between 7 siRNAs and 10 siRNAs, between 9 siRNAs and 10 siRNAs, preferably between 2 siRNAs and 6 siRNAs, between 3 siRNAs and 6 siRNAs, or between 4 siRNAs and 6 siRNAs. In a preferred embodiment, recombinant RNA constructs described herein comprise at least 2 species of siRNAs. In another preferred embodiment, recombinant RNA constructs described herein comprise at least 3 species of siRNAs. In yet another preferred embodiment, recombinant RNA constructs described herein comprise at least 6 species of siRNAs.

Recombinant RNA construct compositions provided herein may further comprise one or more linkers. In one instance, recombinant RNA constructs may comprise nucleic acid sequences comprising at least two genetic elements that modulate expression of one or more target RNAs and one or more linkers, wherein a linker may be present between each of at least two genetic elements that modulate expression of one or more target RNAs. In another instance, recombinant RNA constructs may comprise nucleic acid sequences encoding one or more genes of interest and one or more linkers, wherein a linker may be present between each of one or more genes of interest. In some instances, recombinant RNA constructs may comprise nucleic acid sequences encoding one or more genes of interest, nucleic acid sequences comprising at least two genetic elements that modulate expression of one or more target RNAs, and one or more linkers, wherein a linker may be present between nucleic acid sequences encoding one or more genes of interest and nucleic acid sequences comprising at least two genetic elements that modulate expression of one or more target RNAs; between each of at least two genetic elements that modulate expression of one or more target RNAs; and/or between each of one or more genes of interest.

Provided herein are compositions for modulating expression of two or more genes comprising recombinant RNA constructs comprising at least one nucleic acid sequence encoding a gene of interest and/or at least two nucleic acid sequences each comprising a genetic element that modulates expression of a target RNA. Further provided herein are compositions for treating a disease or a condition comprising recombinant RNA constructs comprising at least one nucleic acid sequence encoding a gene of interest and/or at least two nucleic acid sequences each comprising a genetic element that modulates expression of a target RNA. Recombinant RNA construct compositions provided herein may comprise a first RNA sequence and a second RNA sequence. In one example, the first RNA sequence or the second RNA sequence may comprise one or more messenger RNAs (mRNAs) and can increase the level of proteins encoded by mRNAs. In another example, the first RNA sequence or the second RNA sequence may be a genetic element that modulates expression of a target RNA. In some embodiments, the genetic element that modulates expression of a target RNA may be a small interfering RNA (siRNA) capable of binding to one or more target RNAs. For example, the first RNA sequence or the second RNA sequence may comprise siRNAs capable of binding to one or more target RNAs and can downregulate the levels of protein encoded by target RNAs. In some instances, the genetic element that modulates expression of a target RNA does not inhibit the expression of the gene of interest. In some instances, mRNAs and target RNAs may be of genes associated diseases and conditions described herein. Also provided herein are compositions and methods to modulate expression of two or more genes in parallel using a single RNA transcript.

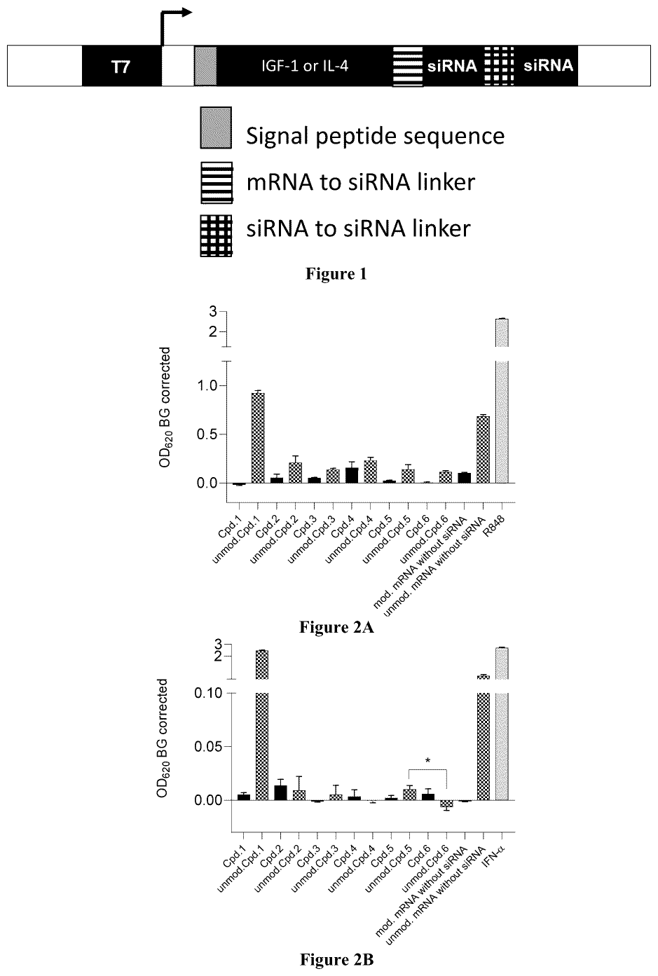

Further provided herein are recombinant polynucleic acid or RNA constructs comprising a gene of interest and at least two genetic elements that reduces expression of another gene, such as siRNA, wherein the gene of interest and the genetic element that reduces expression of another gene such as siRNA may be present in a sequential manner from the 5′ to 3′ direction, as illustrated in FIG. 1, or from 3′ to 5′ direction. In one example, the gene of interest (e.g., IGF-1 or IL-4) can be present 5′ to or upstream of the genetic element that reduces expression of another gene such as siRNA, and the gene of interest can be linked to siRNA by a linker (mRNA to siRNA/shRNA linker, can be also referred as a “spacer”), as illustrated in FIG. 1. In another example, the gene of interest may be present 3′ to or downstream of the genetic element that reduces expression of another gene such as siRNA, and siRNA can be linked to the gene of interest by a linker (siRNA/shRNA to mRNA linker, can be also referred as a “spacer”). Recombinant polynucleic acid or RNA constructs provided herein may comprise more than one species of siRNAs and each of more than one species of siRNAs can be linked by a linker (siRNA to siRNA or shRNA to shRNA linker). In some embodiments, the sequence of mRNA to siRNA (or siRNA to mRNA) linker and the sequence of siRNA to siRNA (or shRNA to shRNA) linker may be different. In some embodiments, the sequence of mRNA to siRNA/shRNA (or siRNA/shRNA to mRNA) linker and the sequence of siRNA to siRNA (or shRNA to shRNA) linker may be the same. Recombinant polynucleic acid or RNA constructs provided herein may comprise more than one gene of interest and each of more than one gene of interest can be linked by a linker (mRNA to mRNA linker). In some instances, a gene of interest may comprise a signal peptide sequence at the N-terminus as shown in FIG. 1. In some instances, a gene of interest may comprise unmodified (WT) signal peptide sequence or modified signal peptide sequence. Recombinant polynucleic acid constructs (e.g., DNA constructs) provided herein may also comprise a promoter sequence for RNA polymerase binding. For example, DNA constructs may comprise a promoter sequence for DNA-dependent RNA polymerase binding to express RNA constructs described herein. As an example, T7 promoter for T7 RNA polymerase binding is shown in FIG. 1. In some embodiments, RNA constructs described herein may not comprise a promoter sequence.

A recombinant polynucleic acid or a recombinant RNA can refer to a polynucleic acid or RNA that is not naturally occurring and is synthesized or manipulated in vitro. A recombinant polynucleic acid or RNA can be synthesized in a laboratory and can be prepared by using recombinant DNA or RNA technology by using enzymatic modification of DNA or RNA, such as enzymatic restriction digestion, ligation, cloning, and/or in vitro transcription. A recombinant polynucleic acid can be transcribed in vitro to produce a messenger RNA (mRNA) and recombinant mRNAs can be isolated, purified, and used for transfection into a cell. A recombinant polynucleic acid or RNA used herein can encode a protein, polypeptide, a target motif, a signal peptide, and/or a non-coding RNA such as small interfering RNA (siRNA). In some embodiments, under suitable conditions, a recombinant polynucleic acid or RNA can be incorporated into a cell and expressed within the cell.

Exogenous nucleic acids such as recombinant polynucleic acids including recombinant RNAs can induce an innate immune response when introduced into cells. Exogenous RNAs can be recognized by different types of innate immune receptors including cell surface, endosomal, and cytosolic innate immune receptors. Endosomal innate immune receptors include, but are not limited to, toll-like receptor 3 (TLR3), TLR7, and TLR8. Examples of cytosolic innate immune receptors include, but are not limited to, retinoic acid inducible gene 1 (RIG1), melanoma differentiation-associated gene 5 (MDA5), NOD-like receptor, pyrin domain containing 3 (NLRP3), and nucleotide-binding and oligomerization domain containing 2 (NOD2). TLR3, RIG1 and MDA5 can recognize double-stranded RNAs (dsRNAs), and TLR7 and TLR8 can recognize single-stranded RNAs (ssRNAs) and RNA degradation products (e.g., uridine and guanosine-uridine-rich fragments). These RNA sensors (e.g., TLR3, TLR7, TLR8, RIG1, MDA5, NOD2, etc.) can activate interferon regulatory factor (IRF)-mediated signaling and nuclear factor KB (NF-κB)-mediated signaling to increase production of type I interferons (IFN-α/β) and pro-inflammatory cytokines or, in case of NLRP3, can initiate inflammasome formation and processes cytokines for maturation. As such, it is of great interest and importance to develop polynucleic acid and RNA therapeutics that induces less innate immune response. Recombinant polynucleic acid or recombinant RNA compositions, as described herein, can provide means to modulate two or more genes in parallel with reduced level of innate immune activity and lower immune response.

Provided herein are compositions and methods for modulating expression of two or more genes, comprising recombinant RNA constructs comprising a first RNA sequence encoding at least one gene of interest and a second RNA sequence comprising at least two genetic elements that modulate expression of one or more target RNAs. Recombinant RNA constructs, as described herein, comprising an RNA sequence encoding at least one gene of interest and an RNA sequence comprising at least two genetic elements that modulate expression of one or more target RNAs and can be advantageous in reducing or inducing lower levels of immune response over recombinant RNA constructs comprising an RNA sequence encoding at least one gene of interest and RNA sequence comprising less than two (e.g., 0 or 1) genetic elements that modulate expression of one or more target RNAs. For example, contacting a cell with recombinant RNA constructs described herein can result in an immune response that is lower than the immune response of the cell contacted with corresponding recombinant RNA constructs comprising the first RNA sequence encoding a gene of interest and a corresponding second RNA sequence comprising at most one genetic element that modulates expression of a target RNA. In some embodiments, the corresponding recombinant RNA constructs may comprise a second RNA sequence comprising one genetic element that modulates expression of a target RNA. In some embodiments, the corresponding recombinant RNA constructs may comprise a second RNA sequence that does not comprise any genetic elements that modulate expression of a target RNA. In some embodiments, the cell or the cells are of human origin.

Immune responses, as described herein, can be measured by any methods known in the art. In one example, immune responses can be assessed by measuring secretion of cytokines, expression of DC activation markers, or the ability to act as an adjuvant for an adaptive immune response. In another example, immune responses can be measured using any kits known in the art, e.g., commercially available kits. Details of methods for measuring immune responses are described in Examples. In some embodiments, the immune response described herein is a human immune response. In some embodiments, the immune response is a human TLR7 immune response, a human TLR3 immune response, or an IFNα/β immune response, or any combination thereof. In some embodiments, the immune response described herein is a human immune response. In some embodiments, the immune response is a human TLR7 immune response, or an IFNα/β immune response, or any combinations thereof. A human TLR7 immune response can be measured by using a human TLR7 immunogenicity assay. In some embodiments, the human TLR7 immunogenicity assay can be performed in HEK293 cells that are engineered to express human TLR7 gene and a reporter gene. In some embodiments, the reporter gene can be a secreted reporter gene. For examples, a secreted reporter gene can include, but is not limited to, secreted embryonic alkaline phosphatase (SEAP). In some embodiments, the reporter gene can be under a control of a promoter with one or more NF-κB and/or activator protein 1 (AP1) binding sites. In some embodiments, the promoter is an IFN-β minimal promoter. In some embodiments, the human TLR7 immunogenicity assay can measure activation of NF-κB and/or AP1.

A human TLR3 immune response can be measured by using a human TLR3 immunogenicity assay. In some embodiments, the human TLR3 immunogenicity assay can be performed in HEK293 cells that are engineered to express human TLR3 gene and a reporter gene. In some embodiments, the reporter gene can be a secreted reporter gene. For examples, a secreted reporter gene can include, but is not limited to, secreted embryonic alkaline phosphatase (SEAP). In some embodiments, the reporter gene can be under a control of a promoter with one or more NF-κB and/or AP1 binding sites. In some embodiments, the promoter is an IFN-β minimal promoter. In some embodiments, the human TLR 3 immunogenicity assay can measure activation of NF-κB and/or AP1.

An IFNα/β immune response can be measured by using an IFNα/β immunogenicity assay. In some embodiments, the IFNα/β immunogenicity assay can be performed in HEK293 cells that are engineered to express human signal transducer and activator of transcription 2 (STAT2) gene and/or IRF9 gene and a reporter gene. In some embodiments, the reporter gene can be a secreted reporter gene. For examples, a secreted reporter gene can include, but is not limited to, secreted embryonic alkaline phosphatase (SEAP). In some embodiments, the reporter gene can be under a control of a promoter with one or more STAT2 and/or IRF9 binding sites. In some embodiments, the promoter is an interferon stimulated gene factor 54 (ISG54) promoter. In some embodiments, the IFNα/β immunogenicity assay can measure activation of Janus kinase (JAK)-STAT and/or ISG3.

In some embodiments, contacting the human cell with recombinant RNA constructs described herein can result in an immune response that is lower than the immune response of the human cell contacted with the corresponding recombinant RNA construct comprising at most one genetic element that modulates expression of one or more target RNAs according to a human TLR7 immunogenicity assay described herein. In some embodiments, contacting the human cell with recombinant RNA constructs described herein can result in an immune response that is lower than the immune response of the human cell contacted with the corresponding recombinant RNA construct comprising at most one genetic element that modulates expression of one or more target RNAs according to a human TLR3 immunogenicity assay described herein. In some embodiments, contacting the human cell with recombinant RNA constructs described herein can result in an immune response that is lower than the immune response of the human cell contacted with the corresponding recombinant RNA construct comprising at most one genetic element that modulates expression of one or more target RNAs according to an IFNα/β immunogenicity assay described herein. In some embodiments, the immune response in the human cell contacted with the recombinant RNA construct may be at least about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.6 fold, about 2.7 fold, about 2.8 fold, about 2.9 fold, about 3.0 fold, about 3.1 fold, about 3.2 fold, about 3.3 fold, about 3.4 fold, about 3.5 fold, about 3.6 fold, about 3.7 fold, about 3.8 fold, about 3.9 fold, about 4.0 fold, about 4.1 fold, about 4.2 fold, about 4.3 fold, about 4.4 fold, about 4.5 fold, about 4.6 fold, about 4.7 fold, about 4.8 fold, about 4.9 fold, about 5.0 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 105 fold, about 110 fold, about 115 fold, about 120 fold, about 125 fold, about 130 fold, about 135 fold, about 140 fold, about 145 fold, or at least about 150 fold less than the immune response in the human cell contacted with the corresponding recombinant RNA construct comprising at most one genetic element that modulates expression of one or more target RNAs. In some embodiments, the immune response in the human cell contacted with the recombinant RNA construct may be from about 1.1 fold to about 10 fold, from about 1.5 fold to about 15 fold, from about 2.0 fold to about 20 fold, from about 2.5 fold to about 25 fold, from about 3.0 fold to about 30 fold, from about 3.5 fold to about 35 fold, from about 4.0 fold to about 40 fold, from about 4.5 fold to about 45 fold, from about 5.0 fold to about 50 fold, from about 5.5 fold to about 55 fold, from about 6.0 fold to about 60 fold, from about 6.5 fold to about 65 fold, from about 7.0 fold to about 70 fold, from about 7.5 fold to about 75 fold, from about 8.0 fold to about 80 fold, from about 8.5 fold to about 85 fold, from about 9.0 fold to about 90 fold, from about 9.5 fold to about 95 fold, from about 10 fold to about 100 fold, from about 15 fold to about 150 fold, from about 20 fold to about 200 fold, from about 25 fold to about 250 fold, from about 30 fold to about 300 fold, from about 35 fold to about 350 fold, from about 40 fold to about 400 fold, from about 45 fold to about 450 fold, or from about 50 fold to about 500 fold less than the immune response in the human cell contacted with the corresponding recombinant RNA construct comprising at most one genetic element that modulates expression of one or more target RNAs. In some embodiments, the immune response in the human cell contacted with the recombinant RNA construct described herein is reduced by at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%. 98%, 99%, 99.5%, or at least 99.9% as compared to the immune response in the human cell contacted with the corresponding recombinant RNA construct comprising at most one genetic element that modulates expression of one or more target RNAs.

In some instances, recombinant RNA constructs described herein may not trigger any detectable immune response as measured by any methods described herein. For example, contacting a human cell with recombinant RNA constructs described herein may not result in a substantial immune response according to a immunogenicity assay described herein (e.g., human TLR3 immunogenicity assay, human TLR7 immunogenicity assay, or IFNα/β immunogenicity assay, etc.).

Provided herein are compositions comprising recombinant RNA constructs comprising a first RNA sequence and a second RNA sequence, wherein the first RNA sequence and/or the second RNA sequence may encode a gene of interest or a genetic element that modulates expression of a target RNA. In one example, the first RNA sequence or the second RNA sequence may be an mRNA encoding a gene of interest. In another example, the first RNA sequence or the second RNA sequence may be a genetic element that reduces expression of a target RNA, such as a small interfering RNA (siRNA) capable of binding to a target RNA. In some embodiments, the siRNA capable of binding to a target RNA is not a part of an intron sequence encoded by the gene of interest. In some instances, the gene of interest is expressed without RNA splicing. In some instances, the siRNA capable of binding to a target RNA is not encoded by or comprised of an intron sequence of the gene of interest. In some instances, the siRNA capable of binding to a target RNA binds to an exon of a target RNA. In some instances, the siRNA capable of binding to a target RNA specifically binds to one target RNA.

Recombinant RNA constructs provided herein may comprise multiple copies of a gene of interest, wherein each of the multiple copies of a gene of interest encodes the same protein. Also provided herein are compositions comprising recombinant RNA constructs comprising multiple genes of interest, wherein each of the multiple genes of interest encodes a different protein. In some embodiments, recombinant RNA constructs provided herein may comprise a combination of multiple copies of a gene of interest encoding the same protein and multiple genes of interest each of which encodes a different protein.

Recombinant RNA constructs provided herein may comprise more than one nucleic acid sequences encoding a gene of interest. For example, recombinant RNA constructs may comprise 2, 3, 4, 5, 6, 7, 8, 9, 10, 11, 12, 13, 14, 15, or more nucleic acid sequences encoding a gene of interest. In some instances, each of the two or more nucleic acid sequences may encode the same gene of interest, wherein the mRNA encoded by the same gene of interest is different from the siRNA target mRNA. In some instances, each of the two or more nucleic acid sequences may encode a different gene of interest, wherein the mRNA encoded by the different gene of interest is not a target of siRNA comprised in the same RNA construct. In some instances, recombinant RNA constructs may comprise three or more nucleic acid sequences encoding a gene of interest, wherein each of the three or more nucleic acid sequences may encode the same gene of interest or a different gene of interest, and wherein mRNAs encoded by the same or the different gene of interest are not a target of siRNA comprised in the same RNA construct. For example, recombinant RNA constructs may comprise four nucleic acid sequences encoding a gene of interest, wherein three of the four nucleic acid sequences encode the same gene of interest and one of the four nucleic acid sequences encodes a different gene of interest, and wherein mRNAs encoded by the same or different gene of interest are not a target of siRNA comprised in the same RNA construct.

Recombinant RNA constructs provided herein may comprise multiple species of siRNAs, wherein each of the multiple species of siRNAs is capable of binding to the same target RNA. In some embodiments, each of the multiple species of siRNAs may bind to the same region of the same target RNA. In some embodiments, each of the multiple species of siRNAs may bind to a different region of the same target RNA. In some embodiments, some of the multiple species of siRNAs may bind to the same target RNA and some of the multiple species of siRNAs may bind to a different region of the same target RNA. Also provided herein are recombinant RNA constructs comprising multiple species of siRNAs, wherein each of the multiple species of siRNAs is capable of binding to a different target RNA. In some embodiments, the target RNA is a noncoding RNA. In some embodiments, the target RNA is a messenger (mRNA).

Recombinant RNA constructs provided herein may comprise at least two species of siRNA targeting an RNA of a gene associated with a disease or a condition described herein. For example, recombinant RNA constructs provided herein may comprise 2-10 species of siRNA targeting the same RNA or different RNAs. In some instances, each of the 2-10 species of siRNA targeting the same RNA may comprise the same sequence, i.e., each of the 2-10 species of siRNA binds to the same region of the target RNA. In some instances, each of the 2-10 species of siRNA targeting the same RNA may comprise different sequences, i.e., each of the 2-10 species of siRNA binds to different regions of the target RNA. For instance, recombinant RNA constructs provided herein, may comprise 3 species of siRNA targeting one RNA and each of the 3 species of siRNA comprise the same nucleic acid sequence to target the same region of the RNA. In this example, each of the 3 species of siRNA may comprise the same nucleic acid sequence to target exon 1. In another example, each of the 3 species of siRNA may comprise different nucleic acid sequence to target different regions of the RNA. In this example, one of the 3 species of siRNA may comprise a nucleic acid sequence targeting exon 1 and another one of the 3 species of siRNA may comprise a nucleic acid sequence targeting exon 2, etc. In yet another example, each of the 3 species of siRNA may comprise different nucleic acid sequence to target different RNAs. In all aspects, siRNAs in recombinant RNA constructs provided herein may not affect the expression of the gene of interest, expressed by the mRNA in the same RNA construct compositions. In some embodiments, recombinant RNA constructs provided herein may comprise 6 species of siRNA capable of binding to one or more target RNAs. In some embodiments, the target RNA is an mRNA.

Provided herein are compositions comprising recombinant RNA constructs may further comprise a linker. In some instances, a linker described herein may have a structure of Formula (I) XₘCAACAAXₙ, wherein X is any nucleotide, m is an integer from 1 to 12, and n is an integer from 0 to 4 (SEQ ID NO: 129). In some instances, a linker described herein may have a structure of Formula (II): XₚTCCCXᵣ, wherein X is any nucleotide, p is an integer from 0 to 17, and r is an integer from 0 to 13 (SEQ ID NO: 130). In one embodiment, the first RNA sequence or the second RNA sequence may comprise at least two genetic elements that modulate the expression of one or more target RNAs and the linker RNA sequence may connect each of the at least two genetic elements that modulate the expression of one or more target RNAs (e.g., siRNA to siRNA linker or shRNA to shRNA linker). In another embodiment, the first RNA sequence may encode a gene of interest and the second RNA sequence may comprise at least two genetic elements that modulate the expression of one or more target RNA, and the linker RNA sequence may connect the gene of interest and the at least two genetic elements that modulate the expression of one or more target RNAs (e.g., mRNA to siRNA linker, siRNA to mRNA, mRNA to shRNA linker, or shRNA to mRNA linker). In some embodiments, the sequence of mRNA to siRNA/shRNA (or siRNA/shRNA to mRNA) linker and the sequence of siRNA to siRNA (or shRNA to shRNA) linker may be different. In some embodiments, the sequence of mRNA to siRNA/shRNA (or siRNA/shRNA to mRNA) linker and the sequence of siRNA to siRNA (or shRNA to shRNA) linker may be the same. In some embodiments, the first RNA sequence may encode a gene of interest and the second RNA sequence may comprise at least two genetic elements that modulate the expression of one or more target RNA, and the same RNA linker sequence may connect the gene of interest and the at least two genetic elements that modulate the expression of one or more target RNAs (e.g., mRNA to siRNA/shRNA linker or siRNA/shRNA to mRNA linker) and between each of the at least two genetic elements that modulate the expression of one or more target RNAs (e.g., siRNA/shRNA to siRNA/shRNA linker).

In some embodiments, the length of a linker is from about 4 to about 50, from about 4 to about 45, or from about 4 to about 40, from about 4 to about 35, or from about 4 to about 30 nucleotides. In some embodiments, the length of a linker is from about 4 to about 27 nucleotides. In some embodiments, the length of a linker is from about 4 to about 18 nucleotides. For example, the length of a linker is about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleotides. In some embodiments, the length of a linker can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or at most about 50 nucleotides. In some embodiments, the length of a linker is 4 nucleotides. In some embodiments, the length of a linker is 7 nucleotides. In some embodiments, the length of a linker is 11 nucleotides. In some embodiments, the length of a linker is 12 nucleotides. In some embodiments, the length of a linker is 18 nucleotides.

In some instances, a linker described herein may have a structure of Formula (I) XₘCAACAAXₙ, wherein X is any nucleotide; m is an integer from 1 to 12, and n is an integer from 0 to 4 (SEQ ID NO: 129); and m is 1 and n is 0. In some instances, a linker described herein may comprise a sequence comprising CAACAA (SEQ ID NO: 91), TCCC (SEQ ID NO: 89), or ACAACAA (SEQ ID NO: 85). In some embodiments, a linker may comprise a sequence selected from the group consisting of ATCCCTACGTACCAACAA (SEQ ID NO: 87), ACGTACCAACAA (SEQ ID NO: 88), TCCC (SEQ ID NO: 89), ACAACAATCCC (SEQ ID NO: 90), and ACAACAA (SEQ ID NO: 85). In some embodiments, a linker may comprise a sequence comprising ACAACAA (SEQ ID NO: 85), ATAGTGAGTCGTATTATCCC (SEQ ID NO: 92), ATAGTGAGTCGTATTAACAACAATCCC (SEQ ID NO: 93), ATAGTGAGTCGTATTAACAACAA (SEQ ID NO: 94), ATAGTGAGTCGTATTAATCCCTACGTACCAACAA (SEQ ID NO: 95), or ATAGTGAGTCGTATTAACGTACCAACAA (SEQ ID NO: 27). In some embodiments, a linker may comprise a sequence comprising ACAACAA (SEQ ID NO: 85). In some embodiments, a linker described herein may comprise a sequence selected from the group consisting of SEQ ID NOs: 27, 28, 85-95. In some embodiments, a linker described herein may not comprise a sequence comprising TTTATCTTAGAGGCATATCCCTACGTACCAACAA (SEQ ID NO: 28). In some embodiments, a linker described herein may not comprise a sequence comprising ATAGTGAGTCGTATTAACGTACCAACAA (SEQ ID NO: 27). In some embodiments, a linker described herein does not comprise

(SEQ ID NO: 28)
TTTATCTTAGAGGCATATCCCTACGTACCAACAA
or
(SEQ ID NO: 27)
ATAGTGAGTCGTATTAACGTACCAACAA.

In some instances, a tRNA linker can be used. The tRNA system is evolutionarily conserved cross living organism and utilizes endogenous RNases P and Z to process multicistronic constructs (Dong et al., 2016). In some instances, tRNA linkers described herein may comprise a nucleic acid sequence comprising AACAAAGCACCAGTGGTCTAGTGGTAGAATAGTACCCTGCCACGGTACAGACCC GGGTTCGATTCCCGGCTGGTGCA (SEQ ID NO: 96). In some instances, a linker comprising a nucleic acid sequence comprising ATAGTGAGTCGTATTAACGTACCAACAA (SEQ ID NO: 27) may be used to link the first RNA sequence and the second RNA sequence. In some embodiments, a linker comprising a nucleic acid sequence comprising TTTATCTTAGAGGCATATCCCTACGTACCAACAA (SEQ ID NO: 28) may be used to connect each of the 1-20 or more siRNA species.

In some instances, linkers described herein may not form a secondary structure. For example, linkers described herein may not bind to or basepairs with a nucleic acid sequence of recombinant RNA constructs provided herein. In some embodiments, a inker RNA sequence described herein does not form a secondary structure according to RNAfold WebServer. In some embodiments, an siRNA sequence described herein may form a secondary structure according to RNAfold WebServer.

Further provided herein are recombinant RNA construct compositions comprising 1-20 or more siRNA species, wherein each of the 1-20 or more siRNA species are connected by a linker having a structure selected from the group consisting of Formula (I): XₘCAACAAXₙ, wherein X is any nucleotide, m is an integer from 1 to 12, and n is an integer from 0 to 4 (SEQ ID NO: 129); and Formula (II): XₚTCCCXᵣ, wherein X is any nucleotide, p is an integer from 0 to 17, and r is an integer from 0 to 13 (SEQ ID NO: 130).

In some instances, recombinant RNA constructs provided herein may be cleaved. For example, recombinant RNA constructs provided herein may be cleaved endogenously after cellular uptake. In some embodiments, recombinant RNA constructs may be cleaved by an intracellular protein or an endogenous protein. In some embodiments, recombinant RNA constructs may be cleaved by DICER, e.g., an endogenous DICER. In some embodiments, recombinant RNA constructs comprising a first RNA sequence, a second RNA sequence, and a linker RNA sequence, wherein the linker RNA sequence links the first RNA sequence and the second RNA sequence, may be cleaved between the first RNA sequence and the second RNA sequence. In some embodiments, recombinant RNA constructs provided herein comprise a first RNA sequence, a second RNA sequence, and a linker. In this embodiment, the first RNA sequence or the second RNA sequence may comprise at least two genetic elements that modulate the expression of one or more target RNAs and recombinant RNA constructs may be cleaved between each of at least two genetic elements that modulate the expression of one or more target RNAs. In another embodiment, the first RNA sequence may encode a gene of interest and the second RNA sequence may comprise at least two genetic elements that modulate the expression of one or more target RNA, and recombinant RNA constructs may be cleaved between the gene of interest and the at least two genetic elements that modulate the expression of one or more target RNAs. In some embodiments, the first RNA sequence may encode a gene of interest and the second RNA sequence may comprise at least two genetic elements that modulate the expression of one or more target RNA, and recombinant RNA constructs may be cleaved between the gene of interest and the at least two genetic elements that modulate the expression of one or more target RNAs and/or between each of the at least two genetic elements that modulate the expression of one or more target RNAs.

In some instances, the cleavage of recombinant RNA constructs is enhanced compared to the cleavage of a corresponding RNA construct that does not comprise a linker described herein. For example, the cleavage of recombinant RNA constructs comprising a first RNA sequence, a second RNA sequence, and one or more of linkers described herein is enhanced compared to the cleavage of an RNA construct that does not comprise a linker described herein. For example, the cleavage of recombinant RNA constructs comprising a first RNA sequence, a second RNA sequence, and a linker RNA sequence described herein is enhanced compared to the cleavage of an RNA construct that comprises a linker that does not have a structure selected from the group consisting of Formula (I): XₘCAACAAXₙ, wherein X is any nucleotide, m is an integer from 1 to 12, and n is an integer from 0 to 4 (SEQ ID NO: 129); and Formula (II): XₚTCCCXᵣ, wherein X is any nucleotide, p is an integer from 0 to 17, and r is an integer from 0 to 13 (SEQ ID NO: 130). For example, the cleavage of recombinant RNA constructs comprising a first RNA sequence, a second RNA sequence, and a linker RNA sequence described herein is enhanced compared to the cleavage of an RNA construct that comprises a linker that does not comprise a sequence comprising ACAACAA (SEQ ID NO: 85). For example, the cleavage of recombinant RNA constructs comprising a first RNA sequence, a second RNA sequence, and a linker RNA sequence described herein is enhanced compared to the cleavage of an RNA construct comprising a linker that forms a secondary structure.

In some instances, the expression of a gene of interest from recombinant RNA constructs provided herein is enhanced compared to the expression of a gene of interest from a corresponding recombinant RNA construct that does not comprise a linker described herein.

For example, the expression of a gene of interest from recombinant RNA constructs comprising a first RNA sequence, a second RNA sequence, and a linker RNA sequence described herein is enhanced compared to the expression of a gene of interest from an RNA construct that does not comprise a linker described herein. For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not have a structure selected from the group consisting of Formula (I): XₘCAACAAXₙ, wherein X is any nucleotide, m is an integer from 1 to 12, and n is an integer from 0 to 4 (SEQ ID NO: 129); and Formula (II): XₚTCCCXᵣ, wherein X is any nucleotide, p is an integer from 0 to 17, and r is an integer from 0 to 13 (SEQ ID NO: 130). For example, the expression of a gene of interest from recombinant RNA constructs comprising a first RNA sequence, a second RNA sequence, and a linker RNA sequence described herein is enhanced compared to the expression of a gene of interest from an RNA construct that comprises a linker that does not comprise a sequence comprising ACAACAA (SEQ ID NO: 85). For example, the expression of a gene of interest from recombinant RNA constructs comprising a first RNA sequence, a second RNA sequence, and a linker RNA sequence described herein is enhanced compared to the expression of a gene of interest from an RNA construct comprising a linker that forms a secondary structure.

In some embodiments, the relative increase or enhancement in the expression of a gene of interest or in the cleavage of recombinant RNA constructs is at least about 1.3 fold, about 1.4 fold, about 1.5 fold, about 1.6 fold, about 1.7 fold, about 1.8 fold, about 1.9 fold, about 2 fold, about 3 fold, about 4 fold, about 5 fold, about 6 fold, about 7 fold, about 8 fold, about 9 fold, about 10 fold, about 15 fold, about 17 fold, about 18 fold, about 19 fold, about 20 fold, about 21 fold, about 22 fold, or at least about 25 fold. In some embodiments, the relative increase in the expression of the gene of interest or in the cleavage of recombinant RNA constructs is from about 1.3 fold to about 3 fold, from about 1.5 fold to about 4 fold, from about 2 fold to about 5 fold, from about 2 fold to about 10 fold, from about 2 fold to about 15 fold, from about 2 fold to about 17 fold, from about 2 fold to about 18 fold, from about 2 fold to about 19 fold, from about 2 fold to about 20 fold, from about 2 fold to about 21 fold, from about 2 fold to about 22 fold, from about 2 fold to about 25 fold, from about 2 fold to about 30 fold, from about 5 fold to about 10 fold, from about 5 fold to about 15 fold, from about 5 fold to about 17 fold, from about 5 fold to about 18 fold, from about 5 fold to about 19 fold, from about 5 fold to about 20 fold, from about 5 fold to about 21 fold, from about 5 fold to about 22 fold, from about 5 fold to about 25 fold, from about 5 fold to about 30 fold, from about 10 fold to about 15 fold, from about 10 fold to about 17 fold, from about 10 fold to about 18 fold, from about 10 fold to about 19 fold, from about 10 fold to about 20 fold, from about 10 fold to about 21 fold, from about 10 fold to about 22 fold, from about 10 fold to about 25 fold, from about 10 fold to about 30 fold, from about 15 fold to about 17 fold, from about 15 fold to about 18 fold, from about 15 fold to about 19 fold, from about 15 fold to about 20 fold, from about 15 fold to about 21 fold, from about 15 fold to about 22 fold, from about 15 fold to about 25 fold, from about 15 fold to about 30 fold, from about 17 fold to about 18 fold, from about 17 fold to about 19 fold, from about 17 fold to about 20 fold, from about 17 fold to about 21 fold, from about 17 fold to about 22 fold, from about 17 fold to about 25 fold, from about 17 fold to about 30 fold, from about 18 fold to about 19 fold, from about 18 fold to about 20 fold, from about 18 fold to about 21 fold, from about 18 fold to about 22 fold, from about 18 fold to about 25 fold, from about 18 fold to about 30 fold, from about 19 fold to about 20 fold, from about 19 fold to about 21 fold, from about 19 fold to about 22 fold, from about 19 fold to about 25 fold, from about 19 fold to about 30 fold, from about 20 fold to about 21 fold, from about 20 fold to about 22 fold, from about 20 fold to about 25 fold, from about 20 fold to about 30 fold, from about 21 fold to about 22 fold, from about 21 fold to about 25 fold, from about 21 fold to about 30 fold, from about 22 fold to about 25 fold, from about 22 fold to about 30 fold, or from about 25 fold to about 30 fold. In some embodiments, the relative increase in the expression of the gene of interest or in the cleavage of recombinant RNA constructs is about 1.3 fold, about 1.4 fold, about 1.5 fold, about 1.6 fold, about 1.7 fold, about 1.8 fold, about 1.9 fold, about 2 fold, about 3 fold, about 4 fold, about 5 fold, about 6 fold, about 7 fold, about 8 fold, about 9 fold, about 10 fold, about 15 fold, about 17 fold, about 18 fold, about 19 fold, about 20 fold, about 21 fold, about 22 fold, about 25 fold, or about 30 fold. In some embodiments, the relative increase in the expression of the gene of interest or in the cleavage of recombinant RNA constructs is at most about 2 fold, about 3 fold, about 5 fold, about 10 fold, about 15 fold, about 17 fold, about 18 fold, about 19 fold, about 20 fold, about 21 fold, about 22 fold, about 25 fold, or about 30 fold.

In some embodiments, recombinant RNA constructs provided herein may be naked RNA. In some embodiments, recombinant RNA constructs provided herein may further comprise a 5′ cap, a Kozak sequence, and/or internal ribosome entry site (IRES), and/or a poly(A) tail in a particular in order to improve translation. In some instances, recombinant RNA constructs may further comprise one or more regions promoting translation known to any skilled artisan. Non-limiting examples of the 5′ cap can include an anti-reverse CAP analog, Clean Cap, Cap 0, Cap 1, Cap 2, or Locked Nucleic Acid cap (LNA-cap). In some instances, 5′ cap may comprise m₂^{7,3-O}G(5′)ppp(5′)G, m7G, m7G(5′)G, m7GpppG, or m7GpppGm. In some instances, recombinant RNA constructs provided herein may comprise an IRES upstream or 5′ of the RNA sequence encoding for a gene of interest. In some instances, recombinant RNA constructs provided herein may comprise an IRES immediately upstream or 5′ of the RNA sequence encoding for a gene of interest. In some instances, recombinant RNA constructs provided herein may comprise an IRES downstream or 3′ of the RNA sequence encoding at least one genetic element that modulates expression of a target RNA, wherein the RNA sequence encoding at least one genetic element that modulates expression of a target RNA is present upstream of the RNA sequence encoding for a gene of interest.

Recombinant RNA constructs provided herein may further comprise a poly(A) tail. In some instances, the poly(A) tail comprises 1 to 220 base pairs of poly(A) (SEQ ID NO: 131). For example, the poly(A) tail comprises 1, 3, 5, 8, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, or 220 base pairs of poly(A) (SEQ ID NO: 131). In some embodiments, the poly(A) tail comprises 1 to 20, 1 to 40, 1 to 60, 1 to 80, 1 to 100, 1 to 120, 1 to 140, 1 to 160, 1 to 180, 1 to 200, 1 to 220, 20 to 40, 20 to 60, 20 to 80, 20 to 100, 20 to 120, 20 to 140, 20 to 160, 20 to 180, 20 to 200, 20 to 220, 40 to 60, 40 to 80, 40 to 100, 40 to 120, 40 to 140, 40 to 160, 40 to 180, 40 to 200, 40 to 220, 60 to 80, 60 to 100, 60 to 120, 60 to 140, 60 to 160, 60 to 180, 60 to 200, 60 to 220, 80 to 100, 80 to 120, 80 to 140, 80 to 160, 80 to 180, 80 to 200, 80 to 220, 100 to 120, 100 to 140, 100 to 160, 100 to 180, 100 to 200, 100 to 220, 120 to 140, 120 to 160, 120 to 180, 120 to 200, 120 to 220, 140 to 160, 140 to 180, 140 to 200, 140 to 220, 160 to 180, 160 to 200, 160 to 220, 180 to 200, 180 to 220, or 200 to 220 base pairs of poly(A) (SEQ ID NO: 131). In some embodiments, the poly(A) tail comprises 1, 20, 40, 60, 80, 100, 120, 140, 160, 180, 200, or 220 base pairs of poly(A) (SEQ ID NO: 131). In some embodiments, the poly(A) tail comprises at least 1, 20, 40, 60, 80, 100, 120, 140, 160, 180, or at least 200 base pairs of poly(A) (SEQ ID NO: 132). In some embodiments, the poly(A) tail comprises at most 20, 40, 60, 80, 100, 120, 140, 160, 180, 200, or at most 220 base pairs of poly(A) (SEQ ID NO: 131). In some embodiments, the poly(A) tail comprises 120 base pairs of poly(A) (SEQ ID NO: 133).

Recombinant RNA constructs provided herein may further comprise a Kozak sequence. A Kozak sequence may refer to a nucleic acid sequence motif that functions as a protein translation initiation site. Kozak sequences are described at length in the literature, e.g., by Kozak, M., Gene 299(1-2):1-34, incorporated herein by reference herein in its entirety. In some embodiments, the Kozak sequence described herein may comprise a sequence comprising GCCACC (SEQ ID NO: 25). In some embodiments, recombinant RNA constructs provided herein may further comprise a nuclear localization signal (NLS).

In one aspect, recombinant RNA constructs described herein may not comprise a nucleotide variant. In some instances, recombinant RNA constructs described herein may comprise one or more uridines. In some instances, recombinant RNA constructs described herein may not comprise a modified uridine. In some instances, recombinant RNA constructs described herein may not comprise one or more N1-methylpseudouridines. In some embodiments, between 99% and 1%, between 98% and 2%, between 97% and 3%, between 96% and 4%, between 95% and 2%, between 94% and 6%, between 93% and 7%, between 92% and 8%, between 91% and 9%, between 90% and 10%, between 97% and 3%, of the one or more uridines comprised in the recombinant RNA constructs are unmodified. In some embodiments, at least 70%, 75%, 80%, 85%, 90%, 91%, 92%, 93%, 94%, 95%, 96%, 97%, 98%, 99%, 99.5%, 99.6%, 99.7%, 99.8%, or at least 99.9% of one or more uridines comprised in the recombinant RNA constructs are unmodified. In some embodiments, at most 1%, 5%, 10%, 20%, 30%, 40%, 50%, 60%, 70%, 75%, 80%, 85%, 90%, 91%, 92%, 93%, 94%, 95%, 96%, 97%, 98%, 99%, 99.5%, 99.6%, 99.7%, 99.8%, or at least 99.9% of one or more uridines comprised in the recombinant RNA constructs are modified. In one embodiment, recombinant RNA constructs described herein comprise solely unmodified nucleotides. For example, recombinant RNA constructs described herein comprise only natural nucleotides.

For example, recombinant RNA constructs described herein comprise only canonical nucleotides. In a preferred embodiment, recombinant RNA constructs described herein comprise one or more uridines, wherein all of one or more uridines are unmodified. In another aspect, recombinant RNA constructs described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenosine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine, N1-methylpseudouridine, and the like. In some cases, nucleotides may include modifications in their phosphate moieties, including modifications to a triphosphate moiety. Non-limiting examples of such modifications include phosphate chains of greater length and modifications with thiol moieties. In some embodiments, phosphate chains can comprise 4, 5, 6, 7, 8, 9, 10 or more phosphate moieties. In some embodiments, thiol moieties can include but are not limited to alpha-thiotriphosphate and beta-thiotriphosphates. In some embodiments, a recombinant RNA construct described herein does not comprise 5-methylcytosine and/or N6-methyladenosine.

In some instances, recombinant RNA constructs described herein may be modified at the base moiety, sugar moiety, or phosphate backbone. For example, modifications can be at one or more atoms that typically are available to form a hydrogen bond with a complementary nucleotide and/or at one or more atoms that are not typically capable of forming a hydrogen bond with a complementary nucleotide. In some embodiments, backbone modifications include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) allows prevents nuclease recognition and degradation. In some embodiments, backbone modifications include having peptide bonds instead of phosphorous in the backbone structure, or linking groups including carbamate, amides, and linear and cyclic hydrocarbon groups. For example, N-(2-aminoethyl)-glycine units may be linked by peptide bonds in a peptide nucleic acid. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999.

Recombinant RNA constructs provided herein may comprise a combination of modified and unmodified nucleotides. In some instances, the adenosine-, guanosine-, and cytidine-containing nucleotides are unmodified or partially modified. In some instances, for modified RNA constructs, 1%, 5%, 10%, 20%, 30%, 40%, 50%, 60%, 70%, 80%, 90%, or 100% of uridine nucleotides may be modified. In some embodiments, 5% to 25% of uridine nucleotides are modified in recombinant RNA constructs. Non-limiting examples of the modified uridine nucleotides may comprise pseudouridines, N¹-methylpseudouridines, or N1-methylpseudo-UTP and any modified uridine nucleotides known in the art may be utilized. In some embodiments, recombinant RNA constructs may contain a combination of modified and unmodified nucleotides, wherein 1%, 5%, 10%, 20%, 30%, 40%, 50%, 60%, 70%, 80%, 90%, or 100% of uridine nucleotides may comprise pseudouridines, N¹-methylpseudouridines, N1-methylpseudo-UTP, or any other modified uridine nucleotide known in the art. In some embodiments, recombinant RNA constructs may contain a combination of modified and unmodified nucleotides, wherein 1%, 5%, 10%, 20%, 30%, 40%, 50%, 60%, 70%, 80%, 90%, or 100% of the uridine nucleotides may comprise N¹-methylpseudouridines.

Recombinant RNA constructs provided herein may be codon-optimized. In general, codon optimization refers to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA) which is preferred. In some embodiments, recombinant RNA constructs may not be codon-optimized.

In some instances, recombinant RNA constructs may comprise a nucleic acid sequence comprising a sequence selected from the group consisting of SEQ ID NOs: 1-12 and 97-108.

RNA Interference and Small Interfering RNA (siRNA)

RNA interference (RNAi) or RNA silencing is a process in which RNA molecules inhibit gene expression or translation, by neutralizing target mRNA molecules. RNAi process is described in Mello & Conte (2004) Nature 431, 338-342, Meister & Tuschl (2004) Nature 431, 343-349, Hannon & Rossi (2004) Nature 431, 371-378, and Fire (2007) Angew. Chem. Int. Ed. 46, 6966-6984. Briefly, in a natural process, the reaction initiates with a cleavage of long double-stranded RNA (dsRNA) into small dsRNA fragments or siRNAs with a hairpin structure (i.e., shRNAs) by a dsRNA-specific endonuclease Dicer. These small dsRNA fragments or siRNAs are then integrated into RNA-induced silencing complex (RISC) and guide the RISC to the target mRNA sequence. During interference, the siRNA duplex unwinds, and the anti-sense strand remains in complex with RISC to lead RISC to the target mRNA sequence to induce degradation and subsequent suppression of protein translation. Unlike commercially available synthetic siRNAs, siRNAs in the present invention can utilize endogenous Dicer and RISC pathway in the cytoplasm of a cell to get cleaved from recombinant RNA constructs (e.g., recombinant RNA constructs comprising an mRNA and at least two siRNAs) and follow the natural process detailed above, as siRNAs in the recombinant RNA constructs of the present invention may comprise a hairpin loop structure. In addition, as the rest of the recombinant RNA constructs (i.e., mRNA) is left intact after cleavage of siRNAs by Dicer, the desired protein expression from the gene of interest in the recombinant RNA constructs of the present invention is attained.

Provided herein are compositions comprising recombinant RNA constructs comprising at least two nucleic acid sequence each comprising a genetic element that modulates the expression of one or more target RNAs. In some instances, the genetic element that modulates the expression of one or more target RNAs may be a siRNA capable of binding to a target RNA. In some instances, provided here in are compositions comprising recombinant RNA constructs comprising at least one nucleic acid sequence comprising a siRNA capable of binding to a target RNA. In some instances, the target RNA is a noncoding RNA. In some instances, the target RNA is an mRNA. In some embodiments, the siRNA is capable of binding to a target mRNA in the 5′ untranslated region. In some embodiments, the siRNA is capable of binding to a target mRNA in the 3′ untranslated region. In some embodiments, the siRNA is capable of binding to a target mRNA in an exon.

In some embodiments, recombinant RNA constructs may comprise a nucleic acid sequence comprising a sense siRNA strand. In some embodiments, recombinant RNA constructs may comprise a nucleic acid sequence comprising an anti-sense siRNA strand. In some embodiments, recombinant RNA constructs may comprise a nucleic acid sequence comprising a sense siRNA strand and a nucleic acid sequence comprising an anti-sense siRNA strand. Details of siRNA comprised in the present invention are described in Cheng, et al. (2018) J. Mater. Chem. B., 6, 4638-4644, which is incorporated by reference herein.

In some instances, a genetic element that modulates the expression of one or more target RNAs, e.g., a siRNA, may comprise a nucleic acid sequence comprising a sense siRNA strand and an anti-sense siRNA strand. For example, in some instances, recombinant RNA constructs may comprise at least 1 species of siRNA, i.e., a nucleic acid sequence comprising a sense strand of siRNA and a nucleic acid sequence comprising an anti-sense strand of siRNA. 1 species of siRNA, as described herein, can refer to 1 species of sense strand siRNA and 1 species of anti-sense strand siRNA. In some instances, recombinant RNA constructs may comprise more than 1 species of siRNA, e.g., 2, 3, 4, 5, 6, 7, 8, 9, 10, or more species of siRNA comprising a sense strand of siRNA and an anti-sense strand of siRNA. In some embodiments, recombinant RNA constructs may comprise 1 to 20 species of siRNA. In some embodiments, recombinant RNA constructs may comprise at least 1, 2, 3, 4, 5, 6, 7, 8, 9, or at least 10 species of siRNA. In some embodiments, recombinant RNA constructs may comprise at most 3, 4, 5, 6, 7, 8, 9, 10, 11, 12, 13, 14, 15, 16, 17, 18, 19, or at most 20 species of siRNA. In some embodiments, recombinant RNA constructs may comprise between 2 siRNAs and 10 siRNAs, between 3 siRNAs and 10 siRNAs, between 4 siRNAs and 10 siRNAs, between 5 siRNAs and 10 siRNAs, between 6 siRNAs and 10 siRNAs, between 7 siRNAs and 10 siRNAs, between 9 siRNAs and 10 siRNAs, preferably between 2 siRNAs and 6 siRNAs, between 3 siRNAs and 6 siRNAs, or between 4 siRNAs and 6 siRNAs. In a preferred embodiment, recombinant RNA constructs described herein comprise at least 2 species of siRNA. In another preferred embodiment, recombinant RNA constructs described herein comprise at least 3 species of siRNA. In yet another preferred embodiment, recombinant RNA constructs described herein comprise at least 6 species of siRNA.

Provided herein are compositions of recombinant RNA constructs comprising 1-20 or more siRNA species, wherein each of the 1-20 or more siRNA species is capable of binding to a target RNA. In some embodiments, a target RNA is an mRNA or a non-coding RNA. In some instances, each of the siRNA species binds to the same target RNA. In one instance, each of the siRNA species may comprise the same sequence and bind to the same region or sequence of the same target RNA. For example, recombinant RNA constructs may comprise 1, 2, 3, 4, 5, 6, or more siRNA species and each of the 1, 2, 3, 4, 5, 6, or more siRNA species comprise the same sequence targeting the same region of a target RNA, i.e., recombinant RNA constructs may comprise 1, 2, 3, 4, 5, 6, or more redundant species of siRNA. In another instance, each of the siRNA species may comprise a different sequence and bind to a different region or sequence of the same target RNA. For example, recombinant RNA constructs may comprise 1, 2, 3, 4, 5, 6, or more siRNA species and each of the 1, 2, 3, 4, 5, 6, or more siRNA species may comprise a different sequence targeting a different region of the same target RNA. In this example, one siRNA of the 1, 2, 3, 4, 5, 6, or more siRNA species may target exon 1 and another siRNA of the 1, 2, 3, 4, 5, 6, or more siRNA species may target exon 2 of the same mRNA, etc. In some instances, recombinant RNA constructs may comprise 1, 2, 3, 4, 5, 6, or more siRNA species capable of binding to the same and different regions of the same target RNA. For example, recombinant RNA constructs may comprise 1, 2, 3, 4, 5, 6, or more siRNA species and 2 of the 1, 2, 3, 4, 5, 6, or more siRNA species may comprise the same sequence and bind to the same regions of the target RNA and 3 or more of the 1, 2, 3, 4, 5, 6, or more siRNA species may comprise a different sequence and bind to different regions of the same target RNA. In some instances, each of the siRNA species binds to a different target RNA. In some instances, recombinant RNA constructs may comprise 1, 2, 3, 4, 5, 6, or more siRNA species capable of binding to the same and different target RNAs. For example, recombinant RNA constructs may comprise 1, 2, 3, 4, 5, 6, or more siRNA species and 2 of the 1, 2, 3, 4, 5, 6, or more siRNA species may comprise a sequence capable of binding to the same or different regions of the same target RNA and 3 or more of the 1, 2, 3, 4, 5, 6, or more siRNA species may comprise a sequence capable of binding to a different target RNA. In some embodiments, a target RNA may be an mRNA and/or a non-coding RNA.

Provided herein are compositions of recombinant RNA constructs comprising 1-20 or more siRNA species, wherein each of the 1-20 or more siRNA species are connected by a linker described herein. In some instances, the linker may be a non-cleavable linker. In some instances, the linker may be a cleavable linker such as a self-cleavable linker. In some instances, the linker may be cleaved by a protein, e.g., an intracellular or an endogenous protein. In some instances, the linker has a structure selected from the group consisting of Formula (I): XₘCAACAAXₙ, wherein X is any nucleotide, m is an integer from 1 to 12, and n is an integer from 0 to 4 (SEQ ID NO: 129); and Formula (II): XₚTCCCXᵣ, wherein X is any nucleotide, p is an integer from 0 to 17, and r is an integer from 0 to 13 (SEQ ID NO: 130). In some instances, the linker may comprise a sequence comprising ACAACAA (SEQ ID NO: 85), ATCCCTACGTACCAACAA (SEQ ID NO: 87), ACGTACCAACAA (SEQ ID NO: 88), TCCC (SEQ ID NO: 89), or ACAACAATCCC (SEQ ID NO: 90). In some embodiments, the linker may comprise a sequence comprising ACAACAA (SEQ ID NO: 85), ATAGTGAGTCGTATTATCCC (SEQ ID NO: 92), ATAGTGAGTCGTATTAACAACAATCCC (SEQ ID NO: 93), ATAGTGAGTCGTATTAACAACAA (SEQ ID NO: 94), ATAGTGAGTCGTATTAATCCCTACGTACCAACAA (SEQ ID NO: 95), or ATAGTGAGTCGTATTAACGTACCAACAA (SEQ ID NO: 27). In some embodiments, the linker may comprise a sequence comprising ACAACAA (SEQ ID NO: 85). In some embodiments, the linker does not comprise a sequence comprising

(SEQ ID NO: 28)
TTTATCTTAGAGGCATATCCCTACGTACCAACAA
or
(SEQ ID NO: 27)
ATAGTGAGTCGTATTAACGTACCAACAA.

In some instances, the length of a linker is from about 4 to about 50, from about 4 to about 45, or from about 4 to about 40, from about 4 to about 35, or from about 4 to about 30 nucleotides. In some embodiments, the length of a linker is from about 4 to about 27 nucleotides. In some embodiments, the length of a linker is from about 4 to about 18 nucleotides. For example, the length of a linker is about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleotides. In some embodiments, the length of a linker can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or at most about 50 nucleotides. In some embodiments, the length of a linker is 4 nucleotides. In some embodiments, the length of a linker is 7 nucleotides. In some embodiments, the length of a linker is 11 nucleotides. In some embodiments, the length of a linker is 12 nucleotides. In some embodiments, the length of a linker is 18 nucleotides.

In some instances, the linker may have a structure of Formula (I) XₘCAACAAXₙ, wherein X is any nucleotide; m is an integer from 1 to 12, and n is an integer from 0 to 4 (SEQ ID NO: 129); and m is 1 and n is 0. In some instances, the linker may comprise a sequence comprising CAACAA (SEQ ID NO: 91), TCCC (SEQ ID NO: 89), or ACAACAA (SEQ ID NO: 85). In some embodiments, the linker may comprise a sequence selected from the group consisting of ATCCCTACGTACCAACAA (SEQ ID NO: 87), ACGTACCAACAA (SEQ ID NO: 88), TCCC (SEQ ID NO: 89), ACAACAATCCC (SEQ ID NO: 90), and ACAACAA (SEQ ID NO: 85). In some embodiments, the linker may comprise a sequence comprising ACAACAA (SEQ ID NO: 85), ATAGTGAGTCGTATTATCCC (SEQ ID NO: 92), ATAGTGAGTCGTATTAACAACAATCCC (SEQ ID NO: 93), ATAGTGAGTCGTATTAACAACAA (SEQ ID NO: 94), ATAGTGAGTCGTATTAATCCCTACGTACCAACAA (SEQ ID NO: 95), or ATAGTGAGTCGTATTAACGTACCAACAA (SEQ ID NO: 27). In some embodiments, the linker may comprise a sequence comprising ACAACAA (SEQ ID NO: 85). In some embodiments, the linker may comprise a sequence selected from the group consisting of SEQ ID NOs: 27, 28, 85-95.

In some instances, the linker may be a tRNA linker. The tRNA system is evolutionarily conserved across living organism and utilizes endogenous RNases P and Z to process multicistronic constructs (Dong et al., 2016). In some embodiments, the tRNA linker may comprise a nucleic acid sequence comprising AACAAAGCACCAGTGGTCTAGTGGTAGAATAGTACCCTGCCACGGTACAGACCC GGGTTCGATTCCCGGCTGGTGCA (SEQ ID NO: 96). In some embodiments, a linker comprising a nucleic acid sequence comprising TTTATCTTAGAGGCATATCCCTACGTACCAACAA (SEQ ID NO: 28) may be used to connect each of the 1-20 or more siRNA species.

In some instances, specific binding of an siRNA to its target mRNA results in interference with the normal function of the target mRNA, leading to modulation, e.g., downregulation, of expression level, function, and/or activity of a protein encoded by the target mRNA, and there is a sufficient degree of complementarity to avoid non-specific binding of the siRNA to non-target nucleic acid sequences under conditions in which specific binding is desired, i.e., under physiological conditions in the case of in vivo assays or therapeutic treatment, and under conditions in which assays are performed in the case of in vitro assays.

A protein as used herein can refer to molecules typically comprising one or more peptides or polypeptides. A peptide or polypeptide is typically a chain of amino acid residues, linked by peptide bonds. A peptide usually comprises between 2 and 50 amino acid residues. A polypeptide usually comprises more than 50 amino acid residues. A protein is typically folded into 3-dimensional form, which may be required for the protein to exert its biological function. A protein as used herein can include a fragment of a protein, a functional variant of a protein, and a fusion protein. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence. For example, a variant molecule may comprise or encode a mutant protein, including, but not limited to, a gain-of-function or a loss-of-function mutant. A fragment may be a shorter portion of a full-length sequence of a nucleic acid molecule like DNA or RNA, or a protein. Accordingly, a fragment, typically, comprises a sequence that is identical to the corresponding stretch within the full-length sequence. In some embodiments, a fragment of a sequence may comprise at least 5% to at least 80% of a full-length nucleotide or amino acid sequence from which the fragment is derived. In some embodiments, a protein can be a mammalian protein. In some embodiments, a protein can be a human protein. In some embodiments, a protein may be a protein secreted from a cell. In some embodiments, a protein may be a protein on cell membranes. In some embodiments, a protein as referred to herein can be a protein that is secreted and acts either locally or systemically as a modulator of target cell signaling via receptors on cell surfaces, often involved in immunologic reactions or other host proteins involved in viral infection. Nucleotide and amino acid sequences of proteins useful in the context of the present invention, including proteins that are encoded by a gene of interest, are known in the art and available in the literature. For example, nucleotide and amino acid sequences of proteins useful in the context of the present invention, including proteins that are encoded by a gene of interest are available in the UniProt database.

Provided herein are compositions of recombinant RNA constructs comprising an siRNA capable of binding to a target mRNA to modulate expression of the target mRNA. In some instances, expression of the target mRNA (e.g., the level of protein encoded by the target mRNA) is downregulated by the siRNA capable of binding to the target mRNA. In some embodiments, expression of the target mRNA is inhibited by the siRNA capable of binding to the target mRNA. Inhibition or downregulation of expression of the target mRNA, as described herein, can refer to, but is not limited to, interference with the target mRNA to interfere with translation of the protein from the target mRNA; thus, inhibition or downregulation of expression of the target mRNA can refer to, but is not limited to, a decreased level of proteins expressed from the target mRNA compared to a level of proteins expressed from the target mRNA in the absence of recombinant RNA constructs comprising siRNA capable of binding to the target mRNA. Levels of protein expression can be measured by using any methods well known in the art and these include, but are not limited to Western-blotting, flow cytometry, ELISAs, radioimmunoassays (RIAs), and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are compositions comprising recombinant RNA constructs comprising at least two nucleic acid sequences comprising siRNA capable of binding to one or more target mRNAs and at least one nucleic acid sequence encoding a gene of interest wherein the target mRNA is different from an mRNA encoded by the gene of interest. Provided herein are compositions comprising recombinant RNA constructs comprising at least two nucleic acid sequences comprising siRNA capable of binding to one or more target mRNAs and at least one nucleic acid sequence encoding a gene of interest wherein the siRNA does not affect expression of the gene of interest. In some instances, the siRNA is not capable of binding to an mRNA encoded by the gene of interest. In some instances, the siRNA does not inhibit the expression of the gene of interest. In some instances, the siRNA does not downregulate the expression of the gene of interest. Inhibiting or downregulating the expression of the gene of interest, as described herein, can refer to, but is not limited to, interfering with translation of proteins from recombinant RNA constructs; thus, inhibiting or downregulating the expression of the gene of interest can refer to, but is not limited to, a decreased level of protein compared to a level of protein expressed in the absence of recombinant RNA constructs comprising siRNA capable of binding to the target mRNA. Levels of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are compositions comprising recombinant RNA constructs comprising at least two nucleic acid sequences comprising an siRNA capable of binding to one or more target mRNAs. A list of non-limiting examples of target mRNAs that the siRNA is capable of binding to includes an mRNA of a gene comprising Tumor Necrosis Factor alpha (TNF-alpha or TNF-α), Interleukin 8 (IL-8), Interleukin 1 beta (IL-1beta), Interleukin 17 (IL-17), Turbo Green Fluorescence Protein (Turbo GFP), or a functional variant thereof. In some embodiments, Turbo GFP sequence can be derived from marine copepod Pontellina plumate. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence.

In some embodiments, TNF-alpha comprises a sequence listed in SEQ ID NO: 40. In some embodiments, IL-8 comprises a sequence listed in SEQ ID NO: 37. In some embodiments, IL-1beta comprises a sequence listed in SEQ ID NO: 38. In some embodiments, IL-17 comprises a sequence listed in SEQ ID NO: 39. In some embodiments, Turbo GFP comprises a sequence listed in SEQ ID NO: 41.

In some aspects, the siRNA comprises a sense strand encoded by a sequence selected from SEQ ID NOs: 57-70. In some aspects, the siRNA comprises an anti-sense strand encoded by a sequence selected from SEQ ID NOs: 71-84. In some aspects, the siRNA comprises a sense strand encoded by a sequence selected from SEQ ID NOs: 57-70, and the corresponding anti-sense strand encoded by a sequence selected from SEQ ID NOs: 71-84.

### Gene of Interest

Provided herein are recombinant RNA constructs comprising one or more copies of nucleic acid sequence encoding a gene of interest. For example, recombinant RNA constructs may comprise 1, 2, 3, 4, 5, 6, 7, 8, 9, 10, or more copies of nucleic acid sequence encoding a gene of interest. In some instances, each of the 1, 2, 3, 4, 5, 6, 7, 8, 9, 10, or more copies of nucleic acid sequence encoding a gene of interest encodes the same gene of interest. In some instances, recombinant RNA constructs may comprise 1, 2, 3, 4, 5, 6, 7, 8, 9, 10, or more copies of nucleic acid sequence encoding a cytokine.

Also provided herein are recombinant RNA constructs comprising two or more copies of nucleic acid sequence encoding a gene of interest, wherein each of the two or more nucleic acid sequence may encode a different gene of interest. In some cases, each of the two or more nucleic acid sequences encoding different gene of interest may comprise a nucleic acid sequence encoding a secretory protein. In some cases, each of the two or more nucleic acid sequences encoding different gene of interest may comprise a nucleic acid sequence encoding a cytokine, e.g., Interleukin 4 (IL-4). In some embodiments, each of the two or more nucleic acid sequences encoding different gene of interest may encode a different secretory protein. In some cases, each of the two or more nucleic acid encoding different gene of interest may comprise a nucleic acid sequence encoding Insulin-like Growth Factor 1 (IGF-1). Further provided herein are recombinant RNA constructs comprising a linker described herein. In some embodiments, the linker may connect each of the two or more nucleic acid sequences encoding a gene of interest. In some cases, the linker may be a non-cleavable linker. In some cases, the linker may be a cleavable linker. In some cases, the linker may be a self-cleavable linker. In some cases, the linker may be cleaved by a protein, e.g., an intracellular protein or an endogenous protein. In some instances, the linker is selected from the group consisting of Formula (I): XₘCAACAAXₙ, wherein X is any nucleotide, m is an integer from 1 to 12, and n is an integer from 0 to 4 (SEQ ID NO: 129); and Formula (II): XₚTCCCXᵣ, wherein X is any nucleotide, p is an integer from 0 to 17, and r is an integer from 0 to 13 (SEQ ID NO: 130). In some instances, the linker comprises a sequence comprising ACAACAA (SEQ ID NO: 85). In some embodiments, the linker is selected from the group consisting of SEQ ID NOs: 27, 28, 85-95.

Other examples of the linker include, but are not limited to, a flexible linker, a 2A peptide linker (or 2A self-cleaving peptides) such as T2A, P2A, E2A, or F2A, and a tRNA linker, etc. The tRNA system is evolutionarily conserved across living organism and utilizes endogenous RNases P and Z to process multicistronic constructs (Dong et al., 2016). In some embodiments, the tRNA linker may comprise a nucleic acid sequence comprising

(SEQ ID NO: 96)
AACAAAGCACCAGTGGTCTAGTGGTAGAATAGTACCCTGCCACGGTAC
AGACCCGGGTTCGATTCCCGGCTGGTGCA.

Provided herein are recombinant RNA constructs comprising an RNA encoding for a gene of interest for modulating the expression of the gene of interest. For example, expression of a protein encoded by the mRNA of the gene of interest can be modulated. For example, the expression of the gene of interest is upregulated by expressing a protein encoded by mRNA of the gene of interest in recombinant RNA constructs. For example, the expression of the gene of interest is upregulated by increasing the level of protein encoded by mRNA of the gene of interest in recombinant RNA constructs. The level of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are recombinant RNA constructs comprising an RNA encoding for a gene of interest wherein the gene of the interest encodes a protein of interest. In some instances, the protein of interest is a therapeutic protein. In some instances, the protein of interest is of human origin i.e., is a human protein. In some instances, the gene of interest encodes a secretory protein. In some embodiments, the gene of interest encodes Insulin-like Growth Factor 1 (IGF-1). In some embodiments, the protein of interest is IGF-1. In some instances, the gene of interest encodes a cytokine. In some embodiments, the cytokine comprises an interleukin. In some embodiments, the protein of interest is Interleukin 4 (IL-4) or a functional variant thereof.

In some instances, recombinant RNA constructs comprising a nucleic acid sequence encoding a gene of interest may comprise a nucleic acid sequence encoding human insulin-like growth factor 1 (IGF-1). In some instances, IGF-1 as used herein may refer to the natural sequence of human IGF-1 (Uniprot database: P05019 and in the Genbank database: NM_001111285.3), a fragment, or a functional variant thereof. In one embodiment, recombinant RNA constructs can be naked RNA comprising a nucleic acid sequence encoding IGF-1. In this embodiment, recombinant RNA constructs may comprise a nucleic acid sequence encoding the mature human IGF-1. The natural DNA sequence encoding human IGF-1 may be codon-optimized. The natural sequence of human IGF-1 comprises a signal peptide having 21 amino acids (nucleotides 1-63), a pro-peptide having 27 amino acids (nucleotides 64-144), a mature human IGF-1 having 70 amino acids (nucleotides 145-354), and E-peptide having 77 amino acids (nucleotides 355-585). In some embodiments, recombinant RNA constructs may comprise a nucleic acid sequence encoding a pro-peptide (also called pro-domain) of IGF-1, a nucleic acid sequence encoding a mature protein of IGF-1, or an E-peptide (also called E-domain) of IGF-1 (i.e., IGF-1 with a carboxyl-terminal extension). In some embodiments, recombinant RNA constructs do not comprise a nucleic acid sequence encoding an E-peptide of IGF-1. In some embodiments, recombinant RNA constructs may comprise a nucleic acid sequence encoding a pro-peptide of IGF-1, a nucleic acid sequence encoding a mature protein of IGF-1, and a nucleic acid sequence encoding the signal peptide of brain-derived neurotrophic factor (BDNF). In some embodiments, IGF-1 is a human IGF-1.

In some embodiments, recombinant RNA constructs may comprise a nucleic acid sequence encoding a pro-peptide of IGF-1, preferably of human IGF-1 having 27 amino acids, and a nucleic sequence encoding a mature IGF-1, preferably a mature human IGF-1 having 70 amino acids, and preferably do not comprise a nucleotide sequence encoding an E-peptide of IGF-1, and preferably do not comprise a nucleic acid sequence encoding a human E-peptide of IGF-1. In some embodiments, recombinant RNA constructs may comprise a nucleic acid sequence encoding a pro-peptide of IGF-1, preferably of human IGF-1 having 27 amino acids, a nucleic sequence encoding a mature IGF-1, preferably a mature human IGF-1 having 70 amino acids, and a nucleic acid sequence encoding the signal peptide of brain-derived neurotrophic factor (BDNF). In some embodiments, recombinant RNA constructs do not comprise a nucleic sequence encoding an E-peptide of IGF-1, more preferably does not comprise a nucleic acid sequence encoding a human E-peptide of IGF-1.

In some embodiments, recombinant RNA constructs provided herein may comprise a nucleic acid sequence encoding a pro-peptide of human IGF-1 having 27 amino acids and a nucleic acid sequence encoding a mature human IGF-1 having 70 amino acids, and preferably do not comprise a nucleic acid sequence encoding an E-peptide of human IGF-1, wherein the nucleic acid sequence encoding the pro-peptide of human IGF-1 having 27 amino acids and the nucleic acid sequence encoding the mature human IGF-1 having 70 amino acids, and the nucleic acid sequence encoding the E-peptide are as referred to in the Uniprot database as UniProtKB-P05019. In some embodiments, IGF-1 described herein may have an amino acid sequence comprising SEQ ID NO: 34 or SEQ ID NO: 36.

In some instances, recombinant RNA constructs provided herein may comprise an mRNA encoding IGF-1. In some embodiments, the mRNA encoding IGF-1 may refer to an mRNA comprising a nucleotide sequence encoding the pro-peptide of human IGF-1 having 27 amino acids and/or a nucleotide sequence encoding the mature human IGF-1 having 70 amino acids. The nucleotide sequence encoding the pro-peptide of human IGF-1 and the nucleotide sequence encoding the mature human IGF-1 may be codon-optimized. In some instances, recombinant RNA constructs provided herein may comprise 1 copy of IGF-1 mRNA. In some instances, recombinant RNA constructs provided herein may comprise 2 or more copies of IGF-1 mRNA.

In some instances, Interleukin 4 (IL-4) or IL-4 as used herein may refer to the natural sequence of human IL-4 (Uniprot database: P05112 and in the Genbank database: NM_000589.4), a fragment, or a functional variant thereof. The natural DNA sequence encoding human IL-4 may be codon-optimized. The natural sequence of human IL-4 comprises a signal peptide having 24 amino acids (nucleotides 1-72) and a mature human IL-4 having 153 amino acids (nucleotides 73-459). In some embodiments, the signal peptide is unmodified IL-4 signal peptide. In some embodiments, the signal peptide is IL-4 signal peptide modified by insertion, deletion, and/or substitution of at least one amino acid. In some embodiments, Interleukin 4 (IL-4) or IL-4 as used herein may refer to the mature human IL-4. In some embodiments, a mature protein can refer to a protein synthesized in the endoplasmic reticulum and secreted via the Golgi apparatus in a cell expressing and secreting the protein. In some embodiments, a mature IL-4 may refer to an IL-4 protein synthesized in the endoplasmic reticulum and secreted via the Golgi apparatus in a cell expressing and secreting IL-4. In some embodiments, a mature human IL-4 may refer to an IL-4 protein synthesized in the endoplasmic reticulum and secreted via the Golgi apparatus in a human cell expressing and secreting human IL-4 and normally contains the amino acids encoded by nucleotide as shown in SEQ ID NO: 31. In some embodiments, IL-4 described herein may have an amino acid sequence comprising SEQ ID NO: 32 or SEQ ID NO: 42.

The mRNA encoding IL-4 may refer to an mRNA comprising a nucleotide sequence encoding the pro-peptide of human IL-4 having 153 amino acids or a nucleotide sequence encoding the mature human IL-4 having 129 amino acids. The nucleotide sequence encoding the pro-peptide of human IL-4 and the nucleotide sequence encoding the mature human IL-4 may be codon-optimized. In some instances, recombinant RNA constructs provided herein may comprise 1 copy of IL-4 mRNA. In some instances, recombinant RNA constructs provided herein may comprise 2 or more copies of IL-4 mRNA.

### Target Motif

Provided herein are compositions comprising recombinant RNA constructs comprising a target motif. A target motif or a targeting motif as used herein can refer to any short peptide present in the newly synthesized polypeptides or proteins that are destined to any parts of cell membranes, extracellular compartments, or intracellular compartments, except cytoplasm or cytosol. In some embodiments, a peptide may refer to a series of amino acid residues connected one to the other, typically by peptide bonds between the α-amino and carboxyl groups of adjacent amino acid residues. Intracellular compartments include, but are not limited to, intracellular organelles such as nucleus, nucleolus, endosome, proteasome, ribosome, chromatin, nuclear envelope, nuclear pore, exosome, melanosome, Golgi apparatus, peroxisome, endoplasmic reticulum (ER), lysosome, centrosome, microtubule, mitochondria, chloroplast, microfilament, intermediate filament, or plasma membrane. In some embodiments, a signal peptide can be referred to as a signal sequence, a targeting signal, a localization signal, a localization sequence, a transit peptide, a leader sequence, or a leader peptide. In some embodiments, a target motif is operably linked to a nucleic acid sequence encoding a gene of interest. In some embodiments, the term “operably linked” can refer to a functional relationship between two or more nucleic acid sequences, e.g., a functional relationship of a transcriptional regulatory or signal sequence to a transcribed sequence. For example, a target motif or a nucleic acid encoding a target motif is operably linked to a coding sequence if it is expressed as a preprotein that participates in targeting the polypeptide encoded by the coding sequence to a cell membrane, intracellular, or an extracellular compartment. For example, a signal peptide or a nucleic acid encoding a signal peptide is operably linked to a coding sequence if it is expressed as a preprotein that participates in the secretion of the polypeptide encoded by the coding sequence. For example, a promoter is operably linked if it stimulates or modulates the transcription of the coding sequence. Non-limiting examples of a target motif comprise a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, a centrosomal localization signal (CLS) or any other signal that targets a protein to a certain part of cell membrane, extracellular compartments, or intracellular compartments.

A signal peptide is a short peptide present at the N-terminus of newly synthesized proteins that are destined towards the secretory pathway. The signal peptide of the present invention can be 10-40 amino acids long. A signal peptide can be situated at the N-terminal end of the protein of interest or at the N-terminal end of a pro-protein form of the protein of interest. A signal peptide may be of eukaryotic origin. In some embodiments, a signal peptide may be a mammalian protein. In some embodiments, a signal peptide may be a human protein. In some instances, a signal peptide may be a homologous signal peptide (i.e., from the same protein) or a heterologous signal peptide (i.e., from a different protein or a synthetic signal peptide). In some instances, a signal peptide may be a naturally occurring signal peptide of a protein or a modified signal peptide.

Provided herein are compositions comprising recombinant RNA constructs comprising a target motif, wherein the target motif may be selected from the group consisting of (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest; (d) a target motif homologous to a protein encoded by the gene of interest, wherein the target motif homologous to the protein encoded by the gene of interest is modified by insertion, deletion, and/or substitution of at least one amino acid; and (e) a naturally occurring amino acid sequence which does not have the function of a target motif in nature, wherein the naturally occurring amino acid sequence is optionally modified by insertion, deletion, and/or substitution of at least one amino acid.

Provided herein are compositions comprising recombinant RNA constructs comprising a target motif, wherein the target motif is a signal peptide. In some embodiments, the signal peptide is selected from the group consisting of: (a) a signal peptide heterologous to a protein encoded by the gene of interest; (b) a signal peptide heterologous to a protein encoded by the gene of interest, wherein the signal peptide heterologous to the protein encoded by the gene of interest is modified by insertion, deletion, and/or substitution of at least one amino acid, with proviso that the protein is not an oxidoreductase; (c) a signal peptide homologous to a protein encoded by the gene of interest; (d) a signal peptide homologous to a protein encoded by the gene of interest, wherein the signal peptide homologous to the protein encoded by the gene of interest is modified by insertion, deletion, and/or substitution of at least one amino acid; and (e) a naturally occurring amino acid sequence which does not have the function of a signal peptide in nature, wherein the naturally occurring amino acid sequence is optionally modified by insertion, deletion, and/or substitution of at least one amino acid. In some instances, the amino acids 1-9 of the N-terminal end of the signal peptide have an average hydrophobic score of above 2.

In some instances, a target motif heterologous to a protein encoded by the gene of interest or a signal peptide heterologous to a protein encoded by the gene of interest as used herein can refer to a naturally occurring target motif or signal peptide which is different from the naturally occurring target motif or signal peptide of a protein. For example, the target motif or the signal peptide is not derived from the gene of interest. Usually a target motif or a signal peptide heterologous to a given protein is a target motif or a signal peptide from another protein, which is not related to the given protein. For example, a target motif or a signal peptide heterologous to a given protein has an amino acid sequence that is different from the amino acid sequence of the target motif or the signal peptide of the given protein by more than 50%, 60%, 70%, 80%, 90%, or by more than 95%. Although heterologous sequences may be derived from the same organism, they naturally (in nature) do not occur in the same nucleic acid molecule, such as in the same mRNA. The target motif or the signal peptide heterologous to a protein and the protein to which the target motif or the signal peptide is heterologous can be of the same or different origin. In some embodiments, they are of eukaryotic origin. In some embodiments, they are of the same eukaryotic organism. In some embodiments, they are of mammalian origin. In some embodiments, they are of the same mammalian organism. In some embodiments, they are human origin. For example, an RNA construct may comprise a nucleic acid sequence encoding the human IL-4 gene and a signal peptide of another human protein. In some embodiments, an RNA construct may comprise a signal peptide heterologous to a protein wherein the signal peptide and the protein are of the same origin, namely of human origin.

In some instance, a target motif homologous to a protein encoded by the gene of interest or a signal peptide homologous to a protein encoded by the gene of interest as used herein can refer to a naturally occurring target motif or signal peptide of a protein. A target motif or a signal peptide homologous to a protein is the target motif or the signal peptide encoded by the gene of the protein as it occurs in nature. A target motif or a signal peptide homologous to a protein is usually of eukaryotic origin. In some embodiments, a target motif or a signal peptide homologous to a protein is of mammalian origin. In some embodiments, a target motif or a signal peptide homologous to a protein is of human origin.

In some instances, a naturally occurring amino acid sequence which does not have the function of a target motif in nature or a naturally occurring amino acid sequence which does not have the function of a signal peptide in nature as used herein can refer to an amino acid sequence which occurs in nature and is not identical to the amino acid sequence of any target motif or signal peptide occurring in nature. A naturally occurring amino acid sequence which does not have the function of a target motif or a signal peptide in nature can be between 10-50 amino acids long. In some embodiments, a naturally occurring amino acid sequence which does not have the function of a target motif or a signal peptide in nature is of eukaryotic origin and not identical to any target motif or signal peptide of eukaryotic origin. In some embodiments, a naturally occurring amino acid sequence which does not have the function of a target motif or a signal peptide in nature is of mammalian origin and not identical to any target motif or signal peptide of mammalian origin. In some embodiments, a naturally occurring amino acid sequence which does not have the function of a target motif or a signal peptide in nature is of human origin and not identical to any target motif or signal peptide of human origin occurring in nature. A naturally occurring amino acid sequence which does not have the function of a target motif or a signal peptide in nature is usually an amino acid sequence of the coding sequence of a protein. The terms “naturally occurring,” “natural,” and “in nature” as used herein have the equivalent meaning.

In some instances, amino acids 1-9 of the N-terminal end of the signal peptide as used herein can refer to the first nine amino acids of the N-terminal end of the amino acid sequence of a signal peptide. Analogously, amino acids 1-7 of the N-terminal end of the signal peptide as used herein can refer to the first seven amino acids of the N-terminal end of the amino acid sequence of a signal peptide and amino acids 1-5 of the N-terminal end of the signal peptide can refer to the first five amino acids of the N-terminal end of the amino acid sequence of a signal peptide.

In some instances, amino acid sequence modified by insertion, deletion, and/or substitution of at least one amino acid can refer to an amino acid sequence which includes an amino acid substitution, insertion, and/or deletion of at least one amino acid within the amino acid sequence. For example, target motif heterologous to a protein encoded by the gene of interest is modified by insertion, deletion, and/or substitution of at least one amino acid or signal peptide heterologous to a protein encoded by the gene of interest is modified by insertion, deletion, and/or substitution of at least one amino acid as used herein can refer to an amino acid sequence of a naturally occurring target motif or signal peptide heterologous to a protein which includes an amino acid substitution, insertion, and/or deletion of at least one amino acid within its naturally occurring amino acid sequence. For example, target motif homologous to a protein encoded by the gene of interest is modified by insertion, deletion, and/or substitution of at least one amino acid or signal peptide homologous to a protein encoded by the gene of interest is modified by insertion, deletion, and/or substitution of at least one amino acid as used herein can refer to a naturally occurring target motif or signal peptide homologous to a protein which includes an amino acid substitution, insertion, and/or deletion of at least one amino acid within its naturally occurring amino acid sequence. In some embodiments, naturally occurring amino acid sequence may be modified by insertion, deletion, and/or substitution of at least one amino acid and a naturally occurring amino acid sequence can include an amino acid substitution, insertion, and/or deletion of at least one amino acid within its naturally occurring amino acid sequence. An amino acid substitution or a substitution may refer to replacement of an amino acid at a particular position in an amino acid or polypeptide sequence with another amino acid. For example, the substitution R34K refers to a polypeptide, in which the arginine (Arg or R) at position 34 is replaced with a lysine (Lys or K). For the preceding example, 34K indicates the substitution of an amino acid at position 34 with a lysine (Lys or K). In some embodiments, multiple substitutions are typically separated by a slash. For example, R34K/L38V refers to a variant comprising the substitutions R34K and L38V. An amino acid insertion or an insertion may refer to addition of an amino acid at a particular position in an amino acid or polypeptide sequence. For example, insert −34 designates an insertion at position 34. An amino acid deletion or a deletion may refer to removal of an amino acid at a particular position in an amino acid or polypeptide sequence. For example, R34—designates the deletion of arginine (Arg or R) at position 34.

In some instances, deleted amino acid is an amino acid with a hydrophobic score of below −0.8, −0.7, −0.6, −0.5, −0.4, −0.3, −0.2, −0.1, 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, or below 1.9. In some instances, the substitute amino acid is an amino acid with a hydrophobic score which is higher than the hydrophobic score of the substituted amino acid. For example, the substitute amino acid is an amino acid with a hydrophobic score of 2.8 and higher, or 3.8 and higher. In some instances, the inserted amino acid is an amino acid with a hydrophobic score of 2.8 and higher or 3.8 and higher.

In some instances, an amino acid sequence described herein may comprise 1 to 15 amino acid insertions, deletions, and/or substitutions. In some embodiments, an amino acid sequence described herein may comprise 1 to 7 amino acid insertions, deletions, and/or substitutions. In some instances, an amino acid sequence described herein may not comprise amino acid insertions, deletions, and/or substitutions. In some instances, an amino acid sequence described herein may comprise 1 to 15 amino acid insertions, deletions, and/or substitutions within the amino acids 1-30 of the N-terminal end of the amino acid sequence of the target motif or the signal peptide. In some embodiments, an amino acid sequence described herein may comprise 1 to 9 amino acid insertions, deletions, and/or substitutions within the amino acids 1-30 of the N-terminal end of the amino acid sequence of the target motif or the signal peptide. In some instances, an amino acid sequence described herein may comprise 1 to 15 amino acid insertions, deletions, and/or substitutions within the amino acids 1-20 of the N-terminal end of the amino acid sequence of the target motif or the signal peptide. In some embodiments, an amino acid sequence described herein may comprise 1 to 9 amino acid insertions, deletions, and/or substitutions within the amino acids 1-20 of the N-terminal end of the amino acid sequence of the target motif or the signal peptide. In some instances, at least one amino acid of an amino acid sequence described herein may be optionally modified by deletion, and/or substitution.

In some instances, the average hydrophobic score of the first nine amino acids of the N-terminal end of the amino acid sequence of the modified signal peptide is increased 1.0 unit or above compared to the signal peptide without modification. In some instances, hydrophobic score or hydrophobicity score can be used synonymously to hydropathy score herein and can refer to the degree of hydrophobicity of an amino acid as calculated according to the Kyte-Doolittle scale (Kyte J., Doolittle R. F.; J. Mol. Biol. 157:105-132(1982)). The amino acid hydrophobic scores according to the Kyte-Doolittle scale are as follows:

TABLE A
Amino Acid Hydrophobic Scores

| Amino Acid | One Letter Code | Hydrophobic Score |
|---|---|---|
| Isoleucine | I | 4.5 |
| Valine | V | 4.2 |
| Leucine | L | 3.8 |
| Phenylalanine | F | 2.8 |
| Cysteine | C | 2.5 |
| Methionine | M | 1.9 |
| Alanine | A | 1.8 |
| Glycine | G | −0.4 |
| Threonine | T | −0.7 |
| Serine | S | −0.8 |
| Tryptophan | W | −0.9 |
| Tyrosine | Y | −1.3 |
| Proline | P | −1.6 |
| Histidine | H | −3.2 |
| Glutamic acid | E | −3.5 |
| Glutamine | Q | −3.5 |
| Aspartic acid | D | −3.5 |
| Asparagine | N | −3.5 |
| Lysine | K | −3.9 |
| Arginine | R | −4.5 |

In some instances, average hydrophobic score of an amino acid sequence can be calculated by adding the hydrophobic score according to the Kyte-Doolittle scale of each of the amino acid of the amino acid sequence divided by the number of the amino acids. For example, the average hydrophobic score of the amino acids 1-9 of the N-terminal end of the amino acid sequence of a signal peptide can be calculated by adding the hydrophobic score or each of the nine amino acids divided by nine.

The polarity is calculated according to Zimmerman Polarity index (Zimmerman J. M., Eliezer N., Simha R.; J. Theor. Biol. 21:170-201(1968)). In some embodiments, average polarity of an amino acid sequence can be calculated by adding the polarity value calculated according to Zimmerman Polarity index of each of the amino acid of the amino acid sequence divided by the number of the amino acids. For example, the average polarity of the amino acids 1-9 of the N-terminal end of the amino acid sequence of a signal peptide can be calculated by adding the average polarity of each of the nine amino acids of the amino acids 1-9 of the N-terminal end, divided by nine. The polarity of amino acids according to Zimmerman Polarity index is as follows:

TABLE B
Amino Acid Polarity

| Amino Acid | One Letter Code | Polarity |
|---|---|---|
| Isoleucine | I | 0.13 |
| Valine | V | 0.13 |
| Leucine | L | 0.13 |
| Phenylalanine | F | 0.35 |
| Cysteine | C | 1.48 |
| Methionine | M | 1.43 |
| Alanine | A | 0 |
| Glycine | G | 0 |
| Threonine | T | 1.66 |
| Serine | S | 1.67 |
| Tryptophan | W | 2.1 |
| Tyrosine | Y | 1.61 |
| Proline | P | 1.58 |
| Histidine | H | 51.6 |
| Glutamic acid | E | 49.9 |
| Glutamine | Q | 3.53 |
| Aspartic acid | D | 49.7 |
| Asparagine | N | 3.38 |
| Lysine | K | 49.5 |
| Arginine | R | 52 |

In some instances, a naturally occurring signal peptide of Insulin-like Growth Factor 1 (IGF-1) may be modified by one or more substitutions, deletions, and/or insertions, wherein the naturally occurring signal peptide of IGF-1 is referred to the amino acids 1-20 of the IGF-1 amino acid sequence in the Uniprot database as P05019 and in the Genbank database as NM_001111285.3. In some instances, the amino acid sequence of IGF-1 signal peptide may be modified by the one or more substitutions, deletions, and/or insertions selected from the group consisting of G2L, K3-, S5L, T9L, Q10L, and C15-. In some instances, a naturally occurring signal peptide of IGF-1 may be replaced with a signal peptide of another protein. In some instances, a naturally occurring signal peptide of IGF-1 may be replaced with a naturally occurring signal peptide of another protein. For example, a naturally occurring signal peptide of IGF-1 may be replaced with a naturally occurring signal peptide of brain-derived neurotrophic factor (BDNF). In some embodiments, the wild type (WT) IGF-1 signal peptide amino acid sequence comprises a sequence comprising SEQ ID NO: 49. In some instances, a modified IGF-1 signal peptide has an amino acid sequence comprising a sequence comprising SEQ ID NO: 44 encoded by the DNA sequence as shown in SEQ ID NO: 45. In some embodiments, a modified IGF-1 signal peptide may comprise a signal peptide from another protein. In some instances, a modified IGF-1 signal peptide has an amino acid sequence comprising a sequence comprising SEQ ID NO: 51 encoded by the DNA sequence as shown in SEQ ID NO: 52.

SEQ ID NO: 44
Met-Leu-Ile-Leu-Leu-Leu-Pro-Leu-Leu-Leu-Phe-Lys-
Cys-Phe-Cys-Asp-Phe-Leu-Lys
SEQ ID NO: 45
ATGCTGATTCTGCTGCTGCCCCTGCTGCTGTTCAAGTGCTTCTGCGAC
TTCCTGAAA
SEQ ID NO: 51
MTILFLTMVISYFGCMKA
SEQ ID NO: 52
ATGACCATCCTGTTTCTGACAATGGTCATCAGCTACTTCGGCTGCATG
AAGGCC

In some instances, the pro-peptide of IGF-1 may be modified. In some embodiments, a naturally occurring amino acid sequence of the pro-peptide of IGF-1, which does not have the function of a signal peptide in nature (Uniprot database as P05019), is modified by deletion of ten amino acid residues (VKMHTMSSSH (SEQ ID NO: 48)) flanking 22-31 in the N-terminal end of the pro-peptide and has preferably the amino acid sequence as shown in SEQ ID NO: 46 encoded by the DNA sequence as shown in SEQ ID NO: 45.

SEQ ID NO: 46
Met-Leu-Phe-Tyr-Leu-Ala-Leu-Cys-Leu-Leu-Thr-Phe-
Thr-Ser-Ser-Ala-Thr-Ala
SEQ ID NO: 47
ATGCTGTTCTATCTGGCCCTGTGCCTGCTGACCTTTACCAGCTCTGCT
ACCGCC

In some instances, an mRNA comprising a nucleic acid sequence encoding the pro-peptide of IGF-1 and a nucleic acid sequence encoding the mature IGF-1, but not comprising a nucleic acid sequence encoding an E-peptide of IGF-1 may refer to an mRNA which comprises a nucleotide sequence encoding the pro-peptide of human IGF-1 having 27 amino acids and a nucleotide sequence encoding the mature human IGF-1 having 70 amino acids, but does not comprise a nucleotide sequence encoding an E-peptide of human IGF-1 i.e., does not comprise a nucleotide sequence encoding an Ea-, Eb-, or Ec-domain. The nucleotide sequence encoding the pro-peptide of human IGF-1 having 27 amino acids and the nucleotide sequence encoding the mature human IGF-1 having 70 amino acids may be codon-optimized.

In some instances, a naturally occurring signal peptide of Interleukin 4 (IL-4) may be modified by one or more substitutions, deletions, and/or insertions, wherein the naturally occurring signal peptide of IL-4 is referred to the amino acids 1-24 of the IL-4 amino acid sequence in the Uniprot database as P05112 and in the Genbank database as NM_000589.4. In some instances, the wild type (WT) IL-4 signal peptide amino acid sequence comprises a sequence as shown in SEQ ID NO: 53. In some instances, the WT IL-4 signal peptide is encoded by a DNA sequence as shown in SEQ ID NO: 54. In some instances, a modified IL-4 signal peptide has an amino acid sequence comprising a sequence as shown in SEQ ID NO: 55. In some instances, a modified IL-4 signal peptide is encoded by a DNA sequence as shown in SEQ ID NO: 56.

### Linkers

In some aspects, provided herein, is a composition comprising a recombinant RNA construct comprising a first RNA sequence, a second RNA sequence, and a linker RNA sequence, wherein: (i) the first RNA sequence is a first small interfering RNA (siRNA) sequence; (ii) the second RNA sequence is a second siRNA sequence or a first messenger RNA (mRNA) sequence encoding a gene of interest (GOI); and (iii) the linker RNA sequence links the first RNA sequence and the second RNA sequence, wherein the linker RNA sequence has a structure selected from the group consisting of: Formula (I): XₘCAACAAXₙ, wherein X is any nucleotide, m is an integer from 1 to 12, and n is an integer from 0 to 4 (SEQ ID NO: 129); and Formula (II): XₚTCCCXᵣ, wherein X is any nucleotide, p is an integer from 0 to 17, and r is an integer from 0 to 13 (SEQ ID NO: 130).

In some aspects, provided herein, is a composition comprising a recombinant RNA construct comprising a first RNA sequence, a second RNA sequence, and a linker RNA sequence, wherein: (i) the first RNA sequence is a first small interfering RNA (siRNA) sequence; (ii) the second RNA sequence is a second siRNA sequence or a first messenger RNA (mRNA) sequence encoding a gene of interest (GOI); and (iii) the linker RNA sequence links the first RNA sequence and the second RNA sequence, wherein the linker RNA sequence comprises a sequence comprising ACAACAA (SEQ ID NO: 85).

In some aspects, provided herein, is a composition comprising a recombinant RNA construct comprising a first RNA sequence, a second RNA sequence, and a linker RNA sequence, wherein: (i) the first RNA sequence is a first small interfering (siRNA) sequence; (ii) the second RNA sequence is a second siRNA sequence or a first messenger (mRNA) sequence encoding a gene of interest (GOI); and (iii) the linker RNA sequence links the first RNA sequence and the second RNA sequence, wherein (a) the linker RNA sequence is not TTTATCTTAGAGGCATATCCCTACGTACCAACAA (SEQ ID NO: 28) or ATAGTGAGTCGTATTAACGTACCAACAA (SEQ ID NO: 27); or (b) the linker RNA sequence does not form a secondary structure according to RNAfold WebServer.

In some embodiments, the second RNA sequence is a second siRNA sequence. In some embodiments, the linker RNA sequence comprises ACAACAA (SEQ ID NO: 85), ATCCCTACGTACCAACAA (SEQ ID NO: 87), ACGTACCAACAA (SEQ ID NO: 88), TCCC (SEQ ID NO: 89), or ACAACAATCCC (SEQ ID NO: 90). In some embodiments, the recombinant RNA construct further comprises a first mRNA sequence encoding a GOI. In some embodiments, the second RNA sequence is a first mRNA sequence encoding a GOI.

In some embodiments, the linker RNA sequence comprises ACAACAA (SEQ ID NO: 85), ATAGTGAGTCGTATTATCCC (SEQ ID NO: 92), ATAGTGAGTCGTATTAACAACAATCCC (SEQ ID NO: 93), ATAGTGAGTCGTATTAACAACAA (SEQ ID NO: 94), ATAGTGAGTCGTATTAATCCCTACGTACCAACAA (SEQ ID NO: 95), or ATAGTGAGTCGTATTAACGTACCAACAA (SEQ ID NO: 27).

In some embodiments, the recombinant RNA construct further comprises a second mRNA sequence encoding a GOI. In some embodiments, the recombinant RNA construct further comprises a second siRNA sequence. In some embodiments, the recombinant RNA construct comprises a third siRNA sequence. In some embodiments, the recombinant RNA construct further comprises four, five, or more siRNA sequences. In some embodiments, each of the siRNA sequences binds to a target RNA and modulates the expression of the target RNA.

In some embodiments, each of the siRNA sequences is capable of binding to: (a) different target RNAs; (b) different regions of the same target RNA; (c) the same region of the same target RNA; or (d) any combinations thereof. In some embodiments, the siRNA sequences of (c) are the same. In some embodiments, the recombinant RNA construct comprises three, four, five, or more mRNA sequences, each encoding a GOI. In some embodiments, each of the mRNA sequences encodes the same GOI. In some embodiments, each of the mRNA sequences encodes a different GOI.

In some embodiments, the length of the linker RNA sequence between siRNA sequences is from about 4 to about 27 nucleotides. In some embodiments, the length the linker RNA sequence between siRNA sequences is from about 4 to about 18 nucleotides. In some embodiments, m is 1 and n is 0. In some embodiments, the linker RNA sequence between siRNA sequences is ACAACAATCCC (SEQ ID NO: 90). In some embodiments, the linker RNA sequence is ACAACAA (SEQ ID NO: 85). In some embodiments, the linker RNA sequence comprises a sequence selected from the group consisting of SEQ ID NOs: 85, 87-95. In some embodiments, the linker RNA sequence comprises a sequence according to SEQ ID NO: 85.

In some embodiments, the expression of the GOI is modulated. In some embodiments, the expression of the GOI is upregulated by expressing a protein encoded by the GOI. In some embodiments, the expression of the target RNA is modulated. In some embodiments, the expression of the target RNA is downregulated by the siRNA sequences capable of binding to the target RNA. In some embodiments, the siRNA sequences capable of binding to the target RNA does not inhibit the expression of the GOI.

In some embodiments, the RNA linker sequence between siRNA sequences does not form a secondary structure according to RNAfold WebServer. In some embodiments, an siRNA sequence forms a secondary structure according to RNAfold WebServer.

In some embodiments, the recombinant RNA construct is cleaved. In some embodiments, the recombinant RNA construct is cleaved by an intracellular protein. In some embodiments, the recombinant RNA construct is cleaved by an endogenous protein. In some embodiments, the recombinant RNA construct is cleaved by an endogenous DICER.

In some embodiments, the cleavage of the recombinant RNA construct is enhanced compared to the cleavage of an RNA construct that does not comprise a linker having a structure selected from the group consisting of Formula (I) and Formula (II). In some embodiments, the cleavage of the recombinant RNA construct is enhanced compared to the cleavage of an RNA construct that does not comprise a linker comprising a sequence comprising ACAACAA (SEQ ID NO: 85). In some embodiments, the cleavage of the recombinant RNA construct is enhanced compared to the cleavage of an RNA construct comprising a linker that forms a secondary structure.

In some embodiments, the expression of the gene of interest is enhanced compared to the expression of a gene of interest from an RNA construct that does not comprise a linker having a structure selected from the group consisting of Formula (I) and Formula (II). In some embodiments, the expression of the gene of interest is enhanced compared to the expression of a gene of interest from an RNA construct that does not comprise a linker comprising a sequence comprising ACAACAA (SEQ ID NO: 85).

In some aspects, provided herein, is a composition comprising a recombinant polynucleic acid construct comprising a nucleic acid sequence selected from the group consisting of SEQ ID NOs: 1-24 and 97-108.

### Expression Vector and Production of RNA Constructs

Provided herein are compositions comprising recombinant polynucleic acid constructs encoding recombinant RNA constructs described herein. Provided herein are compositions comprising recombinant polynucleic acid constructs encoding recombinant RNA constructs comprising a first RNA sequence and a second RNA sequence. In some instances, the first RNA sequence or the second RNA sequence may encode a gene of interest. In some embodiments, the first RNA sequence or the second RNA sequence may be an mRNA encoding a gene of interest. In some instances, the first RNA sequence or the second RNA sequence may comprise at least two genetic elements that modulate the expression of one or more target RNA. In some embodiments, the first RNA sequence or the second RNA sequence may comprise at least two siRNAs each capable of binding to a target RNA. For example, an mRNA encoding a gene of interest can be an mRNA of IL-4 or IGF-1. For example, a target RNA can be an mRNA of TNF-alpha, IL-17, IL-8, IL-1beta, or Turbo GFP.

In related aspects, recombinant polynucleic acid constructs encoding recombinant RNA constructs may encode 2, 3, 4, 5, or more siRNA species. In related aspects, recombinant polynucleic acid constructs encoding recombinant RNA constructs may encode 2 siRNA species directed to a target mRNA. In related aspects, recombinant polynucleic acid constructs encoding recombinant RNA constructs may encode 3 siRNAs, each directed to a target mRNA. In related aspects, each of the siRNA species may comprise the same sequence, different sequence, or a combination thereof. For example, recombinant polynucleic acid constructs encoding recombinant RNA constructs may encode 3 siRNAs, each directed to the same region or sequence of the target mRNA. For example, recombinant polynucleic acid constructs encoding recombinant RNA constructs may encode 3 siRNAs, each directed to a different region or sequence of the target mRNA. In some aspects, recombinant polynucleic acid constructs encoding recombinant RNA constructs may encode 3 siRNA species, wherein each of the 3 siRNA species is directed to a different target mRNA. In some embodiments, a target mRNA may be TNF-alpha mRNA, IL-8 mRNA, IL-17 mRNA, Turbo GFP mRNA, or IL-1beta mRNA. In related aspects, recombinant polynucleic acid constructs may comprise a sequence selected from the group consisting of SEQ ID NOs: 13-24.

The polynucleic acid constructs, described herein, can be obtained by any method known in the art, such as by chemically synthesizing the DNA chain, by PCR, or by the Gibson Assembly method. The advantage of constructing polynucleic acid constructs by chemical synthesis or a combination of PCR method or Gibson Assembly method is that the codons may be optimized to ensure that the fusion protein is expressed at a high level in a host cell. Codon optimization can refer to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA). Once obtained polynucleotides can be incorporated into suitable vectors. Vectors as used herein can refer to naturally occurring or synthetically generated constructs for uptake, proliferation, expression or transmission of nucleic acids in vivo or in vitro, e.g., plasmids, minicircles, phagemids, cosmids, artificial chromosomes/mini-chromosomes, bacteriophages, viruses such as baculovirus, retrovirus, adenovirus, adeno-associated virus, herpes simplex virus, bacteriophages. Methods used to construct vectors are well known to a person skilled in the art and described in various publications. In particular techniques for constructing suitable vectors, including a description of the functional and regulatory components such as promoters, enhancers, termination and polyadenylation signals, selection markers, origins of replication, and splicing signals, are known to the person skilled in the art. A variety of vectors are well known in the art and some are commercially available from companies such as Agilent Technologies, Santa Clara, Calif.; Invitrogen, Carlsbad, Calif.; Promega, Madison, Wis.; Thermo Fisher Scientific; or Invivogen, San Diego, Calif A non-limiting examples of vectors for in vitro transcription includes pT7CFE1-CHis, pMX (such as pMA-T, pMA-RQ, pMC, pMK, pMS, pMZ), pEVL, pSP73, pSP72, pSP64, and pGEM (such as pGEM®-4Z, pGEM®-5Zf(+), pGEM®-11Zf(+), pGEM®-9Zf(−), pGEM®-3Zf(+/−), pGEM®-7Zf(+/−)). In some instances, recombinant polynucleic acid constructs may be DNA.

The polynucleic acid constructs, as described herein, can be circular or linear. For example, circular polynucleic acid constructs may include vector system such as pMX, pMA-T, pMA-RQ, or pT7CFE1-CHis. For example, linear polynucleic acid constructs may include linear vector such as pEVL or linearized vectors. In some instances, recombinant polynucleic acid constructs may further comprise a promoter. In some instances, the promoter may be present upstream of or 5′ to the sequence encoding for the first RNA sequence and the second RNA sequence. Non-limiting examples of a promoter can include T3, T7, SP6, P60, Syn5, and KP34. In some instances, recombinant polynucleic acid constructs provided herein may comprise a T7 promoter comprising a sequence comprising TAATACGACTCACTATA (SEQ ID NO: 26). In some instances, recombinant polynucleic acid constructs further comprises a sequence encoding a Kozak sequence. A Kozak sequence may refer to a nucleic acid sequence motif that functions as the protein translation initiation site. Kozak sequences are described at length in the literature, e.g., by Kozak, M., Gene 299(1-2):1-34, incorporated herein by reference herein in its entirety. In some embodiments, recombinant polynucleic acid constructs comprises a sequence encoding a Kozak sequence comprising a sequence comprising GCCACC (SEQ ID NO: 25). In some instances, recombinant polynucleic acid constructs described herein may be codon-optimized.

Provided herein are compositions comprising recombinant polynucleic acid constructs encoding RNA constructs described herein comprising at least two nucleic acid sequences each encoding an siRNA capable of binding to one or more target RNAs and one or more nucleic acid sequence encoding a gene of interest, wherein the siRNA capable of binding to a target RNA is not a part of an intron sequence encoded by the gene of interest. In some instances, the gene of interest is expressed without RNA splicing. In some instances, the siRNA capable of binding to a target RNA is not encoded by or comprised of an intron sequence of the gene of interest. In some instances, the siRNA capable of binding to a target RNA binds to an exon of a target RNA. In some instances, the siRNA capable of binding to a target RNA specifically binds to one target RNA. In some instances, recombinant polynucleic acid constructs may comprise a nucleic acid sequence comprising a sequence selected from the group consisting of SEQ ID NOs: 13-24.

Provided herein are methods of producing RNA construct compositions described herein. For example, recombinant RNA constructs may be produced by in vitro transcription from a polynucleic acid construct comprising a promoter for an RNA polymerase, at least one nucleic acid sequence encoding a gene of interest, at least two nucleic acid sequences encoding siRNAs capable of binding to one or more target mRNAs, and a nucleic acid sequence encoding poly(A) tail. In vitro transcription reaction may further comprise an RNA polymerase, a mixture of nucleotide triphosphates (NTPs), and/or a capping enzyme. Details of producing RNAs using in vitro transcription as well as isolating and purifying transcribed RNAs is well known in the art and can be found, for example, in Beckert & Masquida ((2011) Synthesis of RNA by In vitro Transcription. RNA. Methods in Molecular Biology (Methods and Protocols), vol 703. Humana Press). A non-limiting list of in vitro transcript kits includes MEGAscript™ T3 Transcription Kit, MEGAscript T7 kit, MEGAscript™ SP6 Transcription Kit, MAXIscript™ T3 Transcription Kit, MAXIscript™ T7 Transcription Kit, MAXIscript™ SP6 Transcription Kit, MAXIscript™ T7/T3 Transcription Kit, MAXIscript™ SP6/T7 Transcription Kit, mMESSAGE mMACHINE™ T3 Transcription Kit, mMESSAGE mMACHINE™ T7 Transcription Kit, mMESSAGE mMACHINE™ SP6 Transcription Kit, MEGAshortscript™ T7 Transcription Kit, HiScribe™ T7 High Yield RNA Synthesis Kit, HiScribe™ T7 In Vitro Transcription Kit, AmpliScribe™ T7-Flash™ Transcription Kit, AmpliScribe™ T7 High Yield Transcription Kit, AmpliScribe™ T7-Flash™ Biotin-RNA Transcription Kit, T7 Transcription Kit, HighYield T7 RNA Synthesis Kit, DuraScribe® T7 Transcription Kit, etc.

The in vitro transcription reaction can further comprise a transcription buffer system, nucleotide triphosphates (NTPs), and an RNase inhibitor. In some embodiments, the transcription buffer system may comprise dithiothreitol (DTT) and magnesium ions. The NTPs can be naturally occurring or non-naturally occurring (modified) NTPs. Non-limiting examples of non-naturally occurring (modified) NTPs include N¹-methylpseudouridine, pseudouridine, N¹-ethylpseudouridine, N¹-methoxymethylpseudouridine, N¹-propylpseudouridine, 2-thiouridine, 4-thiouridine, 5-methoxyuridine, 5-methylurdine, 5-carboxymethylesteruridine, 5-formyluridine, 5-carboxyuridine, 5-hydroxyuridine, 5-bromouridine, 5-Iodouridine, 5,6-dihydrouridine, 6-azauridine, thienouridine, 3-methyluridine, 1-carboxymethyl-pseudouridine, 4-thio-1-methyl-pseudouridine, 2-thio-1-methyl-pseudouridine, dihydrouridine, dihydropseudouridine, 2-methoxyuridine, 2-methoxy-4-thio-uridine, 4-methoxy-pseudouridine, 4-methoxy-2-thio-pseudouridine, 5-methylcytidine, 5-methoxycytidine, 5-hydroxymethylcytidine, 5-formylcytidine, 5-carboxycytidine, 5-hydroxycytidine, 5-iodocytidine, 5-bromocytidine, 2-thiocytidine, 5-azacytidine, pseudoisocytidine, 3-methyl-cytidine, N⁴-acetylcytidine, 5-formylcytidine, N⁴-methylcytidine, 5-hydroxymethylcytidine, 1-methyl-pseudoisocytidine, 4-methoxy-pseudoisocytidine, and 4-methoxy-1-methyl-pseudoisocytidine, N¹-methyladenosine, N⁶-methyladenosine, N⁶-methyl-2-aminoadenosine, N⁶-isopentenyladenosine, N⁶,N⁶-dimethyladenosine, 7-methyladenine, 2-methylthio-adenine, and 2-methoxy-adenine. Non-limiting examples of DNA-dependent RNA polymerase include T3, T7, SP6, P60, Syn5, and KP34 RNA polymerases. In some embodiments, the RNA polymerase is selected from the group consisting of T3 RNA polymerase, T7 RNA polymerase, SP6 RNA polymerase, P60 RNA polymerase, Syn5 RNA polymerase, and KP34 RNA polymerase.

Transcribed RNAs, as described herein, may be isolated and purified from the in vitro transcription reaction mixture. For example, transcribed RNAs may be isolated and purified using column purification. Details of isolating and purifying transcribed RNAs from in vitro transcription reaction mixture is well known in the art and any commercially available kits may be used. A non-limiting list of RNA purification kits includes MEGAclear kit, Monarch® RNA Cleanup Kit, EasyPure® RNA Purification Kit, NucleoSpin® RNA Cleanup, etc.

### Therapeutic Applications

Provided herein are compositions useful in the treatment of a disease or condition. In some aspects, compositions are present or administered in an amount sufficient to treat or prevent a disease or condition. In some aspects, provided herein, is a method of treating a disease or condition comprising administering to a subject in need thereof the composition or the pharmaceutical composition described herein. In some aspects, provided herein, is the composition or the pharmaceutical composition described herein for use in a method of treating a disease or a condition in a subject in need thereof. In some aspects, provided herein, is the use of the composition or the pharmaceutical composition described herein for the manufacture of a medicament for treating a disease or a condition in a subject in need thereof. In some embodiments, the disease or condition comprises a skin disease or condition or a joint disease or condition. In some embodiments, the skin disease or condition comprises an inflammatory skin disorder. In some embodiments, an inflammatory skin disorder comprises psoriasis. In some embodiments, the joint degeneration comprises intervertebral disc disease (IVDD) or osteoarthritis (OA). Provided herein are recombinant polynucleic acid or RNA construct compositions comprising a first RNA sequence encoding a gene of interest, and a second RNA sequence comprising at least two genetic elements that modulate expression of one or more target RNAs, wherein contacting a human cell with the recombinant RNA construct results in an immune response that is lower than the immune response of the human cell contacted with a corresponding recombinant RNA construct comprising the first RNA sequence encoding a gene of interest and a corresponding second RNA sequence comprising at most one of the at least two genetic elements that modulate expression of one or more target RNAs. In some instances, the first RNA sequence or the second RNA sequence may encode a gene of interest. In some embodiments, the gene of interest may comprise IL-4 or IGF-1. In some instances, the first RNA sequence or the second RNA sequence may comprise at least two genetic elements that can reduce expression of a gene associated with a disease or condition described herein. In some embodiments, the genetic element that can reduce expression of a gene associated with a disease or condition may comprise siRNA targeting TNF-alpha mRNA or a functional variant. In some embodiments, the genetic element that can reduce expression of a gene associated with a disease or condition may comprise siRNA targeting IL-8 mRNA or a functional variant. In some embodiments, the genetic element that can reduce expression of a gene associated with a disease or condition may comprise siRNA targeting IL-1beta mRNA or a functional variant. In some embodiments, the genetic element that can reduce expression of a gene associated with a disease or condition may comprise siRNA targeting IL-17 mRNA or a functional variant.

Also provided herein are pharmaceutical compositions comprising any recombinant RNA construct composition described herein and a pharmaceutically acceptable excipient. A pharmaceutical composition can denote a mixture or solution comprising a therapeutically effective amount of an active pharmaceutical ingredient together with one or more pharmaceutically acceptable excipients to be administered to a subject in need thereof. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. The term “Pharmaceutically acceptable” can refer to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. A pharmaceutically acceptable excipient can denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products. Pharmaceutical compositions can facilitate administration of the compound to an organism and can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, pharmaceutical compositions can be formulated by dissolving active substances (e.g., recombinant polynucleic acid or RNA constructs described herein) in aqueous solution for injection into disease tissues or disease cells. In some embodiments, pharmaceutical compositions can be formulated by dissolving active substances (e.g., recombinant polynucleic acid or RNA constructs described herein) in aqueous solution for direct injection into disease tissues or disease cells.

Also provided herein are methods of treating a disease or condition in a subject in need thereof, comprising administering to the subject a therapeutically effective amount of polynucleic acid construct or recombinant RNA construct compositions or pharmaceutical compositions described herein. The terms “effective amount” or “therapeutically effective amount,” as used herein, refer to a sufficient amount of an agent or a compound being administered which will relieve to some extent one or more of the symptoms of the disease or the condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The terms “treat,” “treating” or “treatment,” as used herein, include alleviating, abating or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. In some embodiments, treating a disease or condition comprises reducing the size of disease tissues or disease cells. In some embodiments, treating a disease or a condition in a subject comprises increasing the survival of a subject. In some embodiments, treating a disease or condition comprises reducing or ameliorating the severity of a disease, delaying onset of a disease, inhibiting the progression of a disease, reducing hospitalization of or hospitalization length for a subject, improving the quality of life of a subject, reducing the number of symptoms associated with a disease, reducing or ameliorating the severity of a symptom associated with a disease, reducing the duration of a symptom associated with a disease, preventing the recurrence of a symptom associated with a disease, inhibiting the development or onset of a symptom of a disease, or inhibiting of the progression of a symptom associated with a disease.

In some cases, a subject can encompass mammals. Examples of mammals include, but are not limited to, any member of the mammalian class. humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some cases, the mammal is a human. In some cases, the subject may be an animal. In some cases, an animal may comprise human beings and non-human animals. In one embodiment, a non-human animal may be a mammal, for example a rodent such as rat or a mouse. In another embodiment, a non-human animal may be a mouse. In some instances, the subject is a mammal. In some instances, the subject is a human. In some instances, the subject is an adult, a child, or an infant. In some instances, the subject is a companion animal. In some instances, the subject is a feline, a canine, or a rodent. In some instances, the subject is a dog or a cat.

In some aspects, provided herein, is a method of treating a disease or condition in a subject, comprising administering to the subject recombinant RNA construct compositions or pharmaceutical compositions, described herein, comprising an mRNA encoding a gene of interest and at least two siRNAs capable of binding to a target mRNA. In some embodiments, the target mRNA comprises an mRNA of TNF-alpha, IL-1beta, IL-8, IL-17, Turbo GFP, or a functional variant thereof. In some embodiments, the mRNA encoding the gene of interest encodes IGF-1 or a functional variant thereof. In some embodiments, the mRNA encoding the gene of interest encodes a cytokine. In some embodiments, the cytokine is an IL-4 or a functional variant thereof.

In some aspects, provided herein, is a method of treating a disease or condition in a subject, the method comprising administering to the subject recombinant RNA construct compositions or pharmaceutical compositions, described herein, comprising an mRNA encoding IGF-1 and siRNA capable of binding to an mRNA of IL-8. In some aspects, provided herein, is a method of treating a disease or condition in a subject, the method comprising administering to the subject recombinant RNA construct compositions or pharmaceutical compositions, described herein, comprising an mRNA encoding IGF-1 and siRNA capable of binding to an mRNA of IL-1beta. In some aspects, provided herein, is a method of treating a disease or condition in a subject, the method comprising administering to the subject recombinant RNA compositions or pharmaceutical compositions described herein comprising an mRNA encoding IL-4 and siRNA capable of binding to an mRNA of TNF-alpha. In some aspects, provided herein, is a method of treating a disease or condition in a subject, the method comprising administering to the subject recombinant RNA construct compositions or pharmaceutical compositions, described herein, comprising an mRNA encoding IL-4 and siRNA capable of binding to an mRNA of IL-17. In some aspects, provided herein, is a method of treating a disease or condition in a subject, the method comprising administering to the subject recombinant RNA construct compositions or pharmaceutical compositions, described herein, comprising an mRNA encoding IL-4, siRNA capable of binding to an mRNA of TNF-alpha, and siRNA capable of binding to an mRNA of IL-17. In some embodiments, the disease or condition comprises a skin disease or condition or a joint disease or condition. In some embodiments, the skin disease or condition comprises an inflammatory skin disorder. In some embodiments, an inflammatory skin disorder comprises psoriasis. In some embodiments, the joint disease or condition comprises a joint degeneration. In some embodiments, the joint degeneration comprises intervertebral disc disease (IVDD) or osteoarthritis (OA).

In some aspects, compositions or pharmaceutical compositions administered to a subject in need thereof comprise recombinant polynucleic acid constructs or RNA constructs comprising: (i) an mRNA encoding IL-4; and (ii) at least two siRNAs capable of binding to a TNF-alpha mRNA. In related aspects, recombinant polynucleic acid constructs or RNA constructs may encode or comprise at least 2, 3, 4, 5, 6 or more siRNAs. In related aspects, recombinant polynucleic acid constructs or RNA constructs may comprise 2 siRNAs directed to a TNF-alpha mRNA. In related aspects, recombinant polynucleic acid constructs or RNA constructs may comprise 3 siRNAs, each directed to a TNF-alpha mRNA. In related aspects, each of the at least 3 siRNAs may be the same, different, or a combination thereof. In related aspects, recombinant polynucleic acid constructs or RNA constructs may comprise a sequence as set forth in SEQ ID NO: 4, SEQ ID NO: 5, SEQ ID NO: 6, SEQ ID NO: 7, SEQ ID NO: 8, SEQ ID NO: 9, SEQ ID NO: 100, SEQ ID NO: 101, SEQ ID NO: 102, SEQ ID NO:103, SEQ ID NO:104, or SEQ ID NO: 105 (Cpd.4-Cpd.9). In related aspects, recombinant polynucleic acid constructs may comprise a sequence as set forth in SEQ ID NO: 16, SEQ ID NO: 17, SEQ ID NO: 18, SEQ ID NO: 19, SEQ ID NO: 20, or SEQ ID NO: 21 (Cpd.4-Cpd.9).

In some aspects, compositions or pharmaceutical compositions administered to a subject in need thereof comprise recombinant polynucleic acid constructs or RNA constructs comprising: (i) an mRNA encoding IGF-1; and (ii) at least two siRNA capable of binding to an IL-8 mRNA. In related aspects, recombinant polynucleic acid constructs or RNA constructs may encode or comprise at least 2, 3, 4, 5, 6, or more siRNAs. In related aspects, recombinant polynucleic acid constructs or RNA constructs may comprise 2 siRNAs directed to an IL-8 mRNA. In related aspects, recombinant polynucleic acid constructs or RNA constructs may comprise 3 siRNAs, each directed to an IL-8 mRNA. In related aspects, each of the at least 3 siRNAs may be the same, different, or a combination thereof. In related aspects, recombinant polynucleic acid constructs or RNA constructs may comprise a sequence as set forth in SEQ ID NO: 1, SEQ ID NO: 2, SEQ ID NO: 97, or SEQ ID NO: 98 (Cpd.1 or Cpd.2). In related aspects, recombinant polynucleic acid constructs may comprise a sequence as set forth in SEQ ID NO: 13 or SEQ ID NO: 14 (Cpd.1 or Cpd.2).

In some aspects, compositions or pharmaceutical compositions administered to a subject in need thereof comprise recombinant polynucleic acid constructs or RNA constructs comprising: (i) an mRNA encoding IGF-1; and (ii) at least two siRNA capable of binding to an IL-1beta mRNA. In related aspects, recombinant polynucleic acid constructs or RNA constructs may encode or comprise at least 2, 3, 4, 5, 6, or more siRNAs. In related aspects, recombinant polynucleic acid constructs or RNA constructs may comprise 2 siRNAs directed to an IL-1beta mRNA. In related aspects, recombinant polynucleic acid constructs or RNA constructs may comprise 3 siRNAs, each directed to an IL-1beta mRNA. In related aspects, each of the at least 3 siRNAs may be the same, different, or a combination thereof. In related aspects, recombinant polynucleic acid constructs or RNA constructs may comprise a sequence as set forth in SEQ ID NO: 3 or SEQ ID NO: 99 (Cpd.3). In related aspects, recombinant polynucleic acid constructs may comprise a sequence as set forth in SEQ ID NO: 15 (Cpd.3).

In some aspects, compositions or pharmaceutical compositions administered to a subject in need thereof comprise recombinant polynucleic acid constructs or RNA constructs comprising: (i) an mRNA encoding IL-4; and (ii) at least two siRNA capable of binding to an IL-17 mRNA. In related aspects, recombinant polynucleic acid constructs or RNA constructs may encode or comprise at least 2, 3, 4, 5, 6, or more siRNAs. In related aspects, recombinant polynucleic acid constructs or RNA constructs may comprise 2 siRNAs directed to an IL-17 mRNA. In related aspects, recombinant polynucleic acid constructs or RNA constructs may comprise 3 siRNAs, each directed to an IL-17 mRNA. In related aspects, each of the at least 3 siRNAs may be the same, different, or a combination thereof. In related aspects, recombinant polynucleic acid constructs or RNA constructs may comprise a sequence as set forth in SEQ ID NO: 4 or SEQ ID NO: 100 (Cpd.4). In related aspects, recombinant polynucleic acid constructs may comprise a sequence as set forth in SEQ ID NO: 16 (Cpd.4).

In some aspects, compositions or pharmaceutical compositions administered to a subject in need thereof comprise recombinant polynucleic acid constructs or RNA constructs comprising: (i) an mRNA encoding IL-4; (ii) at least two siRNAs capable of binding to a TNF-alpha mRNA; and (iii) at least two siRNAs capable of binding to an IL-17 mRNA. In related aspects, recombinant polynucleic acid constructs or RNA constructs may encode or comprise at least 2, 3, 4, 5, 6 or more siRNAs. In related aspects, recombinant polynucleic acid constructs or RNA constructs may comprise 2 siRNAs directed to a TNF-alpha mRNA and 2 siRNAs directed to an IL-17 mRNA. In related aspects, recombinant polynucleic acid constructs or RNA constructs may comprise 3 siRNAs, each directed to a TNF-alpha mRNA or an IL-17 mRNA. In related aspects, each of the at least 3 siRNAs may be the same, different, or a combination thereof. In related aspects, recombinant polynucleic acid constructs or RNA constructs may comprise 6 siRNAs, each directed to a TNF-alpha mRNA or an IL-17 mRNA. In related aspects, recombinant polynucleic acid constructs or RNA constructs may comprise 6 siRNAs, 3 directed to a TNF-alpha mRNA and 3 an IL-17 mRNA. In related aspects, recombinant polynucleic acid constructs or RNA constructs may comprise a sequence as set forth in SEQ ID NO: 4 or SEQ ID NO: 100 (Cpd.4). In related aspects, recombinant polynucleic acid constructs may comprise a sequence as set forth in SEQ ID NO: 16 (Cpd.4).

In some aspects, compositions or pharmaceutical compositions administered to a subject in need thereof comprise recombinant polynucleic acid constructs or RNA constructs comprising: (i) an IGF-1 mRNA; and (ii) at least one siRNA capable of binding to a Turbo GFP mRNA. In related aspects, recombinant polynucleic acid constructs or RNA constructs may encode or comprise at least 1, 2, 3, 4, 5, 6, or more siRNAs. In related aspects, recombinant polynucleic acid constructs or RNA constructs may comprise 1 siRNA directed to a Turbo GFP mRNA. In related aspects, recombinant polynucleic acid constructs or RNA constructs may comprise 2 or 3 siRNAs, each directed to a Turbo GFP mRNA. In related aspects, each of the at least 2 or at least 3 siRNAs may be the same, different, or a combination thereof. In related aspects, recombinant polynucleic acid constructs or RNA constructs may comprise a sequence as set forth in SEQ ID NO: 10, SEQ ID NO: 11, SEQ ID NO: 12, SEQ ID NO: 106, SEQ ID NO: 107, or SEQ ID NO:108 (Cpd.10-Cpd.12). In related aspects, recombinant polynucleic acid constructs may comprise a sequence as set forth in SEQ ID NO: 22, SEQ ID NO: 23, or SEQ ID NO: 24 (Cpd.10-Cpd.12).

Recombinant RNA construct compositions described herein may be administered as a combination therapy. Combination therapies with two or more therapeutic agents or therapies may use agents and therapies that work by different mechanisms of action. Combination therapies using agents or therapies with different mechanisms of action can result in additive or synergetic effects. Combination therapies may allow for a lower dose of each agent than is used in monotherapy, thereby reducing toxic side effects and/or increasing the therapeutic index of the agent(s). Combination therapies can decrease the likelihood that resistant disease cells will develop. In some instances, combination therapies comprise a therapeutic agent or therapy that affects the immune response (e.g., enhances or activates the response) and a therapeutic agent that affects (e.g., inhibits or kills) the disease cells. In some instances, combination therapies may comprise (i) recombinant RNA compositions or pharmaceutical compositions described herein; and (ii) one or more additional therapies known in the art for the diseases described herein. In some embodiments, recombinant RNA compositions or pharmaceutical compositions described herein may be administered to a subject with a disease or condition prior to, concurrently with, and/or subsequently to, administration of one or more additional therapies for combination therapies. In some embodiments, the one or more additional therapies may comprise 1, 2, 3, or more additional therapeutic agents or therapies.

Compositions and pharmaceutical compositions described herein can be administered to a subject using any suitable methods known in the art. Suitable formulations for use in the present invention and methods of delivery are generally well known in the art. For example, compositions described herein can be administered to the subject in a variety of ways, including parenterally, intravenously, intradermally, intramuscularly, colonically, rectally, or intraperitoneally. In some embodiments, compositions described herein is administered by intraperitoneal injection, intramuscular injection, subcutaneous injection, or intravenous injection of the subject. In some embodiments, compositions described herein can be administered parenterally, intravenously, intramuscularly or orally. In some embodiments, compositions described herein can be administered via injection into disease tissues or cells. In some embodiments, compositions described herein can be administered as an aqueous solution for injection into disease tissues or cells.

Any of compositions and pharmaceutical compositions described herein may be provided together with an instruction manual. The instruction manual may comprise guidance for the skilled person or attending physician how to treat (or prevent) a disease or a disorder as described herein (e.g., a cancer such as a head and neck cancer) in accordance with the present invention. In some embodiments, the instruction manual may comprise guidance as to the herein described mode of delivery/administration and delivery/administration regimen, respectively (e.g., route of delivery/administration, dosage regimen, time of delivery/administration, frequency of delivery/administration, etc.). In some embodiments, the instruction manual may comprise the instruction that how compositions of the present invention is to be administrated or injected and/or is prepared for administration or injection. In principle, what has been described herein elsewhere with respect to the mode of delivery/administration and delivery/administration regimen, respectively, may be comprised as respective instructions in the instruction manual.

Compositions and pharmaceutical compositions described herein can be used in a gene therapy. In certain embodiments, compositions comprising recombinant polynucleic acids or RNA constructs described herein can be delivered to a cell in gene therapy vectors. Gene therapy vectors and methods of gene delivery are well known in the art. Non-limiting examples of these methods include viral vector delivery systems including DNA and RNA viruses, which have either episomal or integrated genomes after delivery to the cell, non-viral vector delivery systems including DNA plasmids, naked nucleic acid, and nucleic acid complexed with a delivery vehicle, transposon system (for delivery and integration into the host genomes; Moriarity, et al. (2013) Nucleic Acids Res 41(8), e92, Aronovich, et al., (2011) Hum. Mol. Genet. 20(R1), R14-R20), retrovirus-mediated DNA transfer (e.g., Moloney Mouse Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus; see e.g., Kay et al. (1993) Science 262, 117-119, Anderson (1992) Science 256, 808-813), and DNA virus-mediated DNA transfer including adenovirus, herpes virus, parvovirus and adeno-associated virus (e.g., Ali et al. (1994) Gene Therapy 1, 367-384). Viral vectors also include but are not limited to adeno-associated virus, adenoviral virus, lentivirus, retroviral, and herpes simplex virus vectors. Vectors capable of integration in the host genome include but are not limited to retrovirus or lentivirus.

In some embodiments, compositions comprising recombinant polynucleic acid or RNA constructs described herein can be delivered to a cell via direct DNA transfer (Wolff et al. (1990) Science 247, 1465-1468). Recombinant polynucleic acid or RNA constructs can be delivered to cells following mild mechanical disruption of the cell membrane, temporarily permeabilizing the cells. Such a mild mechanical disruption of the membrane can be accomplished by gently forcing cells through a small aperture (Sharei et al. PLOS ONE (2015) 10(4), e0118803). In another embodiment, compositions comprising recombinant polynucleic acid or RNA constructs described herein can be delivered to a cell via liposome-mediated DNA transfer (e.g., Gao & Huang (1991) Biochem. Ciophys. Res. Comm. 179, 280-285, Crystal (1995) Nature Med. 1, 15-17, Caplen et al. (1995) Nature Med. 3, 39-46). A liposome can encompass a variety of single and multilamellar lipid vehicles formed by the generation of enclosed lipid bilayers or aggregates. Recombinant polynucleic acid or RNA constructs can be encapsulated in the aqueous interior of a liposome, interspersed within the lipid bilayer of a liposome, attached to a liposome via a linking molecule that is associated with both the liposome and the oligonucleotide, entrapped in a liposome, or complexed with a liposome.

### Modulation of Gene Expression

Provided herein are methods of expressing an mRNA and at least two siRNAs from a single RNA transcript in a cell, comprising introducing into the cell compositions comprising any recombinant polynucleic acid or RNA constructs described herein. Further provided herein are methods of modulating expression of two or more genes in a cell, comprising introducing into the cell compositions comprising recombinant polynucleic acid or RNA constructs encoding or comprising a first RNA sequence encoding a gene of interest, and a second RNA sequence comprising at least two genetic elements that modulate expression of one or more target RNAs, wherein the at least two genetic elements that modulate expression of one or more target RNAs comprises a small interfering RNA (siRNA) capable of binding to a target messenger RNA (mRNA), and wherein the target mRNA is different from an mRNA encoded by the gene of interest, thereby modulating the expression of the target mRNA and the gene of interest from a single RNA transcript. In some instances, expression of a polynucleic acid, gene, DNA, or RNA, as used herein, can refer to transcription and/or translation of the polynucleic acid, gene, DNA, or RNA. In some instances, modulating, increasing, upregulating, decreasing, or downregulating expression of a polynucleic acid, gene such as a gene of interest, DNA, or RNA such as a target mRNA, as used herein, can refer to modulating, increasing, upregulating, decreasing, downregulating the level of protein encoded by a polynucleic acid, gene such as a gene of interest, DNA, or RNA such as a target mRNA by affecting transcription and/or translation of the polynucleic acid, gene such as a gene of interest, DNA, or RNA such as a target mRNA. In some instances, inhibiting expression of a polynucleic acid, gene such as a gene of interest, DNA, or RNA such as a target mRNA can refer to affecting transcription and/or translation of the polynucleic acid, gene such as a gene of interest, DNA, or RNA such as a target mRNA such that the level of protein encoded by the polynucleic acid, gene such as a gene of interest, DNA, or RNA such as a target mRNA is reduced or abolished.

For example, provided herein, are methods of modulating expression of two or more genes in a cell, comprising introducing into the cell compositions comprising recombinant polynucleic acid or RNA constructs encoding or comprising a first RNA sequence encoding a gene of interest, and a second RNA sequence comprising at least two genetic elements that modulate expression of one or more target RNAs, wherein the first RNA sequence encodes a IL-4, and wherein the second RNA sequence encodes a small interfering RNA (siRNA) capable of binding to a TNF-alpha mRNA, thereby modulating the expression of the TNF-alpha mRNA and IL-4 from a single RNA transcript.

For example, provided herein, are methods of modulating expression of two or more genes in a cell, comprising introducing into the cell compositions comprising recombinant polynucleic acid or RNA constructs encoding or comprising a first RNA sequence encoding a gene of interest, and a second RNA sequence comprising at least two genetic elements that modulate expression of one or more target RNAs, wherein the first RNA sequence encodes a IGF-1, and wherein the second RNA sequence encodes a small interfering RNA (siRNA) capable of binding to an IL-8 mRNA, thereby modulating the expression of the IL-8 mRNA and IGF-1 from a single RNA transcript.

For example, provided herein, are methods of modulating expression of two or more genes in a cell, comprising introducing into the cell compositions comprising recombinant polynucleic acid or RNA constructs encoding or comprising a first RNA sequence encoding a gene of interest, and a second RNA sequence comprising at least two genetic elements that modulate expression of one or more target RNAs, wherein the first RNA sequence encodes a IGF-1, and wherein the second RNA sequence encodes a small interfering RNA (siRNA) capable of binding to an IL-1beta mRNA, thereby modulating the expression of the IL-1beta mRNA and IGF-1 from a single RNA transcript.

For example, provided herein, are methods of modulating expression of two or more genes in a cell, comprising introducing into the cell compositions comprising recombinant polynucleic acid or RNA constructs encoding or comprising a first RNA sequence encoding a gene of interest, and a second RNA sequence comprising at least two genetic elements that modulate expression of one or more target RNAs, wherein the first RNA sequence encodes a IL-4, and wherein the second RNA sequence encodes a small interfering RNA (siRNA) capable of binding to an IL-17 mRNA, thereby modulating the expression of the IL-17 mRNA and IL-4 from a single RNA transcript.

For example, provided herein, are methods of modulating expression of two or more genes in a cell, comprising introducing into the cell compositions comprising recombinant polynucleic acid or RNA constructs encoding or comprising a first RNA sequence encoding a gene of interest, and a second RNA sequence comprising at least two genetic elements that modulate expression of one or more target RNAs, wherein the first RNA sequence encodes a IL-4, and wherein the second RNA sequence encodes a small interfering RNA (siRNA) capable of binding to a TNF-alpha mRNA, and siRNA capable of binding to an IL-17 mRNA, thereby modulating the expression of the TNF-alpha mRNA, IL-17 mRNA, and IL-4 from a single RNA transcript.

For example, provided herein, are methods of modulating expression of two or more genes in a cell, comprising introducing into the cell compositions comprising recombinant polynucleic acid or RNA constructs encoding or comprising a first RNA sequence, a second RNA sequence, and a linker RNA sequence, wherein the linker RNA sequence links the first RNA sequence and the second RNA sequence, wherein the first RNA sequence encodes a IGF-1, and wherein the second RNA sequence encodes a small interfering RNA (siRNA) capable of binding to a Turbo GFP mRNA, thereby modulating the expression of the Turbo GFP mRNA and IGF-1 from a single RNA transcript.

Provided herein are methods of modulating expression of two or more genes in a cell, comprising introducing into the cell compositions comprising recombinant polynucleic acid or RNA constructs comprising a first RNA sequence encoding a gene of interest, and a second RNA sequence comprising at least two genetic elements that modulate expression of one or more target RNAs, wherein the first RNA encodes IL-4, and wherein the second RNA encodes a small interfering RNA (siRNA) capable of binding to a TNF-alpha mRNA; wherein the expression of IL-4 and TNF-alpha is modulated simultaneously, i.e., the expression of IL-4 is upregulated and the expression of TNF-alpha is downregulated simultaneously. In related aspects, recombinant polynucleic acid or RNA constructs may encode or comprise at least 2, 3, 4, 5, 6, or more siRNAs. In related aspects, recombinant polynucleic acid or RNA constructs may encode or comprise 3 siRNAs, each directed to the same region of a TNF-alpha mRNA. In related aspects, recombinant polynucleic acid or RNA constructs may encode or comprise 3 siRNAs, each directed to a different region of a TNF-alpha mRNA. In related aspects, each of the at least 3 siRNAs may be directed to the same, different, or a combination thereof. In related aspects, recombinant RNA constructs may comprise a sequence shown in SEQ ID NOs: 4-9 or 100-105 (Cpd.4-Cpd.9). In related aspects, recombinant polynucleic acid constructs may comprise a sequence shown in SEQ ID NOs: 16-21 (Cpd.4-Cpd.9).

Also provided herein are methods of modulating expression of two or more genes in a cell, comprising introducing into the cell compositions comprising recombinant polynucleic acid or RNA constructs comprising a first RNA sequence encoding a gene of interest, and a second RNA sequence comprising at least two genetic elements that modulate expression of one or more target RNAs, wherein the first RNA encodes IGF-1, and wherein the second RNA encodes a small interfering RNA (siRNA) capable of binding to an IL-8 mRNA; wherein the expression of IGF-1 and IL-8 is modulated simultaneously, i.e., the expression of IGF-1 is upregulated and the expression of IL-8 is downregulated simultaneously. In related aspects, recombinant polynucleic acid or RNA constructs may encode or comprise at least 2, 3, 4, 5, 6, or more siRNAs. In related aspects, recombinant polynucleic acid or RNA constructs may encode or comprise 3 siRNAs, each directed to the same region of an IL-8 mRNA. In related aspects, recombinant polynucleic acid or RNA constructs may encode or comprise 3 siRNAs, each directed to a different region of an IL-8 mRNA. In related aspects, each of the at least 3 siRNAs may be directed to the same, different, or a combination thereof. In related aspects, recombinant RNA constructs may comprise a sequence comprising SEQ ID NO: 1 (Cpd.1), SEQ ID NO: 97 (Cpd.1), SEQ ID NO: 2 (Cpd.2), or SEQ ID NO: 98 (Cpd.2). In related aspects, recombinant polynucleic acid constructs may comprise a sequence comprising SEQ ID NO: 13 (Cpd.1) or SEQ ID NO: 14 (Cpd.2).

Also provided herein are methods of modulating expression of two or more genes in a cell, comprising introducing into the cell compositions comprising recombinant polynucleic acid or RNA constructs comprising a first RNA sequence encoding a gene of interest, and a second RNA sequence comprising at least two genetic elements that modulate expression of one or more target RNAs, wherein the first RNA encodes IGF-1, and wherein the second RNA encodes a small interfering RNA (siRNA) capable of binding to an IL-1beta mRNA; wherein the expression of IGF-1 and IL-1beta is modulated simultaneously, i.e., the expression of IGF-1 is upregulated and the expression of IL-1beta is downregulated simultaneously. In related aspects, recombinant polynucleic acid or RNA constructs may encode or comprise at least 2, 3, 4, 5, 6, or more siRNAs. In related aspects, recombinant polynucleic acid or RNA constructs may encode or comprise 3 siRNAs, each directed to the same region of an IL-1beta mRNA. In related aspects, recombinant polynucleic acid or RNA constructs may encode or comprise 3 siRNAs, each directed to a different region of an IL-1beta mRNA. In related aspects, each of the at least 3 siRNAs may be directed to the same, different, or a combination thereof. In related aspects, recombinant polynucleic acid constructs or recombinant RNA constructs may comprise a sequence comprising in SEQ ID NO: 3 or SEQ ID NO: 99 (Cpd.3). In related aspects, recombinant polynucleic acid constructs may comprise a sequence comprising in SEQ ID NO: 15 (Cpd.3).

Also provided herein are methods of modulating expression of two or more genes in a cell, comprising introducing into the cell compositions comprising recombinant polynucleic acid or RNA constructs comprising a first RNA sequence encoding a gene of interest, and a second RNA sequence comprising at least two genetic elements that modulate expression of one or more target RNAs, wherein the first RNA encodes IL-4, and wherein the second RNA encodes a small interfering RNA (siRNA) capable of binding to an IL-17 mRNA; wherein the expression of IL-4 and IL-17 is modulated simultaneously, i.e., the expression of IL-4 is upregulated and the expression of IL-17 is downregulated simultaneously. In related aspects, recombinant polynucleic acid or RNA constructs may encode or comprise at least 2, 3, 4, 5, 6, or more siRNAs. In related aspects, recombinant polynucleic acid or RNA constructs may encode or comprise 3 siRNAs, each directed to the same region of an IL-17 mRNA. In related aspects, recombinant polynucleic acid or RNA constructs may encode or comprise 3 siRNAs, each directed to a different region of an IL-17 mRNA. In related aspects, each of the at least 3 siRNAs may be directed to the same, different, or a combination thereof. In related aspects, recombinant RNA constructs may comprise a sequence comprising in SEQ ID NO: 4 or SEQ ID NO: 100 (Cpd.4). In related aspects, recombinant polynucleic acid constructs may comprise a sequence comprising in SEQ ID NO: 16 (Cpd.4).

Also provided herein are methods of modulating expression of two or more genes in a cell, comprising introducing into the cell compositions comprising recombinant polynucleic acid or RNA constructs comprising a first RNA sequence encoding a gene of interest, and a second RNA sequence comprising at least two genetic elements that modulate expression of one or more target RNAs, wherein the first RNA encodes IL-4, and wherein the second RNA encodes a small interfering RNA (siRNA) capable of binding to a TNF-alpha mRNA and siRNA capable of binding to an IL-17 mRNA; wherein the expression of IL-4, TNF-alpha, and IL-17 is modulated simultaneously, i.e., the expression of IL-4 is upregulated and the expression of TNF-alpha and IL-17 is downregulated simultaneously. In related aspects, recombinant polynucleic acid or RNA constructs may encode or comprise at least 2, 3, 4, 5, 6, or more siRNAs. In related aspects, recombinant polynucleic acid or RNA constructs may encode or comprise 3 siRNAs, each directed to the same region of a TNF-alpha mRNA. In related aspects, recombinant polynucleic acid or RNA constructs may encode or comprise 3 siRNAs, each directed to the same region of an IL-17 mRNA. In related aspects, recombinant polynucleic acid or RNA constructs may encode or comprise 3 siRNAs, each directed to a different region of a TNF-alpha and/or an IL-17 mRNA. In related aspects, each of the at least 3 siRNAs may be directed to the same, different, or a combination thereof. In related aspects, recombinant polynucleic acid or RNA constructs may encode or comprise 6 siRNAs, wherein 3 of 6 siRNAs are directed to a different region of a TNF-alpha and the other 6 siRNAs are directed to a different region of an IL-17 mRNA. In related aspects, recombinant RNA constructs may comprise a sequence comprising in SEQ ID NO: 4 or SEQ ID NO: 100 (Cpd.4). In related aspects, recombinant polynucleic acid constructs may comprise a sequence comprising in SEQ ID NO: 16 (Cpd.4).

Also provided herein are methods of modulating expression of two or more genes in a cell, comprising introducing into the cell compositions comprising recombinant polynucleic acid or RNA constructs comprising a first RNA sequence, a second RNA sequence, and a linker RNA sequence, wherein the linker RNA sequence links the first RNA sequence and the second RNA sequence, wherein the first RNA encodes IGF-1, and wherein the second RNA encodes a small interfering RNA (siRNA) capable of binding to a Turbo GFP mRNA; wherein the expression of IGF-1 and Turbo GFP is modulated simultaneously, i.e., the expression of IGF-1 is upregulated and the expression of Turbo GFP is downregulated simultaneously. In related aspects, recombinant polynucleic acid or RNA constructs may encode or comprise at least 1, 2, 3, 4, 5, 6, or more siRNAs. In related aspects, recombinant polynucleic acid or RNA constructs may encode or comprise 3 siRNAs, each directed to the same region of a Turbo GFP mRNA. In related aspects, recombinant polynucleic acid or RNA constructs may encode or comprise 3 siRNAs, each directed to a different region of a Turbo GFP mRNA. In related aspects, each of the at least 3 siRNAs may be directed to the same, different, or a combination thereof. In related aspects, recombinant RNA constructs may comprise a sequence selected from the group consisting of SEQ ID NOs: 10-12 and 106-108 (Cpd.10-Cpd.12). In related aspects, recombinant polynucleic acid constructs may comprise a sequence selected from the group consisting of SEQ ID NOs: 22-24 (Cpd.10-Cpd.12).

Provided herein are methods of upregulating and downregulating expression of two or more genes in a cell, comprising introducing into the cell compositions comprising recombinant polynucleic acid or RNA constructs encoding or comprising a first RNA sequence encoding a gene of interest (e.g., IL-4 or IGF-1), and a second RNA sequence comprising at least two genetic elements that modulate expression of one or more target RNAs, wherein the second RNA encodes a small interfering RNA (siRNA) capable of binding to a target mRNA (e.g., TNF-alpha IL-8, IL-17, Turbo GFP, or IL-1beta); wherein the target mRNA is different from an mRNA encoded by the gene of interest, and wherein the expression of the target mRNA is downregulated and the expression of the gene of interest is upregulated simultaneously. In some embodiments, the expression of the target mRNA is downregulated by the siRNA capable of binding to the target mRNA. In some embodiments, the expression of the gene of interest is upregulated by expressing an mRNA or a protein encoded by the gene of interest.

### ILLUSTRATIVE EMBODIMENTS

In some aspects, provided herein, is a composition comprising a recombinant RNA construct comprising: (i) a first RNA sequence encoding a gene of interest, and (ii) a second RNA sequence comprising at least two genetic elements that modulate expression of one or more target RNAs, wherein contacting a human cell with the recombinant RNA construct results in an immune response that is lower than the immune response of the human cell contacted with a corresponding recombinant RNA construct comprising the first RNA sequence of (i) and a corresponding second RNA sequence of (ii) with at most one of the at least two genetic elements. In some embodiments, the recombinant RNA construct comprises one or more uridines. In some embodiments, the recombinant RNA construct does not comprise a modified uridine.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct does not comprise a nucleotide variant. In some embodiments, the nucleotide variant comprises a modified uridine. In some embodiments, the modified uridine comprises a N1-methylpseudouridine.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct does not comprise a modified uridine.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct does not comprise a N1-methylpseudouridine.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct comprises solely unmodified nucleotides or natural nucleotides.

In some aspects, provided herein, is a composition comprising a recombinant RNA construct comprising (i) a first RNA sequence encoding a gene of interest, and (ii) a second RNA sequence comprising at least two genetic elements that modulate expression of one or more target RNAs, wherein the recombinant RNA construct comprises uridines, wherein: (a) all uridines comprised by the recombinant RNA constructs are unmodified or natural nucleotide(s); or (b) at least one of the uridines comprised by the recombinant RNA constructs is an unmodified uridine.

In some embodiments, the corresponding recombinant RNA construct does not comprise any of the genetic elements that modulate expression of one or more target RNAs. In some embodiments, the second RNA sequence comprises at least three genetic elements that modulate expression of one or more target RNAs. In some embodiments, the second RNA sequence comprises at least six genetic elements that modulate expression of one or more target RNAs. In some embodiments, the second RNA sequence comprises at least two or at least four genetic elements that modulate expression of one or more target RNAs. In some embodiments, the second RNA sequence comprises two or four or more genetic elements that modulate expression of one or more target RNAs.

In some embodiments, the first RNA sequence is a messenger RNA (mRNA) sequence. In some embodiments, each of the at least two genetic elements of the second RNA sequence comprises a secondary structure. In some embodiments, each of the at least two genetic elements of the second RNA sequence comprises a hairpin structure or a loop structure. In some embodiments, each of the at least two genetic elements of the second RNA sequence is a short or small hairpin RNA (shRNA). In some embodiments, each of the at least two genetic elements of the second RNA sequence is processed or cleaved by an intracellular protein. In some embodiments, each of the at least two genetic elements of the second RNA sequence is processed or cleaved by an endogenous protein of a cell. In some embodiments, each of the at least two genetic elements of the second RNA sequence is processed or cleaved by an endogenous Dicer. In some embodiments, each of the at least two genetic elements of the second RNA sequence comprises a small interfering RNA (siRNA). In some embodiments, each of the at least two genetic elements of the second RNA sequence is capable of binding to the one or more target RNAs. In some embodiments, the second RNA sequence comprises at least two or at least four siRNAs that modulate expression of one or more target RNAs. In some embodiments, the second RNA sequence comprises two or four or more siRNAs that modulate expression of one or more target RNAs.

In some embodiments, the immune response is a human Toll-Like Receptor 7 (TLR7) immune response, an interferon alpha/beta (IFNα/β) immune response, a human Toll-Like Receptor 3 (TLR3) immune response, a human Toll-Like Receptor 8 (TLR8) immune response, or any combination thereof. In some embodiments, contacting the human cell with the recombinant RNA construct results in an immune response that is lower than the immune response of the human cell contacted with the corresponding recombinant RNA construct according to a human Toll-Like Receptor 7 (TLR7) immunogenicity assay. In some embodiments, the human TLR7 immunogenicity assay measures activation of NF-κB and/or AP1. In some embodiments, the human TLR7 immunogenicity assay is performed in HEK293 cells or a derivative thereof. In some embodiments, the HEK293 cells are engineered to express hTLR7 and a reporter gene. In some embodiments, the reporter gene is a secreted reporter gene. In some embodiments, the secreted reporter gene is secreted embryonic alkaline phosphatase (SEAP). In some embodiments, the reporter gene is under the control of a promoter with one or more NF-κB and/or AP1 binding sites. In some embodiments, the promoter is an IFN-β minimal promoter. In some embodiments, the immune response in the human cell contacted with the recombinant RNA construct is at least 1.5 fold or at least 2 fold less than the immune response in the human cell contacted with the corresponding recombinant RNA construct.

In some embodiments, contacting the human cell with the recombinant RNA construct results in an immune response that is lower than the immune response of the human cell contacted with the corresponding recombinant RNA construct according to an interferon alpha/beta (IFNα/β) immunogenicity assay. In some embodiments, the IFNα/β immunogenicity assay measures activation of JAK-STAT and/or ISG3. In some embodiments, the IFNα/β immunogenicity assay is performed in HEK293 cells or a derivative thereof. In some embodiments, the HEK293 cells are engineered to express human STAT2 and/or IRF9 genes and a reporter gene. In some embodiments, the reporter gene is a secreted reporter gene. In some embodiments, the secreted reporter gene is secreted embryonic alkaline phosphatase (SEAP). In some embodiments, the reporter gene is under the control of a promoter with one or more STAT2 and/or IRF9 binding sites. In some embodiments, the promoter is an ISG54 promoter. In some embodiments, the immune response in the human cell contacted with the recombinant RNA construct is at least 1.5 fold, at least 2 fold, or at least 100 fold less than the immune response in the human cell contacted with the corresponding recombinant RNA construct.

In some embodiments, contacting the human cell with the recombinant RNA construct does not result in a substantial immune response according to a human Toll-Like Receptor 3 (TLR3) immunogenicity assay. In some embodiments, the human TLR3 immunogenicity assay measures activation of NF-κB and/or AP1. In some embodiments, the human TLR3 immunogenicity assay is performed in HEK293 cells or a derivative thereof. In some embodiments, the HEK293 cells are engineered to express hTLR3 and a reporter gene. In some embodiments, the reporter gene is a secreted reporter gene. In some embodiments, the secreted reporter gene is secreted embryonic alkaline phosphatase (SEAP). In some embodiments, the reporter gene is under the control of a promoter with one or more NF-κB and/or AP1 binding sites. In some embodiments, the promoter is an IFN-β minimal promoter.

In some embodiments, contacting the human cell with the recombinant RNA construct results in an immune response that is lower than the immune response of the human cell contacted with the corresponding recombinant RNA construct according to a human Toll-Like Receptor 8 (TLR8) immunogenicity assay. In some embodiments, the human TLR8 immunogenicity assay measures activation of NF-κB, AP1, and/or IRF. In some embodiments, the human TLR8 immunogenicity assay is performed in HEK293 cells or a derivative thereof. In some embodiments, the HEK293 cells are engineered to express hTLR8 and a reporter gene. In some embodiments, the reporter gene is a secreted reporter gene. In some embodiments, the secreted reporter gene is secreted embryonic alkaline phosphatase (SEAP). In some embodiments, the reporter gene is under the control of a promoter with one or more NF-κB and/or AP1 binding sites. In some embodiments, the promoter is an IFN-β minimal promoter.

In some embodiments, the immune response induces the expression of a proinflammatory cytokine in a cell. In some embodiments, the proinflammatory cytokine comprises Interleukin 6 (IL-6). In some embodiments, the cell comprises a human lung epithelial carcinoma cell (A549) or a human monocyte leukemia cell (THP-1).

In some embodiments, the second RNA sequence comprises 2, 3, 4, 5, 6, or more species of siRNA, wherein the 2, 3, 4, 5, 6, or more species of siRNA include siRNAs that are capable of binding to: (i) different target RNAs; (ii) different regions of the same target RNA; (iii) the same region of the same target RNA; or (iv) any combination thereof. In some embodiments, the second RNA sequence comprises at least 3 species of siRNA. In some embodiments, the second RNA sequence comprises at least 6 species of siRNA.

In some embodiments, the recombinant RNA construct further comprises one or more linkers. In some embodiments, each of the one or more linkers has a structure selected from the group consisting of: Formula (I): XₘCAACAAXₙ, wherein X is any nucleotide, m is an integer from 1 to 12, and n is an integer from 0 to 4 (SEQ ID NO: 129); and Formula (II): XₚTCCCXᵣ, wherein X is any nucleotide, p is an integer from 0 to 17, and r is an integer from 0 to 13 (SEQ ID NO: 130). In some embodiments, each of the one or more linkers comprises a sequence comprising ACAACAA (SEQ ID NO: 85). In some embodiments, each of the one or more linkers is not (a) TTTATCTTAGAGGCATATCCCTACGTACCAACAA (SEQ ID NO: 28) or ATAGTGAGTCGTATTAACGTACCAACAA (SEQ ID NO: 27); or (b) does not form a secondary structure according to RNAfold WebServer. In some embodiments, each of the one or more linkers is present between (a) the first RNA sequence and the second RNA sequence, (b) each of the 2, 3, 4, 5, 6, or more species of siRNA of the second RNA sequence, or (c) both (a) and (b). In some embodiments, each of the one or more linkers comprises a sequence independently selected from the group consisting of SEQ ID NOs: 27, 28, 85-95.

In some embodiments, the expression of the gene of interest is modulated. In some embodiments, the expression of the gene of interest is upregulated in a cell comprising the recombinant RNA construct. In some embodiments, the expression of a protein encoded by the gene of interest is upregulated in a cell comprising the recombinant RNA construct.

In some embodiments, the expression of the one or more target RNAs is modulated. In some embodiments, the expression of the one or more target RNAs is downregulated by the genetic elements that modulate expression of the one or more target RNAs. In some embodiments, the genetic elements that modulate expression of the one or more target RNAs do not inhibit the expression of the gene of interest.

In some embodiments, the gene of interest is selected from the group consisting of Interleukin 4 (IL-4) and Insulin-like Growth Factor 1 (IGF-1). In some embodiments, the one or more target RNA comprises a noncoding RNA or a messenger RNA (mRNA). In some embodiments, each of the one or more target RNA is a noncoding RNA. In some embodiments, each of the one or more target RNA is an mRNA. In some embodiments, the target RNA is an mRNA encoding a protein comprising Interleukin 8 (IL-8), Interleukin 1 beta (IL-1 beta), Tumor Necrosis Factor alpha (TNF-alpha), Interleukin 17 (IL-17), or a functional variant thereof.

In some embodiments, the genetic elements that modulate expression of the one or more target RNAs binds to an exon of the one or more target RNAs. In some embodiments, the genetic elements that modulate expression of the one or more target RNAs specifically binds to one target RNA. In some embodiments, the genetic elements that modulate expression of the one or more target RNAs are not encoded by or comprised of an intron sequence of the gene of interest. In some embodiments, the gene of interest is expressed without RNA splicing.

In some embodiments, the first RNA sequence is present downstream or 3′ of the second RNA sequence. In some embodiments, the first RNA sequence is present upstream or 5′ of the second RNA sequence. In some embodiments, the RNA construct comprises an internal ribosome entry site (IRES) upstream or 5′ of the first RNA sequence.

In some embodiments, the compositions described herein further comprises a poly(A) tail, a 5′ cap, or a Kozak sequence. In some embodiments, the first RNA sequence and the second RNA sequence are both recombinant. In some embodiments, the siRNA comprises a sense strand sequence selected from SEQ ID NOs: 57-70.

In some aspects, provided herein, is a composition for use in modulating the expression of two or more genes in a cell. In some aspects, provided herein, is a pharmaceutical composition comprising a therapeutically effective amount of any one of the compositions described herein and a pharmaceutically acceptable excipient. In some aspects, provided herein, is a vector comprising a recombinant polynucleic acid construct encoding any one of the compositions described herein. In some aspects, provided herein, is a cell comprising any one of the compositions described herein or any one of the vectors described herein.

In some aspects, provided herein, is a method of simultaneously expressing an siRNA and an mRNA from a single RNA transcript in a cell, comprising introducing into the cell any one of the compositions described herein, or any one of the vectors described herein.

In some aspects, provided herein, is a method of treating a disease or condition comprising administering to a subject in need thereof any one of the compositions described herein or any one of the pharmaceutical compositions described herein.

In some embodiments, the disease or condition comprises a skin disease or condition or a joint disease or condition. In some embodiments, the skin disease or condition comprises an inflammatory skin disorder. In some embodiments, the inflammatory skin disorder comprises psoriasis. In some embodiments, the joint disease or condition comprises a joint degeneration. In some embodiments, the joint degeneration comprises intervertebral disc disease (IVDD) or osteoarthritis (OA). In some embodiments, the subject is a human.

In some aspects, provided herein, is a composition comprising a recombinant RNA construct comprising: (i) a messenger RNA (mRNA) encoding Insulin-like Growth Factor 1 (IGF-1), and (ii) at least two small interfering RNAs (siRNAs) capable of binding to an Interleukin-8 (IL-8) mRNA, wherein contacting a human cell with the recombinant RNA construct results in an immune response that is lower than the immune response of the human cell contacted with a corresponding recombinant RNA construct comprising the mRNA encoding IGF-1 of (i) and at most one of the at least two siRNAs capable of binding to the IL-8 mRNA of (ii).

In some aspects, provided herein, is a composition comprising recombinant RNA construct comprising: (i) a messenger RNA (mRNA) encoding Insulin-like Growth Factor 1 (IGF-1), and (ii) at least two small interfering RNAs (siRNAs) capable of binding to a Interleukin-1 beta (IL-1 beta) mRNA, wherein contacting a human cell with the recombinant RNA construct results in an immune response that is lower than the immune response of the human cell contacted with a corresponding recombinant RNA construct comprising the mRNA encoding IGF-1 of (i) and at most one of the at least two siRNAs capable of binding to the IL-1beta mRNA of (ii).

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA, wherein contacting a human cell with the recombinant RNA construct results in an immune response that is lower than the immune response of the human cell contacted with a corresponding recombinant RNA construct comprising the mRNA encoding IL-4 of (i) and at most one of the at least two siRNAs capable of binding to the TNF-alpha mRNA of (ii).

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA and Interleukin 17 (IL-17), wherein contacting a human cell with the recombinant RNA construct results in an immune response that is lower than the immune response of the human cell contacted with a corresponding recombinant RNA construct comprising the mRNA encoding IL-4 of (i) and at most one of the at least two siRNAs capable of binding to the TNF-alpha mRNA and IL-17 mRNA of (ii).

In some aspects, provided herein, is a composition comprising a recombinant RNA construct comprising: (i) a messenger RNA (mRNA) encoding Insulin-like Growth Factor 1 (IGF-1), and (ii) at least two small interfering RNAs (siRNAs) capable of binding to an Interleukin-8 (IL-8) mRNA, wherein the recombinant RNA construct does not comprise a nucleotide variant.

In some aspects, provided herein, is a composition comprising a recombinant RNA construct comprising: (i) a messenger RNA (mRNA) encoding Insulin-like Growth Factor 1 (IGF-1), and (ii) at least two small interfering RNAs (siRNAs) capable of binding to an Interleukin-8 (IL-8) mRNA, wherein the recombinant RNA construct does not comprise a modified uridine.

In some aspects, provided herein, is a composition comprising a recombinant RNA construct comprising: (i) a messenger RNA (mRNA) encoding Insulin-like Growth Factor 1 (IGF-1), and (ii) at least two small interfering RNAs (siRNAs) capable of binding to an Interleukin-8 (IL-8) mRNA, wherein the recombinant RNA construct does not comprise a N1-methylpseudouridine.

In some aspects, provided herein, is a composition comprising a recombinant RNA construct comprising: (i) a messenger RNA (mRNA) encoding Insulin-like Growth Factor 1 (IGF-1), and (ii) at least two small interfering RNAs (siRNAs) capable of binding to an Interleukin-8 (IL-8) mRNA, wherein the recombinant RNA construct comprises uridines, wherein (a) all uridines comprised by the recombinant RNA constructs are unmodified or natural nucleotide(s); or (b) at least one of the uridines comprised by the recombinant RNA constructs is an unmodified uridine.

In some aspects, provided herein, is a composition comprising recombinant RNA construct comprising: (i) a messenger RNA (mRNA) encoding Insulin-like Growth Factor 1 (IGF-1), and (ii) at least two small interfering RNAs (siRNAs) capable of binding to a Interleukin-1 beta (IL-1 beta) mRNA, wherein the recombinant RNA construct does not comprise a nucleotide variant.

In some aspects, provided herein, is a composition comprising recombinant RNA construct comprising: (i) a messenger RNA (mRNA) encoding Insulin-like Growth Factor 1 (IGF-1), and (ii) at least two small interfering RNAs (siRNAs) capable of binding to a Interleukin-1 beta (IL-1 beta) mRNA, wherein the recombinant RNA construct does not comprise a modified uridine.

In some aspects, provided herein, is a composition comprising recombinant RNA construct comprising: (i) a messenger RNA (mRNA) encoding Insulin-like Growth Factor 1 (IGF-1), and (ii) at least two small interfering RNAs (siRNAs) capable of binding to a Interleukin-1 beta (IL-1 beta) mRNA, wherein the recombinant RNA construct does not comprise a N1-methylpseudouridine.

In some aspects, provided herein, is a composition comprising recombinant RNA construct comprising: (i) a messenger RNA (mRNA) encoding Insulin-like Growth Factor 1 (IGF-1), and (ii) at least two small interfering RNAs (siRNAs) capable of binding to a Interleukin-1 beta (IL-1 beta) mRNA, wherein the recombinant RNA construct comprises uridines, wherein (a) all uridines comprised by the recombinant RNA constructs are unmodified or natural nucleotide(s); or (b) at least one of the uridines comprised by the recombinant RNA constructs is an unmodified uridine.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA, wherein the recombinant RNA construct does not comprise a nucleotide variant.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA, wherein the recombinant RNA construct does not comprise a modified uridine.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA, wherein the recombinant RNA construct does not comprise a N1-methylpseudouridine.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA, wherein the recombinant RNA construct comprises uridines, wherein (a) all uridines comprised by the recombinant RNA constructs are unmodified or natural nucleotide(s); or (b) at least one of the uridines comprised by the recombinant RNA constructs is an unmodified uridine.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA and Interleukin 17 (IL-17), wherein the recombinant RNA construct does not comprise a nucleotide variant.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA and Interleukin 17 (IL-17), wherein the recombinant RNA construct does not comprise a modified uridine.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA and Interleukin 17 (IL-17), wherein the recombinant RNA construct does not comprise a N1-methylpseudouridine.

In some aspects, provided herein, is a composition comprising recombinant RNA construct: (i) a messenger RNA (mRNA) encoding Interleukin-4 (IL-4), and (ii) at least two small interfering RNAs (siRNAs) capable of binding to a Tumor Necrosis Factor alpha (TNF-alpha) mRNA and Interleukin 17 (IL-17), wherein the recombinant RNA construct comprises uridines, wherein (a) all uridines comprised by the recombinant RNA constructs are unmodified or natural nucleotide(s); or (b) at least one of the uridines comprised by the recombinant RNA constructs is an unmodified uridine.

In some aspects, provided herein, is a composition comprising a recombinant polynucleic acid construct comprising a nucleic acid sequence selected from the group consisting of SEQ ID NOs: 1-24, 42, 125, 97-108, 121-122, and 127-128.

In a preferred embodiment, the gene of interest encoded by the first RNA sequence is not an interleukin 2 (IL-2) and the one or more target RNAs modulated by the at least two genetic elements comprised by the second RNA sequence is not vascular endothelial growth factor A (VEGFA), an isoform of VEGFA, MHC class I chain-related sequence A (MICA), or MHC class I chain-related sequence B (MICB).

In a preferred embodiment, the gene of interest encoded by the first RNA sequence is not an interleukin 2 (IL-2), a fragment thereof, or a functional variant thereof and the one or more target RNAs modulated by the at least two genetic elements comprised by the second RNA sequence is not vascular endothelial growth factor A (VEGFA), an isoform of VEGFA, MHC class I chain-related sequence A (MICA), MHC class I chain-related sequence B (MICB), a fragment thereof, or a functional variant thereof.

### EXAMPLES

These examples are provided for illustrative purposes only and not to limit the scope of the claims provided herein.

### Example 1: Construct Design, Sequence, and Synthesis

Construct Design

Both siRNAs and genes of interest are simultaneously expressed from a single transcript generated by in vitro transcription (Table 1; SEQ ID NOs: 1-12 and 97-108). IL-4 and IGF-1 coding sequences originate from Homo sapiens and no changes in the resulting amino acid sequences were introduced for IL-4 (hIL-4: NP_000580.1; SEQ ID NO: 31 and 32; Cpd.4-Cpd.9) and some of IGF-1 constructs (Cpd.10-Cpd.13; IGF-1:NP_000609.1). To increase secretion of mRNA-induced IGF-1 (NP 000609.1) out of the transfected cell, the endogenous IGF-1 pre-domain (signal peptide; SEQ ID NO: 33 and 34) was exchanged by BDNF (NP_733931.1; SEQ ID NO: 35 and 36) signal peptide (BDNF-pro-IGF-1) in Cpd.1 to Cpd.4 mRNA constructs. Furthermore, the construct contained the sequence encoding the full coding sequence of mature human IGF-1 with 70 amino acids (SEQ ID NO: 35 and 36). No C-terminal E-domain was added to the construct. The siRNA target sequence for IL-8 (NM_000584.3; SEQ ID NO: 37), IL-1beta (NM_000576.2; SEQ ID NO: 38), IL-17 (NM_002190.2; SEQ ID NO: 39) and TNF-alpha (NM_000594.3; SEQ ID NO: 40) originate from Homo sapiens and no changes to the sequences introduced. Turbo GFP sequence was derived from marine copepod Pontellina plumate (SEQ ID NO: 41). To compare mRNA with siRNA structure and without siRNA structure, an IL-4 molecule with optimized signal peptide was used (SEQ ID NO: 42 and 43).

A polynucleic acid construct may comprise a Kozak sequence, (5′ GCCACC 3′; SEQ ID NO:25). In addition, a polynucleic acid construct may comprise a T7 promoter sequence (5′ TAATACGACTCACTATA 3′; SEQ ID NO:26) upstream of the gene of interest sequence, for RNA polymerase binding and successful in vitro transcription of both the gene of interest and siRNA in a single transcript. An alternative promoter e.g., SP6, T3, P60, Syn5, and KP34 may be used. A transcription template is generated by PCR to produce mRNA, using primers designed to flank the T7 promoter, gene of interest, and siRNA sequences. The reverse primer includes a stretch of thymidine (T) base (120) (SEQ ID NO: 134) to add the 120 bp length of poly(A) tail (SEQ ID NO: 133) to the mRNA. Some of the polynucleotide or RNA constructs are engineered to include siRNA designs described in Cheng, et al. (2018) J. Mater. Chem. B., 6, 4638-4644, and further comprising one or more gene of interest upstream or downstream of the siRNA sequence with linkers to connect different RNA segments (gene of interest mRNA to siRNA (SEQ ID NO:27) or siRNA to siRNA (SEQ ID NO:28)). Recombinant constructs may encode or comprise more than one siRNA sequence targeting the same or different target mRNA. Likewise, constructs may comprise nucleic acid sequences of two or more genes of interest.

Construct Synthesis

The constructs as shown in Table 1 (Compound ID numbers Cpd.1-Cpd.14) were synthesized by GeneArt, Germany (Thermo Fisher Scientific) in pMA-RQ plasmid-backbone vector containing a T7 RNA polymerase promoter with codon optimization (GeneOptimizer algorithm). Table 1 shows, for each compound (Cpd.), protein to be downregulated through siRNA binding to the corresponding mRNA (siRNA target), siRNA position in the compound, the number of siRNAs in the compound, gene of interest and respective indication. The sequences of each construct are shown in Table 2 and annotated as indicated below the table (SEQ ID NOs: 1-12, 42, 125, 97-108, and 121-122). The plasmid-backbone sequences of each construct are shown in Table 3 and compound sequence are in bold and underlined (SEQ ID NO: 13-24 and 127-128).

TABLE 1
Summary of Compounds

| | siRNA | siRNA | | Protein | Indi- |
|---|---|---|---|---|---|
| Compound ID | Target | Position | # of siRNAs | Target | cation |
| 1 unmodified* | IL-8 | 5′ | 1× | IGF-1 | OA, |
| 1 modified** | | | | | IVDD |
| 2 unmodified* | IL-8 | 5′ | 3× | IGF-1 | OA, |
| 2 modified** | | | | | IVDD |
| 3 unmodified* | IL-1beta | 5′ | 3× | IGF-1 | OA, |
| 3 modified** | | | | | IVDD |
| 4 unmodified* | TNF-α/ | 5′ | 6× | IL-4 | Psoriasis |
| 4 modified** | IL-17 | | | | |
| 5 unmodified* | TNF-α | 5′ | 3× | IL-4 | Psoriasis |
| 5 modified** | | | | | |
| 6 unmodified* | TNF-α | 3′ | 3× | IL-4 | Psoriasis |
| 6 modified** | | | | | |
| 7 unmodified* | TNF-α | 5′ | 1× | IL-4 | Psoriasis |
| 7 modified** | | | | | |
| 8 unmodified* | TNF-α | 3′ | 1× | IL-4 | Psoriasis |
| 8 modified** | | | | | |
| 9 unmodified*# | TNF-α | 3′ | 3× | IL-4 | Psoriasis |
| 9 modified**# | | | | | |
| 10 unmodified* | Turbo GFP | 3′ | 1× | IGF-1 | NA |
| 10 modified** | | | | | |
| 11 unmodified* | Turbo GFP | 3′ | 2× | IGF-1 | NA |
| 11 modified** | | | | | |
| 12 unmodified* | Turbo GFP | 3′ | 3× | IGF-1 | NA |
| 12 modified** | | | | | |
| 13 unmodified* | NA | NA | NA | IGF-1 | NA |
| 13 modified** | | | | | |
| 14 unmodified* | NA | NA | NA | IL-4 | NA |
| 14 modified** | | | | | |

*unmodified: all uridines are unmodified uridines and all other nucleotides are unmodified or natural nucleotides,
**modified: all uridines are modified (N¹-methylpseudouridine) and all other nucleotides are unmodified or natural nucleotides,
#A2 linker, IL-8: Interleukin-8, IGF-1: Insulin like growth factor-1, OA: Osteoarthritis; IVDD: Intervertebral disc disease; IL-1beta Interleukin 1 beta; TNF-α: Tumor necrosis factor-alpha, IL-17: Interleukin-17, IL-4: Interleukin-4, Turbo GFP: Turbo green fluorescent protein (derived from copepod Pontellina plumate).

TABLE 2
Sequences of Compounds

| SEQ | | |
|---|---|---|
| ID NO | Compound | Sequence (5′ to 3′) |
| 1 | Compound 1 | ATAGTGAGTCGTATTAACGTACCAACAACAAGGAAGTGCTAAAGAAACTTG |
| | unmodified | TTCTTTAGCACTTCCTTGTTTATCTTAGAGGCATATCCCTGCCACCATGAC |
| | | CATCCTGTTTCTGACAATGGTCATCAGCTACTTCGGCTGCATGAAGGCCGT |
| | | GAAGATGCACACCATGAGCAGCAGCCACCTGTTCTATCTGGCCCTGTGCCT |
| | | GCTGACCTTTACCAGCTCTGCTACCGCCGGACCTGAGACACTTTGTGGCGC |
| | | TGAACTGGTGGACGCCCTGCAGTTTGTGTGTGGCGACAGAGGCTTCTACTT |
| | | CAACAAGCCCACAGGCTACGGCAGCAGCTCTAGAAGGGCTCCTCAGACCGG |
| | | AATCGTGGACGAGTGCTGCTTCAGAAGCTGCGACCTGCGGCGGCTGGAAAT |
| | | GTATTGTGCCCCTCTGAAGCCTGCCAAGAGCGCCTAATTTATCTTAGAGGC |
| | | ATATCCCT |
| 2 | Compound 2 | ATAGTGAGTCGTATTAACGTACCAACAACAAGGAGTGCTAAAGAAACTTGT |
| | unmodified | TCTTTAGCACTCCTTGTTTATCTTAGAGGCATATCCCTACGTACCAACAAG |
| | | AGAGTGATTGAGAGTGGACTTGCCACTCTCAATCACTCTCTTTATCTTAGA |
| | | GGCATATCCCTACGTACCAACAAGAGAGCTCTGTCTGGACCACTTGGGTCC |
| | | AGACAGAGCTCTCTTTATCTTAGAGGCATATCCCTGCCACCATGACCATCC |
| | | TGTTTCTGACAATGGTCATCAGCTACTTCGGCTGCATGAAGGCCGTGAAGA |
| | | TGCACACCATGAGCAGCAGCCACCTGTTCTATCTGGCCCTGTGCCTGCTGA |
| | | CCTTTACCAGCTCTGCTACCGCCGGACCTGAGACACTTTGTGGCGCTGAAC |
| | | TGGTGGACGCCCTGCAGTTTGTGTGTGGCGACAGAGGCTTCTACTTCAACA |
| | | AGCCCACAGGCTACGGCAGCAGCTCTAGAAGGGCTCCTCAGACCGGAATCG |
| | | TGGACGAGTGCTGCTTCAGAAGCTGCGACCTGCGGCGGCTGGAAATGTATT |
| | | GTGCCCCTCTGAAGCCTGCCAAGAGCGCCTAATTTATCTTAGAGGCATATC |
| | | CCT |
| 3 | Compound 3 | ATAGTGAGTCGTATTAACGTACCAACAAGAAAGATGATAAGCCCACTCTAC |
| | unmodified | TTGAGAGTGGGCTTATCATCTTTCTTTATCTTAGAGGCATATCCCTACGTA |
| | | CCAACAAGGTGATGTCTGGTCCATATGAACTTGTCATATGGACCAGACATC |
| | | ACCTTTATCTTAGAGGCATATCCCTACGTACCAACAAGATGATAAGCCCAC |
| | | TCTAACTTGTAGAGTGGGCTTATCATCTTTATCTTAGAGGCATATCCCTGC |
| | | CACCATGACCATCCTGTTTCTGACAATGGTCATCAGCTACTTCGGCTGCAT |
| | | GAAGGCCGTGAAGATGCACACCATGAGCAGCAGCCACCTGTTCTATCTGGC |
| | | CCTGTGCCTGCTGACCTTTACCAGCTCTGCTACCGCCGGACCTGAGACACT |
| | | TTGTGGCGCTGAACTGGTGGACGCCCTGCAGTTTGTGTGTGGCGACAGAGG |
| | | CTTCTACTTCAACAAGCCCACAGGCTACGGCAGCAGCTCTAGAAGGGCTCC |
| | | TCAGACCGGAATCGTGGACGAGTGCTGCTTCAGAAGCTGCGACCTGCGGCG |
| | | GCTGGAAATGTATTGTGCCCCTCTGAAGCCTGCCAAGAGCGCCTAATTTAT |
| | | CTTAGAGGCATATCCCT |
| 4 | Compound 4 | ATAGTGAGTCGTATTAACGTACCAACAAGGCGTGGAGCTGAGAGATAAACT |
| | unmodified | TGTTATCTCTCAGCTCCACGCCTTTATCTTAGAGGCATATCCCTACGTACC |
| | | AACAAGGGCCTGTACCTCATCTACTACTTGAGTAGATGAGGTACAGGCCCT |
| | | TTATCTTAGAGGCATATCCCTACGTACCAACAAGGTATGAGCCCATCTATC |
| | | TACTTGAGATAGATGGGCTCATACCTTTATCTTAGAGGCATATCCCTACGT |
| | | ACCAACAAGCAATGAGGACCCTGAGAGATACTTGATCTCTCAGGGTCCTCA |
| | | TTGCTTTATCTTAGAGGCATATCCCTACGTACCAACAAGCTGATGGGAACG |
| | | TGGACTAACTTGTAGTCCACGTTCCCATCAGCTTTATCTTAGAGGCATATC |
| | | CCTACGTACCAACAAGGTCCTCAGATTACTACAAACTTGTTGTAGTAATCT |
| | | GAGGACCTTTATCTTAGAGGCATATCCCTGCCACCATGGGACTGACATCTC |
| | | AACTGCTGCCTCCACTGTTCTTTCTGCTGGCCTGCGCCGGCAATTTTGTGC |
| | | ACGGCCACAAGTGCGACATCACCCTGCAAGAGATCATCAAGACCCTGAACA |
| | | GCCTGACCGAGCAGAAAACCCTGTGCACCGAGCTGACCGTGACCGATATCT |
| | | TTGCCGCCAGCAAGAACACAACCGAGAAAGAGACATTCTGCAGAGCCGCCA |
| | | CCGTGCTGAGACAGTTCTACAGCCACCACGAGAAGGACACCAGATGCCTGG |
| | | GAGCTACAGCCCAGCAGTTCCACAGACACAAGCAGCTGATCCGGTTCCTGA |
| | | AGCGGCTGGACAGAAATCTGTGGGGACTCGCCGGCCTGAATAGCTGCCCTG |
| | | TGAAAGAGGCCAACCAGTCTACCCTGGAAAACTTCCTGGAACGGCTGAAAA |
| | | CCATCATGCGCGAGAAGTACAGCAAGTGCAGCAGCTGATTTATCTTAGAGG |
| | | CATATCCCT |
| 5 | Compound 5 | ATAGTGAGTCGTATTAACGTACCAACAAGGCGTGGAGCTGAGAGATAAACT |
| | unmodified | TGTTATCTCTCAGCTCCACGCCTTTATCTTAGAGGCATATCCCTACGTACC |
| | | AACAAGGGCCTGTACCTCATCTACTACTTGAGTAGATGAGGTACAGGCCCT |
| | | TTATCTTAGAGGCATATCCCTACGTACCAACAAGGTATGAGCCCATCTATC |
| | | TACTTGAGATAGATGGGCTCATACCTTTATCTTAGAGGCATATCCCTGCCA |
| | | CCATGGGACTGACATCTCAACTGCTGCCTCCACTGTTCTTTCTGCTGGCCT |
| | | GCGCCGGCAATTTTGTGCACGGCCACAAGTGCGACATCACCCTGCAAGAGA |
| | | TCATCAAGACCCTGAACAGCCTGACCGAGCAGAAAACCCTGTGCACCGAGC |
| | | TGACCGTGACCGATATCTTTGCCGCCAGCAAGAACACAACCGAGAAAGAGA |
| | | CATTCTGCAGAGCCGCCACCGTGCTGAGACAGTTCTACAGCCACCACGAGA |
| | | AGGACACCAGATGCCTGGGAGCTACAGCCCAGCAGTTCCACAGACACAAGC |
| | | AGCTGATCCGGTTCCTGAAGCGGCTGGACAGAAATCTGTGGGGACTCGCCG |
| | | GCCTGAATAGCTGCCCTGTGAAAGAGGCCAACCAGTCTACCCTGGAAAACT |
| | | TCCTGGAACGGCTGAAAACCATCATGCGCGAGAAGTACAGCAAGTGCAGCA |
| | | GCTGATTTATCTTAGAGGCATATCCCT |
| 6 | Compound 6 | GCCACCATGGGACTGACATCTCAACTGCTGCCTCCACTGTTCTTTCTGCTG |
| | unmodified | GCCTGCGCCGGCAATTTTGTGCACGGCCACAAGTGCGACATCACCCTGCAA |
| | | GAGATCATCAAGACCCTGAACAGCCTGACCGAGCAGAAAACCCTGTGCACC |
| | | GAGCTGACCGTGACCGATATCTTTGCCGCCAGCAAGAACACAACCGAGAAA |
| | | GAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACAC |
| | | AAGCAGCTGATCCGGTTCCTGAAGCGGCTGGACAGAAATCTGTGGGGACTC |
| | | GCCGGCCTGAATAGCTGCCCTGTGAAAGAGGCCAACCAGTCTACCCTGGAA |
| | | AACTTCCTGGAACGGCTGAAAACCATCATGCGCGAGAAGTACAGCAAGTGC |
| | | AGCAGCTGAATAGTGAGTCGTATTAACGTACCAACAAGGCGTGGAGCTGAG |
| | | AGATAAACTTGTTATCTCTCAGCTCCACGCCTTTATCTTAGAGGCATATCC |
| | | CTACGTACCAACAAGGGCCTGTACCTCATCTACTACTTGAGTAGATGAGGT |
| | | ACAGGCCCTTTATCTTAGAGGCATATCCCTACGTACCAACAAGGTATGAGC |
| | | CCATCTATCTACTTGAGATAGATGGGCTCATACCTTTATCTTAGAGGCATA |
| | | TCCCTTTTATCTTAGAGGCATATCCCT |
| 7 | Compound 7 | ATAGTGAGTCGTATTAACGTACCAACAAGGCGTGGAGCTGAGAGATAAACT |
| | unmodified | TGTTATCTCTCAGCTCCACGCCTTTATCTTAGAGGCATATCCCTGCCACCA |
| | | TGGGCCTGACATCTCAGTTGCTGCCTCCACTGTTCTTTCTGCTGGCCTGCG |
| | | CCGGCAATTTTGTGCACGGCCACAAGTGCGACATCACCCTGCAAGAGATCA |
| | | TCAAGACCCTGAACAGCCTGACCGAGCAGAAAACCCTGTGCACCGAGCTGA |
| | | CCGTGACCGATATCTTTGCCGCCAGCAAGAACACAACCGAGAAAGAGACAT |
| | | TCTGCAGAGCCGCCACCGTGCTGAGACAGTTCTACAGCCACCACGAGAAGG |
| | | ACACCAGATGCCTGGGAGCTACAGCCCAGCAGTTCCACAGACACAAGCAGC |
| | | TGATCCGGTTCCTGAAGCGGCTGGACAGAAATCTGTGGGGACTCGCCGGCC |
| | | TGAATAGCTGCCCTGTGAAAGAGGCCAACCAGTCTACCCTGGAAAACTTCC |
| | | TGGAACGGCTGAAAACCATCATGCGCGAGAAGTACAGCAAGTGCAGCAGCT |
| | | AGTTTATCTTAGAGGCATATCCCT |
| 8 | Compound 8 | GCCACCATGGGACTGACATCTCAACTGCTGCCTCCACTGTTCTTTCTGCTG |
| | unmodified | GCCTGCGCCGGCAATTTTGTGCACGGCCACAAGTGCGACATCACCCTGCAA |
| | | GAGATCATCAAGACCCTGAACAGCCTGACCGAGCAGAAAACCCTGTGCACC |
| | | GAGCTGACCGTGACCGATATCTTTGCCGCCAGCAAGAACACAACCGAGAAA |
| | | GAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACAC |
| | | AAGCAGCTGATCCGGTTCCTGAAGCGGCTGGACAGAAATCTGTGGGGACTC |
| | | GCCGGCCTGAATAGCTGCCCTGTGAAAGAGGCCAACCAGTCTACCCTGGAA |
| | | AACTTCCTGGAACGGCTGAAAACCATCATGCGCGAGAAGTACAGCAAGTGC |
| | | AGCAGCTGAATAGTGAGTCGTATTAACGTACCAACAAGGCGTGGAGCTGAG |
| | | AGATAAACTTGTTATCTCTCAGCTCCACGCCTTTATCTTAGAGGCATATCC |
| | | CTTTTATCTTAGAGGCATATCCCT |
| 9 | Compound 9 | GCCACCATGGGACTGACATCTCAACTGCTGCCTCCACTGTTCTTTCTGCTG |
| | unmodified | GCCTGCGCCGGCAATTTTGTGCACGGCCACAAGTGCGACATCACCCTGCAA |
| | | GAGATCATCAAGACCCTGAACAGCCTGACCGAGCAGAAAACCCTGTGCACC |
| | | GAGCTGACCGTGACCGATATCTTTGCCGCCAGCAAGAACACAACCGAGAAA |
| | | GAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACAC |
| | | AAGCAGCTGATCCGGTTCCTGAAGCGGCTGGACAGAAATCTGTGGGGACTC |
| | | GCCGGCCTGAATAGCTGCCCTGTGAAAGAGGCCAACCAGTCTACCCTGGAA |
| | | AACTTCCTGGAACGGCTGAAAACCATCATGCGCGAGAAGTACAGCAAGTGC |
| | | AGCAGCTGAACAACAAGGCGTGGAGCTGAGAGATAAACTTGTTATCTCTCA |
| | | GCTCCACGCCACAACAAGGGCCTGTACCTCATCTACTACTTGAGTAGATGA |
| | | GGTACAGGCCCACAACAAGGTATGAGCCCATCTATCTACTTGAGATAGATG |
| | | GGCTCATACCACAACAATTTATCTTAGAGGCATATCCCT |
| 10 | Compound 10 | GCCACCATGGGCAAGATTAGCAGCCTGCCTACACAGCTGTTCAAGTGCTGC |
| | unmodified | TTCTGCGACTTCCTGAAAGTGAAGATGCACACCATGAGCAGCAGCCACCTG |
| | | TTCTATCTGGCCCTGTGCCTGCTGACCTTTACCAGCTCTGCTACCGCCGGA |
| | | CCTGAGACACTTTGTGGCGCTGAACTGGTGGACGCCCTGCAGTTTGTGTGT |
| | | GGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGC |
| | | GACCTGCGGCGGCTGGAAATGTATTGTGCCCCTCTGAAGCCTGCCAAGAGC |
| | | GCCTAAATAGTGAGTCGTATTAACGTACCAACAACAACAAGATGAAGAGCA |
| | | CCAAACTTGTTGGTGCTCTTCATCTTGTTGTTTATCTTAGAGGCATATCCC |
| | | TTTTATCTTAGAGGCATATCCCT |
| 11 | Compound 11 | GCCACCATGGGCAAGATTAGCAGCCTGCCTACACAGCTGTTCAAGTGCTGC |
| | unmodified | TTCTGCGACTTCCTGAAAGTGAAGATGCACACCATGAGCAGCAGCCACCTG |
| | | TTCTATCTGGCCCTGTGCCTGCTGACCTTTACCAGCTCTGCTACCGCCGGA |
| | | CCTGAGACACTTTGTGGCGCTGAACTGGTGGACGCCCTGCAGTTTGTGTGT |
| | | GGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGC |
| | | GACCTGCGGCGGCTGGAAATGTATTGTGCCCCTCTGAAGCCTGCCAAGAGC |
| | | GCCTAAATAGTGAGTCGTATTAACGTACCAACAACAACAAGATGAAGAGCA |
| | | CCAAACTTGTTGGTGCTCTTCATCTTGTTGTTTATCTTAGAGGCATATCCC |
| | | TACGTACCAACAACAACAAGATGAAGAGCACCAAACTTGTTGGTGCTCTTC |
| | | ATCTTGTTGTTTATCTTAGAGGCATATCCCTTTTATCTTAGAGGCATATCC |
| | | CT |
| 12 | Compound 12 | GCCACCATGGGCAAGATTAGCAGCCTGCCTACACAGCTGTTCAAGTGCTGC |
| | unmodified | TTCTGCGACTTCCTGAAAGTGAAGATGCACACCATGAGCAGCAGCCACCTG |
| | | TTCTATCTGGCCCTGTGCCTGCTGACCTTTACCAGCTCTGCTACCGCCGGA |
| | | CCTGAGACACTTTGTGGCGCTGAACTGGTGGACGCCCTGCAGTTTGTGTGT |
| | | GGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGC |
| | | GACCTGCGGCGGCTGGAAATGTATTGTGCCCCTCTGAAGCCTGCCAAGAGC |
| | | GCCTAAATAGTGAGTCGTATTAACGTACCAACAACAACAAGATGAAGAGCA |
| | | CCAAACTTGTTGGTGCTCTTCATCTTGTTGTTTATCTTAGAGGCATATCCC |
| | | TACGTACCAACAACAACAAGATGAAGAGCACCAAACTTGTTGGTGCTCTTC |
| | | ATCTTGTTGTTTATCTTAGAGGCATATCCCTACGTACCAACAACAACAAGA |
| | | TGAAGAGCACCAAACTTGTTGGTGCTCTTCATCTTGTTGTTTATCTTAGAG |
| | | GCATATCCCTTTTATCTTAGAGGCATATCCCT |
| 125 | Compound 13 | GCCACCATGGGCAAGATTAGCAGCCTGCCTACACAGCTGTTCAAGTGCTGC |
| | unmodified | TTCTGCGACTTCCTGAAAGTGAAGATGCACACCATGAGCAGCAGCCACCTG |
| | | TTCTATCTGGCCCTGTGCCTGCTGACCTTTACCAGCTCTGCTACCGCCGGA |
| | | CCTGAGACACTTTGTGGCGCTGAACTGGTGGACGCCCTGCAGTTTGTGTGT |
| | | GGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGC |
| | | GACCTGCGGCGGCTGGAAATGTATTGTGCCCCTCTGAAGCCTGCCAAGAGC |
| | | GCCTAA |
| 42 | Compound 14 | GCCACCATGTTGCTGCTGCCTCTGTTCTTCCTGCTGGCCTGCGCCGGCAAT |
| | unmodified | TTTGTGCACGGCCACAAGTGCGACATCACCCTGCAAGAGATCATCAAGACC |
| | | CTGAACAGCCTGACCGAGCAGAAAACCCTGTGCACCGAGCTGACCGTGACC |
| | | GATATCTTTGCCGCCAGCAAGAACACAACCGAGAAAGAGACATTCTGCAGA |
| | | GCCGCCACCGTGCTGAGACAGTTCTACAGCCACCACGAGAAGGACACCAGA |
| | | TGCCTGGGAGCTACAGCCCAGCAGTTCCACAGACACAAGCAGCTGATCCGG |
| | | TTCCTGAAGCGGCTGGACAGAAATCTGTGGGGACTCGCCGGCCTGAATAGC |
| | | TGCCCTGTGAAAGAGGCCAACCAGTCTACCCTGGAAAACTTCCTGGAACGG |
| | | CTGAAAACCATCATGCGCGAGAAGTACAGCAAGTGCAGCAGCTGA |

Bold = Sense siRNA strand
Bold and Italics = anti-Sense siRNA strand
Underline = Signal peptide
Italics = Kozak sequence

TABLE 3
Plasmid Sequences

| SEQ | | |
|---|---|---|
| ID NO | Compound # | Sequence (5′ → 3′ direction) |
| 13 | Compound 1 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATATAGTGAGTCGTATTAACGTACCAACAACAAGGAAGTGCTAAAGAAA |
| | | CTTGTTCTTTAGCACTTCCTTGTTTATCTTAGAGGCATATCCCTGCCACCAT |
| | | GACCATCCTGTTTCTGACAATGGTCATCAGCTACTTCGGCTGCATGAAGGCC |
| | | GTGAAGATGCACACCATGAGCAGCAGCCACCTGTTCTATCTGGCCCTGTGCC |
| | | TGCTGACCTTTACCAGCTCTGCTACCGCCGGACCTGAGACACTTTGTGGCGC |
| | | TGAACTGGTGGACGCCCTGCAGTTTGTGTGTGGCGACAGAGGCTTCTACTTC |
| | | AACAAGCCCACAGGCTACGGCAGCAGCTCTAGAAGGGCTCCTCAGACCGGAA |
| | | TCGTGGACGAGTGCTGCTTCAGAAGCTGCGACCTGCGGCGGCTGGAAATGTA |
| | | TTGTGCCCCTCTGAAGCCTGCCAAGAGCGCCTAATTTATCTTAGAGGCATAT |
| | | CCCTCTGGGCCTCATGGGCCTTCCGCTCACTGCCCGCTTTCCAGTCGGGAAA |
| | | CCTGTCGTGCCAGCTGCATTAACATGGTCATAGCTGTTTCCTTGCGTATTGG |
| | | GCGCTCTCCGCTTCCTCGCTCACTGACTCGCTGCGCTCGGTCGTTCGGGTAA |
| | | AGCCTGGGGTGCCTAATGAGCAAAAGGCCAGCAAAAGGCCAGGAACCGTAAA |
| | | AAGGCCGCGTTGCTGGCGTTTTTCCATAGGCTCCGCCCCCCTGACGAGCATC |
| | | ACAAAAATCGACGCTCAAGTCAGAGGTGGCGAAACCCGACAGGACTATAAAG |
| | | ATACCAGGCGTTTCCCCCTGGAAGCTCCCTCGTGCGCTCTCCTGTTCCGACC |
| | | CTGCCGCTTACCGGATACCTGTCCGCCTTTCTCCCTTCGGGAAGCGTGGCGC |
| | | TTTCTCATAGCTCACGCTGTAGGTATCTCAGTTCGGTGTAGGTCGTTCGCTC |
| | | CAAGCTGGGCTGTGTGCACGAACCCCCCGTTCAGCCCGACCGCTGCGCCTTA |
| | | TCCGGTAACTATCGTCTTGAGTCCAACCCGGTAAGACACGACTTATCGCCAC |
| | | TGGCAGCAGCCACTGGTAACAGGATTAGCAGAGCGAGGTATGTAGGCGGTGC |
| | | TACAGAGTTCTTGAAGTGGTGGCCTAACTACGGCTACACTAGAAGAACAGTA |
| | | TTTGGTATCTGCGCTCTGCTGAAGCCAGTTACCTTCGGAAAAAGAGTTGGTA |
| | | GCTCTTGATCCGGCAAACAAACCACCGCTGGTAGCGGTGGTTTTTTTGTTTG |
| | | CAAGCAGCAGATTACGCGCAGAAAAAAAGGATCTCAAGAAGATCCTTTGATC |
| | | TTTTCTACGGGGTCTGACGCTCAGTGGAACGAAAACTCACGTTAAGGGATTT |
| | | TGGTCATGAGATTATCAAAAAGGATCTTCACCTAGATCCTTTTAAATTAAAA |
| | | ATGAAGTTTTAAATCAATCTAAAGTATATATGAGTAAACTTGGTCTGACAGT |
| | | TACCAATGCTTAATCAGTGAGGCACCTATCTCAGCGATCTGTCTATTTCGTT |
| | | CATCCATAGTTGCCTGACTCCCCGTCGTGTAGATAACTACGATACGGGAGGG |
| | | CTTACCATCTGGCCCCAGTGCTGCAATGATACCGCGAGAACCACGCTCACCG |
| | | GCTCCAGATTTATCAGCAATAAACCAGCCAGCCGGAAGGGCCGAGCGCAGAA |
| | | GTGGTCCTGCAACTTTATCCGCCTCCATCCAGTCTATTAATTGTTGCCGGGA |
| | | AGCTAGAGTAAGTAGTTCGCCAGTTAATAGTTTGCGCAACGTTGTTGCCATT |
| | | GCTACAGGCATCGTGGTGTCACGCTCGTCGTTTGGTATGGCTTCATTCAGCT |
| | | CCGGTTCCCAACGATCAAGGCGAGTTACATGATCCCCCATGTTGTGCAAAAA |
| | | AGCGGTTAGCTCCTTCGGTCCTCCGATCGTTGTCAGAAGTAAGTTGGCCGCA |
| | | GTGTTATCACTCATGGTTATGGCAGCACTGCATAATTCTCTTACTGTCATGC |
| | | CATCCGTAAGATGCTTTTCTGTGACTGGTGAGTACTCAACCAAGTCATTCTG |
| | | AGAATAGTGTATGCGGCGACCGAGTTGCTCTTGCCCGGCGTCAATACGGGAT |
| | | AATACCGCGCCACATAGCAGAACTTTAAAAGTGCTCATCATTGGAAAACGTT |
| | | CTTCGGGGCGAAAACTCTCAAGGATCTTACCGCTGTTGAGATCCAGTTCGAT |
| | | GTAACCCACTCGTGCACCCAACTGATCTTCAGCATCTTTTACTTTCACCAGC |
| | | GTTTCTGGGTGAGCAAAAACAGGAAGGCAAAATGCCGCAAAAAAGGGAATAA |
| | | GGGCGACACGGAAATGTTGAATACTCATACTCTTCCTTTTTCAATATTATTG |
| | | AAGCATTTATCAGGGTTATTGTCTCATGAGCGGATACATATTTGAATGTATT |
| | | TAGAAAAATAAACAAATAGGGGTTCCGCGCACATTTCCCCGAAAAGTGCCAC |
| 14 | Compound 2 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATATAGTGAGTCGTATTAACGTACCAACAACAAGGAGTGCTAAAGAAAC |
| | | TTGTTCTTTAGCACTCCTTGTTTATCTTAGAGGCATATCCCTACGTACCAAC |
| | | AAGAGAGTGATTGAGAGTGGACTTGCCACTCTCAATCACTCTCTTTATCTTA |
| | | GAGGCATATCCCTACGTACCAACAAGAGAGCTCTGTCTGGACCACTTGGGTC |
| | | CAGACAGAGCTCTCTTTATCTTAGAGGCATATCCCTGCCACCATGACCATCC |
| | | TGTTTCTGACAATGGTCATCAGCTACTTCGGCTGCATGAAGGCCGTGAAGAT |
| | | GCACACCATGAGCAGCAGCCACCTGTTCTATCTGGCCCTGTGCCTGCTGACC |
| | | TTTACCAGCTCTGCTACCGCCGGACCTGAGACACTTTGTGGCGCTGAACTGG |
| | | TGGACGCCCTGCAGTTTGTGTGTGGCGACAGAGGCTTCTACTTCAACAAGCC |
| | | CACAGGCTACGGCAGCAGCTCTAGAAGGGCTCCTCAGACCGGAATCGTGGAC |
| | | GAGTGCTGCTTCAGAAGCTGCGACCTGCGGCGGCTGGAAATGTATTGTGCCC |
| | | CTCTGAAGCCTGCCAAGAGCGCCTAATTTATCTTAGAGGCATATCCCTCTGG |
| | | GCCTCATGGGCCTTCCGCTCACTGCCCGCTTTCCAGTCGGGAAACCTGTCGT |
| | | GCCAGCTGCATTAACATGGTCATAGCTGTTTCCTTGCGTATTGGGCGCTCTC |
| | | CGCTTCCTCGCTCACTGACTCGCTGCGCTCGGTCGTTCGGGTAAAGCCTGGG |
| | | GTGCCTAATGAGCAAAAGGCCAGCAAAAGGCCAGGAACCGTAAAAAGGCCGC |
| | | GTTGCTGGCGTTTTTCCATAGGCTCCGCCCCCCTGACGAGCATCACAAAAAT |
| | | CGACGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCCGCT |
| | | TACCGGATACCTGTCCGCCTTTCTCCCTTCGGGAAGCGTGGCGCTTTCTCAT |
| | | AGCTCACGCTGTAGGTATCTCAGTTCGGTGTAGGTCGTTCGCTCCAAGCTGG |
| | | GCTGTGTGCACGAACCCCCCGTTCAGCCCGACCGCTGCGCCTTATCCGGTAA |
| | | CTATCGTCTTGAGTCCAACCCGGTAAGACACGACTTATCGCCACTGGCAGCA |
| | | GCCACTGGTAACAGGATTAGCAGAGCGAGGTATGTAGGCGGTGCTACAGAGT |
| | | TCTTGAAGTGGTGGCCTAACTACGGCTACACTAGAAGAACAGTATTTGGTAT |
| | | CTGCGCTCTGCTGAAGCCAGTTACCTTCGGAAAAAGAGTTGGTAGCTCTTGA |
| | | TCCGGCAAACAAACCACCGCTGGTAGCGGTGGTTTTTTTGTTTGCAAGCAGC |
| | | AGATTACGCGCAGAAAAAAAGGATCTCAAGAAGATCCTTTGATCTTTTCTAC |
| | | GGGGTCTGACGCTCAGTGGAACGAAAACTCACGTTAAGGGATTTTGGTCATG |
| | | AGATTATCAAAAAGGATCTTCACCTAGATCCTTTTAAATTAAAAATGAAGTT |
| | | TTAAATCAATCTAAAGTATATATGAGTAAACTTGGTCTGACAGTTACCAATG |
| | | CTTAATCAGTGAGGCACCTATCTCAGCGATCTGTCTATTTCGTTCATCCATA |
| | | GTTGCCTGACTCCCCGTCGTGTAGATAACTACGATACGGGAGGGCTTACCAT |
| | | CTGGCCCCAGTGCTGCAATGATACCGCGAGAACCACGCTCACCGGCTCCAGA |
| | | TTTATCAGCAATAAACCAGCCAGCCGGAAGGGCCGAGCGCAGAAGTGGTCCT |
| | | GCAACTTTATCCGCCTCCATCCAGTCTATTAATTGTTGCCGGGAAGCTAGAG |
| | | TAAGTAGTTCGCCAGTTAATAGTTTGCGCAACGTTGTTGCCATTGCTACAGG |
| | | CATCGTGGTGTCACGCTCGTCGTTTGGTATGGCTTCATTCAGCTCCGGTTCC |
| | | CAACGATCAAGGCGAGTTACATGATCCCCCATGTTGTGCAAAAAAGCGGTTA |
| | | GCTCCTTCGGTCCTCCGATCGTTGTCAGAAGTAAGTTGGCCGCAGTGTTATC |
| | | ACTCATGGTTATGGCAGCACTGCATAATTCTCTTACTGTCATGCCATCCGTA |
| | | AGATGCTTTTCTGTGACTGGTGAGTACTCAACCAAGTCATTCTGAGAATAGT |
| | | GTATGCGGCGACCGAGTTGCTCTTGCCCGGCGTCAATACGGGATAATACCGC |
| | | GCCACATAGCAGAACTTTAAAAGTGCTCATCATTGGAAAACGTTCTTCGGGG |
| | | CGAAAACTCTCAAGGATCTTACCGCTGTTGAGATCCAGTTCGATGTAACCCA |
| | | CTCGTGCACCCAACTGATCTTCAGCATCTTTTACTTTCACCAGCGTTTCTGG |
| | | GTGAGCAAAAACAGGAAGGCAAAATGCCGCAAAAAAGGGAATAAGGGCGACA |
| | | CGGAAATGTTGAATACTCATACTCTTCCTTTTTCAATATTATTGAAGCATTT |
| | | ATCAGGGTTATTGTCTCATGAGCGGATACATATTTGAATGTATTTAGAAAAA |
| | | TAAACAAATAGGGGTTCCGCGCACATTTCCCCGAAAAGTGCCAC |
| 15 | Compound 3 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATATAGTGAGTCGTATTAACGTACCAACAAGAAAGATGATAAGCCCACT |
| | | CTACTTGAGAGTGGGCTTATCATCTTTCTTTATCTTAGAGGCATATCCCTAC |
| | | GTACCAACAAGGTGATGTCTGGTCCATATGAACTTGTCATATGGACCAGACA |
| | | TCACCTTTATCTTAGAGGCATATCCCTACGTACCAACAAGATGATAAGCCCA |
| | | CTCTAACTTGTAGAGTGGGCTTATCATCTTTATCTTAGAGGCATATCCCTGC |
| | | CACCATGACCATCCTGTTTCTGACAATGGTCATCAGCTACTTCGGCTGCATG |
| | | AAGGCCGTGAAGATGCACACCATGAGCAGCAGCCACCTGTTCTATCTGGCCC |
| | | TGTGCCTGCTGACCTTTACCAGCTCTGCTACCGCCGGACCTGAGACACTTTG |
| | | TGGCGCTGAACTGGTGGACGCCCTGCAGTTTGTGTGTGGCGACAGAGGCTTC |
| | | TACTTCAACAAGCCCACAGGCTACGGCAGCAGCTCTAGAAGGGCTCCTCAGA |
| | | CCGGAATCGTGGACGAGTGCTGCTTCAGAAGCTGCGACCTGCGGCGGCTGGA |
| | | AATGTATTGTGCCCCTCTGAAGCCTGCCAAGAGCGCCTAGTTTATCTTAGAG |
| | | GCATATCCCTCTGGGCCTCATGGGCCTTCCGCTCACTGCCCGCTTTCCAGTC |
| | | GGGAAACCTGTCGTGCCAGCTGCATTAACATGGTCATAGCTGTTTCCTTGCG |
| | | TATTGGGCGCTCTCCGCTTCCTCGCTCACTGACTCGCTGCGCTCGGTCGTTC |
| | | GGGTAAAGCCTGGGGTGCCTAATGAGCAAAAGGCCAGCAAAAGGCCAGGAAC |
| | | CGTAAAAAGGCCGCGTTGCTGGCGTTTTTCCATAGGCTCCGCCCCCCTGACG |
| | | AGCATCACAAAAATCGACGCTCAAGTCAGAGGTGGCGAAACCCGACAGGACT |
| | | ATAAAGATACCAGGCGTTTCCCCCTGGAAGCTCCCTCGTGCGCTCTCCTGTT |
| | | CCGACCCTGCCGCTTACCGGATACCTGTCCGCCTTTCTCCCTTCGGGAAGCG |
| | | TGGCGCTTTCTCATAGCTCACGCTGTAGGTATCTCAGTTCGGTGTAGGTCGT |
| | | TCGCTCCAAGCTGGGCTGTGTGCACGAACCCCCCGTTCAGCCCGACCGCTGC |
| | | GCCTTATCCGGTAACTATCGTCTTGAGTCCAACCCGGTAAGACACGACTTAT |
| | | CGCCACTGGCAGCAGCCACTGGTAACAGGATTAGCAGAGCGAGGTATGTAGG |
| | | CGGTGCTACAGAGTTCTTGAAGTGGTGGCCTAACTACGGCTACACTAGAAGA |
| | | ACAGTATTTGGTATCTGCGCTCTGCTGAAGCCAGTTACCTTCGGAAAAAGAG |
| | | TTGGTAGCTCTTGATCCGGCAAACAAACCACCGCTGGTAGCGGTGGTTTTTT |
| | | TGTTTGCAAGCAGCAGATTACGCGCAGAAAAAAAGGATCTCAAGAAGATCCT |
| | | TTGATCTTTTCTACGGGGTCTGACGCTCAGTGGAACGAAAACTCACGTTAAG |
| | | GGATTTTGGTCATGAGATTATCAAAAAGGATCTTCACCTAGATCCTTTTAAA |
| | | TTAAAAATGAAGTTTTAAATCAATCTAAAGTATATATGAGTAAACTTGGTCT |
| | | GACAGTTACCAATGCTTAATCAGTGAGGCACCTATCTCAGCGATCTGTCTAT |
| | | TTCGTTCATCCATAGTTGCCTGACTCCCCGTCGTGTAGATAACTACGATACG |
| | | GGAGGGCTTACCATCTGGCCCCAGTGCTGCAATGATACCGCGAGAACCACGC |
| | | TCACCGGCTCCAGATTTATCAGCAATAAACCAGCCAGCCGGAAGGGCCGAGC |
| | | GCAGAAGTGGTCCTGCAACTTTATCCGCCTCCATCCAGTCTATTAATTGTTG |
| | | CCGGGAAGCTAGAGTAAGTAGTTCGCCAGTTAATAGTTTGCGCAACGTTGTT |
| | | GCCATTGCTACAGGCATCGTGGTGTCACGCTCGTCGTTTGGTATGGCTTCAT |
| | | TCAGCTCCGGTTCCCAACGATCAAGGCGAGTTACATGATCCCCCATGTTGTG |
| | | CAAAAAAGCGGTTAGCTCCTTCGGTCCTCCGATCGTTGTCAGAAGTAAGTTG |
| | | GCCGCAGTGTTATCACTCATGGTTATGGCAGCACTGCATAATTCTCTTACTG |
| | | TCATGCCATCCGTAAGATGCTTTTCTGTGACTGGTGAGTACTCAACCAAGTC |
| | | ATTCTGAGAATAGTGTATGCGGCGACCGAGTTGCTCTTGCCCGGCGTCAATA |
| | | CGGGATAATACCGCGCCACATAGCAGAACTTTAAAAGTGCTCATCATTGGAA |
| | | AACGTTCTTCGGGGCGAAAACTCTCAAGGATCTTACCGCTGTTGAGATCCAG |
| | | TTCGATGTAACCCACTCGTGCACCCAACTGATCTTCAGCATCTTTTACTTTC |
| | | ACCAGCGTTTCTGGGTGAGCAAAAACAGGAAGGCAAAATGCCGCAAAAAAGG |
| | | GAATAAGGGCGACACGGAAATGTTGAATACTCATACTCTTCCTTTTTCAATA |
| | | TTATTGAAGCATTTATCAGGGTTATTGTCTCATGAGCGGATACATATTTGAA |
| | | TGTATTTAGAAAAATAAACAAATAGGGGTTCCGCGCACATTTCCCCGAAAAG |
| | | TGCCAC |
| 16 | Compound 4 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATATAGTGAGTCGTATTAACGTACCAACAAGGCGTGGAGCTGAGAGATA |
| | | AACTTGTTATCTCTCAGCTCCACGCCTTTATCTTAGAGGCATATCCCTACGT |
| | | ACCAACAAGGGCCTGTACCTCATCTACTACTTGAGTAGATGAGGTACAGGCC |
| | | CTTTATCTTAGAGGCATATCCCTACGTACCAACAAGGTATGAGCCCATCTAT |
| | | CTACTTGAGATAGATGGGCTCATACCTTTATCTTAGAGGCATATCCCTACGT |
| | | ACCAACAAGCAATGAGGACCCTGAGAGATACTTGATCTCTCAGGGTCCTCAT |
| | | TGCTTTATCTTAGAGGCATATCCCTACGTACCAACAAGCTGATGGGAACGTG |
| | | GACTAACTTGTAGTCCACGTTCCCATCAGCTTTATCTTAGAGGCATATCCCT |
| | | ACGTACCAACAAGGTCCTCAGATTACTACAAACTTGTTGTAGTAATCTGAGG |
| | | ACCTTTATCTTAGAGGCATATCCCTGCCACCATGGGACTGACATCTCAACTG |
| | | CTGCCTCCACTGTTCTTTCTGCTGGCCTGCGCCGGCAATTTTGTGCACGGCC |
| | | ACAAGTGCGACATCACCCTGCAAGAGATCATCAAGACCCTGAACAGCCTGAC |
| | | CGAGCAGAAAACCCTGTGCACCGAGCTGACCGTGACCGATATCTTTGCCGCC |
| | | AGCAAGAACACAACCGAGAAAGAGACATTCTGCAGAGCCGCCACCGTGCTGA |
| | | GACAGTTCTACAGCCACCACGAGAAGGACACCAGATGCCTGGGAGCTACAGC |
| | | CCAGCAGTTCCACAGACACAAGCAGCTGATCCGGTTCCTGAAGCGGCTGGAC |
| | | AGAAATCTGTGGGGACTCGCCGGCCTGAATAGCTGCCCTGTGAAAGAGGCCA |
| | | ACCAGTCTACCCTGGAAAACTTCCTGGAACGGCTGAAAACCATCATGCGCGA |
| | | GAAGTACAGCAAGTGCAGCAGCTGATTTATCTTAGAGGCATATCCCTCTGGG |
| | | CCTCATGGGCCTTCCGCTCACTGCCCGCTTTCCAGTCGGGAAACCTGTCGTG |
| | | CCAGCTGCATTAACATGGTCATAGCTGTTTCCTTGCGTATTGGGCGCTCTCC |
| | | GCTTCCTCGCTCACTGACTCGCTGCGCTCGGTCGTTCGGGTAAAGCCTGGGG |
| | | TGCCTAATGAGCAAAAGGCCAGCAAAAGGCCAGGAACCGTAAAAAGGCCGCG |
| | | TTGCTGGCGTTTTTCCATAGGCTCCGCCCCCCTGACGAGCATCACAAAAATC |
| | | GACGCTCAAGTCAGAGGTGGCGAAACCCGACAGGACTATAAAGATACCAGGC |
| | | GTTTCCCCCTGGAAGCTCCCTCGTGCGCTCTCCTGTTCCGACCCTGCCGCTT |
| | | ACCGGATACCTGTCCGCCTTTCTCCCTTCGGGAAGCGTGGCGCTTTCTCATA |
| | | GCTCACGCTGTAGGTATCTCAGTTCGGTGTAGGTCGTTCGCTCCAAGCTGGG |
| | | CTGTGTGCACGAACCCCCCGTTCAGCCCGACCGCTGCGCCTTATCCGGTAAC |
| | | TATCGTCTTGAGTCCAACCCGGTAAGACACGACTTATCGCCACTGGCAGCAG |
| | | CCACTGGTAACAGGATTAGCAGAGCGAGGTATGTAGGCGGTGCTACAGAGTT |
| | | CTTGAAGTGGTGGCCTAACTACGGCTACACTAGAAGAACAGTATTTGGTATC |
| | | TGCGCTCTGCTGAAGCCAGTTACCTTCGGAAAAAGAGTTGGTAGCTCTTGAT |
| | | CCGGCAAACAAACCACCGCTGGTAGCGGTGGTTTTTTTGTTTGCAAGCAGCA |
| | | GATTACGCGCAGAAAAAAAGGATCTCAAGAAGATCCTTTGATCTTTTCTACG |
| | | GGGTCTGACGCTCAGTGGAACGAAAACTCACGTTAAGGGATTTTGGTCATGA |
| | | GATTATCAAAAAGGATCTTCACCTAGATCCTTTTAAATTAAAAATGAAGTTT |
| | | TAAATCAATCTAAAGTATATATGAGTAAACTTGGTCTGACAGTTACCAATGC |
| | | TTAATCAGTGAGGCACCTATCTCAGCGATCTGTCTATTTCGTTCATCCATAG |
| | | TTGCCTGACTCCCCGTCGTGTAGATAACTACGATACGGGAGGGCTTACCATC |
| | | TGGCCCCAGTGCTGCAATGATACCGCGAGAACCACGCTCACCGGCTCCAGAT |
| | | TTATCAGCAATAAACCAGCCAGCCGGAAGGGCCGAGCGCAGAAGTGGTCCTG |
| | | CAACTTTATCCGCCTCCATCCAGTCTATTAATTGTTGCCGGGAAGCTAGAGT |
| | | AAGTAGTTCGCCAGTTAATAGTTTGCGCAACGTTGTTGCCATTGCTACAGGC |
| | | ATCGTGGTGTCACGCTCGTCGTTTGGTATGGCTTCATTCAGCTCCGGTTCCC |
| | | AACGATCAAGGCGAGTTACATGATCCCCCATGTTGTGCAAAAAAGCGGTTAG |
| | | CTCCTTCGGTCCTCCGATCGTTGTCAGAAGTAAGTTGGCCGCAGTGTTATCA |
| | | CTCATGGTTATGGCAGCACTGCATAATTCTCTTACTGTCATGCCATCCGTAA |
| | | GATGCTTTTCTGTGACTGGTGAGTACTCAACCAAGTCATTCTGAGAATAGTG |
| | | TATGCGGCGACCGAGTTGCTCTTGCCCGGCGTCAATACGGGATAATACCGCG |
| | | CCACATAGCAGAACTTTAAAAGTGCTCATCATTGGAAAACGTTCTTCGGGGC |
| | | GAAAACTCTCAAGGATCTTACCGCTGTTGAGATCCAGTTCGATGTAACCCAC |
| | | TCGTGCACCCAACTGATCTTCAGCATCTTTTACTTTCACCAGCGTTTCTGGG |
| | | TGAGCAAAAACAGGAAGGCAAAATGCCGCAAAAAAGGGAATAAGGGCGACAC |
| | | GGAAATGTTGAATACTCATACTCTTCCTTTTTCAATATTATTGAAGCATTTA |
| | | TCAGGGTTATTGTCTCATGAGCGGATACATATTTGAATGTATTTAGAAAAAT |
| | | AAACAAATAGGGGTTCCGCGCACATTTCCCCGAAAAGTGCCAC |
| 17 | Compound 5 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATATAGTGAGTCGTATTAACGTACCAACAAGGCGTGGAGCTGAGAGATA |
| | | AACTTGTTATCTCTCAGCTCCACGCCTTTATCTTAGAGGCATATCCCTACGT |
| | | ACCAACAAGGGCCTGTACCTCATCTACTACTTGAGTAGATGAGGTACAGGCC |
| | | CTTTATCTTAGAGGCATATCCCTACGTACCAACAAGGTATGAGCCCATCTAT |
| | | CTACTTGAGATAGATGGGCTCATACCTTTATCTTAGAGGCATATCCCTGCCA |
| | | CCATGGGACTGACATCTCAACTGCTGCCTCCACTGTTCTTTCTGCTGGCCTG |
| | | CGCCGGCAATTTTGTGCACGGCCACAAGTGCGACATCACCCTGCAAGAGATC |
| | | ATCAAGACCCTGAACAGCCTGACCGAGCAGAAAACCCTGTGCACCGAGCTGA |
| | | CCGTGACCGATATCTTTGCCGCCAGCAAGAACACAACCGAGAAAGAGACATT |
| | | CTGCAGAGCCGCCACCGTGCTGAGACAGTTCTACAGCCACCACGAGAAGGAC |
| | | ACCAGATGCCTGGGAGCTACAGCCCAGCAGTTCCACAGACACAAGCAGCTGA |
| | | TCCGGTTCCTGAAGCGGCTGGACAGAAATCTGTGGGGACTCGCCGGCCTGAA |
| | | TAGCTGCCCTGTGAAAGAGGCCAACCAGTCTACCCTGGAAAACTTCCTGGAA |
| | | CGGCTGAAAACCATCATGCGCGAGAAGTACAGCAAGTGCAGCAGCTGATTTA |
| | | TCTTAGAGGCATATCCCTCTGGGCCTCATGGGCCTTCCGCTCACTGCCCGCT |
| | | TTCCAGTCGGGAAACCTGTCGTGCCAGCTGCATTAACATGGTCATAGCTGTT |
| | | TCCTTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAAAGG |
| | | CCAGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACCCG |
| | | ACAGGACTATAAAGATACCAGGCGTTTCCCCCTGGAAGCTCCCTCGTGCGCT |
| | | CTCCTGTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTTCGGTG |
| | | TAGGTCGTTCGCTCCAAGCTGGGCTGTGTGCACGAACCCCCCGTTCAGCCCG |
| | | ACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGG |
| | | TATGTAGGCGGTGCTACAGAGTTCTTGAAGTGGTGGCCTAACTACGGCTACA |
| | | CTAGAAGAACAGTATTTGGTATCTGCGCTCTGCTGAAGCCAGTTACCTTCGG |
| | | AAAAAGAGTTGGTAGCTCTTGATCCGGCAAACAAACCACCGCTGGTAGCGGT |
| | | GGTTTTTTTGTTTGCAAGCAGCAGATTACGCGCAGAAAAAAAGGATCTCAAG |
| | | AAGATCCTTTGATCTTTTCTACGGGGTCTGACGCTCAGTGGAACGAAAACTC |
| | | ACGTTAAGGGATTTTGGTCATGAGATTATCAAAAAGGATCTTCACCTAGATC |
| | | CTTTTAAATTAAAAATGAAGTTTTAAATCAATCTAAAGTATATATGAGTAAA |
| | | CTTGGTCTGACAGTTACCAATGCTTAATCAGTGAGGCACCTATCTCAGCGAT |
| | | CTGTCTATTTCGTTCATCCATAGTTGCCTGACTCCCCGTCGTGTAGATAACT |
| | | ACGATACGGGAGGGCTTACCATCTGGCCCCAGTGCTGCAATGATACCGCGAG |
| | | AACCACGCTCACCGGCTCCAGATTTATCAGCAATAAACCAGCCAGCCGGAAG |
| | | GGCCGAGCGCAGAAGTGGTCCTGCAACTTTATCCGCCTCCATCCAGTCTATT |
| | | AATTGTTGCCGGGAAGCTAGAGTAAGTAGTTCGCCAGTTAATAGTTTGCGCA |
| | | ACGTTGTTGCCATTGCTACAGGCATCGTGGTGTCACGCTCGTCGTTTGGTAT |
| | | GGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAATTC |
| | | TCTTACTGTCATGCCATCCGTAAGATGCTTTTCTGTGACTGGTGAGTACTCA |
| | | ACCAAGTCATTCTGAGAATAGTGTATGCGGCGACCGAGTTGCTCTTGCCCGG |
| | | CGTCAATACGGGATAATACCGCGCCACATAGCAGAACTTTAAAAGTGCTCAT |
| | | CATTGGAAAACGTTCTTCGGGGCGAAAACTCTCAAGGATCTTACCGCTGTTG |
| | | AGATCCAGTTCGATGTAACCCACTCGTGCACCCAACTGATCTTCAGCATCTT |
| | | TTACTTTCACCAGCGTTTCTGGGTGAGCAAAAACAGGAAGGCAAAATGCCGC |
| | | AAAAAAGGGAATAAGGGCGACACGGAAATGTTGAATACTCATACTCTTCCTT |
| | | TTTCAATATTATTGAAGCATTTATCAGGGTTATTGTCTCATGAGCGGATACA |
| | | TATTTGAATGTATTTAGAAAAATAAACAAATAGGGGTTCCGCGCACATTTCC |
| | | CCGAAAAGTGCCAC |
| 18 | Compound 6 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGACTGACATCTCAACTGCTGCCTCCACTGTTCTTTCT |
| | | GCTGGCCTGCGCCGGCAATTTTGTGCACGGCCACAAGTGCGACATCACCCTG |
| | | CAAGAGATCATCAAGACCCTGAACAGCCTGACCGAGCAGAAAACCCTGTGCA |
| | | CCGAGCTGACCGTGACCGATATCTTTGCCGCCAGCAAGAACACAACCGAGAA |
| | | AGAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACACA |
| | | AGCAGCTGATCCGGTTCCTGAAGCGGCTGGACAGAAATCTGTGGGGACTCGC |
| | | CGGCCTGAATAGCTGCCCTGTGAAAGAGGCCAACCAGTCTACCCTGGAAAAC |
| | | TTCCTGGAACGGCTGAAAACCATCATGCGCGAGAAGTACAGCAAGTGCAGCA |
| | | GCTGAATAGTGAGTCGTATTAACGTACCAACAAGGCGTGGAGCTGAGAGATA |
| | | AACTTGTTATCTCTCAGCTCCACGCCTTTATCTTAGAGGCATATCCCTACGT |
| | | ACCAACAAGGGCCTGTACCTCATCTACTACTTGAGTAGATGAGGTACAGGCC |
| | | CTTTATCTTAGAGGCATATCCCTACGTACCAACAAGGTATGAGCCCATCTAT |
| | | CTACTTGAGATAGATGGGCTCATACCTTTATCTTAGAGGCATATCCCTTTTA |
| | | TCTTAGAGGCATATCCCTCTGGGCCTCATGGGCCTTCCGCTCACTGCCCGCT |
| | | TTCCAGTCGGGAAACCTGTCGTGCCAGCTGCATTAACATGGTCATAGCTGTT |
| | | TCCTTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAAAGG |
| | | CCAGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACCCG |
| | | ACAGGACTATAAAGATACCAGGCGTTTCCCCCTGGAAGCTCCCTCGTGCGCT |
| | | CTCCTGTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTTCGGTG |
| | | TAGGTCGTTCGCTCCAAGCTGGGCTGTGTGCACGAACCCCCCGTTCAGCCCG |
| | | ACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGG |
| | | TATGTAGGCGGTGCTACAGAGTTCTTGAAGTGGTGGCCTAACTACGGCTACA |
| | | CTAGAAGAACAGTATTTGGTATCTGCGCTCTGCTGAAGCCAGTTACCTTCGG |
| | | AAAAAGAGTTGGTAGCTCTTGATCCGGCAAACAAACCACCGCTGGTAGCGGT |
| | | GGTTTTTTTGTTTGCAAGCAGCAGATTACGCGCAGAAAAAAAGGATCTCAAG |
| | | AAGATCCTTTGATCTTTTCTACGGGGTCTGACGCTCAGTGGAACGAAAACTC |
| | | ACGTTAAGGGATTTTGGTCATGAGATTATCAAAAAGGATCTTCACCTAGATC |
| | | CTTTTAAATTAAAAATGAAGTTTTAAATCAATCTAAAGTATATATGAGTAAA |
| | | CTTGGTCTGACAGTTACCAATGCTTAATCAGTGAGGCACCTATCTCAGCGAT |
| | | CTGTCTATTTCGTTCATCCATAGTTGCCTGACTCCCCGTCGTGTAGATAACT |
| | | ACGATACGGGAGGGCTTACCATCTGGCCCCAGTGCTGCAATGATACCGCGAG |
| | | AACCACGCTCACCGGCTCCAGATTTATCAGCAATAAACCAGCCAGCCGGAAG |
| | | GGCCGAGCGCAGAAGTGGTCCTGCAACTTTATCCGCCTCCATCCAGTCTATT |
| | | AATTGTTGCCGGGAAGCTAGAGTAAGTAGTTCGCCAGTTAATAGTTTGCGCA |
| | | ACGTTGTTGCCATTGCTACAGGCATCGTGGTGTCACGCTCGTCGTTTGGTAT |
| | | GGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAATTC |
| | | TCTTACTGTCATGCCATCCGTAAGATGCTTTTCTGTGACTGGTGAGTACTCA |
| | | ACCAAGTCATTCTGAGAATAGTGTATGCGGCGACCGAGTTGCTCTTGCCCGG |
| | | CGTCAATACGGGATAATACCGCGCCACATAGCAGAACTTTAAAAGTGCTCAT |
| | | CATTGGAAAACGTTCTTCGGGGCGAAAACTCTCAAGGATCTTACCGCTGTTG |
| | | AGATCCAGTTCGATGTAACCCACTCGTGCACCCAACTGATCTTCAGCATCTT |
| | | TTACTTTCACCAGCGTTTCTGGGTGAGCAAAAACAGGAAGGCAAAATGCCGC |
| | | AAAAAAGGGAATAAGGGCGACACGGAAATGTTGAATACTCATACTCTTCCTT |
| | | TTTCAATATTATTGAAGCATTTATCAGGGTTATTGTCTCATGAGCGGATACA |
| | | TATTTGAATGTATTTAGAAAAATAAACAAATAGGGGTTCCGCGCACATTTCC |
| | | CCGAAAAGTGCCAC |
| 19 | Compound 7 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATATAGTGAGTCGTATTAACGTACCAACAAGGCGTGGAGCTGAGAGATA |
| | | AACTTGTTATCTCTCAGCTCCACGCCTTTATCTTAGAGGCATATCCCTGCCA |
| | | CCATGGGCCTGACATCTCAGTTGCTGCCTCCACTGTTCTTTCTGCTGGCCTG |
| | | CGCCGGCAATTTTGTGCACGGCCACAAGTGCGACATCACCCTGCAAGAGATC |
| | | ATCAAGACCCTGAACAGCCTGACCGAGCAGAAAACCCTGTGCACCGAGCTGA |
| | | CCGTGACCGATATCTTTGCCGCCAGCAAGAACACAACCGAGAAAGAGACATT |
| | | CTGCAGAGCCGCCACCGTGCTGAGACAGTTCTACAGCCACCACGAGAAGGAC |
| | | ACCAGATGCCTGGGAGCTACAGCCCAGCAGTTCCACAGACACAAGCAGCTGA |
| | | TCCGGTTCCTGAAGCGGCTGGACAGAAATCTGTGGGGACTCGCCGGCCTGAA |
| | | TAGCTGCCCTGTGAAAGAGGCCAACCAGTCTACCCTGGAAAACTTCCTGGAA |
| | | CGGCTGAAAACCATCATGCGCGAGAAGTACAGCAAGTGCAGCAGCTAGTTTA |
| | | TCTTAGAGGCATATCCCTCTGGGCCTCATGGGCCTTCCGCTCACTGCCCGCT |
| | | TTCCAGTCGGGAAACCTGTCGTGCCAGCTGCATTAACATGGTCATAGCTGTT |
| | | TCCTTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAAAGG |
| | | CCAGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACCCG |
| | | ACAGGACTATAAAGATACCAGGCGTTTCCCCCTGGAAGCTCCCTCGTGCGCT |
| | | CTCCTGTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTTCGGTG |
| | | TAGGTCGTTCGCTCCAAGCTGGGCTGTGTGCACGAACCCCCCGTTCAGCCCG |
| | | ACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGG |
| | | TATGTAGGCGGTGCTACAGAGTTCTTGAAGTGGTGGCCTAACTACGGCTACA |
| | | CTAGAAGAACAGTATTTGGTATCTGCGCTCTGCTGAAGCCAGTTACCTTCGG |
| | | AAAAAGAGTTGGTAGCTCTTGATCCGGCAAACAAACCACCGCTGGTAGCGGT |
| | | GGTTTTTTTGTTTGCAAGCAGCAGATTACGCGCAGAAAAAAAGGATCTCAAG |
| | | AAGATCCTTTGATCTTTTCTACGGGGTCTGACGCTCAGTGGAACGAAAACTC |
| | | ACGTTAAGGGATTTTGGTCATGAGATTATCAAAAAGGATCTTCACCTAGATC |
| | | CTTTTAAATTAAAAATGAAGTTTTAAATCAATCTAAAGTATATATGAGTAAA |
| | | CTTGGTCTGACAGTTACCAATGCTTAATCAGTGAGGCACCTATCTCAGCGAT |
| | | CTGTCTATTTCGTTCATCCATAGTTGCCTGACTCCCCGTCGTGTAGATAACT |
| | | ACGATACGGGAGGGCTTACCATCTGGCCCCAGTGCTGCAATGATACCGCGAG |
| | | AACCACGCTCACCGGCTCCAGATTTATCAGCAATAAACCAGCCAGCCGGAAG |
| | | GGCCGAGCGCAGAAGTGGTCCTGCAACTTTATCCGCCTCCATCCAGTCTATT |
| | | AATTGTTGCCGGGAAGCTAGAGTAAGTAGTTCGCCAGTTAATAGTTTGCGCA |
| | | ACGTTGTTGCCATTGCTACAGGCATCGTGGTGTCACGCTCGTCGTTTGGTAT |
| | | GGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAATTC |
| | | TCTTACTGTCATGCCATCCGTAAGATGCTTTTCTGTGACTGGTGAGTACTCA |
| | | ACCAAGTCATTCTGAGAATAGTGTATGCGGCGACCGAGTTGCTCTTGCCCGG |
| | | CGTCAATACGGGATAATACCGCGCCACATAGCAGAACTTTAAAAGTGCTCAT |
| | | CATTGGAAAACGTTCTTCGGGGCGAAAACTCTCAAGGATCTTACCGCTGTTG |
| | | AGATCCAGTTCGATGTAACCCACTCGTGCACCCAACTGATCTTCAGCATCTT |
| | | TTACTTTCACCAGCGTTTCTGGGTGAGCAAAAACAGGAAGGCAAAATGCCGC |
| | | AAAAAAGGGAATAAGGGCGACACGGAAATGTTGAATACTCATACTCTTCCTT |
| | | TTTCAATATTATTGAAGCATTTATCAGGGTTATTGTCTCATGAGCGGATACA |
| | | TATTTGAATGTATTTAGAAAAATAAACAAATAGGGGTTCCGCGCACATTTCC |
| | | CCGAAAAGTGCCAC |
| 20 | Compound 8 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGACTGACATCTCAACTGCTGCCTCCACTGTTCTTTCT |
| | | GCTGGCCTGCGCCGGCAATTTTGTGCACGGCCACAAGTGCGACATCACCCTG |
| | | CAAGAGATCATCAAGACCCTGAACAGCCTGACCGAGCAGAAAACCCTGTGCA |
| | | CCGAGCTGACCGTGACCGATATCTTTGCCGCCAGCAAGAACACAACCGAGAA |
| | | AGAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACACA |
| | | AGCAGCTGATCCGGTTCCTGAAGCGGCTGGACAGAAATCTGTGGGGACTCGC |
| | | CGGCCTGAATAGCTGCCCTGTGAAAGAGGCCAACCAGTCTACCCTGGAAAAC |
| | | TTCCTGGAACGGCTGAAAACCATCATGCGCGAGAAGTACAGCAAGTGCAGCA |
| | | GCTGAATAGTGAGTCGTATTAACGTACCAACAAGGCGTGGAGCTGAGAGATA |
| | | AACTTGTTATCTCTCAGCTCCACGCCTTTATCTTAGAGGCATATCCCTTTTA |
| | | TCTTAGAGGCATATCCCTCTGGGCCTCATGGGCCTTCCGCTCACTGCCCGCT |
| | | TTCCAGTCGGGAAACCTGTCGTGCCAGCTGCATTAACATGGTCATAGCTGTT |
| | | TCCTTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAAAGG |
| | | CCAGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACCCG |
| | | ACAGGACTATAAAGATACCAGGCGTTTCCCCCTGGAAGCTCCCTCGTGCGCT |
| | | CTCCTGTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTTCGGTG |
| | | TAGGTCGTTCGCTCCAAGCTGGGCTGTGTGCACGAACCCCCCGTTCAGCCCG |
| | | ACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGG |
| | | TATGTAGGCGGTGCTACAGAGTTCTTGAAGTGGTGGCCTAACTACGGCTACA |
| | | CTAGAAGAACAGTATTTGGTATCTGCGCTCTGCTGAAGCCAGTTACCTTCGG |
| | | AAAAAGAGTTGGTAGCTCTTGATCCGGCAAACAAACCACCGCTGGTAGCGGT |
| | | GGTTTTTTTGTTTGCAAGCAGCAGATTACGCGCAGAAAAAAAGGATCTCAAG |
| | | AAGATCCTTTGATCTTTTCTACGGGGTCTGACGCTCAGTGGAACGAAAACTC |
| | | ACGTTAAGGGATTTTGGTCATGAGATTATCAAAAAGGATCTTCACCTAGATC |
| | | CTTTTAAATTAAAAATGAAGTTTTAAATCAATCTAAAGTATATATGAGTAAA |
| | | CTTGGTCTGACAGTTACCAATGCTTAATCAGTGAGGCACCTATCTCAGCGAT |
| | | CTGTCTATTTCGTTCATCCATAGTTGCCTGACTCCCCGTCGTGTAGATAACT |
| | | ACGATACGGGAGGGCTTACCATCTGGCCCCAGTGCTGCAATGATACCGCGAG |
| | | AACCACGCTCACCGGCTCCAGATTTATCAGCAATAAACCAGCCAGCCGGAAG |
| | | GGCCGAGCGCAGAAGTGGTCCTGCAACTTTATCCGCCTCCATCCAGTCTATT |
| | | AATTGTTGCCGGGAAGCTAGAGTAAGTAGTTCGCCAGTTAATAGTTTGCGCA |
| | | ACGTTGTTGCCATTGCTACAGGCATCGTGGTGTCACGCTCGTCGTTTGGTAT |
| | | GGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAATTC |
| | | TCTTACTGTCATGCCATCCGTAAGATGCTTTTCTGTGACTGGTGAGTACTCA |
| | | ACCAAGTCATTCTGAGAATAGTGTATGCGGCGACCGAGTTGCTCTTGCCCGG |
| | | CGTCAATACGGGATAATACCGCGCCACATAGCAGAACTTTAAAAGTGCTCAT |
| | | CATTGGAAAACGTTCTTCGGGGCGAAAACTCTCAAGGATCTTACCGCTGTTG |
| | | AGATCCAGTTCGATGTAACCCACTCGTGCACCCAACTGATCTTCAGCATCTT |
| | | TTACTTTCACCAGCGTTTCTGGGTGAGCAAAAACAGGAAGGCAAAATGCCGC |
| | | AAAAAAGGGAATAAGGGCGACACGGAAATGTTGAATACTCATACTCTTCCTT |
| | | TTTCAATATTATTGAAGCATTTATCAGGGTTATTGTCTCATGAGCGGATACA |
| | | TATTTGAATGTATTTAGAAAAATAAACAAATAGGGGTTCCGCGCACATTTCC |
| | | CCGAAAAGTGCCAC |
| 21 | Compound 9 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGACTGACATCTCAACTGCTGCCTCCACTGTTCTTTCT |
| | | GCTGGCCTGCGCCGGCAATTTTGTGCACGGCCACAAGTGCGACATCACCCTG |
| | | CAAGAGATCATCAAGACCCTGAACAGCCTGACCGAGCAGAAAACCCTGTGCA |
| | | CCGAGCTGACCGTGACCGATATCTTTGCCGCCAGCAAGAACACAACCGAGAA |
| | | AGAGACATTCTGCAGAGCCGCCACCGTGCTGAGACAGTTCTACAGCCACCAC |
| | | GAGAAGGACACCAGATGCCTGGGAGCTACAGCCCAGCAGTTCCACAGACACA |
| | | AGCAGCTGATCCGGTTCCTGAAGCGGCTGGACAGAAATCTGTGGGGACTCGC |
| | | CGGCCTGAATAGCTGCCCTGTGAAAGAGGCCAACCAGTCTACCCTGGAAAAC |
| | | TTCCTGGAACGGCTGAAAACCATCATGCGCGAGAAGTACAGCAAGTGCAGCA |
| | | GCTGAACAACAAGGCGTGGAGCTGAGAGATAAACTTGTTATCTCTCAGCTCC |
| | | ACGCCACAACAAGGGCCTGTACCTCATCTACTACTTGAGTAGATGAGGTACA |
| | | GGCCCACAACAAGGTATGAGCCCATCTATCTACTTGAGATAGATGGGCTCAT |
| | | ACCACAACAATTTATCTTAGAGGCATATCCCTCTGGGCCTCATGGGCCTTCC |
| | | GCTCACTGCCCGCTTTCCAGTCGGGAAACCTGTCGTGCCAGCTGCATTAACA |
| | | TGGTCATAGCTGTTTCCTTGCGTATTGGGCGCTCTCCGCTTCCTCGCTCACT |
| | | GACTCGCTGCGCTCGGTCGTTCGGGTAAAGCCTGGGGTGCCTAATGAGCAAA |
| | | AGGCCAGCAAAAGGCCAGGAACCGTAAAAAGGCCGCGTTGCTGGCGTTTTTC |
| | | CATAGGCTCCGCCCCCCTGACGAGCATCACAAAAATCGACGCTCAAGTCAGA |
| | | GGTGGCGAAACCCGACAGGACTATAAAGATACCAGGCGTTTCCCCCTGGAAG |
| | | CTCCCTCGTGCGCTCTCCTGTTCCGACCCTGCCGCTTACCGGATACCTGTCC |
| | | GCCTTTCTCCCTTCGGGAAGCGTGGCGCTTTCTCATAGCTCACGCTGTAGGT |
| | | ATCTCAGTTCGGTGTAGGTCGTTCGCTCCAAGCTGGGCTGTGTGCACGAACC |
| | | CCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGA |
| | | TTAGCAGAGCGAGGTATGTAGGCGGTGCTACAGAGTTCTTGAAGTGGTGGCC |
| | | TAACTACGGCTACACTAGAAGAACAGTATTTGGTATCTGCGCTCTGCTGAAG |
| | | CCAGTTACCTTCGGAAAAAGAGTTGGTAGCTCTTGATCCGGCAAACAAACCA |
| | | CCGCTGGTAGCGGTGGTTTTTTTGTTTGCAAGCAGCAGATTACGCGCAGAAA |
| | | AAAAGGATCTCAAGAAGATCCTTTGATCTTTTCTACGGGGTCTGACGCTCAG |
| | | TGGAACGAAAACTCACGTTAAGGGATTTTGGTCATGAGATTATCAAAAAGGA |
| | | TCTTCACCTAGATCCTTTTAAATTAAAAATGAAGTTTTAAATCAATCTAAAG |
| | | TATATATGAGTAAACTTGGTCTGACAGTTACCAATGCTTAATCAGTGAGGCA |
| | | CCTATCTCAGCGATCTGTCTATTTCGTTCATCCATAGTTGCCTGACTCCCCG |
| | | TCGTGTAGATAACTACGATACGGGAGGGCTTACCATCTGGCCCCAGTGCTGC |
| | | AATGATACCGCGAGAACCACGCTCACCGGCTCCAGATTTATCAGCAATAAAC |
| | | CAGCCAGCCGGAAGGGCCGAGCGCAGAAGTGGTCCTGCAACTTTATCCGCCT |
| | | CCATCCAGTCTATTAATTGTTGCCGGGAAGCTAGAGTAAGTAGTTCGCCAGT |
| | | TAATAGTTTGCGCAACGTTGTTGCCATTGCTACAGGCATCGTGGTGTCACGC |
| | | TCGTCGTTTGGTATGGCTTCATTCAGCTCCGGTTCCCAACGATCAAGGCGAG |
| | | TTACATGATCCCCCATGTTGTGCAAAAAAGCGGTTAGCTCCTTCGGTCCTCC |
| | | GATCGTTGTCAGAAGTAAGTTGGCCGCAGTGTTATCACTCATGGTTATGGCA |
| | | GCACTGCATAATTCTCTTACTGTCATGCCATCCGTAAGATGCTTTTCTGTGA |
| | | CTGGTGAGTACTCAACCAAGTCATTCTGAGAATAGTGTATGCGGCGACCGAG |
| | | TTGCTCTTGCCCGGCGTCAATACGGGATAATACCGCGCCACATAGCAGAACT |
| | | TTAAAAGTGCTCATCATTGGAAAACGTTCTTCGGGGCGAAAACTCTCAAGGA |
| | | TCTTACCGCTGTTGAGATCCAGTTCGATGTAACCCACTCGTGCACCCAACTG |
| | | ATCTTCAGCATCTTTTACTTTCACCAGCGTTTCTGGGTGAGCAAAAACAGGA |
| | | AGGCAAAATGCCGCAAAAAAGGGAATAAGGGCGACACGGAAATGTTGAATAC |
| | | TCATACTCTTCCTTTTTCAATATTATTGAAGCATTTATCAGGGTTATTGTCT |
| | | CATGAGCGGATACATATTTGAATGTATTTAGAAAAATAAACAAATAGGGGTT |
| | | CCGCGCACATTTCCCCGAAAAGTGCCAC |
| 22 | Compound 10 | CTTCGCTATTACGCCAGCTGGCGAAAGGGGGATGTGCTGCAAGGCGATTAAG |
| | in pMA-RQ | TTGGGTAACGCCAGGGTTTTCCCAGTCACGACGTTGTAAAACGACGGCCAGT |
| | | GAGCGCGACGTAATACGACTCACTATAGGGCGAATTGGCGGAAGGCCGTCAA |
| | | GGCCGCATGCCACCATGGGCAAGATTAGCAGCCTGCCTACACAGCTGTTCAA |
| | | GTGCTGCTTCTGCGACTTCCTGAAAGTGAAGATGCACACCATGAGCAGCAGC |
| | | CACCTGTTCTATCTGGCCCTGTGCCTGCTGACCTTTACCAGCTCTGCTACCG |
| | | CCGGACCTGAGACACTTTGTGGCGCTGAACTGGTGGACGCCCTGCAGTTTGT |
| | | GTGTGGCGACAGAGGCTTCTACTTCAACAAGCCCACAGGCTACGGCAGCAGC |
| | | TCTAGAAGGGCTCCTCAGACCGGAATCGTGGACGAGTGCTGTTTCAGAAGCT |
| | | GCGACCTGCGGCGGCTGGAAATGTATTGTGCCCCTCTGAAGCCTGCCAAGAG |
| | | CGCCTAAATAGTGAGTCGTATTAACGTACCAACAACAACAAGATGAAGAGCA |
| | | CCAAACTTGTTGGTGCTCTTCATCTTGTTGTTTATCTTAGAGGCATATCCCT |
| | | TTTATCTTAGAGGCATATCCCTCTGGGCCTCATGGGCCTTCCGCTCACTGCC |
| | | CGCTTTCCAGTCGGGAAACCTGTCGTGCCAGCTGCATTAACATGGTCATAGC |
| | | TGTTTCCTTGCGTATTGGGCGCTCTCCGCTTCCTCGCTCACTGACTCGCTGC |
| | | GCTCGGTCGTTCGGGTAAAGCCTGGGGTGCCTAATGAGCAAAAGGCCAGCAA |
| | | AAGGCCAGGAACCGTAAAAAGGCCGCGTTGCTGGCGTTTTTCCATAGGCTCC |
| | | GCCCCCCTGACGAGCATCACAAAAATCGACGCTCAAGTCAGAGGTGGCGAAA |
| | | CCCGACAGGACTATAAAGATACCAGGCGTTTCCCCCTGGAAGCTCCCTCGTG |
| | | CGCTCTCCTGTTCCGACCCTGCCGCTTACCGGATACCTGTCCGCCTTTCTCC |
| | | CTTCGGGAAGCGTGGCGCTTTCTCATAGCTCACGCTGTAGGTATCTCAGTTC |
| | | GGTGTAGGTCGTTCGCTCCAAGCTGGGCTGTGTGCACGAACCCCCCGTTCAG |
| | | CCCGACCGCTGCGCCTTATCCGGTAACTATCGTCTTGAGTCCAACCCGGTAA |
| | | GACACGACTTATCGCCACTGGCAGCAGCCACTGGTAACAGGATTAGCAGAGC |
| | | GAGGTATGTAGGCGGTGCTACAGAGTTCTTGAAGTGGTGGCCTAACTACGGC |
| | | TACACTAGAAGAACAGTATTTGGTATCTGCGCTCTGCTGAAGCCAGTTACCT |
| | | TCGGAAAAAGAGTTGGTAGCTCTTGATCCGGCAAACAAACCACCGCTGGTAG |
| | | CGGTGGTTTTTTTGTTTGCAAGCAGCAGATTACGCGCAGAAAAAAAGGATCT |
| | | CAAGAAGATCCTTTGATCTTTTCTACGGGGTCTGACGCTCAGTGGAACGAAA |
| | | ACTCACGTTAAGGGATTTTGGTCATGAGATTATCAAAAAGGATCTTCACCTA |
| | | GATCCTTTTAAATTAAAAATGAAGTTTTAAATCAATCTAAAGTATATATGAG |
| | | TAAACTTGGTCTGACAGTTACCAATGCTTAATCAGTGAGGCACCTATCTCAG |
| | | CGATCTGTCTATTTCGTTCATCCATAGTTGCCTGACTCCCCGTCGTGTAGAT |
| | | AACTACGATACGGGAGGGCTTACCATCTGGCCCCAGTGCTGCAATGATACCG |
| | | CGAGAACCACGCTCACCGGCTCCAGATTTATCAGCAATAAACCAGCCAGCCG |
| | | GAAGGGCCGAGCGCAGAAGTGGTCCTGCAACTTTATCCGCCTCCATCCAGTC |
| | | TATTAATTGTTGCCGGGAAGCTAGAGTAAGTAGTTCGCCAGTTAATAGTTTG |
| | | CGCAACGTTGTTGCCATTGCTACAGGCATCGTGGTGTCACGCTCGTCGTTTG |
| | | GTATGGCTTCATTCAGCTCCGGTTCCCAACGATCAAGGCGAGTTACATGATC |
| | | CCCCATGTTGTGCAAAAAAGCGGTTAGCTCCTTCGGTCCTCCGATCGTTGTC |
| | | AGAAGTAAGTTGGCCGCAGTGTTATCACTCATGGTTATGGCAGCACTGCATA |
| | | ATTCTCTTACTGTCATGCCATCCGTAAGATGCTTTTCTGTGACTGGTGAGTA |
| | | CTCAACCAAGTCATTCTGAGAATAGTGTATGCGGCGACCGAGTTGCTCTTGC |
| | | CCGGCGTCAATACGGGATAATACCGCGCCACATAGCAGAACTTTAAAAGTGC |
| | | TCATCATTGGAAAACGTTCTTCGGGGCGAAAACTCTCAAGGATCTTACCGCT |
| | | GTTGAGATCCAGTTCGATGTAACCCACTCGTGCACCCAACTGATCTTCAGCA |
| | | TCTTTTACTTTCACCAGCGTTTCTGGGTGAGCAAAAACAGGAAGGCAAAATG |
| | | CCGCAAAAAAGGGAATAAGGGCGACACGGAAATGTTGAATACTCATACTCTT |
| | | CCTTTTTCAATATTATTGAAGCATTTATCAGGGTTATTGTCTCATGAGCGGA |
| | | TACATATTTGAATGTATTTAGAAAAATAAACAAATAGGGGTTCCGCGCACAT |
| | | TTCCCCGAAAAGTGCCAC |
| 23 | Compound 11 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGCAAGATTAGCAGCCTGCCTACACAGCTGTTCAAGTG |
| | | CTGCTTCTGCGACTTCCTGAAAGTGAAGATGCACACCATGAGCAGCAGCCAC |
| | | CTGTTCTATCTGGCCCTGTGCCTGCTGACCTTTACCAGCTCTGCTACCGCCG |
| | | GACCTGAGACACTTTGTGGCGCTGAACTGGTGGACGCCCTGCAGTTTGTGTG |
| | | TGGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGCG |
| | | ACCTGCGGCGGCTGGAAATGTATTGTGCCCCTCTGAAGCCTGCCAAGAGCGC |
| | | CTAAATAGTGAGTCGTATTAACGTACCAACAACAACAAGATGAAGAGCACCA |
| | | AACTTGTTGGTGCTCTTCATCTTGTTGTTTATCTTAGAGGCATATCCCTACG |
| | | TACCAACAACAACAAGATGAAGAGCACCAAACTTGTTGGTGCTCTTCATCTT |
| | | GTTGTTTATCTTAGAGGCATATCCCTTTTATCTTAGAGGCATATCCCTCTGG |
| | | GCCTCATGGGCCTTCCGCTCACTGCCCGCTTTCCAGTCGGGAAACCTGTCGT |
| | | GCCAGCTGCATTAACATGGTCATAGCTGTTTCCTTGCGTATTGGGCGCTCTC |
| | | CGCTTCCTCGCTCACTGACTCGCTGCGCTCGGTCGTTCGGGTAAAGCCTGGG |
| | | GTGCCTAATGAGCAAAAGGCCAGCAAAAGGCCAGGAACCGTAAAAAGGCCGC |
| | | GTTGCTGGCGTTTTTCCATAGGCTCCGCCCCCCTGACGAGCATCACAAAAAT |
| | | CGACGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCCGCT |
| | | TACCGGATACCTGTCCGCCTTTCTCCCTTCGGGAAGCGTGGCGCTTTCTCAT |
| | | AGCTCACGCTGTAGGTATCTCAGTTCGGTGTAGGTCGTTCGCTCCAAGCTGG |
| | | GCTGTGTGCACGAACCCCCCGTTCAGCCCGACCGCTGCGCCTTATCCGGTAA |
| | | CTATCGTCTTGAGTCCAACCCGGTAAGACACGACTTATCGCCACTGGCAGCA |
| | | GCCACTGGTAACAGGATTAGCAGAGCGAGGTATGTAGGCGGTGCTACAGAGT |
| | | TCTTGAAGTGGTGGCCTAACTACGGCTACACTAGAAGAACAGTATTTGGTAT |
| | | CTGCGCTCTGCTGAAGCCAGTTACCTTCGGAAAAAGAGTTGGTAGCTCTTGA |
| | | TCCGGCAAACAAACCACCGCTGGTAGCGGTGGTTTTTTTGTTTGCAAGCAGC |
| | | AGATTACGCGCAGAAAAAAAGGATCTCAAGAAGATCCTTTGATCTTTTCTAC |
| | | GGGGTCTGACGCTCAGTGGAACGAAAACTCACGTTAAGGGATTTTGGTCATG |
| | | AGATTATCAAAAAGGATCTTCACCTAGATCCTTTTAAATTAAAAATGAAGTT |
| | | TTAAATCAATCTAAAGTATATATGAGTAAACTTGGTCTGACAGTTACCAATG |
| | | CTTAATCAGTGAGGCACCTATCTCAGCGATCTGTCTATTTCGTTCATCCATA |
| | | GTTGCCTGACTCCCCGTCGTGTAGATAACTACGATACGGGAGGGCTTACCAT |
| | | CTGGCCCCAGTGCTGCAATGATACCGCGAGAACCACGCTCACCGGCTCCAGA |
| | | TTTATCAGCAATAAACCAGCCAGCCGGAAGGGCCGAGCGCAGAAGTGGTCCT |
| | | GCAACTTTATCCGCCTCCATCCAGTCTATTAATTGTTGCCGGGAAGCTAGAG |
| | | TAAGTAGTTCGCCAGTTAATAGTTTGCGCAACGTTGTTGCCATTGCTACAGG |
| | | CATCGTGGTGTCACGCTCGTCGTTTGGTATGGCTTCATTCAGCTCCGGTTCC |
| | | CAACGATCAAGGCGAGTTACATGATCCCCCATGTTGTGCAAAAAAGCGGTTA |
| | | GCTCCTTCGGTCCTCCGATCGTTGTCAGAAGTAAGTTGGCCGCAGTGTTATC |
| | | ACTCATGGTTATGGCAGCACTGCATAATTCTCTTACTGTCATGCCATCCGTA |
| | | AGATGCTTTTCTGTGACTGGTGAGTACTCAACCAAGTCATTCTGAGAATAGT |
| | | GTATGCGGCGACCGAGTTGCTCTTGCCCGGCGTCAATACGGGATAATACCGC |
| | | GCCACATAGCAGAACTTTAAAAGTGCTCATCATTGGAAAACGTTCTTCGGGG |
| | | CGAAAACTCTCAAGGATCTTACCGCTGTTGAGATCCAGTTCGATGTAACCCA |
| | | CTCGTGCACCCAACTGATCTTCAGCATCTTTTACTTTCACCAGCGTTTCTGG |
| | | GTGAGCAAAAACAGGAAGGCAAAATGCCGCAAAAAAGGGAATAAGGGCGACA |
| | | CGGAAATGTTGAATACTCATACTCTTCCTTTTTCAATATTATTGAAGCATTT |
| | | ATCAGGGTTATTGTCTCATGAGCGGATACATATTTGAATGTATTTAGAAAAA |
| | | TAAACAAATAGGGGTTCCGCGCACATTTCCCCGAAAAGTGCCAC |
| 24 | Compound 12 | CTAAATTGTAAGCGTTAATATTTTGTTAAAATTCGCGTTAAATTTTTGTTAA |
| | in pMA-RQ | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CGCATGCCACCATGGGCAAGATTAGCAGCCTGCCTACACAGCTGTTCAAGTG |
| | | CTGCTTCTGCGACTTCCTGAAAGTGAAGATGCACACCATGAGCAGCAGCCAC |
| | | CTGTTCTATCTGGCCCTGTGCCTGCTGACCTTTACCAGCTCTGCTACCGCCG |
| | | GACCTGAGACACTTTGTGGCGCTGAACTGGTGGACGCCCTGCAGTTTGTGTG |
| | | TGGCGACAGAGGCTTCTACTTCAACAAGCCCACAGGCTACGGCAGCAGCTCT |
| | | AGAAGGGCTCCTCAGACCGGAATCGTGGACGAGTGCTGTTTCAGAAGCTGCG |
| | | ACCTGCGGCGGCTGGAAATGTATTGTGCCCCTCTGAAGCCTGCCAAGAGCGC |
| | | CTAAATAGTGAGTCGTATTAACGTACCAACAACAACAAGATGAAGAGCACCA |
| | | AACTTGTTGGTGCTCTTCATCTTGTTGTTTATCTTAGAGGCATATCCCTACG |
| | | TACCAACAACAACAAGATGAAGAGCACCAAACTTGTTGGTGCTCTTCATCTT |
| | | GTTGTTTATCTTAGAGGCATATCCCTACGTACCAACAACAACAAGATGAAGA |
| | | GCACCAAACTTGTTGGTGCTCTTCATCTTGTTGTTTATCTTAGAGGCATATC |
| | | CCTTTTATCTTAGAGGCATATCCCTCTGGGCCTCATGGGCCTTCCGCTCACT |
| | | GCCCGCTTTCCAGTCGGGAAACCTGTCGTGCCAGCTGCATTAACATGGTCAT |
| | | AGCTGTTTCCTTGCGTATTGGGCGCTCTCCGCTTCCTCGCTCACTGACTCGC |
| | | TGCGCTCGGTCGTTCGGGTAAAGCCTGGGGTGCCTAATGAGCAAAAGGCCAG |
| | | CAAAAGGCCAGGAACCGTAAAAAGGCCGCGTTGCTGGCGTTTTTCCATAGGC |
| | | TCCGCCCCCCTGACGAGCATCACAAAAATCGACGCTCAAGTCAGAGGTGGCG |
| | | AAACCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCTTTC |
| | | TCCCTTCGGGAAGCGTGGCGCTTTCTCATAGCTCACGCTGTAGGTATCTCAG |
| | | TTCGGTGTAGGTCGTTCGCTCCAAGCTGGGCTGTGTGCACGAACCCCCCGTT |
| | | CAGCCCGACCGCTGCGCCTTATCCGGTAACTATCGTCTTGAGTCCAACCCGG |
| | | TAAGACACGACTTATCGCCACTGGCAGCAGCCACTGGTAACAGGATTAGCAG |
| | | AGCGAGGTATGTAGGCGGTGCTACAGAGTTCTTGAAGTGGTGGCCTAACTAC |
| | | GGCTACACTAGAAGAACAGTATTTGGTATCTGCGCTCTGCTGAAGCCAGTTA |
| | | CCTTCGGAAAAAGAGTTGGTAGCTCTTGATCCGGCAAACAAACCACCGCTGG |
| | | TAGCGGTGGTTTTTTTGTTTGCAAGCAGCAGATTACGCGCAGAAAAAAAGGA |
| | | TCTCAAGAAGATCCTTTGATCTTTTCTACGGGGTCTGACGCTCAGTGGAACG |
| | | AAAACTCACGTTAAGGGATTTTGGTCATGAGATTATCAAAAAGGATCTTCAC |
| | | CTAGATCCTTTTAAATTAAAAATGAAGTTTTAAATCAATCTAAAGTATATAT |
| | | GAGTAAACTTGGTCTGACAGTTACCAATGCTTAATCAGTGAGGCACCTATCT |
| | | CAGCGATCTGTCTATTTCGTTCATCCATAGTTGCCTGACTCCCCGTCGTGTA |
| | | GATAACTACGATACGGGAGGGCTTACCATCTGGCCCCAGTGCTGCAATGATA |
| | | CCGCGAGAACCACGCTCACCGGCTCCAGATTTATCAGCAATAAACCAGCCAG |
| | | CCGGAAGGGCCGAGCGCAGAAGTGGTCCTGCAACTTTATCCGCCTCCATCCA |
| | | GTCTATTAATTGTTGCCGGGAAGCTAGAGTAAGTAGTTCGCCAGTTAATAGT |
| | | TTGCGCAACGTTGTTGCCATTGCTACAGGCATCGTGGTGTCACGCTCGTCGT |
| | | TTGGTATGGCTTCATTCAGCTCCGGTTCCCAACGATCAAGGCGAGTTACATG |
| | | ATCCCCCATGTTGTGCAAAAAAGCGGTTAGCTCCTTCGGTCCTCCGATCGTT |
| | | GTCAGAAGTAAGTTGGCCGCAGTGTTATCACTCATGGTTATGGCAGCACTGC |
| | | ATAATTCTCTTACTGTCATGCCATCCGTAAGATGCTTTTCTGTGACTGGTGA |
| | | GTACTCAACCAAGTCATTCTGAGAATAGTGTATGCGGCGACCGAGTTGCTCT |
| | | TGCCCGGCGTCAATACGGGATAATACCGCGCCACATAGCAGAACTTTAAAAG |
| | | TGCTCATCATTGGAAAACGTTCTTCGGGGCGAAAACTCTCAAGGATCTTACC |
| | | GCTGTTGAGATCCAGTTCGATGTAACCCACTCGTGCACCCAACTGATCTTCA |
| | | GCATCTTTTACTTTCACCAGCGTTTCTGGGTGAGCAAAAACAGGAAGGCAAA |
| | | ATGCCGCAAAAAAGGGAATAAGGGCGACACGGAAATGTTGAATACTCATACT |
| | | CTTCCTTTTTCAATATTATTGAAGCATTTATCAGGGTTATTGTCTCATGAGC |
| | | GGATACATATTTGAATGTATTTAGAAAAATAAACAAATAGGGGTTCCGCGCA |
| | | CATTTCCCCGAAAAGTGCCAC |
| 127 | Compound 13 | CTAAATTGTAAGCGTTAATATTTTGTTAAAATTCGCGTTAAATTTTTGTTAA |
| | in pMA-T | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CACGTGTCTTGTCCAGAGCTCGGATCCGCCACCATGGGCAAGATTAGCAGCC |
| | | TGCCTACACAGCTGTTCAAGTGCTGCTTCTGCGACTTCCTGAAAGTGAAGAT |
| | | GCACACCATGAGCAGCAGCCACCTGTTCTATCTGGCCCTGTGCCTGCTGACC |
| | | TTTACCAGCTCTGCTACCGCCGGACCTGAGACACTTTGTGGCGCTGAACTGG |
| | | TGGACGCCCTGCAGTTTGTGTGTGGCGACAGAGGCTTCTACTTCAACAAGCC |
| | | CACAGGCTACGGCAGCAGCTCTAGAAGGGCTCCTCAGACCGGAATCGTGGAC |
| | | GAGTGCTGTTTCAGAAGCTGCGACCTGCGGCGGCTGGAAATGTATTGTGCCC |
| | | CTCTGAAGCCTGCCAAGAGCGCCTAAGAATTCGGTACCTGGAGCACAAGACT |
| | | GGCCTCATGGGCCTTCCGCTCACTGCCCGCTTTCCAGTCGGGAAACCTGTCG |
| | | TGCCAGCTGCATTAACATGGTCATAGCTGTTTCCTTGCGTATTGGGCGCTCT |
| | | CCGCTTCCTCGCTCACTGACTCGCTGCGCTCGGTCGTTCGGGTAAAGCCTGG |
| | | GGTGCCTAATGAGCAAAAGGCCAGCAAAAGGCCAGGAACCGTAAAAAGGCCG |
| | | CGTTGCTGGCGTTTTTCCATAGGCTCCGCCCCCCTGACGAGCATCACAAAAA |
| | | TCGACGCTCAAGTCAGAGGTGGCGAAACCCGACAGGACTATAAAGATACCAG |
| | | GCGTTTCCCCCTGGAAGCTCCCTCGTGCGCTCTCCTGTTCCGACCCTGCCGC |
| | | TTACCGGATACCTGTCCGCCTTTCTCCCTTCGGGAAGCGTGGCGCTTTCTCA |
| | | TAGCTCACGCTGTAGGTATCTCAGTTCGGTGTAGGTCGTTCGCTCCAAGCTG |
| | | GGCTGTGTGCACGAACCCCCCGTTCAGCCCGACCGCTGCGCCTTATCCGGTA |
| | | ACTATCGTCTTGAGTCCAACCCGGTAAGACACGACTTATCGCCACTGGCAGC |
| | | AGCCACTGGTAACAGGATTAGCAGAGCGAGGTATGTAGGCGGTGCTACAGAG |
| | | TTCTTGAAGTGGTGGCCTAACTACGGCTACACTAGAAGAACAGTATTTGGTA |
| | | TCTGCGCTCTGCTGAAGCCAGTTACCTTCGGAAAAAGAGTTGGTAGCTCTTG |
| | | ATCCGGCAAACAAACCACCGCTGGTAGCGGTGGTTTTTTTGTTTGCAAGCAG |
| | | CAGATTACGCGCAGAAAAAAAGGATCTCAAGAAGATCCTTTGATCTTTTCTA |
| | | CGGGGTCTGACGCTCAGTGGAACGAAAACTCACGTTAAGGGATTTTGGTCAT |
| | | GAGATTATCAAAAAGGATCTTCACCTAGATCCTTTTAAATTAAAAATGAAGT |
| | | TTTAAATCAATCTAAAGTATATATGAGTAAACTTGGTCTGACAGTTACCAAT |
| | | GCTTAATCAGTGAGGCACCTATCTCAGCGATCTGTCTATTTCGTTCATCCAT |
| | | AGTTGCCTGACTCCCCGTCGTGTAGATAACTACGATACGGGAGGGCTTACCA |
| | | TCTGGCCCCAGTGCTGCAATGATACCGCGAGAACCACGCTCACCGGCTCCAG |
| | | ATTTATCAGCAATAAACCAGCCAGCCGGAAGGGCCGAGCGCAGAAGTGGTCC |
| | | TGCAACTTTATCCGCCTCCATCCAGTCTATTAATTGTTGCCGGGAAGCTAGA |
| | | GTAAGTAGTTCGCCAGTTAATAGTTTGCGCAACGTTGTTGCCATTGCTACAG |
| | | GCATCGTGGTGTCACGCTCGTCGTTTGGTATGGCTTCATTCAGCTCCGGTTC |
| | | CCAACGATCAAGGCGAGTTACATGATCCCCCATGTTGTGCAAAAAAGCGGTT |
| | | AGCTCCTTCGGTCCTCCGATCGTTGTCAGAAGTAAGTTGGCCGCAGTGTTAT |
| | | CACTCATGGTTATGGCAGCACTGCATAATTCTCTTACTGTCATGCCATCCGT |
| | | AAGATGCTTTTCTGTGACTGGTGAGTACTCAACCAAGTCATTCTGAGAATAG |
| | | TGTATGCGGCGACCGAGTTGCTCTTGCCCGGCGTCAATACGGGATAATACCG |
| | | CGCCACATAGCAGAACTTTAAAAGTGCTCATCATTGGAAAACGTTCTTCGGG |
| | | GCGAAAACTCTCAAGGATCTTACCGCTGTTGAGATCCAGTTCGATGTAACCC |
| | | ACTCGTGCACCCAACTGATCTTCAGCATCTTTTACTTTCACCAGCGTTTCTG |
| | | GGTGAGCAAAAACAGGAAGGCAAAATGCCGCAAAAAAGGGAATAAGGGCGAC |
| | | ACGGAAATGTTGAATACTCATACTCTTCCTTTTTCAATATTATTGAAGCATT |
| | | TATCAGGGTTATTGTCTCATGAGCGGATACATATTTGAATGTATTTAGAAAA |
| | | ATAAACAAATAGGGGTTCCGCGCACATTTCCCCGAAAAGTGCCAC |
| 128 | Compound 14 | CTAAATTGTAAGCGTTAATATTTTGTTAAAATTCGCGTTAAATTTTTGTTAA |
| | in pMA-T | ATCAGCTCATTTTTTAACCAATAGGCCGAAATCGGCAAAATCCCTTATAAAT |
| | | CAAAAGAATAGACCGAGATAGGGTTGAGTGGCCGCTACAGGGCGCTCCCATT |
| | | CGCCATTCAGGCTGCGCAACTGTTGGGAAGGGCGTTTCGGTGCGGGCCTCTT |
| | | CGCTATTACGCCAGCTGGCGAAAGGGGGATGTGCTGCAAGGCGATTAAGTTG |
| | | GGTAACGCCAGGGTTTTCCCAGTCACGACGTTGTAAAACGACGGCCAGTGAG |
| | | CGCGACGTAATACGACTCACTATAGGGCGAATTGGCGGAAGGCCGTCAAGGC |
| | | CACGTGTCTTGTCCAGAGCTCGCCACCATGTTGCTGCTGCCTCTGTTCTTCC |
| | | TGCTGGCCTGCGCCGGCAATTTTGTGCACGGCCACAAGTGCGACATCACCCT |
| | | GCAAGAGATCATCAAGACCCTGAACAGCCTGACCGAGCAGAAAACCCTGTGC |
| | | ACCGAGCTGACCGTGACCGATATCTTTGCCGCCAGCAAGAACACAACCGAGA |
| | | AAGAGACATTCTGCAGAGCCGCCACCGTGCTGAGACAGTTCTACAGCCACCA |
| | | CGAGAAGGACACCAGATGCCTGGGAGCTACAGCCCAGCAGTTCCACAGACAC |
| | | AAGCAGCTGATCCGGTTCCTGAAGCGGCTGGACAGAAATCTGTGGGGACTCG |
| | | CCGGCCTGAATAGCTGCCCTGTGAAAGAGGCCAACCAGTCTACCCTGGAAAA |
| | | CTTCCTGGAACGGCTGAAAACCATCATGCGCGAGAAGTACAGCAAGTGCAGC |
| | | AGCTGAGGTACCTGGAGCACAAGACTGGCCTCATGGGCCTTCCGCTCACTGC |
| | | CCGCTTTCCAGTCGGGAAACCTGTCGTGCCAGCTGCATTAACATGGTCATAG |
| | | CTGTTTCCTTGCGTATTGGGCGCTCTCCGCTTCCTCGCTCACTGACTCGCTG |
| | | CGCTCGGTCGTTCGGGTAAAGCCTGGGGTGCCTAATGAGCAAAAGGCCAGCA |
| | | AAAGGCCAGGAACCGTAAAAAGGCCGCGTTGCTGGCGTTTTTCCATAGGCTC |
| | | CGCCCCCCTGACGAGCATCACAAAAATCGACGCTCAAGTCAGAGGTGGCGAA |
| | | ACCCGACAGGACTATAAAGATACCAGGCGTTTCCCCCTGGAAGCTCCCTCGT |
| | | GCGCTCTCCTGTTCCGACCCTGCCGCTTACCGGATACCTGTCCGCCTTTCTC |
| | | CCTTCGGGAAGCGTGGCGCTTTCTCATAGCTCACGCTGTAGGTATCTCAGTT |
| | | CGGTGTAGGTCGTTCGCTCCAAGCTGGGCTGTGTGCACGAACCCCCCGTTCA |
| | | GCCCGACCGCTGCGCCTTATCCGGTAACTATCGTCTTGAGTCCAACCCGGTA |
| | | AGACACGACTTATCGCCACTGGCAGCAGCCACTGGTAACAGGATTAGCAGAG |
| | | CGAGGTATGTAGGCGGTGCTACAGAGTTCTTGAAGTGGTGGCCTAACTACGG |
| | | CTACACTAGAAGAACAGTATTTGGTATCTGCGCTCTGCTGAAGCCAGTTACC |
| | | TTCGGAAAAAGAGTTGGTAGCTCTTGATCCGGCAAACAAACCACCGCTGGTA |
| | | GCGGTGGTTTTTTTGTTTGCAAGCAGCAGATTACGCGCAGAAAAAAAGGATC |
| | | TCAAGAAGATCCTTTGATCTTTTCTACGGGGTCTGACGCTCAGTGGAACGAA |
| | | AACTCACGTTAAGGGATTTTGGTCATGAGATTATCAAAAAGGATCTTCACCT |
| | | AGATCCTTTTAAATTAAAAATGAAGTTTTAAATCAATCTAAAGTATATATGA |
| | | GTAAACTTGGTCTGACAGTTACCAATGCTTAATCAGTGAGGCACCTATCTCA |
| | | GCGATCTGTCTATTTCGTTCATCCATAGTTGCCTGACTCCCCGTCGTGTAGA |
| | | TAACTACGATACGGGAGGGCTTACCATCTGGCCCCAGTGCTGCAATGATACC |
| | | GCGAGAACCACGCTCACCGGCTCCAGATTTATCAGCAATAAACCAGCCAGCC |
| | | GGAAGGGCCGAGCGCAGAAGTGGTCCTGCAACTTTATCCGCCTCCATCCAGT |
| | | CTATTAATTGTTGCCGGGAAGCTAGAGTAAGTAGTTCGCCAGTTAATAGTTT |
| | | GCGCAACGTTGTTGCCATTGCTACAGGCATCGTGGTGTCACGCTCGTCGTTT |
| | | GGTATGGCTTCATTCAGCTCCGGTTCCCAACGATCAAGGCGAGTTACATGAT |
| | | CCCCCATGTTGTGCAAAAAAGCGGTTAGCTCCTTCGGTCCTCCGATCGTTGT |
| | | CAGAAGTAAGTTGGCCGCAGTGTTATCACTCATGGTTATGGCAGCACTGCAT |
| | | AATTCTCTTACTGTCATGCCATCCGTAAGATGCTTTTCTGTGACTGGTGAGT |
| | | ACTCAACCAAGTCATTCTGAGAATAGTGTATGCGGCGACCGAGTTGCTCTTG |
| | | CCCGGCGTCAATACGGGATAATACCGCGCCACATAGCAGAACTTTAAAAGTG |
| | | CTCATCATTGGAAAACGTTCTTCGGGGCGAAAACTCTCAAGGATCTTACCGC |
| | | TGTTGAGATCCAGTTCGATGTAACCCACTCGTGCACCCAACTGATCTTCAGC |
| | | ATCTTTTACTTTCACCAGCGTTTCTGGGTGAGCAAAAACAGGAAGGCAAAAT |
| | | GCCGCAAAAAAGGGAATAAGGGCGACACGGAAATGTTGAATACTCATACTCT |
| | | TCCTTTTTCAATATTATTGAAGCATTTATCAGGGTTATTGTCTCATGAGCGG |
| | | ATACATATTTGAATGTATTTAGAAAAATAAACAAATAGGGGTTCCGCGCACA |
| | | TTTCCCCGAAAAGTGCCAC |

Bold and underline = compound sequence

### Example 2: In Vitro Transcription of RNA Constructs and Data Analysis

PCR-based in vitro transcription is carried out using the pMA-RQ/pMA-T vectors encoding Cpd.1-Cpd.14 to produce mRNA. A transcription template was generated by PCR using the forward and reverse primers in Table 4 (SEQ ID NO: 29 and 30). The poly(A) tail was encoded in the template resulting in a 120 bp poly(A) tail (SEQ ID NO: 133). Optimizations were made as needed to achieve specific amplification given the repetitive sequence of siRNA flanking regions. Optimizations include: 1) decreasing the amount of plasmid DNA of vector, 2) changing the DNA polymerase (Q5 hot start polymerase, New England Biolabs), 3) reducing denaturation time (30 seconds to 10 seconds) and extension time (45 seconds/kb to 10 seconds/kb) for each cycle of PCR, 4) increasing the annealing (10 seconds to 30 seconds) for each cycle of PCR, and 5) increasing the final extension time (up to 15 minutes) for each cycle of PCR. In addition, to avoid non-specific primer binding, the PCR reaction mixture was prepared on ice including thawing reagents, and the number of PCR cycles was reduced to 25.

For in vitro transcription, T7 RNA polymerase (MEGAscript kit, Thermo Fisher Scientific) was used at 37° C. for 2 hours. Synthesized RNAs were chemically modified with 100% N1-methylpseudo-UTP (modified RNA) to reduce immunogenicity or unmodified using canonical UTP (unmodified RNA). All synthesized RNAs were co-transcriptionally capped with an anti-reverse CAP analog (ARCA; [m₂^{7,3′-O}G(5′)ppp(5′)G]) at the 5′ end (Jena Bioscience). After in vitro transcription, the RNAs were column-purified using MEGAclear kit (Thermo Fisher Scientific) and quantified using Nanophotometer-N60 (Implen).

TABLE 4
Primers for Template Generation

| SEQ | Primer | |
|---|---|---|
| ID NO | Direction | Sequence (5′ to 3′) |
| 29 | Forward | GCTGCAAGGCGATTAAGTTG |
| 30 | Reverse | U(2′OMe)U(2′OMe)U(2′OMe)TTTTT |
| | | TTTTTTTTTTTTTTTTTTTTTTTTTTTTT |
| | | TTTTTTTTTTTTTTTTTTTTTTTTTTTTT |
| | | TTTTTTTTTTTTTTTTTTTTTTTTTTTTT |
| | | TTTTTTTTTTTTTTTTTTTTTTTTTCAGC |
| | | TATGACCATGTTAATGCAG |

Using in vitro transcription, Cpd.1-Cpd.14 were generated as RNA constructs and tested in various in vitro models for immunogenicity modulation.

Determination of Molecular weight of constructs was performed as below. The molecular weight of each construct was determined from each sequence by determining the total number of each base (A, C, G, T or N1-UTP) present in each sequence and multiply the number by respective molecular weight (e.g., A: 347.2 g/mol; C 323.2 g/mol; G 363.2 g/mol; N1-UTP:338.2 g/mol). The molecular weight was determined by the sum of all weights obtained for each base and ARCA molecular weight of 817.4 g/mol. The molecular weight of each construct was used to calculate the amount of RNA used for transfection in each well to nanomolar (nM) concentration. The SEAP activity was assessed using QUANTI-Blue™ and reading the optical density (O.D.) at 620 nm. Supernatant of untransfected cells was used as background control and subtracted from obtained O.D. values in tested samples. Data were analyzed using GraphPad Prism 8 (San Diego, USA). Statistical analysis was carried out using Student's t-tests to compare significant difference between groups.

### Example 3: Immunogenicity Assays

HEK-Blue™ hTLR7 Immunogenicity Assay for NF-κB Activation

The immunogenicity of different compounds with modified RNA and unmodified RNA performed in two different cell lines represent major pathways of immunogenicity. The first assay utilizes HEK-Blue™ hTLR7 (Invivogen, Cat. Code: hkb-htlr7) cells which are designed for studying the activation of human TLR7 (hTLR7) by monitoring the activation of NF-κB/AP1. Endosomal TLR7 receptor detect uridine and guanosine bases in single stranded RNA and elicit an immune reaction as an anti-viral response. These cells are derived from the human embryonic kidney HEK293 cell line and engineered to express hTLR7 and a secreted embryonic alkaline phosphatase (SEAP) reporter gene under the control of the IFN-β minimal promoter with five NF-κB and AP-1-binding sites. Upon TLR7 activation, SEAP is produced and can be determined in real-time with HEK-Blue™ Detection cell culture medium in cell culture supernatant. Stimulation of HEK-Blue™ hTLR7 cells was achieved by direct transfection of modified or unmodified compounds (Cpd.1 to Cpd.12; 0.3-0.45 μg/well).

HEK-Blue™ hTLR7 cells were maintained in Dulbecco's Modified Eagle's medium (DMEM, Sigma Aldrich) supplemented with 10% (v/v) Fetal Bovine Serum (FBS). The antibiotics Blasticidin (10 μg/mL) and Zeocin (100 μg/mL) were added to the media to select cells containing hTLR7 and SEAP transgene plasmids. Cells were seeded at 40,000 cell/well in a 96 well culture plate and incubated at 37° C. in a humidified atmosphere containing 5% CO₂ for 24 hours prior to transfection. Cells were grown in DMEM growth medium containing 10% of FBS to reach confluency <80% before transfection. Thereafter, HEK-293 cells were transfected with 0.3-0.45 μg/well of specific RNA constructs using Lipofectamine 2000 (Thermo Fisher Scientific) following the manufacturer's instructions with the RNA to Lipofectamine ratio of 1:1 w/v. 100 μl of DMEM was removed and replaced with 50 μl of Opti-MEM and 50 μl RNA and Lipofectamine 2000 complex in Opti-MEM (Thermo Fisher Scientific). After 5 hours, the medium was replaced by fresh growth medium and the plates were incubated at 37° C. in a humidified atmosphere containing 5% CO₂ for 24 hours. R848 (Resiquimod; 1 μg/ml; Invivogen, Cat. Code: tlr1-r848) hTLR7 agonist was used as positive control. After 24 hours of incubation, SEAP activity was assessed using QUANTI-Blue™ (20 μl cell culture supernatant+180 μl QUANTI-Blue™ solution) and reading the optical density (O.D.) at 620 nm in SpectraMax i3 multi-mode plate reader (Molecular Device). Untransfected samples were used as background control.

HEK-Blue™ IFN-α/β Cells Immunogenicity Assay to Monitor the Activation of the ISGF3 Pathway

The HEK-Blue™ IFN-α/β cells (Invivogen, Cat. Code: hkb-ifnab) which are designed for studying the activation the JAK-STAT and ISG3 induced by type I interferons. The activation of type I IFN is a primary immunogenicity pathway of IVT mRNA as it is detected by major RNA sensors. HEK293 cells were engineered to stably express human STAT2 and IRF9 genes to activate type I IFN signaling pathway. The activation of the IFN pathway led to the secretion of SEAP which is under the control of the ISG54 promoter, and its subsequent detection in the cell culture supernatant. HEK-Blue™ IFN-α/β cells were maintained in Dulbecco's Modified Eagle's medium (DMEM, Sigma Aldrich) supplemented with 10% (v/v) Fetal Bovine Serum (FBS). The antibiotics Blasticidin (10 μg/mL) and Zeocin (100 μg/mL) were added to the media to select cells containing STAT2, IRF9 and SEAP transgene plasmids. Cells were seeded at 40,000 cell/well in a 96 well culture plate and incubated at 37° C. in a humidified atmosphere containing 5% CO₂ for 24 hours prior to transfection. Cells were grown in DMEM growth medium containing 10% of FBS to reach confluency <80% before stimulation. Stimulation of HEK-Blue™ IFN-α/β cells was achieved by recombinant IFN-α (1 μg/ml) or IFN-α/β derived from 20 μl HEK293 cells supernatant which was previously transfected with modified and unmodified compounds (Cpd.1 to Cpd.12; 0.3-0.6 μg/well) with details below.

Human embryonic kidney cells 293 (HEK293T; ATCC, CRL-1573, Rockville, MD, USA) were maintained in Dulbecco's Modified Eagle's medium (DMEM, Sigma Aldrich) supplemented with 10% (v/v) Fetal Bovine Serum (FBS). Cells were seeded at 20,000 or 40,000 cell/well in a 96 well culture plate and incubated at 37° C. in a humidified atmosphere containing 5% CO₂ for 24 hours prior to transfection. Cells were grown in DMEM growth medium containing 10% of FBS to reach confluency <80% before transfection. Thereafter, HEK293 cells were transfected with modified and unmodified RNA compounds (Cpd.1 to Cpd.12; 0.3-0.6 μg/well) using Lipofectamine 2000 (Thermo Fisher Scientific) following the manufacturer's instructions with the RNA to Lipofectamine ratio of 1:1 w/v. 100 μl of DMEM was removed and replaced with 50 μl of Opti-MEM and 50 μl RNA and Lipofectamine 2000 complex in Opti-MEM (Thermo Fisher Scientific). After 5 hours, the medium was replaced by fresh growth medium and the plates were incubated at 37° C. in a humidified atmosphere containing 5% CO₂ for 24 hours. Cell culture supernatant (20 μl) were collected and added to culturing media of HEK-Blue™ IFN-α/β cells to measure secreted IFNs activity through the stimulation of JAK-STAT and ISG3 pathway. Recombinant human IFN-α10 (1 μg/ml) used as positive control (Invivogen, Cat. Code: rcyc-hifna10). After 2 hours of incubation, SEAP activity was assessed using QUANTI-Blue™ (20 μl cell culture supernatant+180 μl QUANTI-Blue™ solution) and reading the optical density (O.D.) at 620 nm in SpectraMax i3 multi-mode plate reader (Molecular Device). Untransfected samples were used as background control and subtracted from obtained O.D. values in tested samples.

Results

Immunogenicity Evaluation of Cpd.1 to Cpd.6

The activation of the NF-κB pathway in HEK-Blue™ hTLR7 cells by direct transfection of modified or unmodified compounds (Cpd.1 to Cpd.6; 0.3 μg/well) was confirmed experimentally. R848 (Resiquimod; 1 μg/ml), a hTLR7 agonist used as a positive control, induced high level (O.D. >2) of TLR7 activation as expected. The immunogenicity assay using HEK-Blue™ hTLR7 cells with modified and unmodified Cpd.1-Cpd.6 demonstrated that chemical modification with N1-methylpseudo-UTP significantly diminishes the immune activation compared to their unmodified counterparts (**, P<0.01; FIG. 2A). Unmodified compounds with 3×siRNAs (Cpd.2, Cpd.3, Cpd.5 and Cpd.6) or 6×siRNAs (Cpd.4) showed reduced hTLR7 activation compared to the unmodified compound without siRNA (IL-4 mRNA only) or Cpd. 1 with 1×siRNA (***, P<0.001; FIG. 2A). Likewise, stimulation of HEK-Blue™ IFN-α/β cells with recombinant IFN-α (1 μg/ml) or IFN-α/β derived from supernatant of HEK293 cells previously transfected with modified or unmodified Cpd.1-Cpd.6 (0.6 μg) was confirmed experimentally with IFNα as positive control. In line with hTLR7 activation, the modified compounds showed significantly reduced activation of IFNα/β pathway compared to their unmodified counterparts (***, P<0.001; FIG. 2B). The assay revealed that supernatants of cells transfected with unmodified compounds with 3× or 6×siRNAs (Cpd.2-Cpd.6) displayed decreased activation of JAK-STAT and ISG3 pathway compared to unmodified compounds without siRNA or Cpd.1 with 1×siRNA (***, P<0.001; FIG. 2B). In addition, Cpd.5, with 3×siRNA targeting TNF-alpha present downstream of or 3′ to IL-4, lowered immunogenicity in a significant manner, compared to Cpd. 6, with 3×siRNA targeting TNF-alpha present upstream of or 5′ to IL-4 (*, P<0.05; FIG. 2B). In summary, both assays demonstrated strongly reduced in vitro immunogenicity providing a potentially better safety profile of unmodified compounds.

Immunogenicity Evaluation of Cpd.6 to Cpd.9 and Cpd.4

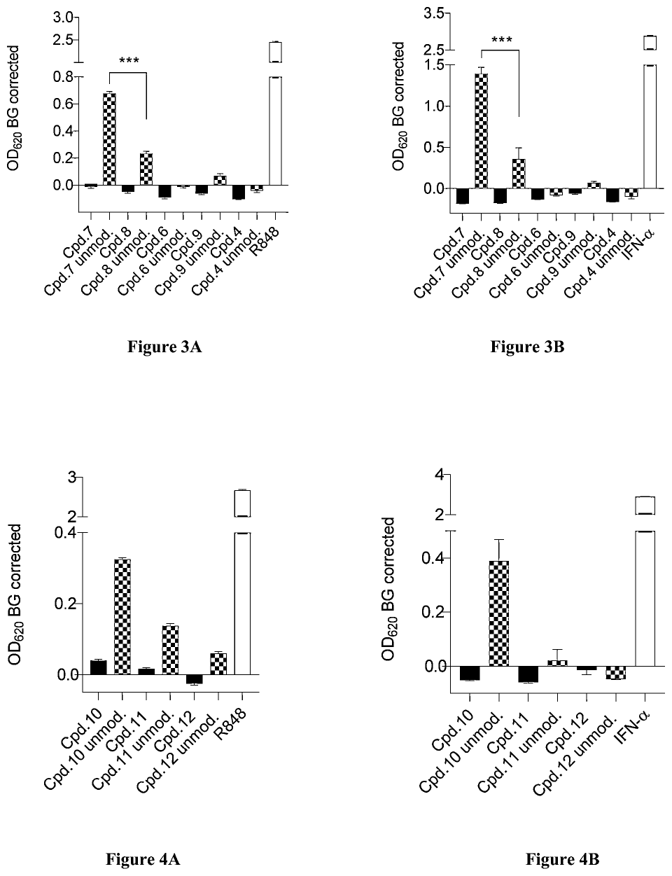

Cpd.6-Cpd.9 and Cpd.4 represent constructs with IL-4 encoding mRNA with increasing number siRNAs targeting TNF-alpha. Cpd.7 and Cpd.8 contain 1×siRNA at 5′ (or upstream) and 3′ (or downstream) to IL-4 encoding sequence, respectively. Cpd.6 contains 3×siRNAs with A1 linkers whereas Cpd.9 comprises 3×siRNA with A2 linkers. Cpd.4 contains 6×siRNAs (3× targeting TNF-alpha and 3× targeting IL-17). Stimulation of HEK-Blue™ hTLR7 cells with direct transfection (0.3 μg/well) of modified or unmodified Cpd.6-Cpd.9 and Cpd.4 displayed varying activation level of NF-κB pathway, as measured by SEAP secretion in transfected cells 24 hours later. Modified Compounds displayed reduced immunogenicity for all tested constructs (***, P<0.001; FIG. 3A). Although unmodified Cpd.7 with 1×siRNA present at the 5′ to (or upstream of) IL-4 encoding sequence was found to be highly immunogenic, repositioning the same siRNA to 3′ to (or downstream of) IL-4 resulted in 3-fold decline in the NF-κB pathway activation (***, P<0.001; FIG. 3A). The addition of 3× or 6× siRNA in Cpd.6, Cpd.9, and Cpd.4 eliminated the need of using modified bases for RNA constructs to escape from hTLR7 binding (FIG. 3A). The positive control R848 induced high level of TLR7 activation as expected. The immunogenicity assessment of Cpd.6-Cpd.8 and Cpd.4 in HEK-Blue™ IFN-α/β cells using supernatant of cell culture transfected with unmodified compounds with increased number of siRNA (3× in Cpd.6 or Cpd.9 or 6× in Cpd.4) showed that the level of IFN signaling was undetectable compared to using supernatant of cell culture transfected with unmodified compounds with 1×siRNA (Cpd.7 and Cpd.8; FIG. 3A). As observed with hTRL7 activation, the alteration of the position of 1×siRNA in the compound (5′ to 3′ of gene of interest) caused 4-fold reduced immunogenicity (***, P<0.001; FIG. 3B). In summary, both assays demonstrated siRNA copy number dependent (e.g., number of siRNA) decline in immune activation of unmodified compounds.

Immunogenicity Evaluation of Cpd.10 to Cpd.12

Cpd.10 to Cpd.12 represent constructs with IGF-1 encoding mRNA and increasing number (1×, 2× and 3×, respectively) of siRNA targeting Turbo GFP. Immune stimulation of unmodified Cpd.10-Cpd.12 in HEK-Blue™ hTLR7 cells displayed dose dependent (number of siRNAs in the construct) reduction in activation of NF-κB pathway as measured by SEAP secretion as shown in FIG. 4A (***, P<0.001). Likewise, the immunogenicity assessment of Cpd.9-Cpd.11 in HEK-Blue™ IFN-α/β cells showed dose dependent (number of siRNAs in the construct) decrease in IFN signaling (FIG. 4B). In summary, compounds with at least 2×siRNA reduces the level of the hTLR7 activation moderately and reduces IFN signaling significantly.

HEK-Blue™ hTLR3 Immunogenicity Assay for NF-κB/AP-1 Activation

The combination constructs (mRNA+siRNA) possess a hairpin loop with shorter dsRNA structure (about 20 nucleotides) and could potentially induce unwanted immune response through endosomal TLR3 ligands that detect double stranded RNA (dsRNA) motifs. Endosomal TLR3 receptors detect dsRNA and elicit an immune reaction as an anti-microbial response. The HEK-Blue™ hTLR3 (Invivogen, Cat. Code: hkb-htlr3) cells which are designed for studying the activation of human TLR3 (hTLR3) by monitoring the activation of NF-κB/AP1 signalling cascade. These cells are derived from the human embryonic kidney HEK293 cell line and engineered to express hTLR3 and a secreted embryonic alkaline phosphatase (SEAP) reporter gene under the control of an NF-κB and AP-1-inducible promoter. Upon TLR3 activation, SEAP is produced and can be determined in real-time with HEK-Blue™ Detection cell culture medium in cell culture supernatant. Stimulation of HEK-Blue™ hTLR3 cells was achieved by direct transfection of modified or unmodified compounds (Cpd.3, Cpd.13 and Cpd.14; 0.6 μg/well).

HEK-Blue™ hTLR3 cells were maintained in Dulbecco's Modified Eagle's medium (DMEM, Sigma Aldrich) supplemented with 10% (v/v) Fetal Bovine Serum (FBS). The antibiotics Blasticidin (10 μg/mL) and Zeocin (100 μg/mL) were added to the media to select cells containing hTLR3 and SEAP transgene plasmids. Cells were seeded at 40,000 cell/well in a 96 well culture plate and incubated at 37° C. in a humidified atmosphere containing 5% CO₂ for 24 hours prior to transfection. Cells were grown in DMEM growth medium containing 10% of FBS to reach confluency <80% before transfection. Thereafter, HEK-293 cells were transfected with 0.6 μg/well of specific RNA constructs using Lipofectamine 2000 (Thermo Fisher Scientific) following the manufacturer's instructions with the RNA to Lipofectamine ratio of 1:1 w/v. 100 μl of DMEM was removed and replaced with 90 μl of Opti-MEM and 10 μl RNA and Lipofectamine 2000 complex in Opti-MEM (Thermo Fisher Scientific). The plates were incubated at 37° C. in a humidified atmosphere containing 5% CO₂ for 24 hours. Poly(I:C) HMW (1 μg/ml; Invivogen, Cat. Code: tlra-pic) was used as a positive control. After 24 hours of incubation, SEAP activity was assessed using QUANTI-Blue™ (20 μl cell culture supernatant+180 μl QUANTI-Blue™ solution) and reading the optical density (O.D.) at 620 nm in SpectraMax i3 multi-mode plate reader (Molecular Device). Untransfected samples were used as background control.

Results

Immunogenicity Evaluation of Cpd.3, Cpd.13 and Cpd. 14

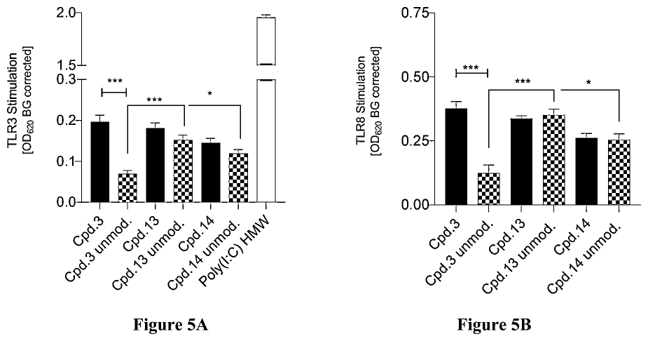

The activation of the NF-κB/AP1 pathway in HEK-Blue™ hTLR3 cells by direct transfection of modified or unmodified compounds (Cpd.3 and Cpd.13; 0.6 μg/well) was confirmed experimentally. Poly(I:C) HMW (1 μg/ml), a hTLR3 agonist was used as a positive control, induced high level (O.D. >1.95) of TLR3 activation as expected. The immunogenicity assay using HEK-Blue™ hTLR3 cells with modified and unmodified Cpd.3, Cpd.13 and Cpd.14 demonstrated that chemical modification with N1-methylpseudo-UTP does not have any impact on TLR3 activation compared to their unmodified counterparts as the endosomal TLR3 ligand directed towards dsRNA instead of sensing uridine (U) or guanosine (G) motifs. Unmodified Cpd.3 with 3×siRNA targeting IL-1 beta with IGF-1 showed significantly reduced hTLR3 activation compared to the unmodified compound without siRNA Cpd.13 (***, P<0.001; FIG. 5A) or Cpd.14 (*, P<0.05; FIG. 5A). Similarly, the presence of 3×siRNA in unmodified Cpd.3 diminished the TLR8 mediated immune activation compared to modified Cpd.3 (***, P<0.001; FIG. 5B).

HEK-Blue™ hTLR8 Immunogenicity Assay for NF-κB/AP-1/IRF Activation

The HEK-Blue™ hTLR8 (Invivogen, Cat. Code: hkb-htlr8) cells are designed for studying the activation of human TLR8 (hTLR8) by monitoring the activation of NF-κB/AP1/IRF signalling cascade. Endosomal TLR8 receptors detect uridine and guanosine motif in single-stranded RNA and elicit an immune reaction as an anti-viral response. HEK-Blue™ hTLR8 cells are derived from the human embryonic kidney HEK293 cell line and engineered to express hTLR8 and a secreted embryonic alkaline phosphatase (SEAP) reporter gene under the control of the IFN-β minimal promoter with five NF-κB and AP-1-binding sites. Upon TLR8 activation, SEAP is produced and can be determined in real-time with HEK-Blue™ Detection cell culture medium in cell culture supernatant. Stimulation of HEK-Blue™ hTLR7 cells was achieved by direct transfection of modified or unmodified compounds (Cpd.3, Cpd.13 and Cpd.14; 0.6 μg/well).

HEK-Blue™ hTLR8 cells were maintained in Dulbecco's Modified Eagle's medium (DMEM, Sigma Aldrich) supplemented with 10% (v/v) Fetal Bovine Serum (FBS). The antibiotics Blasticidin (10 μg/mL) and Zeocin (100 μg/mL) were added to the media to select cells containing hTLR8 and SEAP transgene plasmids. Cells were seeded at 40,000 cell/well in a 96 well culture plate and incubated at 37° C. in a humidified atmosphere containing 5% CO₂ for 24 hours prior to transfection. Cells were grown in DMEM growth medium containing 10% of FBS to reach confluency <80% before transfection. Thereafter, HEK-293 cells were transfected with 0.6 μg/well of specific RNA constructs using Lipofectamine 2000 (Thermo Fisher Scientific) following the manufacturer's instructions with the RNA to Lipofectamine ratio of 1:1 w/v. 100 μl of DMEM was removed and replaced with 90 μl of Opti-MEM and 10 μl RNA and Lipofectamine 2000 complex in Opti-MEM (Thermo Fisher Scientific). The plates were incubated at 37° C. in a humidified atmosphere containing 5% CO₂ for 24 hours. R848 (Resiquimod; 1 μg/ml; Invivogen, Cat. Code: tlr1-r848) hTLR7/8 agonist was used as positive control. After 24 hours of incubation, SEAP activity was assessed using QUANTI-Blue™ (20 μl cell culture supernatant+180 μl QUANTI-Blue™ solution) and reading the optical density (O.D.) at 620 nm in SpectraMax i3 multi-mode plate reader (Molecular Device). Untransfected samples were used as background control.

Results

Immunogenicity Evaluation of Cpd.3, Cpd.13 and Cpd. 14

The activation of the NF-κB/AP1/IRF pathway in HEK-Blue™ hTLR8 cells by direct transfection of modified or unmodified compounds (Cpd.3 and Cpd.13; 0.6 μg/well) was confirmed experimentally. R848 (1 μg/ml), a hTLR7/8 agonist was used as a positive control, induced moderate level of TLR8 activation (O.D. >0.45) unlike with HEK-Blue™ hTLR7 cells. The immunogenicity assay using HEK-Blue™ hTLR8 cells with modified and unmodified Cpd.3, Cpd.13 and Cpd.14 demonstrated that chemical modification with N1-methylpseudo-UTP does not have any impact on TLR8 activation compared to their unmodified counterparts. Although TLR8 ligands are known to detect the uridine (U) motifs, there was no difference in TLR8 activation observed for modified and unmodified constructs. This may be explained by the report that the binding affinity of TLR8 is observed to be guanosine motif specific in HEK-Blue™ hTLR8 cells (Hu et al., Bioorg Med Chem. 2018 Jan. 1; 26(1): 77-83). Unmodified Cpd.3 with 3×siRNA targeting IL-1 beta with IGF-1 showed significantly reduced hTLR8 activation compared to the unmodified compounds without siRNA i.e., Cpd.13 (***, P<0.001; FIG. 5B) or Cpd.14 (*, P<0.05; FIG. 5B). Similarly, the presence of 3×siRNA in unmodified Cpd.3 diminished the TLR8 mediated immune activation compared to modified Cpd.3 (***, P<0.001; FIG. 5B).

Immunogenicity Assessment by Stimulating Endogenous IL-6 Expression in A549 Cells

Human lung epithelial carcinoma cells (A549; Sigma-Aldrich Cat. #6012804) cells were utilized for studying the immune response against in vitro transcribed mRNA (IVT mRNA) by monitoring the endogenous expression of Interleukin 6 (IL-6) upon direct transfection of mRNA. Stimulation of immunogenicity against IVT mRNA are triggered by RNA sensors (e.g. TLRs, PKRs) and induces the expression of several proinflammatory cytokines including IL-6, which plays a central role in the immune activation against IVT mRNA. A549 cells were maintained in Dulbecco's Modified Eagle's medium high glucose (DMEM, Sigma-Aldrich) supplemented with 10% (v/v) Fetal Bovine Serum (FBS, VWR, Cat #97068-091). A549 cells were seeded at 10,000 cell/well in a 96 well culture plate and incubated at 37° C. in a humidified atmosphere containing 5% CO₂ for 24 hours prior to transfection. Cells were then grown in DMEM growth medium containing 10% of FBS to reach confluency <70% before transfection. Thereafter, A549 cells were transfected with specific mRNA constructs (0.3 μg) using Lipofectamine 2000 (Invitrogen) following the manufacturer's instructions with the mRNA to Lipofectamine ratio of 1:1 w/v. 100 μl of DMEM was removed and 50 μl of Opti-MEM (Thermo Fisher Scientific) was added to each well followed by 50 μl mRNA and Lipofectamine 2000 complex in Opti-MEM. After 5 hours of incubation, the medium was replaced by fresh growth medium and the plates were incubated for 24 hours at 37° C. in a humidified atmosphere containing 5% CO₂. Cell culture supernatant were collected to measure secreted human IL-6 using ELISA (ThermoFisher Cat. #887066).

Results

Immunogenicity Evaluation of Cpd.3, Cpd.4, Cpd.13 and Cpd. 14

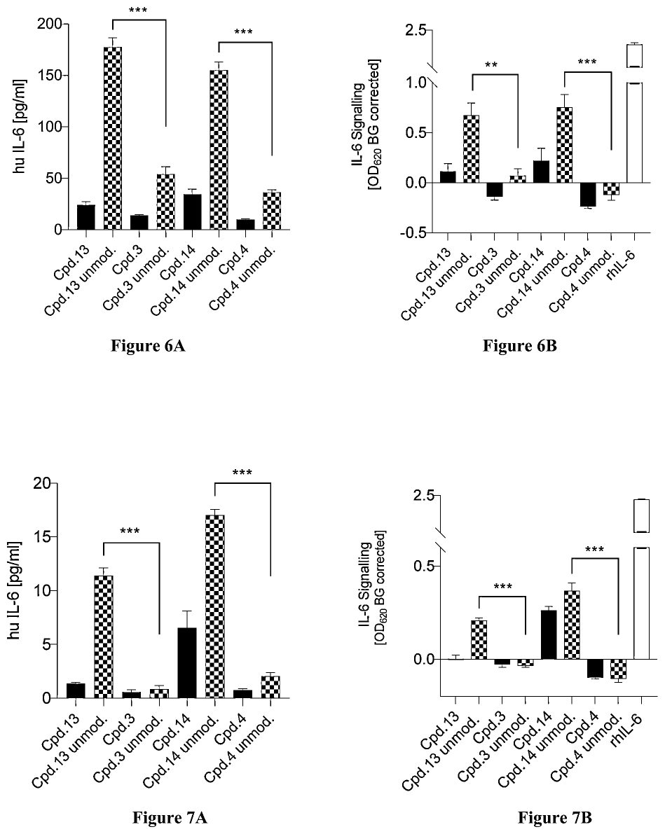

The endogenous expression of IL-6 in A549 cells by direct transfection of modified or unmodified compounds (Cpd.3, Cpd.4, Cpd.13 and Cpd.14; 0.3 μg/well) was confirmed experimentally. Distinct differences in IL-6 levels between cells transfected with modified or unmodified RNA compounds (FIG. 6A) demonstrated that chemical modification with N1-methylpseudo-UTP diminishes the immune activation in A549 cells. For modified compounds, the presence of siRNA structures (3× in Cpd.3; 6× in Cpd.4) significantly further reduced IL-6 levels relative to the respective compounds without siRNA (see, e.g. Cpd.3 vs. Cpd.13 (**, <0.01); Cpd.4 vs. Cpd.14 (***, <0.001)). Remarkable reduction in IL-6 levels was noted for unmodified compounds in relation to the presence or absence of siRNA. Unmodified Cpd.3 with 3×siRNA targeting IL-1 beta with IGF-1 showed significantly reduced IL-6 levels compared to the unmodified compound comprising IGF-1 without siRNA i.e., Cpd.13 (***, P<0.001; FIG. 6A). Similarly, the presence of 6×siRNA (3× targeting TNF-alpha and 3× targeting IL-17) along with IL-4 mRNA in unmodified Cpd.4 demonstrated decreased IL-6 levels compared to unmodified Cpd.14 comprising IL-4-encoding mRNA without siRNA (***, P<0.001; FIG. 6A). The comparison of unmodified Cpd.3 (3×) and Cpd.4 (6×) shows that increased siRNA decreases the IL-6 stimulation (*, P<0.05; FIG. 6A).

Immunogenicity Assessment by Stimulating Endogenous IL-6 Expression in THP-1 Cells

Human monocyte leukemia cell line THP-1 (Sigma-Aldrich, Cat. #88081201) were utilized for studying the immune response against in vitro transcribed mRNA (IVT mRNA) by monitoring the endogenous expression of IL-6 upon direct transfection of mRNA. Stimulation of immunogenicity against IVT mRNA are triggered by RNA sensors (e.g. TLRs, PKRs) and induces the expression of several proinflammatory cytokines including IL-6, which plays a central role in the immune activation against IVT mRNA. THP-1 cells were maintained in growth medium (RPMI 1640 supplemented with 10% FBS and 2 mM glutamine). The cells were seeded at 40,000 THP-1 cells in a 96 well cell culture plate and were reverse-transfected with specific mRNA (0.3 μg/well) using Lipofectamine MessengerMax (Thermo Fisher Scientific). The plates were incubated at 37° C. in a humidified atmosphere containing 5% CO₂ for 24 hours. Post transfection, the cell culture supernatant was collected and quantified for human IL-6 using ELISA (ThermoFisher Cat. #887066).

Results

Immunogenicity Evaluation of Cpd.3, Cpd.4, Cpd.13 and Cpd. 14

The endogenous expression of IL-6 in THP-1 cells by direct transfection of modified or unmodified compounds (Cpd.3, Cpd.4, Cpd.13, and Cpd.14; 0.3 μg/well) was confirmed experimentally, however the expression levels were low compared to the expression levels in A549 cells. Similar to A549 cells, differences in IL-6 levels were noted between modified and unmodified RNA compounds (FIG. 7A). A significant reduction in IL-6 levels was observed for unmodified compounds in relation to the presence or absence of siRNA. Unmodified Cpd.3 with 3×siRNA targeting IL-1 beta with IGF-1 mRNA showed significantly reduced IL-6 levels compared to the unmodified compound comprising IGF-1 mRNA alone, i.e. Cpd.13 (***, P<0.001; FIG. 7A). Similarly, the presence of 6×siRNA (3× targeting TNF-alpha and 3× targeting IL-17) along with IL-4 mRNA in unmodified Cpd.4 demonstrated decreased IL-6 levels compared to unmodified Cpd.14 comprising IL-4-encoding mRNA without siRNA (***, P<0.001; FIG. 7A).

HEK-Blue™ IL-6 Reporter Assay for STAT-3 Activation

The functional activity of secreted IL-6 in A549 and THP-1 in vitro model were tested in HEK-Blue™ IL-6 reporter cells (Invivogen, Cat. Code: hkb-i16), which are designed for studying the IL-6 signaling by monitoring the activation of STAT-3 pathway. The HEK-Blue™ IL-6 cells are derived from the human embryonic kidney HEK293 cell line and engineered to express IL-6 receptor (IL-6R) and signal transducer activator of transcription 3 (STAT3). IL-6 signalling is detected by a reporter gene expressing a secreted embryonic alkaline phosphatase (SEAP) under the control of the IFN-β minimal promoter fused to four STAT3 binding sites. Upon IL-6 stimulation, HEK-Blue™ IL-6 cells trigger the activation of STAT3 and the subsequent secretion of SEAP can be determined in real-time with HEK-Blue™ Detection cell culture medium in cell culture supernatant. Stimulation of HEK-Blue™ IL-6 cells were achieved by equivalent volume (20 μl) of IL-6 derived from cell culture supernatant of A549 cell or THP-1 cells which had been transfected with Cpd.3, Cpd.4, Cpd.13, or Cpd.14 (0.3 μg/well) with below details.

HEK-Blue™ IL-6 cells were maintained in Dulbecco's Modified Eagle's medium (DMEM, Sigma Aldrich) supplemented with 10% (v/v) Fetal Bovine Serum (FBS). The antibiotics HEK-Blue™ selection (1:250 dilution with media) were added to the media to select cells containing IL6R, STAT3 and SEAP transgene plasmids. Cells were seeded at 40,000 cell/well in a 96 well culture plate and incubated at 37° C. in a humidified atmosphere containing 5% CO₂ for 24 hours prior to transfection. Cells were grown in DMEM growth medium containing 10% of FBS to reach confluency <80% before transfection. Equivalent volume of (20 μl) of cell culture supernatant from A549 and THP-1 cells which had been transfected with Cpd.3, Cpd.4, Cpd.13, or Cpd.14 (0.3 μg/well) were collected and added to culture media of HEK-Blue™ IL-6 cells to measure IL-6 receptor recruitment followed by STAT3 pathway activation. rhIL-6 (100 ng/mL) was used as a positive control. After 24 hours of incubation, SEAP activity was assessed using QUANTI-Blue™ (20 μl cell culture supernatant+180 μl QUANTI-Blue™ solution) and reading the optical density (O.D.) at 620 nm in SpectraMax i3 multi-mode plate reader (Molecular Device). Untransfected samples were used as background control and subtracted from obtained O.D. values in tested samples.

Results

Immunogenicity Evaluation of Cpd.3, Cpd.4, Cpd.13 and Cpd. 14

Stimulation of HEK-Blue™ IL-6 cells with rhIL-6 or IL-6 derived from cell culture supernatant of A549 or THP-1 cells that had been transfected with modified or unmodified Cpd.3, Cpd.4, Cpd.13 or Cpd.14 was functional as SEAP production was observed in all samples (FIGS. 6B and 7B). As expected, the supernatant derived from A549 and THP-1 cells transfected with the RNA compounds modified with N1-methylpseudouridine induced less STAT-3 signaling compared to the supernatant derived from A549 and THP-1 cells transfected with unmodified compounds (FIGS. 6B and 7B). The supernatant derived from A549 and THP-1 cells transfected with unmodified Cpd.3 with 3×siRNA targeting IL-1 beta with IGF-1 mRNA showed significantly reduced IL-6 signaling compared to the supernatant derived from A549 and THP-1 cells transfected with unmodified compound comprising IGF-1 mRNA alone, i.e. Cpd.13 (FIGS. 6B and 7B). Likewise, the presence of 6×siRNA (3× targeting TNF-alpha and 3× targeting IL-17) along with IL-4 mRNA in unmodified Cpd.4 demonstrated a decreased IL-6 mediated STAT-3 activation compared to unmodified Cpd.14 comprising IL-4-encoding mRNA without siRNA (***, P<0.001; FIGS. 6B and 7B) for the supernatant derived from both A549 and THP-1 cells. The comparison of unmodified Cpd.3 (3×) and Cpd.4 (6×) shows that increased number of siRNA from 3× to 6× decreases the IL-6 mediated immune activation (A549 cells, *, P<0.05; FIG. 6B; THP-1 cells, **, P<0.01; FIG. 7B).

The examples and embodiments described herein are for illustrative purposes only and various modifications or changes suggested to persons skilled in the art are to be included within the spirit and purview of this application and scope of the appended claims.

TABLE 5
Table of Sequences Listed

| Protein or | | SEQ ID |
|---|---|---|
| Nucleic Acid | Sequence | NO: |
| Compound 1-14 | See Table 2 and Table 3 | 1-24, |
| nucleic acid | | 42, 125, |
| sequences | | and |
| | | 127-128 |
| Kozak sequence | GCCACC | 25 |
| T7 promoter | TAATACGACTCACTATA | 26 |
| A1-linker: | ATAGTGAGTCGTATTAACGTACCAACAA | 27 |
| mRNA to | | |
| siRNA linker | | |
| A1-linker: | TTTATCTTAGAGGCATATCCCTACGTACCAACAA | 28 |
| siRNA to | | |
| siRNA linker | | |
| Forward Primer | GCTGCAAGGCGATTAAGTTG | 29 |
| Reverse Primer | U(2′OMe)U(2′OMe)U(2′OMe)TTTTTTTTTTTTTTTTTTTTTTTTT | 30 |
| | TTTTTTTTTTTTTTTTTTTTTTTTTTTTTTTTTTTTTTTTTTTTTTTTT | |
| | TTTTTTTTTTTTTTTTTTTTTTTTTTTTTTTTTTTTTTTTTTTCAGCTA | |
| | TGACCATGTTAATGCAG | |
| IL-4 | ATCGTTAGCTTCTCCTGATAAACTAATTGCCTCACATTGTCACTGCAAA | 31 |
| Human IL-4 | TCGACACCTATTAATGGGTCTCACCTCCCAACTGCTTCCCCCTCTGTTC | |
| Nucleotide | TTCCTGCTAGCATGTGCCGGCAACTTTGTCCACGGACACAAGTGCGATA | |
| (Genbank | TCACCTTACAGGAGATCATCAAAACTTTGAACAGCCTCACAGAGCAGAA | |
| NM_000589.4) | GACTCTGTGCACCGAGTTGACCGTAACAGACATCTTTGCTGCCTCCAAG | |
| | AACACAACTGAGAAGGAAACCTTCTGCAGGGCTGCGACTGTGCTCCGGC | |
| | AGTTCTACAGCCACCATGAGAAGGACACTCGCTGCCTGGGTGCGACTGC | |
| | ACAGCAGTTCCACAGGCACAAGCAGCTGATCCGATTCCTGAAACGGCTC | |
| | GACAGGAACCTCTGGGGCCTGGCGGGCTTGAATTCCTGTCCTGTGAAGG | |
| | AAGCCAACCAGAGTACGTTGGAAAACTTCTTGGAAAGGCTAAAGACGAT | |
| | CATGAGAGAGAAATATTCAAAGTGTTCGAGCTGAATATTTTAATTTATG | |
| | AGTTTTTGATAGCTTTATTTTTTAAGTATTTATATATTTATAACTCATC | |
| | ATAAAATAAAGTATATATAGAATCTAA | |
| IL-4 | MGLTSQLLPPLFFLLACAGNFVHGHKCDITLQEIIKTLNSLTEQKTLCT | 32 |
| Human IL-4 | ELTVTDIFAASKNTTEKETFCRAATVLRQFYSHHEKDTRCLGATAQQFH | |
| amino acid | RHKQLIRFLKRLDRNLWGLAGLNSCPVKEANQSTLENFLERLKTIMREK | |
| (Genbank | YSKCSS | |
| NP_000580.1) | | |
| Underlined: | | |
| signal sequence | | |
| IGF-1 | ATGGGAAAAATCAGCAGTCTTCCAACCCAATTATTTAAGTGCTGCTTTT | 33 |
| Human IGF-1 | GTGATTTCTTGAAGGTGAAGATGCACACCATGTCCTCCTCGCATCTCTT | |
| Nucleotide | CTACCTGGCGCTGTGCCTGCTCACCTTCACCAGCTCTGCCACGGCTGGA | |
| (Genbank | CCGGAGACGCTCTGCGGGGCTGAGCTGGTGGATGCTCTTCAGTTCGTGT | |
| NM_000618.4) | GTGGAGACAGGGGCTTTTATTTCAACAAGCCCACAGGGTATGGCTCCAG | |
| | CAGTCGGAGGGCGCCTCAGACAGGCATCGTGGATGAGTGCTGCTTCCGG | |
| | AGCTGTGATCTAAGGAGGCTGGAGATGTATTGCGCACCCCTCAAGCCTG | |
| | CCAAGTCAGCTCGCTCTGTCCGTGCCCAGCGCCACACCGACATGCCCAA | |
| | GACCCAGAAGGAAGTACATTTGAAGAACGCAAGTAGAGGGAGTGCAGGA | |
| | AACAAGAACTACAGGATGTAG | |
| IGF-1 | MGKISSLPTQLFKCCFCDFLKVKMHTMSSSHLFYLALCLLTFTSSATAG | 34 |
| Human IGF-1 | PETLCGAELVDALQFVCGDRGFYFNKPTGYGSSSRRAPQTGIVDECCFR | |
| amino acid | SCDLRRLEMYCAPLKPAKSARSVRAQRHTDMPKTQKEVHLKNASRGSAG | |
| (Genbank | NKNYRM | |
| NP_000609.1) | | |
| Underlined: | | |
| signal sequence | | |
| IGF-1 | ATGACCATCCTGTTTCTGACAATGGTCATCAGCTACTTCGGCTGCATGA | 35 |
| Human IGF-1 | AGGCCGTGAAGATGCACACCATGAGCAGCAGCCACCTGTTCTATCTGGC | |
| Nucleotide | CCTGTGCCTGCTGACCTTTACCAGCTCTGCTACCGCCGGACCTGAGACA | |
| (Optimized IGF- | CTTTGTGGCGCTGAACTGGTGGACGCCCTGCAGTTTGTGTGTGGCGACA | |
| 1 with BDNF | GAGGCTTCTACTTCAACAAGCCCACAGGCTACGGCAGCAGCTCTAGAAG | |
| SP and without | GGCTCCTCAGACCGGAATCGTGGACGAGTGCTGCTTCAGAAGCTGCGAC | |
| E-peptide) | CTGCGGCGGCTGGAAATGTATTGTGCCCCTCTGAAGCCTGCCAAGAGCG | |
| | CCTAA | |
| IGF-1 | MTILFLTMVISYFGCMKAVKMHTMSSSHLFYLALCLLTFTSSATAGPET | 36 |
| Human IGF-1 | LCGAELVDALQFVCGDRGFYFNKPTGYGSSSRRAPQTGIVDECCFRSCD | |
| amino acid | LRRLEMYCAPLKPAKSA | |
| (Optimized IGF- | | |
| 1 with BDNF | | |
| SP and without | | |
| E-peptide) | | |
| Underlined: | | |
| signal sequence | | |
| Human IL-8 | ATGACTTCCAAGCTGGCCGTGGCTCTCTTGGCAGCCTTCCTGATTTCTG | 37 |
| Nucleotide | CAGCTCTGTGTGAAGGTGCAGTTTTGCCAAGGAGTGCTAAAGAACTTAG | |
| (Genbank | ATGTCAGTGCATAAAGACATACTCCAAACCTTTCCACCCCAAATTTATC | |
| NM_000584.3) | AAAGAACTGAGAGTGATTGAGAGTGGACCACACTGCGCCAACACAGAAA | |
| Bold and | TTATTGTAAAGCTTTCTGATGGAAGAGAGCTCTGTCTGGACCCCAAGGA | |
| italicized: | AAACTGGGTGCAGAGGGTTGTGGAGAAGTTTTTGAAGAGGGCTGAGAAT | |
| siRNA binding | TCATAA | |
| regions | | |
| Human IL-1beta | ATGGCAGAAGTACCTGAGCTCGCCAGTGAAATGATGGCTTATTACAGTG | 38 |
| Nucleotide | GCAATGAGGATGACTTGTTCTTTGAAGCTGATGGCCCTAAACAGATGAA | |
| (Genbank | GTGCTCCTTCCAGGACCTGGACCTCTGCCCTCTGGATGGCGGCATCCAG | |
| NM_000594.3) | CTACGAATCTCCGACCACCACTACAGCAAGGGCTTCAGGCAGGCCGCGT | |
| Bold and | CAGTTGTTGTGGCCATGGACAAGCTGAGGAAGATGCTGGTTCCCTGCCC | |
| italicized: | ACAGACCTTCCAGGAGAATGACCTGAGCACCTTCTTTCCCTTCATCTTT | |
| siRNA binding | GAAGAAGAACCTATCTTCTTCGACACATGGGATAACGAGGCTTATGTGC | |
| regions | ACGATGCACCTGTACGATCACTGAACTGCACGCTCCGGGACTCACAGCA | |
| Second siRNA | AAAAAGCTTGGTGATGTCTGGTCCATATGAACTGAAAGCTCTCCACCTC | |
| biding site | CAGGGACAGGATATGGAGCAACAAGTGGTGTTCTCCATGTCCTTTGTAC | |
| underlined | AAGGAGAAGAAAGTAATGACAAAATACCTGTGGCCTTGGGCCTCAAGGA | |
| | AAAGAATCTGTACCTGTCCTGCGTGTTGAAAGATGATAAGCCCACTCTA | |
| | CAGCTGGAGAGTGTAGATCCCAAAAATTACCCAAAGAAGAAGATGGAAA | |
| | AGCGATTTGTCTTCAACAAGATAGAAATCAATAACAAGCTGGAATTTGA | |
| | GTCTGCCCAGTTCCCCAACTGGTACATCAGCACCTCTCAAGCAGAAAAC | |
| | ATGCCCGTCTTCCTGGGAGGGACCAAAGGCGGCCAGGATATAACTGACT | |
| | TCACCATGCAATTTGTGTCTTCCTAA | |
| Human IL-17 | ATGACTCCTGGGAAGACCTCATTGGTGTCACTGCTACTGCTGCTG | 39 |
| Nucleotide | AGCCTGGAGGCCATAGTGAAGGCAGGAATCACAATCCCACGAAAT | |
| (Genbank | CCAGGATGCCCAAATTCTGAGGACAAGAACTTCCCCCGGACTGTG | |
| NM_002190.2) | ATGGTCAACCTGAACATCCATAACCGGAATACCAATACCAATCCC | |
| Bold and | AAAAGGTCCTCAGATTACTACAACCGATCCACCTCACCTTGGAAT | |
| italicized: | CTCCACCGCAATGAGGACCCTGAGAGATATCCCTCTGTGATCTGG | |
| siRNA binding | GAGGCAAAGTGCCGCCACTTGGGCTGCATCAACGCTGATGGGAAC | |
| regions | GTGGACTACCACATGAACTCTGTCCCCATCCAGCAAGAGATCCTG | |
| | GTCCTGCGCAGGGAGCCTCCACACTGCCCCAACTCCTTCCGGCTG | |
| | GAGAAGATACTGGTGTCCGTGGGCTGCACCTGTGTCACCCCGATT | |
| | GTCCACCATGTGGCCTAA | |
| Human TNF-α | ATGAGCACTGAAAGCATGATCCGGGACGTGGAGCTGGCCGAGGAG | 40 |
| Nucleotide | GCGCTCCCCAAGAAGACAGGGGGGCCCCAGGGCTCCAGGCGGTGC | |
| (Genbank | TTGTTCCTCAGCCTCTTCTCCTTCCTGATCGTGGCAGGCGCCACC | |
| NM_000594.3) | ACGCTCTTCTGCCTGCTGCACTTTGGAGTGATCGGCCCCCAGAGG | |
| Bold and | GAAGAGTTCCCCAGGGACCTCTCTCTAATCAGCCCTCTGGCCCAG | |
| italicized: | GCAGTCAGATCATCTTCTCGAACCCCGAGTGACAAGCCTGTAGCC | |
| siRNA binding | CATGTTGTAGCAAACCCTCAAGCTGAGGGGCAGCTCCAGTGGCTG | |
| regions | AACCGCCGGGCCAATGCCCTCCTGGCCAATGGCGTGGAGCTGAGA | |
| | GATAACCAGCTGGTGGTGCCATCAGAGGGCCTGTACCTCATCTAC | |
| | TCCCAGGTCCTCTTCAAGGGCCAAGGCTGCCCCTCCACCCATGTG | |
| | CTCCTCACCCACACCATCAGCCGCATCGCCGTCTCCTACCAGACC | |
| | AAGGTCAACCTCCTCTCTGCCATCAAGAGCCCCTGCCAGAGGGAG | |
| | ACCCCAGAGGGGGCTGAGGCCAAGCCCTGGTATGAGCCCATCTAT | |
| | CTGGGAGGGGTCTTCCAGCTGGAGAAGGGTGACCGACTCAGCGCT | |
| | GAGATCAATCGGCCCGACTATCTCGACTTTGCCGAGTCTGGGCAG | |
| | GTCTACTTTGGGATCATTGCCCTGTGA | |
| Turbo GFP | ATGGAGAGCGACGAGAGCGGCCTGCCCGCCATGGAGATCGAGTGC | 41 |
| Bold and | CGCATCACCGGCACCCTGAACGGCGTGGAGTTCGAGCTGGTGGGC | |
| italicized: | GGCGGAGAGGGCACCCCCGAGCAGGGCCGCATGACCAACAAGATG | |
| siRNA binding | AAGAGCACCAAAGGCGCCCTGACCTTCAGCCCCTACCTGCTGAGC | |
| regions | CACGTGATGGGCTACGGCTTCTACCACTTCGGCACCTACCCCAGC | |
| | GGCTACGAGAACCCCTTCCTGCACGCCATCAACAACGGCGGCTAC | |
| | ACCAACACCCGCATCGAGAAGTACGAGGACGGCGGCGTGCTGCAC | |
| | GTGAGCTTCAGCTACCGCTACGAGGCCGGCCGCGTGATCGGCGAC | |
| | TTCAAGGTGATGGGCACCGGCTTCCCCGAGGACAGCGTGATCTTC | |
| | ACCGACAAGATCATCCGCAGCAACGCCACCGTGGAGCACCTGCAC | |
| | CCCATGGGCGATAACGATCTGGATGGCAGCTTCACCCGCACCTTC | |
| | AGCCTGCGCGACGGCGGCTACTACAGCTCCGTGGTGGACAGCCAC | |
| | ATGCACTTCAAGAGCGCCATCCACCCCAGCATCCTGCAGAACGGG | |
| | GGCCCCATGTTCGCCTTCCGCCGCGTGGAGGAGGATCACAGCAAC | |
| | ACCGAGCTGGGCATCGTGGAGTACCAGCACGCCTTCAAGACCCCG | |
| | GATGCAGATGCCGGTGAAGAATAA | |
| IL-4 | GCCACCATGTTGCTGCTGCCTCTGTTCTTCCTGCTGGCCTGCGCC | 42 |
| Human IL-4 | GGCAATTTTGTGCACGGCCACAAGTGCGACATCACCCTGCAAGAG | |
| Nucleotide | ATCATCAAGACCCTGAACAGCCTGACCGAGCAGAAAACCCTGTGC | |
| (Optimized IL-4 | ACCGAGCTGACCGTGACCGATATCTTTGCCGCCAGCAAGAACACA | |
| with modified | ACCGAGAAAGAGACATTCTGCAGAGCCGCCACCGTGCTGAGACAG | |
| SP) | TTCTACAGCCACCACGAGAAGGACACCAGATGCCTGGGAGCTACA | |
| | GCCCAGCAGTTCCACAGACACAAGCAGCTGATCCGGTTCCTGAAG | |
| | CGGCTGGACAGAAATCTGTGGGGACTCGCCGGCCTGAATAGCTGC | |
| | CCTGTGAAAGAGGCCAACCAGTCTACCCTGGAAAACTTCCTGGAA | |
| | CGGCTGAAAACCATCATGCGCGAGAAGTACAGCAAGTGCAGCAGC | |
| | TGA | |
| IL-4 | MLLLPLFFLLACAGNFVHGHKCDITLQEIIKTLNSLTEQKTLCTE | 43 |
| Human IL-4 | LTVTDIFAASKNTTEKETFCRAATVLRQFYSHHEKDTRCLGATAQ | |
| with modified | QFHRHKQLIRFLKRLDRNLWGLAGLNSCPVKEANQSTLENFLERL | |
| SP | KTIMREKYSKCSS | |
| Underlined: | | |
| signal sequence | | |
| A modified | MLILLLPLLLFKCFCDFLK | 44 |
| signal peptide of | | |
| IGF-1 | | |
| A modified | ATGCTGATTCTGCTGCTGCCCCTGCTGCTGTTCAAGTGCTTCTGCGACT | 45 |
| signal peptide of | TCCTGAAA | |
| IGF-1-coding | | |
| sequence | | |
| A modified | MLFYLALCLLTFTSSATA | 46 |
| IGF-1 pro | | |
| domain | | |
| A modified | ATGCTGTTCTATCTGGCCCTGTGCCTGCTGACCTTTACCAGCTCTGCTACCGCC | 47 |
| IGF-1 pro | | |
| domain-coding | | |
| sequence | | |
| IGF-1 pro | VKMHTMSSSH | 48 |
| domain | | |
| sequence that is | | |
| deleted | | |
| WT IGF-1 | MGKISSLPTQLFKCCFCDFLK | 49 |
| signal peptide | | |
| WT IGF-1 | ATGGGAAAAATCAGCAGTCTTCCAACCCAATTATTTAAGTGCTGCTTTT | 50 |
| signal peptide | GTGATTTCTTGAAG | |
| coding sequence | | |
| A modified | MTILFLTMVISYFGCMKA | 51 |
| signal peptide of | | |
| IGF-1 | | |
| A modified | ATGACCATCCTGTTTCTGACAATGGTCATCAGCTACTTCGGCTGCATGA | 52 |
| signal peptide of | AGGCC | |
| IGF-1-coding | | |
| sequence | | |
| WT IL-4 signal | MGLTSQLLPPLFFLLACAGNFVHG | 53 |
| peptide | | |
| WT IL-4 signal | ATGGGTCTCACCTCCCAACTGCTTCCCCCTCTGTTCTTCCTGCTAGCAT | 54 |
| peptide coding | GTGCCGGCAACTTTGTCCACGGA | |
| sequence | | |
| A modified | MLLLPLFFLLACAGNFVHG | 55 |
| signal peptide of | | |
| IL-4 | | |
| A modified | ATGTTGCTGCTGCCTCTGTTCTTCCTGCTGGCCTGCGCCGGCAATTTTG | 56 |
| signal peptide of | TGCACGGC | |
| IL-4-coding | | |
| sequence | | |
| IL-8 siRNA | CAAGGAAGTGCTAAAGAA | 57 |
| sense strand | | |
| (Cpd.1) | | |
| IL-8 siRNA | CAAGGAGTGCTAAAGAA | 58 |
| sense strand | | |
| (Cpd.2-1) | | |
| IL-8 siRNA | GAGAGTGATTGAGAGTGG | 59 |
| sense strand | | |
| (Cpd.2-2) | | |
| IL-8 siRNA | GAGAGCTCTGTCTGGACC | 60 |
| sense strand | | |
| (Cpd.2-3) | | |
| IL-1beta siRNA | GAAAGATGATAAGCCCACTCT | 61 |
| sense strand | | |
| (Cpd.3-1) | | |
| IL-1beta siRNA | GGTGATGTCTGGTCCATATGA | 62 |
| sense strand | | |
| (Cpd.3-2) | | |
| IL-1beta siRNA | GATGATAAGCCCACTCTA | 63 |
| sense strand | | |
| (Cpd.3-3) | | |
| TNF-alpha | GGCGTGGAGCTGAGAGATAA | 64 |
| siRNA sense | | |
| strand (Cpd.4-1, | | |
| Cpd.5-1, Cpd.6-1, | | |
| Cpd.7 and | | |
| Cpd.8) | | |
| TNF-alpha | GGGCCTGTACCTCATCTACT | 65 |
| siRNA sense | | |
| strand (Cpd.4-2, | | |
| Cpd.5-2 and | | |
| Cpd.6-2) | | |
| TNF-alpha | GGTATGAGCCCATCTATCT | 66 |
| siRNA sense | | |
| strand (Cpd.4-3, | | |
| Cpd.5-3 and | | |
| Cpd.6-3) | | |
| IL-17 siRNA | GCAATGAGGACCCTGAGAGAT | 67 |
| sense strand | | |
| (Cpd.4-1) | | |
| IL-17 siRNA | GCTGATGGGAACGTGGACTA | 68 |
| sense strand | | |
| (Cpd.4-2) | | |
| IL-17 siRNA | GGTCCTCAGATTACTACAA | 69 |
| sense strand | | |
| (Cpd.4-3) | | |
| Turbo GFP | AACAAGATGAAGAGCACCAA | 70 |
| siRNA sense | | |
| strand (Cpd.9, | | |
| Cpd.10-1, | | |
| Cpd.10-2, | | |
| Cpd.11-1, | | |
| Cpd.11-2, | | |
| Cpd.11-3) | | |
| IL-8 siRNA | TTCTTTAGCACTTCCTTG | 71 |
| anti-sense strand | | |
| (Cpd.1) | | |
| IL-8 siRNA | TTCTTTAGCACTCCTTG | 72 |
| anti-sense strand | | |
| (Cpd.2-1) | | |
| IL-8 siRNA | CCACTCTCAATCACTCTC | 73 |
| anti-sense strand | | |
| (Cpd.2-2) | | |
| IL-8 siRNA | GGTCCAGACAGAGCTCTC | 74 |
| anti-sense strand | | |
| (Cpd.2-3) | | |
| IL-1beta siRNA | AGAGTGGGCTTATCATCTTTC | 75 |
| anti-sense strand | | |
| (Cpd.3-1) | | |
| IL-1beta siRNA | TCATATGGACCAGACATCACC | 76 |
| anti-sense strand | | |
| (Cpd.3-2) | | |
| IL-1beta siRNA | TAGAGTGGGCTTATCATC | 77 |
| anti-sense strand | | |
| (Cpd.3-3) | | |
| TNF-alpha | TTATCTCTCAGCTCCACGCC | 78 |
| siRNA anti- | | |
| sense strand | | |
| (Cpd.4-1, | | |
| Cpd.5-1, Cpd.6-1, | | |
| Cpd.7 and | | |
| Cpd.8) | | |
| TNF-alpha | AGTAGATGAGGTACAGGCCC | 79 |
| siRNA anti- | | |
| sense strand | | |
| (Cpd.4-2, | | |
| Cpd.5-2 and | | |
| Cpd.6-2) | | |
| TNF-alpha | AGATAGATGGGCTCATACC | 80 |
| siRNA anti- | | |
| sense strand | | |
| (Cpd.4-3, | | |
| Cpd.5-3 and | | |
| Cpd.6-3) | | |
| IL-17 siRNA | ATCTCTCAGGGTCCTCATTGC | 81 |
| anti-sense strand | | |
| (Cpd.4-1) | | |
| IL-17 siRNA | TAGTCCACGTTCCCATCAGC | 82 |
| anti-sense strand | | |
| (Cpd.4-2) | | |
| IL-17 siRNA | TTGTAGTAATCTGAGGACC | 83 |
| anti-sense strand | | |
| (Cpd.4-3) | | |
| Turbo GFP | TTGGTGCTCTTCATCTTGTTG | 84 |
| siRNA anti- | | |
| sense strand | | |
| (Cpd.9, Cpd.10-1, | | |
| Cpd.10-2, | | |
| Cpd.11-1, | | |
| Cpd.11-2, | | |
| Cpd.11-3) | | |
| A2-linker | ACAACAA | 85 |
| Linker | ATAGTGAGTCGTATTA | 86 |
| Linker | ATCCCTACGTACCAACAA | 87 |
| Linker | ACGTACCAACAA | 88 |
| Linker | TCCC | 89 |
| Linker | ACAACAATCCC | 90 |
| Linker | CAACAA | 91 |
| Linker | ATAGTGAGTCGTATTATCCC | 92 |
| Linker | ATAGTGAGTCGTATTAACAACAATCCC | 93 |
| Linker | ATAGTGAGTCGTATTAACAACAA | 94 |
| Linker | ATAGTGAGTCGTATTAATCCCTACGTACCAACAA | 95 |
| tRNA linker | AACAAAGCACCAGTGGTCTAGTGGTAGAATAGTACCCTGCCACGGTACA | 96 |
| | GACCCGGGTTCGATTCCCGGCTGGTGCA | |
| Compound 1 | AUAGUGAGUCGUAUUAACGUACCAACAACAAGGAAGUGCUAAAGAAACUUGUUC | 97 |
| unmodified | UUUAGCACUUCCUUGUUUAUCUUAGAGGCAUAUCCCUGCCACCAUGACCAUCCU | |
| RNA sequence | GUUUCUGACAAUGGUCAUCAGCUACUUCGGCUGCAUGAAGGCCGUGAAGAUGCA | |
| Bold = Sense siRNA | CACCAUGAGCAGCAGCCACCUGUUCUAUCUGGCCCUGUGCCUGCUGACCUUUAC | |
| strand | CAGCUCUGCUACCGCCGGACCUGAGACACUUUGUGGCGCUGAACUGGUGGACGC | |
| Bold and Italics = | CCUGCAGUUUGUGUGUGGCGACAGAGGCUUCUACUUCAACAAGCCCACAGGCUA | |
| anti-Sense siRNA | CGGCAGCAGCUCUAGAAGGGCUCCUCAGACCGGAAUCGUGGACGAGUGCUGCUU | |
| strand | CAGAAGCUGCGACCUGCGGCGGCUGGAAAUGUAUUGUGCCCCUCUGAAGCCUGC | |
| Underline = Signal | CAAGAGCGCCUAAUUUAUCUUAGAGGCAUAUCCCU | |
| peptide | | |
| Italics = Kozak | | |
| sequence | | |
| Compound 2 | AUAGUGAGUCGUAUUAACGUACCAACAACAAGGAGUGCUAAAGAAACUUGUUCU | 98 |
| unmodified | UUAGCACUCCUUGUUUAUCUUAGAGGCAUAUCCCUACGUACCAACAAGAGAGUG | |
| RNA sequence | AUUGAGAGUGGACUUGCCACUCUCAAUCACUCUCUUUAUCUUAGAGGCAUAUCC | |
| Bold = Sense siRNA | CUACGUACCAACAAGAGAGCUCUGUCUGGACCACUUGGGUCCAGACAGAGCUCU | |
| strand | CUUUAUCUUAGAGGCAUAUCCCUGCCACCAUGACCAUCCUGUUUCUGACAAUGG | |
| Bold and Italics = | UCAUCAGCUACUUCGGCUGCAUGAAGGCCGUGAAGAUGCACACCAUGAGCAGCA | |
| anti-Sense siRNA | GCCACCUGUUCUAUCUGGCCCUGUGCCUGCUGACCUUUACCAGCUCUGCUACCG | |
| strand | CCGGACCUGAGACACUUUGUGGCGCUGAACUGGUGGACGCCCUGCAGUUUGUGU | |
| Underline = Signal | GUGGCGACAGAGGCUUCUACUUCAACAAGCCCACAGGCUACGGCAGCAGCUCUA | |
| peptide | GAAGGGCUCCUCAGACCGGAAUCGUGGACGAGUGCUGCUUCAGAAGCUGCGACC | |
| Italics = Kozak | UGCGGCGGCUGGAAAUGUAUUGUGCCCCUCUGAAGCCUGCCAAGAGCGCCUAAU | |
| sequence | UUAUCUUAGAGGCAUAUCCCU | |
| Compound 3 | AUAGUGAGUCGUAUUAACGUACCAACAAGAAAGAUGAUAAGCCCACUCUACUUG | 99 |
| unmodified | AGAGUGGGCUUAUCAUCUUUCUUUAUCUUAGAGGCAUAUCCCUACGUACCAACA | |
| RNA sequence | AGGUGAUGUCUGGUCCAUAUGAACUUGUCAUAUGGACCAGACAUCACCUUUAUC | |
| Bold = Sense siRNA | UUAGAGGCAUAUCCCUACGUACCAACAAGAUGAUAAGCCCACUCUAACUUGUAG | |
| strand | AGUGGGCUUAUCAUCUUUAUCUUAGAGGCAUAUCCCUGCCACCAUGACCAUCCU | |
| Bold and Italics = | GUUUCUGACAAUGGUCAUCAGCUACUUCGGCUGCAUGAAGGCCGUGAAGAUGCA | |
| anti-Sense siRNA | CACCAUGAGCAGCAGCCACCUGUUCUAUCUGGCCCUGUGCCUGCUGACCUUUAC | |
| strand | CAGCUCUGCUACCGCCGGACCUGAGACACUUUGUGGCGCUGAACUGGUGGACGC | |
| Underline = Signal | CCUGCAGUUUGUGUGUGGCGACAGAGGCUUCUACUUCAACAAGCCCACAGGCUA | |
| peptide | CGGCAGCAGCUCUAGAAGGGCUCCUCAGACCGGAAUCGUGGACGAGUGCUGCUU | |
| Italics = Kozak | CAGAAGCUGCGACCUGCGGCGGCUGGAAAUGUAUUGUGCCCCUCUGAAGCCUGC | |
| sequence | CAAGAGCGCCUAAUUUAUCUUAGAGGCAUAUCCCU | |
| Compound 4 | AUAGUGAGUCGUAUUAACGUACCAACAAGGCGUGGAGCUGAGAGAUAAACUUGU | 100 |
| unmodified | UAUCUCUCAGCUCCACGCCUUUAUCUUAGAGGCAUAUCCCUACGUACCAACAAG | |
| RNA sequence | GGCCUGUACCUCAUCUACUACUUGAGUAGAUGAGGUACAGGCCCUUUAUCUUAG | |
| Bold = Sense siRNA | AGGCAUAUCCCUACGUACCAACAAGGUAUGAGCCCAUCUAUCUACUUGAGAUAG | |
| strand | AUGGGCUCAUACCUUUAUCUUAGAGGCAUAUCCCUACGUACCAACAAGCAAUGA | |
| Bold and Italics = | GGACCCUGAGAGAUACUUGAUCUCUCAGGGUCCUCAUUGCUUUAUCUUAGAGGC | |
| anti-Sense siRNA | AUAUCCCUACGUACCAACAAGCUGAUGGGAACGUGGACUAACUUGUAGUCCACG | |
| strand | UUCCCAUCAGCUUUAUCUUAGAGGCAUAUCCCUACGUACCAACAAGGUCCUCAG | |
| Underline = Signal | AUUACUACAAACUUGUUGUAGUAAUCUGAGGACCUUUAUCUUAGAGGCAUAUCC | |
| peptide | CUGCCACCAUGGGACUGACAUCUCAACUGCUGCCUCCACUGUUCUUUCUGCUGG | |
| Italics = Kozak | CCUGCGCCGGCAAUUUUGUGCACGGCCACAAGUGCGACAUCACCCUGCAAGAGA | |
| sequence | UCAUCAAGACCCUGAACAGCCUGACCGAGCAGAAAACCCUGUGCACCGAGCUGA | |
| | CCGUGACCGAUAUCUUUGCCGCCAGCAAGAACACAACCGAGAAAGAGACAUUCU | |
| | GCAGAGCCGCCACCGUGCUGAGACAGUUCUACAGCCACCACGAGAAGGACACCA | |
| | GAUGCCUGGGAGCUACAGCCCAGCAGUUCCACAGACACAAGCAGCUGAUCCGGU | |
| | UCCUGAAGCGGCUGGACAGAAAUCUGUGGGGACUCGCCGGCCUGAAUAGCUGCC | |
| | CUGUGAAAGAGGCCAACCAGUCUACCCUGGAAAACUUCCUGGAACGGCUGAAAA | |
| | CCAUCAUGCGCGAGAAGUACAGCAAGUGCAGCAGCUGAUUUAUCUUAGAGGCAU | |
| | AUCCCU | |
| Compound 5 | AUAGUGAGUCGUAUUAACGUACCAACAAGGCGUGGAGCUGAGAGAUAAACUUGU | 101 |
| unmodified | UAUCUCUCAGCUCCACGCCUUUAUCUUAGAGGCAUAUCCCUACGUACCAACAAG | |
| RNA sequence | GGCCUGUACCUCAUCUACUACUUGAGUAGAUGAGGUACAGGCCCUUUAUCUUAG | |
| Bold = Sense siRNA | AGGCAUAUCCCUACGUACCAACAAGGUAUGAGCCCAUCUAUCUACUUGAGAUAG | |
| strand | AUGGGCUCAUACCUUUAUCUUAGAGGCAUAUCCCUGCCACCAUGGGACUGACAU | |
| Bold and Italics = | CUCAACUGCUGCCUCCACUGUUCUUUCUGCUGGCCUGCGCCGGCAAUUUUGUGC | |
| anti-Sense siRNA | ACGGCCACAAGUGCGACAUCACCCUGCAAGAGAUCAUCAAGACCCUGAACAGCC | |
| strand | UGACCGAGCAGAAAACCCUGUGCACCGAGCUGACCGUGACCGAUAUCUUUGCCG | |
| Underline = Signal | CCAGCAAGAACACAACCGAGAAAGAGACAUUCUGCAGAGCCGCCACCGUGCUGA | |
| peptide | GACAGUUCUACAGCCACCACGAGAAGGACACCAGAUGCCUGGGAGCUACAGCCC | |
| Italics = Kozak | AGCAGUUCCACAGACACAAGCAGCUGAUCCGGUUCCUGAAGCGGCUGGACAGAA | |
| sequence | AUCUGUGGGGACUCGCCGGCCUGAAUAGCUGCCCUGUGAAAGAGGCCAACCAGU | |
| | CUACCCUGGAAAACUUCCUGGAACGGCUGAAAACCAUCAUGCGCGAGAAGUACA | |
| | GCAAGUGCAGCAGCUGAUUUAUCUUAGAGGCAUAUCCCU | |
| Compound 6 | GCCACCAUGGGACUGACAUCUCAACUGCUGCCUCCACUGUUCUUUCUGCUGGCC | 102 |
| unmodified | UGCGCCGGCAAUUUUGUGCACGGCCACAAGUGCGACAUCACCCUGCAAGAGAUC | |
| RNA sequence | AUCAAGACCCUGAACAGCCUGACCGAGCAGAAAACCCUGUGCACCGAGCUGACC | |
| Bold = Sense siRNA | GUGACCGAUAUCUUUGCCGCCAGCAAGAACACAACCGAGAAAGAGACAUUCUGC | |
| strand | AGAGCCGCCACCGUGCUGAGACAGUUCUACAGCCACCACGAGAAGGACACCAGA | |
| Bold and Italics = | UGCCUGGGAGCUACAGCCCAGCAGUUCCACAGACACAAGCAGCUGAUCCGGUUC | |
| anti-Sense siRNA | CUGAAGCGGCUGGACAGAAAUCUGUGGGGACUCGCCGGCCUGAAUAGCUGCCCU | |
| strand | GUGAAAGAGGCCAACCAGUCUACCCUGGAAAACUUCCUGGAACGGCUGAAAACC | |
| Underline = Signal | AUCAUGCGCGAGAAGUACAGCAAGUGCAGCAGCUGAAUAGUGAGUCGUAUUAAC | |
| peptide | GUACCAACAAGGCGUGGAGCUGAGAGAUAAACUUGUUAUCUCUCAGCUCCACGC | |
| Italics = Kozak | CUUUAUCUUAGAGGCAUAUCCCUACGUACCAACAAGGGCCUGUACCUCAUCUAC | |
| sequence | UACUUGAGUAGAUGAGGUACAGGCCCUUUAUCUUAGAGGCAUAUCCCUACGUAC | |
| | CAACAAGGUAUGAGCCCAUCUAUCUACUUGAGAUAGAUGGGCUCAUACCUUUAU | |
| | CUUAGAGGCAUAUCCCUUUUAUCUUAGAGGCAUAUCCCU | |
| Compound 7 | AUAGUGAGUCGUAUUAACGUACCAACAAGGCGUGGAGCUGAGAGAUAAACUUGU | 103 |
| unmodified | UAUCUCUCAGCUCCACGCCUUUAUCUUAGAGGCAUAUCCCUGCCACCAUGGGCC | |
| RNA sequence | UGACAUCUCAGUUGCUGCCUCCACUGUUCUUUCUGCUGGCCUGCGCCGGCAAUU | |
| Bold = Sense siRNA | UUGUGCACGGCCACAAGUGCGACAUCACCCUGCAAGAGAUCAUCAAGACCCUGA | |
| strand | ACAGCCUGACCGAGCAGAAAACCCUGUGCACCGAGCUGACCGUGACCGAUAUCU | |
| Bold and Italics = | UUGCCGCCAGCAAGAACACAACCGAGAAAGAGACAUUCUGCAGAGCCGCCACCG | |
| anti-Sense siRNA | UGCUGAGACAGUUCUACAGCCACCACGAGAAGGACACCAGAUGCCUGGGAGCUA | |
| strand | CAGCCCAGCAGUUCCACAGACACAAGCAGCUGAUCCGGUUCCUGAAGCGGCUGG | |
| Underline = Signal | ACAGAAAUCUGUGGGGACUCGCCGGCCUGAAUAGCUGCCCUGUGAAAGAGGCCA | |
| peptide | ACCAGUCUACCCUGGAAAACUUCCUGGAACGGCUGAAAACCAUCAUGCGCGAGA | |
| Italics = Kozak | AGUACAGCAAGUGCAGCAGCUAGUUUAUCUUAGAGGCAUAUCCCU | |
| sequence | | |
| Compound 8 | GCCACCAUGGGACUGACAUCUCAACUGCUGCCUCCACUGUUCUUUCUGCUGGCC | 104 |
| unmodified | UGCGCCGGCAAUUUUGUGCACGGCCACAAGUGCGACAUCACCCUGCAAGAGAUC | |
| RNA sequence | AUCAAGACCCUGAACAGCCUGACCGAGCAGAAAACCCUGUGCACCGAGCUGACC | |
| Bold = Sense siRNA | GUGACCGAUAUCUUUGCCGCCAGCAAGAACACAACCGAGAAAGAGACAUUCUGC | |
| strand | AGAGCCGCCACCGUGCUGAGACAGUUCUACAGCCACCACGAGAAGGACACCAGA | |
| Bold and Italics = | UGCCUGGGAGCUACAGCCCAGCAGUUCCACAGACACAAGCAGCUGAUCCGGUUC | |
| anti-Sense siRNA | CUGAAGCGGCUGGACAGAAAUCUGUGGGGACUCGCCGGCCUGAAUAGCUGCCCU | |
| strand | GUGAAAGAGGCCAACCAGUCUACCCUGGAAAACUUCCUGGAACGGCUGAAAACC | |
| Underline = Signal | AUCAUGCGCGAGAAGUACAGCAAGUGCAGCAGCUGAAUAGUGAGUCGUAUUAAC | |
| peptide | GUACCAACAAGGCGUGGAGCUGAGAGAUAAACUUGUUAUCUCUCAGCUCCACGC | |
| Italics = Kozak | CUUUAUCUUAGAGGCAUAUCCCUUUUAUCUUAGAGGCAUAUCCCU | |
| sequence | | |
| Compound 9 | GCCACCAUGGGACUGACAUCUCAACUGCUGCCUCCACUGUUCUUUCUGCUGGCC | 105 |
| unmodified | UGCGCCGGCAAUUUUGUGCACGGCCACAAGUGCGACAUCACCCUGCAAGAGAUC | |
| RNA sequence | AUCAAGACCCUGAACAGCCUGACCGAGCAGAAAACCCUGUGCACCGAGCUGACC | |
| Bold = Sense siRNA | GUGACCGAUAUCUUUGCCGCCAGCAAGAACACAACCGAGAAAGAGACAUUCUGC | |
| strand | AGAGCCGCCACCGUGCUGAGACAGUUCUACAGCCACCACGAGAAGGACACCAGA | |
| Bold and Italics = | UGCCUGGGAGCUACAGCCCAGCAGUUCCACAGACACAAGCAGCUGAUCCGGUUC | |
| anti-Sense siRNA | CUGAAGCGGCUGGACAGAAAUCUGUGGGGACUCGCCGGCCUGAAUAGCUGCCCU | |
| strand | GUGAAAGAGGCCAACCAGUCUACCCUGGAAAACUUCCUGGAACGGCUGAAAACC | |
| Underline = Signal | AUCAUGCGCGAGAAGUACAGCAAGUGCAGCAGCUGAACAACAAGGCGUGGAGCU | |
| peptide | GAGAGAUAAACUUGUUAUCUCUCAGCUCCACGCCACAACAAGGGCCUGUACCUC | |
| Italics = Kozak | AUCUACUACUUGAGUAGAUGAGGUACAGGCCCACAACAAGGUAUGAGCCCAUCU | |
| sequence | AUCUACUUGAGAUAGAUGGGCUCAUACCACAACAAUUUAUCUUAGAGGCAUAUC | |
| | CCU | |
| Compound 10 | GCCACCAUGGGCAAGAUUAGCAGCCUGCCUACACAGCUGUUCAAGUGCUGCUUC | 106 |
| unmodified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Bold = Sense siRNA | CUUUGUGGCGCUGAACUGGUGGACGCCCUGCAGUUUGUGUGUGGCGACAGAGGC | |
| strand | UUCUACUUCAACAAGCCCACAGGCUACGGCAGCAGCUCUAGAAGGGCUCCUCAG | |
| Bold and Italics = | ACCGGAAUCGUGGACGAGUGCUGUUUCAGAAGCUGCGACCUGCGGCGGCUGGAA | |
| anti-Sense siRNA | AUGUAUUGUGCCCCUCUGAAGCCUGCCAAGAGCGCCUAAAUAGUGAGUCGUAUU | |
| strand | AACGUACCAACAACAACAAGAUGAAGAGCACCAAACUUGUUGGUGCUCUUCAUC | |
| Underline = Signal | UUGUUGUUUAUCUUAGAGGCAUAUCCCUUUUAUCUUAGAGGCAUAUCCCU | |
| peptide | | |
| Italics = Kozak | | |
| sequence | | |
| Compound 11 | GCCACCAUGGGCAAGAUUAGCAGCCUGCCUACACAGCUGUUCAAGUGCUGCUUC | 107 |
| unmodified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Bold = Sense siRNA | CUUUGUGGCGCUGAACUGGUGGACGCCCUGCAGUUUGUGUGUGGCGACAGAGGC | |
| strand | UUCUACUUCAACAAGCCCACAGGCUACGGCAGCAGCUCUAGAAGGGCUCCUCAG | |
| Bold and Italics = | ACCGGAAUCGUGGACGAGUGCUGUUUCAGAAGCUGCGACCUGCGGCGGCUGGAA | |
| anti-Sense siRNA | AUGUAUUGUGCCCCUCUGAAGCCUGCCAAGAGCGCCUAAAUAGUGAGUCGUAUU | |
| strand | AACGUACCAACAACAACAAGAUGAAGAGCACCAAACUUGUUGGUGCUCUUCAUC | |
| Underline = Signal | UUGUUGUUUAUCUUAGAGGCAUAUCCCUACGUACCAACAACAACAAGAUGAAGA | |
| peptide | GCACCAAACUUGUUGGUGCUCUUCAUCUUGUUGUUUAUCUUAGAGGCAUAUCCC | |
| Italics = Kozak | UUUUAUCUUAGAGGCAUAUCCCU | |
| sequence | | |
| Compound 12 | GCCACCAUGGGCAAGAUUAGCAGCCUGCCUACACAGCUGUUCAAGUGCUGCUUC | 108 |
| unmodified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Bold = Sense siRNA | CUUUGUGGCGCUGAACUGGUGGACGCCCUGCAGUUUGUGUGUGGCGACAGAGGC | |
| strand | UUCUACUUCAACAAGCCCACAGGCUACGGCAGCAGCUCUAGAAGGGCUCCUCAG | |
| Bold and Italics = | ACCGGAAUCGUGGACGAGUGCUGUUUCAGAAGCUGCGACCUGCGGCGGCUGGAA | |
| anti-Sense siRNA | AUGUAUUGUGCCCCUCUGAAGCCUGCCAAGAGCGCCUAAAUAGUGAGUCGUAUU | |
| strand | AACGUACCAACAACAACAAGAUGAAGAGCACCAAACUUGUUGGUGCUCUUCAUC | |
| Underline = Signal | UUGUUGUUUAUCUUAGAGGCAUAUCCCUACGUACCAACAACAACAAGAUGAAGA | |
| peptide | GCACCAAACUUGUUGGUGCUCUUCAUCUUGUUGUUUAUCUUAGAGGCAUAUCCC | |
| Italics = Kozak | UACGUACCAACAACAACAAGAUGAAGAGCACCAAACUUGUUGGUGCUCUUCAUC | |
| sequence | UUGUUGUUUAUCUUAGAGGCAUAUCCCUUUUAUCUUAGAGGCAUAUCCCU | |
| Compound 1 | AUAGUGAGUCGUAUUAACGUACCAACAACAAGGAAGUGCUAAAGAAACUUGUUC | 109 |
| modified | UUUAGCACUUCCUUGUUUAUCUUAGAGGCAUAUCCCUGCCACCAUGACCAUCCU | |
| Bold = Sense siRNA | GUUUCUGACAAUGGUCAUCAGCUACUUCGGCUGCAUGAAGGCCGUGAAGAUGCA | |
| strand | CACCAUGAGCAGCAGCCACCUGUUCUAUCUGGCCCUGUGCCUGCUGACCUUUAC | |
| Bold and Italics = | CAGCUCUGCUACCGCCGGACCUGAGACACUUUGUGGCGCUGAACUGGUGGACGC | |
| anti-Sense siRNA | CCUGCAGUUUGUGUGUGGCGACAGAGGCUUCUACUUCAACAAGCCCACAGGCUA | |
| strand | CGGCAGCAGCUCUAGAAGGGCUCCUCAGACCGGAAUCGUGGACGAGUGCUGCUU | |
| Underline = Signal | CAGAAGCUGCGACCUGCGGCGGCUGGAAAUGUAUUGUGCCCCUCUGAAGCCUGC | |
| peptide | CAAGAGCGCCUAAUUUAUCUUAGAGGCAUAUCCCU | |
| Italics = Kozak | (all Us are modified; N¹-methylpseudouridine) | |
| sequence | | |
| RNA sequence | | |
| Bold = Sense siRNA | | |
| strand | | |
| Bold and Italics = | | |
| anti-Sense siRNA | | |
| strand | | |
| Underline = Signal | | |
| peptide | | |
| Italics = Kozak | | |
| sequence | | |
| Compound 2 | AUAGUGAGUCGUAUUAACGUACCAACAACAAGGAGUGCUAAAGAAACUUGUUCU | 110 |
| modified | UUAGCACUCCUUGUUUAUCUUAGAGGCAUAUCCCUACGUACCAACAAGAGAGUG | |
| RNA sequence | AUUGAGAGUGGACUUGCCACUCUCAAUCACUCUCUUUAUCUUAGAGGCAUAUCC | |
| Bold = Sense siRNA | CUACGUACCAACAAGAGAGCUCUGUCUGGACCACUUGGGUCCAGACAGAGCUCU | |
| strand | CUUUAUCUUAGAGGCAUAUCCCUGCCACCAUGACCAUCCUGUUUCUGACAAUGG | |
| Bold and Italics = | UCAUCAGCUACUUCGGCUGCAUGAAGGCCGUGAAGAUGCACACCAUGAGCAGCA | |
| anti-Sense siRNA | GCCACCUGUUCUAUCUGGCCCUGUGCCUGCUGACCUUUACCAGCUCUGCUACCG | |
| strand | CCGGACCUGAGACACUUUGUGGCGCUGAACUGGUGGACGCCCUGCAGUUUGUGU | |
| Underline = Signal | GUGGCGACAGAGGCUUCUACUUCAACAAGCCCACAGGCUACGGCAGCAGCUCUA | |
| peptide | GAAGGGCUCCUCAGACCGGAAUCGUGGACGAGUGCUGCUUCAGAAGCUGCGACC | |
| Italics = Kozak | UGCGGCGGCUGGAAAUGUAUUGUGCCCCUCUGAAGCCUGCCAAGAGCGCCUAAU | |
| sequence | UUAUCUUAGAGGCAUAUCCCU | |
| | (all Us are modified; N¹-methylpseudouridine) | |
| Compound 3 | AUAGUGAGUCGUAUUAACGUACCAACAAGAAAGAUGAUAAGCCCACUCUACUUG | 111 |
| modified | AGAGUGGGCUUAUCAUCUUUCUUUAUCUUAGAGGCAUAUCCCUACGUACCAACA | |
| RNA sequence | AGGUGAUGUCUGGUCCAUAUGAACUUGUCAUAUGGACCAGACAUCACCUUUAUC | |
| Bold = Sense siRNA | UUAGAGGCAUAUCCCUACGUACCAACAAGAUGAUAAGCCCACUCUAACUUGUAG | |
| strand | AGUGGGCUUAUCAUCUUUAUCUUAGAGGCAUAUCCCUGCCACCAUGACCAUCCU | |
| Bold and Italics = | GUUUCUGACAAUGGUCAUCAGCUACUUCGGCUGCAUGAAGGCCGUGAAGAUGCA | |
| anti-Sense siRNA | CACCAUGAGCAGCAGCCACCUGUUCUAUCUGGCCCUGUGCCUGCUGACCUUUAC | |
| strand | CAGCUCUGCUACCGCCGGACCUGAGACACUUUGUGGCGCUGAACUGGUGGACGC | |
| Underline = Signal | CCUGCAGUUUGUGUGUGGCGACAGAGGCUUCUACUUCAACAAGCCCACAGGCUA | |
| peptide | CGGCAGCAGCUCUAGAAGGGCUCCUCAGACCGGAAUCGUGGACGAGUGCUGCUU | |
| Italics = Kozak | CAGAAGCUGCGACCUGCGGCGGCUGGAAAUGUAUUGUGCCCCUCUGAAGCCUGC | |
| sequence | CAAGAGCGCCUAAUUUAUCUUAGAGGCAUAUCCCU | |
| | (all Us are modified; N¹-methylpseudouridine) | |
| Compound 4 | AUAGUGAGUCGUAUUAACGUACCAACAAGGCGUGGAGCUGAGAGAUAAACUUGU | 112 |
| modified | UAUCUCUCAGCUCCACGCCUUUAUCUUAGAGGCAUAUCCCUACGUACCAACAAG | |
| RNA sequence | GGCCUGUACCUCAUCUACUACUUGAGUAGAUGAGGUACAGGCCCUUUAUCUUAG | |
| Bold = Sense siRNA | AGGCAUAUCCCUACGUACCAACAAGGUAUGAGCCCAUCUAUCUACUUGAGAUAG | |
| strand | AUGGGCUCAUACCUUUAUCUUAGAGGCAUAUCCCUACGUACCAACAAGCAAUGA | |
| Bold and Italics = | GGACCCUGAGAGAUACUUGAUCUCUCAGGGUCCUCAUUGCUUUAUCUUAGAGGC | |
| anti-Sense siRNA | AUAUCCCUACGUACCAACAAGCUGAUGGGAACGUGGACUAACUUGUAGUCCACG | |
| strand | UUCCCAUCAGCUUUAUCUUAGAGGCAUAUCCCUACGUACCAACAAGGUCCUCAG | |
| Underline = Signal | AUUACUACAAACUUGUUGUAGUAAUCUGAGGACCUUUAUCUUAGAGGCAUAUCC | |
| peptide | CUGCCACCAUGGGACUGACAUCUCAACUGCUGCCUCCACUGUUCUUUCUGCUGG | |
| Italics = Kozak | CCUGCGCCGGCAAUUUUGUGCACGGCCACAAGUGCGACAUCACCCUGCAAGAGA | |
| sequence | UCAUCAAGACCCUGAACAGCCUGACCGAGCAGAAAACCCUGUGCACCGAGCUGA | |
| | CCGUGACCGAUAUCUUUGCCGCCAGCAAGAACACAACCGAGAAAGAGACAUUCU | |
| | GCAGAGCCGCCACCGUGCUGAGACAGUUCUACAGCCACCACGAGAAGGACACCA | |
| | GAUGCCUGGGAGCUACAGCCCAGCAGUUCCACAGACACAAGCAGCUGAUCCGGU | |
| | UCCUGAAGCGGCUGGACAGAAAUCUGUGGGGACUCGCCGGCCUGAAUAGCUGCC | |
| | CUGUGAAAGAGGCCAACCAGUCUACCCUGGAAAACUUCCUGGAACGGCUGAAAA | |
| | CCAUCAUGCGCGAGAAGUACAGCAAGUGCAGCAGCUGAUUUAUCUUAGAGGCAU | |
| | AUCCCU | |
| | (all Us are modified; N¹-methylpseudouridine) | |
| Compound 5 | AUAGUGAGUCGUAUUAACGUACCAACAAGGCGUGGAGCUGAGAGAUAAACUUGU | 113 |
| modified | UAUCUCUCAGCUCCACGCCUUUAUCUUAGAGGCAUAUCCCUACGUACCAACAAG | |
| RNA sequence | GGCCUGUACCUCAUCUACUACUUGAGUAGAUGAGGUACAGGCCCUUUAUCUUAG | |
| Bold = Sense siRNA | AGGCAUAUCCCUACGUACCAACAAGGUAUGAGCCCAUCUAUCUACUUGAGAUAG | |
| strand | AUGGGCUCAUACCUUUAUCUUAGAGGCAUAUCCCUGCCACCAUGGGACUGACAU | |
| Bold and Italics = | CUCAACUGCUGCCUCCACUGUUCUUUCUGCUGGCCUGCGCCGGCAAUUUUGUGC | |
| anti-Sense siRNA | ACGGCCACAAGUGCGACAUCACCCUGCAAGAGAUCAUCAAGACCCUGAACAGCC | |
| strand | UGACCGAGCAGAAAACCCUGUGCACCGAGCUGACCGUGACCGAUAUCUUUGCCG | |
| Underline = Signal | CCAGCAAGAACACAACCGAGAAAGAGACAUUCUGCAGAGCCGCCACCGUGCUGA | |
| peptide | GACAGUUCUACAGCCACCACGAGAAGGACACCAGAUGCCUGGGAGCUACAGCCC | |
| Italics = Kozak | AGCAGUUCCACAGACACAAGCAGCUGAUCCGGUUCCUGAAGCGGCUGGACAGAA | |
| sequence | AUCUGUGGGGACUCGCCGGCCUGAAUAGCUGCCCUGUGAAAGAGGCCAACCAGU | |
| | CUACCCUGGAAAACUUCCUGGAACGGCUGAAAACCAUCAUGCGCGAGAAGUACA | |
| | GCAAGUGCAGCAGCUGAUUUAUCUUAGAGGCAUAUCCCU | |
| | (all Us are modified; N¹-methylpseudouridine) | |
| Compound 6 | GCCACCAUGGGACUGACAUCUCAACUGCUGCCUCCACUGUUCUUUCUGCUGGCC | 114 |
| modified | UGCGCCGGCAAUUUUGUGCACGGCCACAAGUGCGACAUCACCCUGCAAGAGAUC | |
| RNA sequence | AUCAAGACCCUGAACAGCCUGACCGAGCAGAAAACCCUGUGCACCGAGCUGACC | |
| Bold = Sense siRNA | GUGACCGAUAUCUUUGCCGCCAGCAAGAACACAACCGAGAAAGAGACAUUCUGC | |
| strand | AGAGCCGCCACCGUGCUGAGACAGUUCUACAGCCACCACGAGAAGGACACCAGA | |
| Bold and Italics = | UGCCUGGGAGCUACAGCCCAGCAGUUCCACAGACACAAGCAGCUGAUCCGGUUC | |
| anti-Sense siRNA | CUGAAGCGGCUGGACAGAAAUCUGUGGGGACUCGCCGGCCUGAAUAGCUGCCCU | |
| strand | GUGAAAGAGGCCAACCAGUCUACCCUGGAAAACUUCCUGGAACGGCUGAAAACC | |
| Underline = Signal | AUCAUGCGCGAGAAGUACAGCAAGUGCAGCAGCUGAAUAGUGAGUCGUAUUAAC | |
| peptide | GUACCAACAAGGCGUGGAGCUGAGAGAUAAACUUGUUAUCUCUCAGCUCCACGC | |
| Italics = Kozak | CUUUAUCUUAGAGGCAUAUCCCUACGUACCAACAAGGGCCUGUACCUCAUCUAC | |
| sequence | UACUUGAGUAGAUGAGGUACAGGCCCUUUAUCUUAGAGGCAUAUCCCUACGUAC | |
| | CAACAAGGUAUGAGCCCAUCUAUCUACUUGAGAUAGAUGGGCUCAUACCUUUAU | |
| | CUUAGAGGCAUAUCCCUUUUAUCUUAGAGGCAUAUCCCU | |
| | (all Us are modified; N¹-methylpseudouridine) | |
| Compound 7 | AUAGUGAGUCGUAUUAACGUACCAACAAGGCGUGGAGCUGAGAGAUAAACUUGU | 115 |
| modified | UAUCUCUCAGCUCCACGCCUUUAUCUUAGAGGCAUAUCCCUGCCACCAUGGGCC | |
| RNA sequence | UGACAUCUCAGUUGCUGCCUCCACUGUUCUUUCUGCUGGCCUGCGCCGGCAAUU | |
| Bold = Sense siRNA | UUGUGCACGGCCACAAGUGCGACAUCACCCUGCAAGAGAUCAUCAAGACCCUGA | |
| strand | ACAGCCUGACCGAGCAGAAAACCCUGUGCACCGAGCUGACCGUGACCGAUAUCU | |
| Bold and Italics = | UUGCCGCCAGCAAGAACACAACCGAGAAAGAGACAUUCUGCAGAGCCGCCACCG | |
| anti-Sense siRNA | UGCUGAGACAGUUCUACAGCCACCACGAGAAGGACACCAGAUGCCUGGGAGCUA | |
| strand | CAGCCCAGCAGUUCCACAGACACAAGCAGCUGAUCCGGUUCCUGAAGCGGCUGG | |
| Underline = Signal | ACAGAAAUCUGUGGGGACUCGCCGGCCUGAAUAGCUGCCCUGUGAAAGAGGCCA | |
| peptide | ACCAGUCUACCCUGGAAAACUUCCUGGAACGGCUGAAAACCAUCAUGCGCGAGA | |
| Italics = Kozak | AGUACAGCAAGUGCAGCAGCUAGUUUAUCUUAGAGGCAUAUCCCU | |
| sequence | (all Us are modified; N¹-methylpseudouridine) | |
| Compound 8 | GCCACCAUGGGACUGACAUCUCAACUGCUGCCUCCACUGUUCUUUCUGCUGGCC | 116 |
| modified | UGCGCCGGCAAUUUUGUGCACGGCCACAAGUGCGACAUCACCCUGCAAGAGAUC | |
| RNA sequence | AUCAAGACCCUGAACAGCCUGACCGAGCAGAAAACCCUGUGCACCGAGCUGACC | |
| Bold = Sense siRNA | GUGACCGAUAUCUUUGCCGCCAGCAAGAACACAACCGAGAAAGAGACAUUCUGC | |
| strand | AGAGCCGCCACCGUGCUGAGACAGUUCUACAGCCACCACGAGAAGGACACCAGA | |
| Bold and Italics = | UGCCUGGGAGCUACAGCCCAGCAGUUCCACAGACACAAGCAGCUGAUCCGGUUC | |
| anti-Sense siRNA | CUGAAGCGGCUGGACAGAAAUCUGUGGGGACUCGCCGGCCUGAAUAGCUGCCCU | |
| strand | GUGAAAGAGGCCAACCAGUCUACCCUGGAAAACUUCCUGGAACGGCUGAAAACC | |
| Underline = Signal | AUCAUGCGCGAGAAGUACAGCAAGUGCAGCAGCUGAAUAGUGAGUCGUAUUAAC | |
| peptide | GUACCAACAAGGCGUGGAGCUGAGAGAUAAACUUGUUAUCUCUCAGCUCCACGC | |
| Italics = Kozak | CUUUAUCUUAGAGGCAUAUCCCUUUUAUCUUAGAGGCAUAUCCCU | |
| sequence | (all Us are modified; N¹-methylpseudouridine) | |
| Compound 9 | GCCACCAUGGGACUGACAUCUCAACUGCUGCCUCCACUGUUCUUUCUGCUGGCC | 117 |
| modified | UGCGCCGGCAAUUUUGUGCACGGCCACAAGUGCGACAUCACCCUGCAAGAGAUC | |
| RNA sequence | AUCAAGACCCUGAACAGCCUGACCGAGCAGAAAACCCUGUGCACCGAGCUGACC | |
| Bold = Sense siRNA | GUGACCGAUAUCUUUGCCGCCAGCAAGAACACAACCGAGAAAGAGACAUUCUGC | |
| strand | AGAGCCGCCACCGUGCUGAGACAGUUCUACAGCCACCACGAGAAGGACACCAGA | |
| Bold and Italics = | UGCCUGGGAGCUACAGCCCAGCAGUUCCACAGACACAAGCAGCUGAUCCGGUUC | |
| anti-Sense siRNA | CUGAAGCGGCUGGACAGAAAUCUGUGGGGACUCGCCGGCCUGAAUAGCUGCCCU | |
| strand | GUGAAAGAGGCCAACCAGUCUACCCUGGAAAACUUCCUGGAACGGCUGAAAACC | |
| Underline = Signal | AUCAUGCGCGAGAAGUACAGCAAGUGCAGCAGCUGAACAACAAGGCGUGGAGCU | |
| peptide | GAGAGAUAAACUUGUUAUCUCUCAGCUCCACGCCACAACAAGGGCCUGUACCUC | |
| Italics = Kozak | AUCUACUACUUGAGUAGAUGAGGUACAGGCCCACAACAAGGUAUGAGCCCAUCU | |
| sequence | AUCUACUUGAGAUAGAUGGGCUCAUACCACAACAAUUUAUCUUAGAGGCAUAUC | |
| | CCU | |
| | (all Us are modified; N¹-methylpseudouridine) | |
| Compound 10 | GCCACCAUGGGCAAGAUUAGCAGCCUGCCUACACAGCUGUUCAAGUGCUGCUUC | 118 |
| modified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Bold = Sense siRNA | CUUUGUGGCGCUGAACUGGUGGACGCCCUGCAGUUUGUGUGUGGCGACAGAGGC | |
| strand | UUCUACUUCAACAAGCCCACAGGCUACGGCAGCAGCUCUAGAAGGGCUCCUCAG | |
| Bold and Italics = | ACCGGAAUCGUGGACGAGUGCUGUUUCAGAAGCUGCGACCUGCGGCGGCUGGAA | |
| anti-Sense siRNA | AUGUAUUGUGCCCCUCUGAAGCCUGCCAAGAGCGCCUAAAUAGUGAGUCGUAUU | |
| strand | AACGUACCAACAACAACAAGAUGAAGAGCACCAAACUUGUUGGUGCUCUUCAUC | |
| Underline = Signal | UUGUUGUUUAUCUUAGAGGCAUAUCCCUUUUAUCUUAGAGGCAUAUCCCU | |
| peptide | (all Us are modified; N¹-methylpseudouridine) | |
| Italics = Kozak | | |
| sequence | | |
| Compound 11 | GCCACCAUGGGCAAGAUUAGCAGCCUGCCUACACAGCUGUUCAAGUGCUGCUUC | 119 |
| modified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Bold = Sense siRNA | CUUUGUGGCGCUGAACUGGUGGACGCCCUGCAGUUUGUGUGUGGCGACAGAGGC | |
| strand | UUCUACUUCAACAAGCCCACAGGCUACGGCAGCAGCUCUAGAAGGGCUCCUCAG | |
| Bold and Italics = | ACCGGAAUCGUGGACGAGUGCUGUUUCAGAAGCUGCGACCUGCGGCGGCUGGAA | |
| anti-Sense siRNA | AUGUAUUGUGCCCCUCUGAAGCCUGCCAAGAGCGCCUAAAUAGUGAGUCGUAUU | |
| strand | AACGUACCAACAACAACAAGAUGAAGAGCACCAAACUUGUUGGUGCUCUUCAUC | |
| Underline = Signal | UUGUUGUUUAUCUUAGAGGCAUAUCCCUACGUACCAACAACAACAAGAUGAAGA | |
| peptide | GCACCAAACUUGUUGGUGCUCUUCAUCUUGUUGUUUAUCUUAGAGGCAUAUCCC | |
| Italics = Kozak | UUUUAUCUUAGAGGCAUAUCCCU | |
| sequence | (all Us are modified; N¹-methylpseudouridine) | |
| Compound 12 | GCCACCAUGGGCAAGAUUAGCAGCCUGCCUACACAGCUGUUCAAGUGCUGCUUC | 120 |
| modified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Bold = Sense siRNA | CUUUGUGGCGCUGAACUGGUGGACGCCCUGCAGUUUGUGUGUGGCGACAGAGGC | |
| strand | UUCUACUUCAACAAGCCCACAGGCUACGGCAGCAGCUCUAGAAGGGCUCCUCAG | |
| Bold and Italics = | ACCGGAAUCGUGGACGAGUGCUGUUUCAGAAGCUGCGACCUGCGGCGGCUGGAA | |
| anti-Sense siRNA | AUGUAUUGUGCCCCUCUGAAGCCUGCCAAGAGCGCCUAAAUAGUGAGUCGUAUU | |
| strand | AACGUACCAACAACAACAAGAUGAAGAGCACCAAACUUGUUGGUGCUCUUCAUC | |
| Underline = Signal | UUGUUGUUUAUCUUAGAGGCAUAUCCCUACGUACCAACAACAACAAGAUGAAGA | |
| peptide | GCACCAAACUUGUUGGUGCUCUUCAUCUUGUUGUUUAUCUUAGAGGCAUAUCCC | |
| Italics = Kozak | UACGUACCAACAACAACAAGAUGAAGAGCACCAAACUUGUUGGUGCUCUUCAUC | |
| sequence | UUGUUGUUUAUCUUAGAGGCAUAUCCCUUUUAUCUUAGAGGCAUAUCCCU | |
| | (all Us are modified; N¹-methylpseudouridine) | |
| Compound 13 | GCCACCAUGGGCAAGAUUAGCAGCCUGCCUACACAGCUGUUCAAGUGCUGCUUC | 121 |
| unmodified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Underline = Signal | CUUUGUGGCGCUGAACUGGUGGACGCCCUGCAGUUUGUGUGUGGCGACAGAGGC | |
| peptide | UUCUACUUCAACAAGCCCACAGGCUACGGCAGCAGCUCUAGAAGGGCUCCUCAG | |
| Italics = Kozak | ACCGGAAUCGUGGACGAGUGCUGUUUCAGAAGCUGCGACCUGCGGCGGCUGGAA | |
| sequence | AUGUAUUGUGCCCCUCUGAAGCCUGCCAAGAGCGCCUAA | |
| Compound 14 | GCCACCAUGUUGCUGCUGCCUCUGUUCUUCCUGCUGGCCUGCGCCGGCAAUUUU | 122 |
| unmodified | GUGCACGGCCACAAGUGCGACAUCACCCUGCAAGAGAUCAUCAAGACCCUGAAC | |
| RNA sequence | AGCCUGACCGAGCAGAAAACCCUGUGCACCGAGCUGACCGUGACCGAUAUCUUU | |
| Underline = Signal | GCCGCCAGCAAGAACACAACCGAGAAAGAGACAUUCUGCAGAGCCGCCACCGUG | |
| peptide | CUGAGACAGUUCUACAGCCACCACGAGAAGGACACCAGAUGCCUGGGAGCUACA | |
| Italics = Kozak | GCCCAGCAGUUCCACAGACACAAGCAGCUGAUCCGGUUCCUGAAGCGGCUGGAC | |
| sequence | AGAAAUCUGUGGGGACUCGCCGGCCUGAAUAGCUGCCCUGUGAAAGAGGCCAAC | |
| | CAGUCUACCCUGGAAAACUUCCUGGAACGGCUGAAAACCAUCAUGCGCGAGAAG | |
| | UACAGCAAGUGCAGCAGCUGA | |
| Compound 13 | GCCACCAUGGGCAAGAUUAGCAGCCUGCCUACACAGCUGUUCAAGUGCUGCUUC | 123 |
| modified | UGCGACUUCCUGAAAGUGAAGAUGCACACCAUGAGCAGCAGCCACCUGUUCUAU | |
| RNA sequence | CUGGCCCUGUGCCUGCUGACCUUUACCAGCUCUGCUACCGCCGGACCUGAGACA | |
| Underline = Signal | CUUUGUGGCGCUGAACUGGUGGACGCCCUGCAGUUUGUGUGUGGCGACAGAGGC | |
| peptide | UUCUACUUCAACAAGCCCACAGGCUACGGCAGCAGCUCUAGAAGGGCUCCUCAG | |
| Italics = Kozak | ACCGGAAUCGUGGACGAGUGCUGUUUCAGAAGCUGCGACCUGCGGCGGCUGGAA | |
| sequence | AUGUAUUGUGCCCCUCUGAAGCCUGCCAAGAGCGCCUAA | |
| | (all Us are modified; N¹-methylpseudouridine) | |
| Compound 14 | GCCACCAUGUUGCUGCUGCCUCUGUUCUUCCUGCUGGCCUGCGCCGGCAAUUUU | 124 |
| modified | GUGCACGGCCACAAGUGCGACAUCACCCUGCAAGAGAUCAUCAAGACCCUGAAC | |
| RNA sequence | AGCCUGACCGAGCAGAAAACCCUGUGCACCGAGCUGACCGUGACCGAUAUCUUU | |
| Underline = Signal | GCCGCCAGCAAGAACACAACCGAGAAAGAGACAUUCUGCAGAGCCGCCACCGUG | |
| peptide | CUGAGACAGUUCUACAGCCACCACGAGAAGGACACCAGAUGCCUGGGAGCUACA | |
| Italics = Kozak | GCCCAGCAGUUCCACAGACACAAGCAGCUGAUCCGGUUCCUGAAGCGGCUGGAC | |
| sequence | AGAAAUCUGUGGGGACUCGCCGGCCUGAAUAGCUGCCCUGUGAAAGAGGCCAAC | |
| | CAGUCUACCCUGGAAAACUUCCUGGAACGGCUGAAAACCAUCAUGCGCGAGAAG | |
| | UACAGCAAGUGCAGCAGCUGA | |
| | (all Us are modified; N¹-methylpseudouridine) | |
| IGF-1 | GCCACCATGGGCAAGATTAGCAGCCTGCCTACACAGCTGTTCAAGTGCTGCTTC | 125 |
| Human IGF-1 | TGCGACTTCCTGAAAGTGAAGATGCACACCATGAGCAGCAGCCACCTGTTCTAT | |
| Nucleotide | CTGGCCCTGTGCCTGCTGACCTTTACCAGCTCTGCTACCGCCGGACCTGAGACA | |
| (Optimized IGF-1 | CTTTGTGGCGCTGAACTGGTGGACGCCCTGCAGTTTGTGTGTGGCGACAGAGGC | |
| with endogenous | TTCTACTTCAACAAGCCCACAGGCTACGGCAGCAGCTCTAGAAGGGCTCCTCAG | |
| SP and without E- | ACCGGAATCGTGGACGAGTGCTGTTTCAGAAGCTGCGACCTGCGGCGGCTGGAA | |
| peptide) | ATGTATTGTGCCCCTCTGAAGCCTGCCAAGAGCGCCTAA | |
| IGF-1 | MGKISSLPTQLFKCCFCDFLKVKMHTMSSSHLFYLALCLLTFTSSATAG | 126 |
| Human IGF-1 | PETLCGAELVDALQFVCGDRGFYFNKPTGYGSSSRRAPQTGIVDECCFR | |
| amino acid | SCDLRRLEMYCAPLKPAKSA | |
| (Optimized IGF-1 | | |
| with BDNF SP | | |
| and without E- | | |
| peptide) | | |
| Underlined: signal | | |
| sequence | | |

What is claimed is:

1. A composition comprising a recombinant RNA construct comprising:
(i) a first RNA sequence encoding a gene of interest, and
(ii) a second RNA sequence comprising at least two genetic elements that modulate expression of one or more target RNAs,
wherein contacting a human cell with the recombinant RNA construct results in an immune response that is lower than the immune response of the human cell contacted with a corresponding recombinant RNA construct comprising the first RNA sequence of (i) and a corresponding second RNA sequence of (ii) comprising at most one of the at least two genetic elements.

2. The composition of claim 1, wherein the recombinant RNA construct comprises one or more uridines.

3. The composition of claim 1 or 2, wherein the recombinant RNA construct does not comprise a modified uridine.

4. A composition comprising a recombinant RNA construct comprising:
(i) a first RNA sequence encoding a gene of interest, and
(ii) a second RNA sequence comprising at least two genetic elements that modulate expression of one or more target RNAs,
wherein the recombinant RNA construct does not comprise a nucleotide variant.

5. A composition comprising a recombinant RNA construct comprising:
(i) a first RNA sequence encoding a gene of interest, and
(ii) a second RNA sequence comprising at least two genetic elements that modulate expression of one or more target RNAs,
wherein the recombinant RNA construct does not comprise a modified uridine.

6. A composition comprising a recombinant RNA construct comprising:
(i) a first RNA sequence encoding a gene of interest, and
(ii) a second RNA sequence comprising at least two genetic elements that modulate expression of one or more target RNAs,
wherein the recombinant RNA construct does not comprise a N1-methylpseudouridine.

7. A composition comprising a recombinant RNA construct comprising:
(i) a first RNA sequence encoding a gene of interest, and
(ii) a second RNA sequence comprising at least two genetic elements that modulate expression of one or more target RNAs,
wherein the recombinant RNA construct comprises solely unmodified nucleotides or natural nucleotides.

8. A composition comprising a recombinant RNA construct comprising:
(i) a first RNA sequence encoding a gene of interest, and
(ii) a second RNA sequence comprising at least two genetic elements that modulate expression of one or more target RNAs,
wherein the recombinant RNA construct comprises uridines, wherein:
(a) all uridines comprised by the recombinant RNA constructs are unmodified or natural nucleotide(s); or
(b) at least one of the uridines comprised by the recombinant RNA constructs is an unmodified uridine.

9. The composition of claim 4, wherein the nucleotide variant comprises a modified uridine.

10. The composition of any one of claims 3, 5, and 9, wherein the modified uridine comprises a N1-methylpseudouridine.

11. The composition of any one of claims 1-3, wherein the corresponding recombinant RNA construct does not comprise any of the genetic elements that modulate expression of one or more target RNAs.

12. The composition of any one of the preceding claims, wherein the second RNA sequence comprises at least three genetic elements that modulate expression of one or more target RNAs.

13. The composition of any one of the preceding claims, wherein the second RNA sequence comprises at least six genetic elements that modulate expression of one or more target RNAs.

14. The composition of any one of the preceding claims, wherein the first RNA sequence is a messenger RNA (mRNA) sequence.

15. The composition of any one of the preceding claims, wherein each of the at least two genetic elements of the second RNA sequence comprises a secondary structure.

16. The composition of any one of the preceding claims, wherein each of the at least two genetic elements of the second RNA sequence comprises a hairpin structure or a loop structure.

17. The composition of any one of the preceding claims, wherein each of the at least two genetic elements of the second RNA sequence is a short or small hairpin RNA (shRNA).

18. The composition of any one of the preceding claims, wherein each of the at least two genetic elements of the second RNA sequence is processed or cleaved by an intracellular protein.

19. The composition of any one of the preceding claims, wherein each of the at least two genetic elements of the second RNA sequence is processed or cleaved by an endogenous protein of a cell.

20. The composition of any one of the preceding claims, wherein each of the at least two genetic elements of the second RNA sequence is processed or cleaved by an endogenous DICER.

21. The composition of any one of the preceding claims, wherein each of the at least two genetic elements of the second RNA sequence comprises a small interfering RNA (siRNA).

22. The composition of any one of the preceding claims, wherein each of the at least two genetic elements of the second RNA sequence is capable of binding to the one or more target RNAs.

23. The composition of any one of claims 1-3 or 11-22, wherein the immune response is a human Toll-Like Receptor 7 (TLR7) immune response, an interferon alpha/beta (IFNα/β) immune response, a human Toll-Like Receptor 3 (TLR3) immune response, a human Toll-Like Receptor 8 (TLR8) immune response, or any combination thereof.

24. The composition of any one of claims 1-3 or 11-23, wherein contacting the human cell with the recombinant RNA construct results in an immune response that is lower than the immune response of the human cell contacted with the corresponding recombinant RNA construct according to a human Toll-Like Receptor 7 (TLR7) immunogenicity assay.

25. The composition of claim 24, wherein the human TLR7 immunogenicity assay measures activation of NF-κB and/or AP1.

26. The composition of claim 24 or 25, wherein the human TLR7 immunogenicity assay is performed in HEK293 cells or a derivative thereof.

27. The composition of claim 26, wherein the HEK293 cells are engineered to express hTLR7 and a reporter gene.

28. The composition of claim 27, wherein the reporter gene is a secreted reporter gene.

29. The composition of claim 28, wherein the secreted reporter gene is secreted embryonic alkaline phosphatase (SEAP).

30. The composition of any one of claims 27-29, wherein the reporter gene is under the control of a promoter with one or more NF-κB and/or AP1 binding sites.

31. The composition of claim 30, wherein the promoter is an IFN-β minimal promoter.

32. The composition of any one of claims 24-31, wherein the immune response in the human cell contacted with the recombinant RNA construct is at least 1.5 fold or at least 2 fold less than the immune response in the human cell contacted with the corresponding recombinant RNA construct.

33. The composition of any one of claims 1-3 or 11-23, wherein contacting the human cell with the recombinant RNA construct results in an immune response that is lower than the immune response of the human cell contacted with the corresponding recombinant RNA construct according to an interferon alpha/beta (IFNα/β) immunogenicity assay.

34. The composition of claim 33, wherein the IFNα/β immunogenicity assay measures activation of JAK-STAT and/or ISG3.

35. The composition of claim 33 or 34, wherein the IFNα/β immunogenicity assay is performed in HEK293 cells or a derivative thereof.

36. The composition of claim 35, wherein the HEK293 cells are engineered to express human STAT2 and/or IRF9 genes and a reporter gene.

37. The composition of claim 36, wherein the reporter gene is a secreted reporter gene.

38. The composition of claim 37, wherein the secreted reporter gene is secreted embryonic alkaline phosphatase (SEAP).

39. The composition of any one of claims 36-38, wherein the reporter gene is under the control of a promoter with one or more STAT2 and/or IRF9 binding sites.

40. The composition of claim 39, wherein the promoter is an ISG54 promoter.

41. The composition of any one of claims 33-40, wherein the immune response in the human cell contacted with the recombinant RNA construct is at least 1.5 fold, at least 2 fold, or at least 100 fold less than the immune response in the human cell contacted with the corresponding recombinant RNA construct.

42. The composition of any one of claims 1-3 or 11-23, wherein contacting the human cell with the recombinant RNA construct does not result in a substantial immune response according to a human Toll-Like Receptor 3 (TLR3) immunogenicity assay.

43. The composition of claim 42, wherein the human TLR3 immunogenicity assay measures activation of NF-κB and/or APT.

44. The composition of claim 42 or 43, wherein the human TLR3 immunogenicity assay is performed in HEK293 cells or a derivative thereof.

45. The composition of claim 44, wherein the HEK293 cells are engineered to express hTLR3 and a reporter gene.

46. The composition of claim 45, wherein the reporter gene is a secreted reporter gene.

47. The composition of claim 46, wherein the secreted reporter gene is secreted embryonic alkaline phosphatase (SEAP).

48. The composition of any one of claims 45-47, wherein the reporter gene is under the control of a promoter with one or more NF-κB and/or AP1 binding sites.

49. The composition of claim 48, wherein the promoter is an IFN-β minimal promoter.

50. The composition of any one of claims 1-3 or 11-23, wherein contacting the human cell with the recombinant RNA construct results in an immune response that is lower than the immune response of the human cell contacted with the corresponding recombinant RNA construct according to a human Toll-Like Receptor 8 (TLR8) immunogenicity assay.

51. The composition of claim 50, wherein the human TLR8 immunogenicity assay measures activation of NF-κB, APT, and/or IRF.

52. The composition of claim 50 or 51, wherein the human TLR8 immunogenicity assay is performed in HEK293 cells or a derivative thereof.

53. The composition of claim 52, wherein the HEK293 cells are engineered to express hTLR8 and a reporter gene.

54. The composition of claim 53, wherein the reporter gene is a secreted reporter gene.

55. The composition of claim 54, wherein the secreted reporter gene is secreted embryonic alkaline phosphatase (SEAP).

56. The composition of any one of claims 53-55, wherein the reporter gene is under the control of a promoter with one or more NF-κB and/or AP1 binding sites.

57. The composition of claim 56, wherein the promoter is an IFN-β minimal promoter.

58. The composition of any one of claims 1-3 or 11-22, wherein the immune response induces the expression of a proinflammatory cytokine in a cell.

59. The composition of claim 58, wherein the proinflammatory cytokine comprises Interleukin 6 (IL-6).

60. The composition of claim 58 or 59, wherein the cell comprises a human lung epithelial carcinoma cell (A549) or a human monocyte leukemia cell (THP-1).

61. The composition of any one of claims 21-60, wherein the second RNA sequence comprises 2, 3, 4, 5, 6, or more species of siRNA, wherein the 2, 3, 4, 5, 6, or more species of siRNA include siRNAs that are capable of binding to:
(i) different target RNAs;
(ii) different regions of the same target RNA;
(iii) the same region of the same target RNA; or
(iv) any combination thereof.

62. The composition of claim 61, wherein the second RNA sequence comprises at least 3 species of siRNA.

63. The composition of claim 61, wherein the second RNA sequence comprises at least 6 species of siRNA.

64. The composition of any one of the preceding claims, wherein the recombinant RNA construct further comprises one or more linkers.

65. The composition of claim 64, wherein each of the one or more linkers has a structure selected from the group consisting of:
Formula (I): XₘCAACAAXₙ,
wherein X is any nucleotide, m is an integer from 1 to 12, and n is an integer from 0 to 4 (SEQ ID NO: 129); and
Formula (II): XₚTCCCXᵣ,
wherein X is any nucleotide, p is an integer from 0 to 17, and r is an integer from 0 to 13 (SEQ ID NO: 130).

66. The composition of claim 64, wherein each of the one or more linkers comprises a sequence comprising ACAACAA (SEQ ID NO: 85).

67. The composition of claim 64, wherein each of the one or more linkers is not (a) TTTATCTTAGAGGCATATCCCTACGTACCAACAA (SEQ ID NO: 28) or ATAGTGAGTCGTATTAACGTACCAACAA (SEQ ID NO: 27); or (b) does not form a secondary structure according to RNAfold WebServer.

68. The composition of any one of claims 64-67, wherein each of the one or more linkers is present between (a) the first RNA sequence and the second RNA sequence, (b) each of the 2, 3, 4, 5, 6, or more species of siRNA of the second RNA sequence, or (c) both (a) and (b).

69. The composition of any one of claims 64-68, wherein each of the one or more linkers comprises a sequence independently selected from the group consisting of SEQ ID NOs: 27, 28, 85-95.

70. The composition of any one of the preceding claims, wherein the expression of the gene of interest is modulated.

71. The composition of claim 70, wherein the expression of the gene of interest is upregulated in a cell comprising the recombinant RNA construct.

72. The composition of claim 70, wherein the expression of a protein encoded by the gene of interest is upregulated in a cell comprising the recombinant RNA construct.

73. The composition of any one of the preceding claims, wherein the expression of the one or more target RNAs is modulated.

74. The composition of claim 73, wherein the expression of the one or more target RNAs is downregulated by the genetic elements that modulate expression of the one or more target RNAs.

75. The composition of any one of the preceding claims, wherein the genetic elements that modulate expression of the one or more target RNAs do not inhibit the expression of the gene of interest.

76. The composition of any one of the preceding claims, wherein the gene of interest is selected from the group consisting of Interleukin 4 (IL-4) and Insulin-like Growth Factor 1 (IGF-1).

77. The composition of any one of the preceding claims, wherein the one or more target RNA comprises a noncoding RNA or a messenger RNA (mRNA).

78. The composition of any one of the preceding claims, wherein each of the one or more target RNA is a noncoding RNA.

79. The composition of any one of claims 1-77, wherein each of the one or more target RNA is an mRNA.

80. The composition of any one of claims 1-77, wherein the target RNA is an mRNA encoding a protein comprising Interleukin 8 (IL-8), Interleukin 1 beta (IL-1 beta), Tumor Necrosis Factor alpha (TNF-alpha), Interleukin 17 (IL-17), or a functional variant thereof.

81. The composition of any one of the preceding claims, wherein the genetic elements that modulate expression of the one or more target RNAs binds to an exon of the one or more target RNAs.

82. The composition of any one of the preceding claims, wherein the genetic elements that modulate expression of the one or more target RNAs specifically binds to one target RNA.

83. The composition of any one of the preceding claims, wherein the genetic elements that modulate expression of the one or more target RNAs are not encoded by or comprised of an intron sequence of the gene of interest.

84. The composition of any one of the preceding claims, wherein the gene of interest is expressed without RNA splicing.

85. The composition of any one of the preceding claims, wherein the first RNA sequence is present downstream or 3′ of the second RNA sequence.

86. The composition of any one of the preceding claims, wherein the first RNA sequence is present upstream or 5′ of the second RNA sequence.

87. The composition of any one of the preceding claims, wherein the RNA construct comprises an internal ribosome entry site (IRES) upstream or 5′ of the first RNA sequence.

88. The composition of any one of the preceding claims, further comprising a poly(A) tail, a 5′ cap, or a Kozak sequence.

89. The composition of any one of the preceding claims, wherein the first RNA sequence and the second RNA sequence are both recombinant.

90. The composition of any one of the preceding claims, wherein the siRNA comprises a sense strand sequence selected from SEQ ID NOs: 57-70.

91. The composition of any one of the preceding claims for use in modulating the expression of two or more genes in a cell.

92. A pharmaceutical composition comprising a therapeutically effective amount of the composition of any one of claims 1-90 and a pharmaceutically acceptable excipient.

93. A vector comprising a recombinant polynucleic acid construct encoding the composition of any one of claims 1-90.

94. A cell comprising the composition of any one of claims 1-90 or the vector of claim 93.

95. A method of simultaneously expressing an siRNA and an mRNA from a single RNA transcript in a cell, comprising introducing into the cell the composition of any one of claims 1-90, or the vector of claim 93.

96. A method of treating a disease or condition comprising administering to a subject in need thereof the composition of any one of claims 1-90 or the pharmaceutical composition of claim 92.

97. The method of claim 96, wherein the disease or condition comprises a skin disease or condition or a joint disease or condition.

98. The method of claim 97, wherein the skin disease or condition comprises an inflammatory skin disorder.

99. The method of claim 98, wherein the inflammatory skin disorder comprises psoriasis.

100. The method of claim 97, wherein the joint disease or condition comprises a joint degeneration.

101. The method of claim 100, wherein the joint degeneration comprises intervertebral disc disease (IVDD) or osteoarthritis (OA).

102. The method of any one of claims 96-101, wherein the subject is a human.

103. A composition comprising a recombinant RNA construct comprising:
(i) a messenger RNA (mRNA) encoding Insulin-like Growth Factor 1 (IGF-1), and
(ii) at least two small interfering RNAs (siRNAs) capable of binding to an Interleukin-8 (IL-8) mRNA,
wherein contacting a human cell with the recombinant RNA construct results in an immune response that is lower than the immune response of the human cell contacted with a corresponding recombinant RNA construct comprising the mRNA encoding IGF-1 of (i) and at most one of the at least two siRNAs capable of binding to the IL-8 mRNA of (ii).

104. A composition comprising recombinant RNA construct comprising:
(i) a messenger RNA (mRNA) encoding Insulin-like Growth Factor 1 (IGF-1), and
(ii) at least two small interfering RNAs (siRNAs) capable of binding to a Interleukin-1 beta (IL-1 beta) mRNA,
wherein contacting a human cell with the recombinant RNA construct results in an immune response that is lower than the immune response of the human cell contacted with a corresponding recombinant RNA construct comprising the mRNA encoding IGF-1 of (i) and at most one of the at least two siRNAs capable of binding to the IL-1beta mRNA of (ii).

105. A composition comprising recombinant RNA construct:
(i) a messenger RNA (mRNA) encoding Interleukin-4 (IL-4), and
(ii) at least two small interfering RNAs (siRNAs) capable of binding to a Tumor Necrosis Factor alpha (TNF-alpha) mRNA,
wherein contacting a human cell with the recombinant RNA construct results in an immune response that is lower than the immune response of the human cell contacted with a corresponding recombinant RNA construct comprising the mRNA encoding IL-4 of (i) and at most one of the at least two siRNAs capable of binding to the TNF-alpha mRNA of (ii).

106. A composition comprising recombinant RNA construct:
(i) a messenger RNA (mRNA) encoding Interleukin-4 (IL-4), and
(ii) at least two small interfering RNAs (siRNAs) capable of binding to a Tumor Necrosis Factor alpha (TNF-alpha) mRNA and Interleukin 17 (IL-17),
wherein contacting a human cell with the recombinant RNA construct results in an immune response that is lower than the immune response of the human cell contacted with a corresponding recombinant RNA construct comprising the mRNA encoding IL-4 of (i) and at most one of the at least two siRNAs capable of binding to the TNF-alpha mRNA and IL-17 mRNA of (ii).

107. A composition comprising a recombinant polynucleic acid construct comprising a nucleic acid sequence selected from the group consisting of SEQ ID NOs: 1-24, 42, 125, 97-108, 121-122, and 127-128.
